SEQUENCE LISTING (65 sequences)

SEQ ID NO: 1 - protein, 10 aa, Artificial Sequence
  VH CDR1
GYTFTGYWIE

SEQ ID NO: 2 - protein, 17 aa, Artificial Sequence
  VH CDR2
EILPGSGTTNPNEKFKG

SEQ ID NO: 3 - protein, 13 aa, Artificial Sequence
  VH CDR3
ADYYGSDYVKFDY

SEQ ID NO: 4 - protein, 11 aa, Artificial Sequence
  VL CDR1
KASQHVGTHVT

SEQ ID NO: 5 - protein, 7 aa, Artificial Sequence
  VL CDR2
STSYRYS

SEQ ID NO: 6 - protein, 9 aa, Artificial Sequence
  VL CDR3
QHFYSYPLT

SEQ ID NO: 7 - protein, 122 aa, Artificial Sequence
  VH domain of antibody 4D4
QVQLVQSGAEVKKPGASVKVSCKASGYTFTGYWIEWVRQAPGQGLEWMGEILPGSGTTNYNEKFKGRVTMTRDTSTSTVYMELSSLRSEDTAVYYCARADYYGSDYVKFDYWGQGTLVTVSS

SEQ ID NO: 8 - protein, 107 aa, Artificial Sequence
  VL domain of antibody 4D4 and 4D4 H2-1 D11
DIQMTQSPSSLSASVGDRVTITCKASQHVGTHVTWYQQKPGKAPKLLIYSTSYRYSGVPSRFSGSGSGTDFTLTISSLQPEDFATYYCQHFYSYPLTFGGGTKVEIK

SEQ ID NO: 9 - protein, 122 aa, Artificial Sequence
  VH domain of antibody 4D4 H2-1 D11
QVQLVQSGAEVKKPGASVKVSCKASGYTFTGYWIEWVRQAPGQGLEWMGEWLPGSGTTNYNEKFKGRVTMTRDTSTSTVYMELSSLRSEDTAVYYCARADYYGSDYVKFDYWGQGTLVTVSS

SEQ ID NO: 10 - protein, 17 aa, Artificial Sequence
  VH CDR2
EWLPGSGTTNYNEKFKG

SEQ ID NO: 11 - protein, 10 aa, Artificial Sequence
  VH CDR1
GYTFTYYWIE

SEQ ID NO: 12 - protein, 13 aa, Artificial Sequence
  VH CDR3
ADYYGSDHVKFDY

SEQ ID NO: 13 - protein, 11 aa, Artificial Sequence
  VL CDR1
LASQHVGTHVT

SEQ ID NO: 14 - protein, 7 aa, Artificial Sequence
  VL CDR2
GTSYRYS

SEQ ID NO: 15 - protein, 122 aa, Artificial Sequence
  VH domain of antibody 4D4com-XF-9
QVQLVQSGAEVKKPGASVKVSCKASGYTFTYYWIEWVRQAPGQGLEWMGEWLPGSGTTNYNEKFKGRVTMTRDTSTSTVYMELSSLRSEDTAVYYCARADYYGSDHVKFDYWGQGTLVTVSS

SEQ ID NO: 16 - protein, 107 aa, Artificial Sequence
  VL domain of antibody 4D4com-XF-9
DIQMTQSPSSLSASVGDRVTITCLASQHVGTHVTWYQQKPGKAPKLLIYGTSYRYSGVPSRFSGSGSGTDFTLTISSLQPEDFATYYCQHFYDYPLTFGGGTKVEIK

SEQ ID NO: 17 - protein, 122 aa, Artificial Sequence
  VH domain of antibody 4D4com-2F9
QVQLVQSGAEVKKPGASVKVSCKASGYTFTGYWIEWVRQAPGQGLEWMGEWLPGSGTTNYNEKFKGRVTMTRDTSTSTVYMELSSLRSEDTAVYYCARADYYGSDHVKFDYWGQGTLVTVSS

SEQ ID NO: 18 - protein, 107 aa, Artificial Sequence
  VL domain of antibody 4D4com-2F9
DIQMTQSPSSLSASVGDRVTITCKASQHVGTHVTWYQQKPGKAPKLLIYGTSYRYSGVPSRFSGSGSGTDFTLTISSLQPEDFATYYCQHFYEYPLTFGGGTKVEIK

SEQ ID NO: 19 - protein, 10 aa, Artificial Sequence
  VH CDR1
GGTFSGYWIE

SEQ ID NO: 20 - protein, 9 aa, Artificial Sequence
  VL CDR3
QQFYEYPLT

SEQ ID NO: 21 - protein, 122 aa, Artificial Sequence
  VH domain of antibody 7F3 and 7F3 22D3
QVQLVQSGAEVKKPGSSVKVSCKASGGTFSGYWIEWVRQAPGQGLEWMGEILPGSGTTNYNEKFKGRVTITADESTSTAYMELSSLRSEDTAVYYCARADYYGSDYVKFDYWGQGTLVTVSS

SEQ ID NO: 22 - protein, 107 aa, Artificial Sequence
  VL domain of antibody 7F3
DIQMTQSPSSLSASVGDRVTITCKASQHVGTHVTWYQQKPGKAPKLLIYSTSYRYSGVPSRFSGSGSGTDFTLTISSLQPEDFATYYCQQFYEYPLTFGGGTKVEIK

SEQ ID NO: 23 - protein, 122 aa, Artificial Sequence
  VH domain of antibody 71A10
QVQLVQSGAEVKKPGSSVKVSCKASGGTFSGYWIEWVRQAPGQGLEWMGEILPGSGTTNPNEKFKGRVTITADESTSTAYMELSSLRSEDTAVYYCARADYYGSDYVKFDYWGQGTLVTVSS

SEQ ID NO: 24 - protein, 107 aa, Artificial Sequence; identical to SEQ ID NO: 22
  VL domain of antibody 71A10

SEQ ID NO: 25 - protein, 107 aa, Artificial Sequence
  VL domain of antibody 7F3 22D3
DIQMTQSPSSLSASVGDRVTITCKASQHVGTHVTWYQQKPGKAPKLLIYGTSYRYSGVPSRFSGSGSGTDFTLTISSLQPEDFATYYCQQFYEYPLTFGGGTKVEIK

SEQ ID NO: 26 - protein, 10 aa, Artificial Sequence
  VH CDR1
GGTFSYYWIE

SEQ ID NO: 27 - protein, 122 aa, Artificial Sequence
  VH domain of antibody 7F3com-2H2
QVQLVQSGAEVKKPGSSVKVSCKASGGTFSYYWIEWVRQAPGQGLEWMGEILPGSGTTNPNEKFKGRVTITADESTSTAYMELSSLRSEDTAVYYCARADYYGSDYVKFDYWGQGTLVTVSS

SEQ ID NO: 28 - protein, 107 aa, Artificial Sequence
  VL domain of antibody 7F3com-2H2
DIQMTQSPSSLSASVGDRVTITCKASQHVITHVTWYQQKPGKAPKLLIYGTSYSYSGVPSRFSGSGSGTDFTLTISSLQPEDFATYYCQQFYEYPLTFGGGTKVEIK

SEQ ID NO: 29 - protein, 122 aa, Artificial Sequence; identical to SEQ ID NO: 23
  VH domain of antibody 7F3com-3H5

SEQ ID NO: 30 - protein, 107 aa, Artificial Sequence; identical to SEQ ID NO: 25
  VL domain of antibody 7F3com-3H5

SEQ ID NO: 31 - protein, 122 aa, Artificial Sequence; identical to SEQ ID NO: 27
  VH domain of antibody 7F3com-3D4

SEQ ID NO: 32 - protein, 107 aa, Artificial Sequence
  VL domain of antibody 7F3com-3D4
DIQMTQSPSSLSASVGDRVTITCKASQHVITHVTWYQQKPGKAPKLLIYGTSYRYSGVPSRFSGSGSGTDFTLTISSLQPEDFATYYCQQFYEYPLTFGGGTKVEIK

SEQ ID NO: 33 - protein, 25 aa, Artificial Sequence [sequence omitted]

SEQ ID NO: 34 - protein, 14 aa, Artificial Sequence
  VH framework region 2
WVRQAPGQGLEWMG

SEQ ID NO: 35 - protein, 32 aa, Artificial Sequence
  VH framework region 3
RVTMTRDTSTSTVYMELSSLRSEDTAVYYCAR

SEQ ID NO: 36 - protein, 11 aa, Artificial Sequence
  VH framework region 4
WGQGTLVTVSS

SEQ ID NO: 37 - protein, 25 aa, Artificial Sequence [sequence omitted]

SEQ ID NO: 38 - protein, 32 aa, Artificial Sequence
  VH framework region 3
RVTITADESTSTAYMELSSLRSEDTAVYYCAR

SEQ ID NO: 39 - protein, 23 aa, Artificial Sequence
  VL framework region 1
DIQMTQSPSSLSASVGDRVTITC

SEQ ID NO: 40 - protein, 15 aa, Artificial Sequence
  VH framework region 2
WYQQKPGKAPKLLIY

SEQ ID NO: 41 - protein, 32 aa, Artificial Sequence
  VH framework region 3
GVPSRFSGSGSGTDFTLTISSLQPEDFATYYC

SEQ ID NO: 42 - protein, 10 aa, Artificial Sequence
  VH framework region 4
FGGGTKVEIK

SEQ ID NO: 43 - DNA, 591 bp, Artificial Sequence
  VH domain of 7F3com-2H2
ccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 44 - DNA, 30 bp, Artificial Sequence
  VH CDR1
ggaggcaccttcagctattactggatagag

SEQ ID NO: 45 - DNA, 51 bp, Artificial Sequence
  VH CDR2
gagattttacctggaagtggtactactaacccgaatgagaagttcaagggc

SEQ ID NO: 46 - DNA, 39 bp, Artificial Sequence
  VH CDR3
gcggattactacggtagtgattacgtcaagtttgactac

SEQ ID NO: 47 - DNA, 321 bp, Artificial Sequence
  VL domain of 7F3com-2H2
gacatccagatgacccagtctccatcctccctgtctgcatctgtaggagacagagtcaccatcacttgcaaggcaagtcagcatgtgattactcatgtaacctggtatcagcagaaaccagggaaagcccctaagctcctgatctatgggacatcctacagctacagtggggtcccatcaaggttcagtggcagtggatctgggacagatttcactctcaccatcagcagtctgcaacctgaagattttgcaacttattactgtcagcaattttacgagtatcctctcacgttcggcggagggaccaaggtggagatcaaa

SEQ ID NO: 48 - DNA, 33 bp, Artificial Sequence
  VL CDR1
aaggcaagtcagcatgtgattactcatgtaacc

SEQ ID NO: 49 - DNA, 15 bp, Artificial Sequence
  VL CDR2
gggacatcctacagc

SEQ ID NO: 50 - DNA, 27 bp, Artificial Sequence
  VL CDR3
cagcaattttacgagtatcctctcacg

SEQ ID NO: 51 - DNA, 591 bp, Homo sapiens; identical to SEQ ID NO: 43

SEQ ID NO: 52 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 53 - protein, 808 aa, Homo sapiens
MAELLASAGSACSWDFPRAPPSFPPPAASRGGLGGTRSFRPHRGAESPRPGRDRDGVRVPMASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 54 - protein, 140 aa, Homo sapiens
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 55 - DNA, 2171 bp, Homo sapiens
agcagctctgtaatgcgcttgtggtttcagatgtgggcggcctgtgtgaacctgtcgtgcaaagctcacgtcaccaactgctgcagttatctcctgaatcaggctgagggtctttgctgtgcacccagagatagttgggtgacaaatcacctccaggttggggatgcctcagacttgtgatgggactgggcagatgcatctgggaaggctggaccttggagagtgaggccctgaggcgagacatgggcacctggctcctggcctgcatctgcatctgcacctgtgtctgcttgggagtctctgtcacaggggaaggacaagggccaaggtctagaaccttcacctgcctcaccaacaacattctcaggatcgattgccactggtctgccccagagctgggacagggctccagcccctggctcctcttcaccagcaaccaggctcctggcggcacacataagtgcatcttgcggggcagtgagtgcaccgtcgtgctgccacctgaggcagtgctcgtgccatctgacaatttcaccatcactttccaccactgcatgtctgggagggagcaggtcagcctggtggacccggagtacctgccccggagacacgttaagctggacccgccctctgacttgcagagcaacatcagttctggccactgcatcctgacctggagcatcagtcctgccttggagccaatgaccacacttctcagctatgagctggccttcaagaagcaggaagaggcctgggagcaggcccagcacagggatcacattgtcggggtgacctggcttatacttgaagcctttgagctggaccctggctttatccatgaggccaggctgcgtgtccagatggccacactggaggatgatgtggtagaggaggagcgttatacaggccagtggagtgagtggagccagcctgtgtgcttccaggctccccagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattctaggtccctgactcgccagatgcatcatgtccattttgggaaaatggactgaagtttctggagcccttgtctgagactgaacctcctgagaaggggcccctagcagcggtcagaggtcctgtctggatggaggctggaggctcccccctcaacccctctgctcagtgcctgtggggagcagcctctaccctcagcatcctggccacaagttcttccttccattgtcccttttctttatccctgacctctctgagaagtggggtgtggtctctcagctgttctgccctcatacccttaaagggccagcctgggcccagtggacacaggtaaggcaccatgaccacctggtgtgacctctctgtgccttactgaggcacctttctagagattaaaaggggcttgatggctgttaaaaaaaaaaaaaaaaaa

SEQ ID NO: 56 - DNA, 2175 bp, Homo sapiens
agcagctctgtaatgcgcttgtggtttcagatgtgggcggcctgtgtgaacctgtcgtgcaaagctcacgtcaccaactgctgcagttatctcctgaatcaggctgagggtctttgctgtgcacccagagatagttgggtgacaaatcacctccaggttggggatgcctcagacttgtgatgggactgggcagatgcatctgggaagtaactgctgcaagaacggacagacactgctgcagagaacttgccacggtgtttcatgctgtggctggtggttccaggctgcacgctccattctaggaaaggggccctcagcccagtcccttgcaggctggaccttggagagtgaggccctgaggcgagacatgggcacctggctcctggcctgcatctgcatctgcacctgtgtctgcttgggagtctctgtcacaggggaaggacaagggccaaggtctagaaccttcacctgcctcaccaacaacattctcaggatcgattgccactggtctgccccagagctgggacagggctccagcccctggctcctcttcaccaggctcctggcggcacacataagtgcatcttgcggggcagtgagtgcaccgtcgtgctgccacctgaggcagtgctcgtgccatctgacaatttcaccatcactttccaccactgcatgtctgggagggagcaggtcagcctggtggacccggagtacctgccccggagacacgagcaacatcagttctggccactgcatcctgacctggagcatcagtcctgccttggagccaatgaccacacttctcagctatgagctggccttcaagaagcaggaagaggcctgggagcaggcccagcacagggatcacattgtcggggtgacctggcttatacttgaagcctttgagctggaccctggctttatccatgaggccaggctgcgtgtccagatggccacactggaggatgatgtggtagaggaggagcgttatacaggccagtggagtgagtggagccagcctgtgtgcttccaggctccccagagacaaggccctctgatcccaccctgggggtggccaggcaacacccttgttgctgtgtccatctttctcctgctgactggcccgacctacctcctgttcaagctgtcgcccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattctaggtccctgactcgccagatgcatcatgtccattttgggaaaatggactgaagtttctggagcccttgtctgagactgaacctcctgagaaggggcccctagcagcggtcagaggtcctgtctggatggaggctggaggctcccccctcaacccctctgctcagtgcctgtggggagcagcctctaccctcagcatcctggccacaagttcttccttccattgtcccttttctttatccctgacctctctgagaagtggggtgtggtctctcagctgttctgccctcatacccttaaagggccagcctgggcccagtggacacaggtaaggcaccatgaccacctggtgtgacctctctgtgccttactgaggcacctttctagagattaaaaggggcttgatggctgttaaaaaaaaaaaaaaaaaa

SEQ ID NO: 57 - DNA, 1451 bp, Homo sapiens
gaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatc

SEQ ID NO: 58 - protein, 521 aa, Homo sapiens
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 59 - protein, 332 aa, Homo sapiens
MHLGSNCCKNGQTLLQRTCHGVSCCGWWFQAARSILGKGPSAQSLAGWTLESEALRRDMGTWLLACICICTCVCLGVSVTGEGQGPRSRTFTCLTNNILRIDCHWSAPELGQGSSPWLLFTRLLAAHISASCGAVSAPSCCHLRQCSCHLTISPSLSTTACLGGSRSAWWTRSTCPGDTSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRLGWGPTGPVCC

SEQ ID NO: 60 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 61 - protein, 17 aa, Artificial Sequence
  VH CDR2
EILPGSGTTNYNEKFKG

SEQ ID NO: 62 - protein, 11 aa, Artificial Sequence
  VL CDR1
KASQHVITHVT

SEQ ID NO: 63 - protein, 9 aa, Artificial Sequence
  VL CDR3
QHFYDYPLT

SEQ ID NO: 64 - protein, 9 aa, Artificial Sequence
  VL CDR3
QHFYEYPLT

SEQ ID NO: 65 - protein, 7 aa, Artificial Sequence
  VL CDR2
GTSYSYS

(19) United States
(12) Patent Application Publication
(10) Pub. No.: US 2010/0129379 A1
(43) Pub. Date: May 27, 2010

(54) STABILIZED ANTIBODY FORMULATIONS AND USES THEREOF

(76) Inventors: John Carpenter, Littleton, CO (US); Hasige Sathish, Longmont, CO (US); Theodore Randolph, Niwot, CO (US); Branden Salinas, Louisville, CO (US); Christian Allan, Brookeville, MD (US); Steven Bishop, Frederick, MD (US)

Correspondence Address: MEDIMMUNE, LLC;Patrick Scott Alban, ONE MEDIMMUNE WAY, GAITHERSBURG, MD, 20878, US

(21) Appl. No.: 12/442,655

(22) PCT Filed: Sep. 25, 2007
(86) PCT No.: PCT/US07/79403

§ 371 Date: Jan. 7, 2010

Related U.S. Application Data
(60) Provisional application No. 60/847,239, filed on Sep. 25, 2006.
(60) Provisional application No. 60/949,999, filed on Jul. 16, 2007.

(51) Int. Cl. A61K 39/395; A61P 35/00; A61P 37/02

(52) U.S. Cl. 424/158.1; 424/130.1

(57) ABSTRACT

The present invention provides methods of optimizing certain stable liquid formulations of antibodies that immunospecifically bind to antigens of interest. Such formulations are suitable for parenteral administration to a subject, and exhibit increased stability, low to undetectable levels of aggregation, low to undetectable levels of antibody fragmentation/degradation, and very little to no loss of the biological activities of the antibodies, even during long periods of storage. The methods of the invention provide formulations that offer multiple advantages over formulations produced by non-optimized methods, including less stringent or more readily available transportation and storage conditions, less frequent dosing, and/or smaller dosage amounts in the therapeutic, prophylactic and diagnostic uses of such formulations. The invention further provides methods of identifying antibodies exhibiting certain phase behaviors such that the antibodies can be formulated by the methods of the invention. Also provided are prophylactic, therapeutic, and diagnostic uses of such antibody formulations.

### 1. INTRODUCTION

The present invention provides methods of optimizing certain formulations of antibodies that immunospecifically bind to antigens of interest. Such formulations are suitable for parenteral administration to a subject, and exhibit increased stability, low to undetectable levels of aggregation, low to undetectable levels of antibody fragmentation/degradation, and very little to no loss of the biological activities of the antibodies, even during long periods of storage. The methods of the invention provide formulations that offer multiple advantages over formulations produced by non-optimized methods, including less stringent or more readily available transportation and storage conditions, less frequent dosing, and/or smaller dosage amounts in the therapeutic, prophylactic and diagnostic uses of such formulations. The invention further provides methods of identifying antibodies exhibiting certain phase behaviors such that the antibodies can be formulated by the methods of the invention. Also provided are prophylactic, therapeutic, and diagnostic uses of such antibody formulations.

### 2. BACKGROUND OF THE INVENTION

The instabilities of proteins are a major obstruction to commercial development of protein drugs. In particular, protein aggregation, which often arises because of this instability, is one of the major obstacles in all phases of drug development. The presence of a transient population of partially unfolded intermediates and conditions in which they are formed is thus important in predicting and understanding protein aggregation. This is particularly important for multi-domain proteins such as monoclonal antibodies (mAbs), which are popular drug candidates due to their high binding affinities and specificities, the ease with which they can be targeted to specific antigens, and their general resistance to aggregation. Since antibodies produced in the same manner have high sequence identities, it is often thought that they will exhibit similar phase behaviors and stabilities during processing and storage.

Currently, many antibodies are provided as lyophilized formulations. Lyophilized formulations of antibodies have a number of limitations, including a prolonged process for lyophilization and resulting high cost for manufacturing. In addition, a lyophilized formulation has to be reconstituted aseptically and accurately by healthcare practitioners prior to administering to patients. Thus, a need exists for liquid formulations of antibodies, at a concentration comparable to or higher than the reconstituted lyophilized formulations so that there is no need to reconstitute the formulation prior to administration. Such formulations thereby allow healthcare practitioners to make much quicker and easier administration of antibodies to a patient.

Moreover, certain prior liquid antibody preparations have short shelf lives and may lose biological activity of the antibodies resulting from chemical and physical instabilities during the storage. Chemical instability may be caused by deamidation, racemization, hydrolysis, oxidation, beta elimination or disulfide exchange, and physical instability may be caused by antibody denaturation, aggregation, precipitation or adsorption. Among those, aggregation, deamidation and oxidation are known to be the most common causes of the antibody degradation (Wang et al., 1988, J. of Parenteral Science & Technology 42(Suppl):S4-S26; Cleland et al., 1993, Critical Reviews in Therapeutic Drug Carrier Systems 10(4):307-377). Accordingly, there exists a need for a stable liquid formulation of antibodies that bind to antigens of interest, such formulations exhibiting increased stability, low to undetectable levels of aggregation, low to undetectable levels of antibody fragmentation/degradation, and very little to no loss of the biological activities of the antibodies, even during long periods of storage.

### 3. SUMMARY OF INVENTION

The present invention provides stable liquid antibody formulations of antibodies comprising non-zwitterionic buffers such as phosphate (e.g., Na₃PO₄), tris, citrate, succinate, and acetate buffers. In specific embodiments, the invention provides stable liquid formulations comprising antibodies at a high concentration. In other specific embodiments, the antibody formulations of the present invention are formulated for parenteral administration (e.g., intradermally, intramuscularly, intraperitoneally, intravenously and subcutaneously) in a subject. In another specific embodiment, the subject is human.

The present invention provides stable liquid antibody formulations, said formulations comprising phosphate at a concentration in the range from about 10 mM to about 100 mM or higher, with pH in the range of about 4.0 to about 8.0; NaCl at a concentration in the range from about 0 mM to about 200 mM; and an antibody of interest, e.g., an antibody that immunospecifically binds to an IL-9 polypeptide (including antibody fragments thereof), for example, 7F3com-2H2, at a concentration of about 10 mg/ml or higher. The stable liquid formulations of the present invention may further comprise one or more excipients such as a saccharide, a surfactant, and a polyol. In specific embodiments, the liquid formulations of the invention comprise surfactants (e.g., Tween-20 or Tween-80) at a concentration in the range of about 0% to about 0.1%; sucrose at a concentration range from about 0% to about 10%; and/or trehalose at a concentration in the range from about 0% to about 10%. In certain embodiments, the stable liquid formulations may be used for subcutaneous delivery and comprise phosphate at a concentration in the range from about 25 mM to about 75 mM (e.g., at a concentration of about 50 mM), with a pH in the range of about 6.0 to about 6.5; NaCl at a concentration in the range of about 100 mM to about 200 mM (e.g., at a concentration of about 150 mM); and an antibody of interest, e.g., an antibody that immunospecifically binds to an IL-9 polypeptide (including antibody fragments thereof), e.g., 7F3com-2H2, at a concentration in the range of about 50 mg/ml to about 150 mg/ml (e.g., about 100 mg/ml).

The antibody formulations of the invention preferably maintain improved aggregation profiles upon storage, for example, for extended periods (for example, but not limited to 6 months, 1 year, 2 years, 3 years or 5 years) at room temperature or 4° C. or for periods (such as, but not limited to 1 week, 2 weeks, 3 weeks, 1 month, 2 months, 3 months, 6 months or 1 year) at elevated temperatures such as 38° C.-42° C. Such formulations may be at a pH in the range of 4.0 to 8.0, e.g., at pH 6.2.

The methods of the present invention can be used to concentrate and produce stable liquid formulations of any type of antibody. The antibodies for use in accordance with the methods of the present invention may be therapeutic or prophylactic antibodies, and are useful in the treatment and/or management of various diseases, including but not limited to, diseases or disorders associated with or characterized by aberrant expression and/or activity of an IL-9 polypeptide, diseases or disorders associated with or characterized by aberrant expression and/or activity of the IL-9 receptor (“IL-9R”) or one or more subunits thereof, autoimmune diseases, inflammatory diseases, proliferative diseases, or infections (e.g., respiratory infections), or one or more symptoms thereof (e.g., wheezing). Examples of autoimmune diseases include, but are not limited to: diabetes, Hashimoto's disease, autoimmune adrenal insufficiency, pure red cell anemia, multiple sclerosis, rheumatoid carditis, systemic lupus erythematosus, rheumatoid arthritis, chronic inflammation, Sjogren's syndrome polymyositis, dermatomyositis and scleroderma. Examples of inflammatory disorders include, but are not limited to, asthma and allergic reactions (Types I-IV). Examples of respiratory infections include, but are not limited to, infections of the upper and lower respiratory tracts, including viral infections, bacterial infections and/or fungal infections. Examples of viral infections include parainfluenza virus infection, influenza virus infection, metapneumovirus infection, or respiratory syncytial virus (RSV) infection. The antibody formulations of the invention may also be used to treat subjects that have or previously had bronchopulmonary dysplasia, congenital heart disease, cystic fibrosis or acquired or congenital immunodeficiency.

Non-limiting examples of therapeutic or prophylactic antibodies that can be formulated and used in the methods of the present invention are listed in Section 5.1.1, infra, and include antibodies that immunospecifically bind to an IL-9 polypeptide or fragments thereof, such as, 4D4, 4D4 H2-1 D11, 4D4com-XF-9, 4D4com-2F9, 7F3, 71A10, 7F3 22D3, 7F3com-2H2, 7F3com-3H5, or 7F3com-3D4; antibodies that specifically bind to the αvβ₃integrin, such as MEDI-522 (Vitaxin®); antibodies that specifically bind to an RSV antigen, such as Synagis® (palivizumab), MEDI-524 (motavizumab; Numax®); antibodies that specifically bind to CD2, such as MEDI-507 (siplizumab); antibodies that bind to CD19, such as MT-103; antibodies that bind to EphA2, such as EA2, EA5, B233 (including human and humanized forms thereof); and antibodies that bind to EphA4, such as EA44 (including human and humanized forms thereof). See also U.S. application Ser. No. 11/473,537, filed Jun. 23, 2006, entitled “Antibody Formulations Having Optimized Aggregation and Fragmentation Profiles,” which is herein incorporated by reference in its entirety, for other therapeutic and prophylactic antibodies that can be formulated and used in the methods of the present invention.

In specific embodiments, the present invention provides stable liquid formulations of antibodies (e.g., monoclonal antibodies) that exhibit stability, low to undetectable levels of antibody fragmentation and/or aggregation, and very little to no loss of the biological activities of the antibodies (including antibody fragments thereof) during manufacture, preparation, transportation, and storage, as assessed by, for example, high performance size exclusion chromatography (HPSEC). In specific embodiments, the stable liquid formulations of the invention comprise antibodies, for example, monoclonal antibodies (e.g., monoclonal antidies that immunospecifically bind to an IL-9 polypeptide, e.g., 7F3com-2H2). In further embodiments, the stable liquid formulations of the present invention facilitate the administration of antibodies (including antibody fragments thereof) that immunospecifically bind to an IL-9 polypeptide (e.g., 7F3com-2H2) for the prevention, treatment and/or management of diseases or disorders associated with or characterized by aberrant expression and/or activity of an IL-9 polypeptide, diseases or disorders associated with or characterized by aberrant expression and/or activity of the IL-9 receptor (“IL-9R”) or one or more subunits thereof, autoimmune diseases, inflammatory diseases, proliferative diseases, or infections (e.g., respiratory infections), or one or more symptoms thereof (e.g., wheezing). Examples of autoimmune diseases include, but are not limited to: diabetes, Hashimoto's disease, autoimmune adrenal insufficiency, pure red cell anemia, multiple sclerosis, rheumatoid carditis, systemic lupus erythematosus, rheumatoid arthritis, chronic inflammation, Sjogren's syndrome polymyositis, dermatomyositis and scleroderma. Examples of inflammatory disorders include, but are not limited to, asthma and allergic reactions (Types I-IV). Examples of respiratory infections include, but are not limited to, infections of the upper and lower respiratory tracts, including viral infections, bacterial infections and/or fungal infections. Examples of viral infections include parainfluenza virus infection, influenza virus infection, metapenumovirus infection, or respiratory syncytial virus (RSV) infection. The antibody formulations of the invention may also be used to treat subjects that have or previously had bronchopulmonary dysplasia, congenital heart disease, cysteic fibrosis or acquired or congenital immunodeficiency. In particular, the stable liquid formulations of the present invention enable a healthcare professional to quickly administer a sterile dosage of an antibody (including antibody fragment thereof) without having to accurately and sterilely reconstitute the antibody (including antibody fragment thereof) prior to administration.

In other specific embodiments, the present invention encompasses stable liquid formulations of antibodies that immunospecifically bind to an IL-9 polypeptide, including but not limited to 4D4, 4D4 H2-1 D11, 4D4com-XF-9, 4D4com-2F9, 7F3, 71A10, 7F3 22D3, 7F3com-2H2, 7F3com-3H5, or 7F3com-3D4 (for amino acid sequences, see U.S. application Ser. No. 11/823,253, filed Apr. 12, 2004, and published as U.S. Patent Publication No. US 2005/0002934 A1, each of which is incorporated by reference herein in its entirety), which exhibit low to undetectable levels of antibody aggregation and/or fragmentation with very little to no loss of the biological activities of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 during manufacture, preparation, transportation, and long periods of storage. The present invention also encompasses stable liquid formulations of antibodies that immunospecifically bind to an IL-9 polypeptide and have increased in vivo half-lives relative to known antibodies such as, e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4, said formulations exhibiting low to undetectable levels of antibody aggregation and/or fragmentation, and very little to no loss of the biological activities of the antibodies (including antibody fragments thereof). The present invention also encompasses stable liquid formulations of antibodies that immunospecifically bind to an IL-9 polypeptide, said antibodies (including antibody fragments thereof) comprising a variable heavy (VH) and/or variable light (VL) domain having the amino acid sequence of the VH and/or VL domain of 4D4, 4D4 H2-1 D11, 4D4com-XF-9, 4D4com-2F9, 7F3, 71A10, 7F3 22D3, 7F3com-2H2, 7F3com-3H5, or 7F3com-3D4, said formulations exhibiting low to undetectable levels of antibody aggregation and/or fragmentation, and very little to no loss of the biological activities of the antibodies (including antibody fragments thereof). The present invention further encompasses stable liquid formulations of antibodies (including antibody fragments thereof) that immunospecifically bind to an IL-9 polypeptide, said antibodies (including antibody fragments thereof) comprising one or more VH complementarity determining regions (CDRs) and/or one or more VL CDRs having the amino acid sequence of one or more VH CDRs and/or VL CDRs listed in Table 1, infra, said formulations exhibiting low to undetectable levels of antibody aggregation and/or fragmentation, and very little to no loss of the biological activities of the antibodies (including antibody fragments thereof). In specific embodiments, the invention does not encompass stable liquid formulations of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 antibodies.

TABLE 1

Residues that are different between each amino acid sequence encoding
the various CDRs appear in bold, underlined font.

| Antibody | | | | | |
|---|---|---|---|---|---|
| Name | VH Domain | VH CDR1 | VH CDR2 | VH CDR3 | VL Domain |
| 4D4 | SEQ. ID NO.: | GYTFTGYWI | EILPGSGTTN | ADYYGSDYV | SEQ. ID NO.: |
| | 7 | E | YNEKFKG | KFDY | 8 |
| | | (SEQ. ID NO. | (SEQ. ID | (SEQ. ID | |
| | | 1): | NO.: 61) | NO.: 3) | |
| 4D4 H2-1 D11 | SEQ. ID NO.: | GYTFTGYWI | EWLPGSGTT | ADYYGSDYV | SEQ. ID NO.: |
| | 9 | E | NYNEKFKG | KFDY | 8 |
| | | (SEQ. ID NO.: | (SEQ. ID NO.: | (SEQ. ID NO.: | |
| | | 1) | 10) | 3) | |
| 4D4com-XF-9 | SEQ. ID NO.: | GYTFTYYWI | EWLPGSGTT | ADYYGSDHV | SEQ. ID NO.: |
| | 15 | E | NYNEKFKG | KFDY | 16 |
| | | (SEQ. ID NO.: | (SEQ. ID NO.: | (SEQ. ID NO.: | |
| | | 11) | 10) | 12) | |
| 4D4com-2F9 | SEQ. ID NO.: | GYTFTGYWI | EWLPGSGTT | ADYYGSDHV | SEQ. ID NO.: |
| | 17 | E | NYNEKFKG | KFDY | 18 |
| | | (SEQ. ID NO.: | (SEQ. ID NO.: | (SEQ. ID NO.: | |
| | | 1) | 10) | 12) | |
| 7F3 | SEQ. ID NO.: | GGTFSGYWI | EILPGSGTTN | ADYYGSDYV | SEQ. ID NO.: |
| | 21 | E | YNEKFKG | KFDY | 22 |
| | | (SEQ. ID NO.: | (SEQ. ID | (SEQ. ID NO.: | |
| | | 19) | NO.: 61) | 3) | |
| 71A10 | SEQ. ID NO.: | GGTFSGYWI | EILPGSGTTN | ADYYGSDYV | SEQ. ID NO.: |
| | 23 | E | PNEKFKG | KFDY | 24 |
| | | (SEQ. ID NO.: | (SEQ. ID NO.: | (SEQ. ID NO.: | |
| | | 19) | 2) | 3) | |
| 7F3 22D3 | SEQ. ID NO.: | GGTFSGYWI | EILPGSGTTN | ADYYGSDYV | SEQ. ID NO.: |
| | 21 | E | PNEKFKG | KFDY | 25 |
| | | (SEQ. ID NO.: | (SEQ. ID | (SEQ. ID NO.: | |
| | | 19) | NO.: 61) | 3) | |
| 7F3com-2H2 | SEQ. ID NO.: | GGTFSYYWI | EILPGSGTTN | ADYYGSDYV | SEQ. ID NO.: |
| | 27 | E | PNEKFKG | KFDY (SEQ. | 28 |
| | | (SEQ. ID NO.: | (SEQ. ID NO.: | ID NO.: 3) | |
| | | 26) | 2) | | |
| 7F3com-3H5 | SEQ. ID NO.: | GGTFSGYWI | EILPGSGTTN | ADYYGSDYV | SEQ. ID NO.: |
| | 29 | E | PNEKFKG | KFDY | 30 |
| | | (SEQ. ID NO.: | (SEQ. ID NO.: | (SEQ. ID NO.: | |
| | | 19) | 2) | 3) | |
| 7F3com-3D4 | SEQ. ID NO.: | GGTFSYYWI | EILPGSGTTN | ADYYGSDYV | SEQ. ID NO.: |
| | 31 | E | PNEKFKG | KFDY | 32 |
| | | (SEQ. ID NO.: | (SEQ. ID NO.: | (SEQ. ID NO.: | |
| | | 26) | 2) | 3) | |

| Antibody | | | |
|---|---|---|---|
| Name | VL CDR1 | VL CDR2 | VL CDR3 |
| 4D4 | KASQHVGTH | STSYRYS | QHFYSYPLT |
| | VT | (SEQ. ID NO.: | (SEQ. ID NO.: |
| | (SEQ. ID NO.: | 5) | 6) |
| | 4) | | |
| 4D4 H2-1 D11 | KASQHVGTH | STSYRYS | QHFYSYPLT |
| | VT | (SEQ. ID NO.: | (SEQ. ID NO.: |
| | (SEQ. ID NO.: | 5) | 6) |
| | 4) | | |
| 4D4com-XF-9 | LASQHVGTH | GTSYRYS | QHFYDYPLT |
| | VT (SEQ. ID | (SEQ. ID NO.: | (SEQ. ID |
| | NO.: 13) | 14) | NO.: 63) |
| 4D4com-2F9 | KASQHVGTH | GTSYRYS | QHFYEYPLT |
| | VT | (SEQ. ID NO.: | (SEQ. ID |
| | (SEQ. ID NO.: | 14) | NO.:64) |
| | 4) | | |
| 7F3 | KASQHVGTH | STSYRYS | QQFYEYPLT |
| | VT | (SEQ. ID NO.: | (SEQ. ID NO.: |
| | (SEQ. ID NO.: | 5) | 20) |
| | 4) | | |
| 71A10 | KASQHVGTH | STSYRYS | QQFYEYPLT |
| | VT | (SEQ. ID NO.: | (SEQ. ID NO.: |
| | (SEQ. ID NO.: | 5) | 20) |
| | 4) | | |
| 7F3 22D3 | KASQHVGTH | GTSYRYS | QQFYEYPLT |
| | VT | (SEQ. ID NO.: | (SEQ. ID NO.: |
| | (SEQ. ID NO.: | 14) | 20) |
| | 4) | | |
| 7F3com-2H2 | KASQHVITH | GTSYSYS | QQFYEYPLT |
| | VT | (SEQ. ID | (SEQ. ID NO.: |
| | (SEQ. ID | NO.:65) | 20) |
| | NO.:62) | | |
| 7F3com-3H5 | KASQHVGTH | GTSYRYS | QQFYEYPLT |
| | VT | (SEQ. ID NO.: | (SEQ. ID NO.: |
| | (SEQ. ID NO.: | 14) | 20) |
| | 4) | | |
| 7F3com-3D4 | KASQHVITH | GTSYRYS | QQFYEYPLT |
| | VT | (SEQ. ID NO.: | (SEQ. ID NO.: |
| | (SEQ. ID | 14) | 20) |
| | NO.:62) | | |

The present invention encompasses stable liquid formulations of antibodies (including antibody fragments thereof). The antibodies may exhibit a decrease or reduction in certain phase behaviors (e.g., formation of unfolded intermediates, colloidal instability, soluble association and precipitation) when formulated in the presence of a non-zwitterionic buffer (e.g., phosphate (e.g., Na₃PO₄), tris, citrate, succinate or acetate buffer) at a pH below the pI of the antibodies in the presence of salt (e.g., NaCl), as compared to the antibodies when formulated in the presence of a zwitterionic buffer (e.g., histidine buffer) at the same pH and in the presence of salt at the same concentration, said formulations having stability at 38-42° C. as assessed by high performance size exclusion chromatography (HPSEC). The techniques of static light scattering (SLS), Fourier Transform Infrared

Spectroscopy (FTIR), circular dichroism (CD), urea-induced protein unfolding techniques, intrinsic tryptophan fluorescence, differential scanning calorimetry, and/or ANS protein binding are also used to assess the phase behaviors, other physical properties and stability of the molecule. The liquid formulations of the present invention exhibit stability, as assessed by HPSEC, at temperature ranges of 38-42° C. for at least 15 days but no more than 25 days; at temperature ranges of 20-24° C. for at least 6 months but not more than 1.5 years; and at temperature ranges of 2-8° C. (especially at 4° C.) for at least 1.5 years, at least 2 years, at least 2.5 years, or at least 3 years. The present invention also encompasses liquid formulations of antibodies (including antibody fragments thereof) that immunospecifically bind to an antigen of interest (e.g., an IL-9 polypeptide), said formulations having low to undetectable levels of antibody aggregation as measured by HPSEC. The techniques of static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding are also used to assess the phase behaviors, other physical properties and stability of the molecule. In one embodiment, the liquid formulations of the present invention exhibit stability at 38-42° C. for at least 15 days and exhibit low to undetectable levels of antibody aggregation as measured by HPSEC and, further, exhibit very little to no loss of the biological activity of the antibodies (including antibody fragments thereof) of the formulation compared to the reference antibodies as measured by antibody binding assays such as, e.g., ELISAs.

The present invention provides methods for identifying antibodies, and in particular, therapeutic or prophylactic antibodies, that may exhibit a decrease or reduction in certain phase behaviors (e.g., formation of unfolded intermediates, colloidal instability, soluble association and precipitation) when formulated in the presence of a non-zwitterionic buffer (e.g., phosphate (e.g., Na₃PO₄), tris, citrate, succinate, or acetate buffer) at a pH below the pI of the antibodies in the presence of salt (e.g., NaCl), as compared to the antibodies when formulated in the presence of a zwitterionic buffer (e.g., histidine buffer) at the same pH and in the presence of salt at the same concentration, which make them amenable to formulation using the methods of the present invention. These phase behaviors may contribute to the instability of the antibody formulations. The stability of the antibody formulations may be measured by, for example, high performance size exclusion chromatography (HPSEC). The techniques of static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding are also used to assess the phase behaviors, other physical properties and stability of the antibody molecules.

The present invention provides methods for preparing stable liquid formulations of an antibody (including antibody fragment thereof) that immunospecifically binds to an antigen of interest (e.g., an IL-9 polypeptide), said methods comprising concentrating a fraction containing the purified antibody to a final antibody concentration ranging from about 1 mg/ml, about 5 mg/ml, about 10 mg/ml, about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, or about 200 mg/ml using a semipermeable membrane with an appropriate molecular weight (MW) cutoff (e.g., a 30 kD cutoff for whole antibody molecules and F(ab′)₂ fragments; and a 10 kD cutoff for antibody fragments such as Fab fragments) and diafiltering the concentrated antibody fraction into the formulation buffer using the same membrane. The formulation buffer of the present invention comprises phosphate, tris, citrate, succinate, or acetate at a concentration ranging from about 1 mM to about 100 mM, from about 10 mM to about 100 mM, from about 5 mM to about 50 mM, from about 10 mM to about 25 mM, or from about 25 to about 75 mM. The formulation buffer of the present invention further comprises NaCl at a concentration ranging from about 0 mM to about 200 mM, 10 mM to about 200 mM, from about 50 to about 200 mM, from about 100 to about 150 mM, or from about 100 mM to about 200 mM. The pH of the formulation may range from about 4.0 to about 8.0, e.g., from about 6.0 to about 6.5.

The liquid formulations of the present invention may be sterilized by sterile filtration using a 0.2μ filter. Sterilized liquid formulations of the present invention may be administered to a subject for the prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. The liquid formulations of the present invention may be administered in combination with other therapies (e.g., prophylactic or therapeutic agents other than antibodies that immunospecifically bind to an IL-9 polypeptide, such as anti-inflammatory agents, immunomodulatory agents and anti-cancer agents).

In a specific embodiment, the invention provides an antibody formulation comprising an aqueous carrier, phosphate, and 50 mg/ml or higher of an antibody or antibody fragment, said antibody formulation being formulated for administration to a human subject. In another specific embodiment, the invention provides an antibody formulation comprising an aqueous carrier, phosphate, and 10 mg/ml or higher of an antibody or antibody fragment, wherein said antibody or antibody fragment displays a reduction in one or more of the following phase behaviors when formulated in a phosphate buffer at a pH below the pI of said antibody in the presence of salt, as compared to said antibody when formulated in a histidine buffer at said pH in the presence of salt at the same concentration: (a) formation of unfolded intermediates; (b) colloidal instability; (c) soluble association of the antibody molecules; or (d) precipitation of the antibody molecules;

wherein said at least one or more phase behaviors are measured by techniques selected from the group consisting of high performance size exclusion chromatography (HPSEC), tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and 1-anilino-8-naphthalenesulfonic acid (ANS) protein binding techniques.

The present invention also provides kits comprising the stable liquid formulations of antibodies (including antibody fragments thereof) that immunospecifically bind to an antigen of interest (e.g., an IL-9 polypeptide) for use by, e.g., a healthcare professional. In specific embodiments, the kits comprising the stable formulations of the invention are formulated for parenteral administration (e.g., intradermally, intramuscularly, intraperitoneally, intravenously and subcutaneously) to a human subject. The present invention further provides methods of preventing, treating and/or managing a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms of any of the foregoing. The stable liquid formulations of the invention can be administered to a subject (e.g., a human subject) parenterally (e.g., intradermally, intramuscularly, intraperitoneally, intravenously and subcutaneously) orally, or intranasally to a subject to prevent, treat and/or manage a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms of any of the foregoing. The stable liquid formulations of the present invention can also be used to diagnose, detect or monitor disease or disorder, for example, a disease or disorder associated with or characterized by aberrant expression and/or activity of an IL-9 polypeptide, diseases or disorders associated with or characterized by aberrant expression and/or activity of the IL-9R or one or more subunits thereof, autoimmune diseases, inflammatory diseases, proliferative diseases, or infections (e.g., respiratory infections), or one or more symptoms thereof

3.1. Terminology

All formulations of antibodies and/or antibody fragments that immunospecifically bind to an antigen of interest (e.g., an IL-9 polypeptide described herein), are herein collectively referred to as “liquid formulations of the invention,” “high concentration stable liquid formulations of the invention,” “antibody liquid formulations of the invention,” or “antibody formulations of the invention.”

As used herein the term “aberrant” refers to a deviation from the norm, e.g., the average healthy subject and/or a population of average healthy subjects. The term “aberrant expression,” as used herein, refers to abnormal expression of a gene product (e.g., RNA, protein, polypeptide, or peptide) by a cell or subject relative to a normal, healthy cell or subject and/or a population of normal, healthy cells or subjects. Such aberrant expression may be the result of the amplification of the gene. In a specific embodiment, the term “aberrant expression” refers to abnormal expression of IL-9 and/or an IL-9R or subunit thereof by a cell or subject relative to the expression of the gene product by a normal, healthy cell or subject and/or a population of normal, healthy cells or subjects and encompasses the expression of an IL-9 and/or an IL-9R or subunit thereof gene product at an unusual location within the cell or subject, the expression of an IL-9 and/or an IL-9R or subunit thereof gene product at an altered level in the cell or subject, the expression of a mutated IL-9 and/or an IL-9R or subunit thereof gene product, or a combination thereof.

The term “aberrant activity,” as used herein, refers to an altered level of a gene product, the increase of an activity by a gene product, or the loss of an activity of a gene product in a cell or subject relative to a normal, healthy cell or subject and/or a population of normal healthy cells or subjects. In specific embodiments, the term “aberrant activity” refers to an IL-9 and/or IL-9R or subunit thereof activity that deviates from that normally found in a healthy cell or subject and/or a population of normal, healthy cells or subjects (e.g., an increase in IL-9′s affinity for the IL-9R). Examples of IL-9 activities include, but are not limited to, the phosphorylation of the IL-9R, the activation of Jak3, the activation of MEK, the activation of STAT-1, and the activation of STAT-3.

As used herein, the term “about” in the context of a given numerate value or range refers to a value or range that is within 20%, within 10%, and within 5% of the given value or range.

As used herein, the term “analog” in the context of a proteinaceous agent (e.g., proteins, polypeptides, peptides, and antibodies) refers to a proteinaceous agent that possesses a similar or identical functions as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions x 100%). In one embodiment, the two sequences are the same length.

The determination of percent identity between two sequences can also be accomplished using a mathematical algorithm. One, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the terms “antagonist” and “antagonists” refer to any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

The term “antibody fragment” as used herein refers to a fragment of an antibody that immunospecifically binds to an antigen of interest, (e.g., an IL-9 polypeptide). Antibody fragments may be generated by any technique known to one of skill in the art. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain. Antibody fragments can be also produced by recombinant DNA technologies. Antibody fragments may be one or more complementarity determining regions (CDRs) of antibodies, or one or more antigen-binding fragments of an antibody.

As used herein, the terms “antibody” and “antibodies” refer to monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The term “antibodies or antibody fragments that immunospecifically bind to an antigen of interest” and analogous terms as used herein refer to antibodies or antibody fragments that specifically bind to an antigen of interest or a fragment thereof, and do not specifically bind to other antigens or fragments thereof The term “antibodies or antibody fragments that immunospecifically bind to an IL-9 polypeptide” and analogous terms as used herein refer to antibodies or antibody fragments that specifically bind to an IL-9 polypeptide or a fragment of an IL-9 polypeptide and do not specifically bind to other polypeptides. Preferably, antibodies or antibody fragments that immunospecifically bind to an IL-9 polypeptide have a higher affinity to an IL-9 polypeptide or a fragment of an IL-9 polypeptide when compared to the affinity to other polypeptides or fragments of other polypeptides. The affinity of an antibody is a measure of its bonding with a specific antigen at a single antigen-antibody site, and is in essence the summation of all the attractive and repulsive forces present in the interaction between the antigen-binding site of an antibody and and a particular epitope. The affinity of an antibody to a particular antigen (e.g., an IL-9 polypeptide or fragment of an IL-9 polypeptide) may be expressed by the equilibrium constant K, defined by the equation K=[Ag Ab]/[Ag][Ab], which is the affinity of the antibody-combining site where [Ag] is the concentration of free antigen, [Ab] is the concentration of free antibody and [Ag Ab] is the concentration of the antigen-antibody complex. Where the antigen and antibody react strongly together there will be very little free antigen or free antibody, and hence the equilibrium constant or affinity of the antibody will be high. High affinity antibodies are found where there is a good fit between the antigen and the antibody (for a discussion regarding antibody affinity, see Sigal and Ron ed., 1994, Immunology and Inflammation—Basic Mechanisms and Clinical Consequences, McGraw-Hill, Inc. New York at pages 56-57; and Seymour et al., 1995, Immunology—An Introduction for the Health Sciences, McGraw-Hill Book Company, Australia at pages 31-32). Preferably, antibodies or antibody fragments that immunospecifically bind to an IL-9 polypeptide or fragment thereof do not cross-react with other antigens. That is, antibodies or antibody fragments that immunospecifically bind to an IL-9 polypeptide or fragment thereof with a higher energy than to other polypeptides or fragments of other polypeptides (see, e.g., Paul ed., 1989, Fundamental Immunology, 2^{nd} ed., Raven Press, New York at pages 332-336 for a discussion regarding antibody specificity). Antibodies or antibody fragments that immunospecifically bind to an IL-9 polypeptide can be identified, for example, by immunoassays such as radioimmunoassays (RIAs), enzyme-linked immunosorbent assays (ELISAs), and BIAcore assays (described in Section 5.7, infra) or other techniques known to those of skill in the art (see, e.g., Seymour et al., 1995, Immunology—An Introduction for the Health Sciences, McGraw-Hill Book Company, Australia at pages 33-41 for a discussion of various assays to determine antibody-antigen interactions in vivo). Antibodies or antibody fragments that immunospecifically bind to an IL-9 polypeptide or fragment thereof only antagonize an IL-9 polypeptide and do not significantly antagonize other activities.

As used herein, the term “control IgG antibody” refers to an IgG antibody or other “control antibody” that does not immunospecifically bind to an antigen of interest (e.g., an IL-9 polypeptide) and preferably does not cross-react with the antigen of interest (e.g., an IL-9 polypeptide).

As used herein, the term “cytokine receptor modulator” refers to an agent that modulates the phosphorylation of a cytokine receptor, the activation of a signal transduction pathway associated with a cytokine receptor, and/or the expression of a particular protein such as a cytokine Such an agent may directly or indirectly modulate the phosphorylation of a cytokine receptor, the activation of a signal transduction pathway associated with a cytokine receptor, and/or the expression of a particular protein such as a cytokine Thus, examples of cytokine receptor modulators include, but are not limited to, cytokines, fragments of cytokines, fusion proteins, and antibodies that immunospecifically bind to a cytokine receptor or a fragment of the antibody or cytokine receptor. Further, examples of cytokine receptor modulators include, but are not limited to, peptides, polypeptides (e.g., soluble cytokine receptors), fusion proteins and antibodies that immunospecifically binds to a cytokine or a fragment thereof.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, nitryl, or amine group. An organic molecule may also be esterified, alkylated and/or phosphorylated.

As used herein, the terms “disorder” and “disease” are used interchangeably to refer to a condition in a subject in which the subject differs from a healthy, unaffected subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, e.g., chronic inflammation. Autoimmune disorders may or may not be associated with inflammation. Moreover, inflammation may or may not be caused by an autoimmune disorder. Certain conditions may be characterized as more than one disorder. For example, certain conditions may be characterized as both autoimmune and inflammatory disorders.

As used herein, the term “effective amount” refers to the amount of a therapy (e.g., a prophylactic or therapeutic agent) which is sufficient to reduce and/or ameliorate the severity and/or duration of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, prevent the advancement of said disease or disorder, cause regression of said disease or disorder, prevent the recurrence, development, or onset of one or more symptoms associated with said disease or disorder, or enhance or improve the prophylactic or therapeutic effect(s) of another therapy (e.g., prophylactic or therapeutic agent).

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (e.g., serum albumin), amino acids (e.g., aspartic acid, glutamic acid, lysine, arginine, glycine), surfactants (e.g., SDS, Tween 20, Tween 80, polysorbate and nonionic surfactants), saccharides (e.g., glucose, sucrose, maltose and trehalose), polyols (e.g., mannitol and sorbitol), fatty acids and phospholipids (e.g., alkyl sulfonates and caprylate). For additional information regarding excipients, see Remington's Pharmaceutical Sciences (by Joseph P. Remington, 18^{th} ed., Mack Publishing Co., Easton, Pa.), which is incorporated herein in its entirety.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a second, different polypeptide or protein. In another embodiment, a fragment of a protein or polypeptide retains at least one function of the protein or polypeptide. In another embodiment, a fragment of a polypeptide or protein retains at least two, three, four, or five functions of the polypeptide or protein. By way of example, a fragment of an antibody that immunospecifically binds to an IL-9 polypeptide retains the ability to immunospecifically bind to an IL-9 polypeptide. A “functional fragment” is a fragment that retains at least one function of the protein or polypeptide.

As used herein, the term “fusion protein” refers to a polypeptide or protein that comprises an amino acid sequence of a first polypeptide or protein or fragment, analog or derivative thereof, and an amino acid sequence of a heterologous polypeptide or protein (i.e., a second polypeptide or protein or fragment, analog or derivative thereof different than the first polypeptide or protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides, or peptides with immunomodulatory activity may be fused together to form a fusion protein. In one embodiment, fusion proteins retain or have improved activity relative to the activity of the original polypeptide or protein prior to being fused to a heterologous protein, polypeptide, or peptide.

The terms “high concentration” and “concentrated antibody” as used herein refer to a concentration of 50 mg/ml or higher, or 95 mg/ml or higher of an antibody (including antibody fragment thereof) that immunospecifically binds to an antigen of interest (e.g., an IL-9 polypeptide), in an antibody formulation.

As used herein, the term “host cell” includes a particular subject cell transfected or transformed with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the terms “human child” or “child” or variations thereof refer to a human between 24 months of age and 18 years of age.

As used herein, the terms “elderly human,” “elderly,” or variations thereof refer to a human 65 years old or older, or 70 years old or older.

As used herein, the terms “human infant” or “infant” or variations thereof refer to a human less than 24 months of age, less than 12 months, less than 6 months, less than 3 months, less than 2 months, or less than 1 month of age.

As used herein, the terms “human infant born prematurely,” “preterm infant,” or “premature infant,” or variations thereof refer to a human born at less than 40 weeks of gestational age, less than 35 weeks gestational age, who is less than 6 months old, less than 3 months old, less than 2 months old, or less than 1 month old.

As used herein, the term “hybridizes under stringent conditions” describes conditions for hybridization and washing under which nucleotide sequences at least 30% (at least 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found, for example, in Current Protocols in Molecular Biology, John Wiley & Sons, N.Y. (1989 and updates), 6.3.1-6.3.6.

Generally, stringent conditions are selected to be about 5 to 10° C. lower than the thermal melting point (Tm) for the specific sequence at a defined ionic strength pH.

The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0M sodium ion concentration (or other salts) at pH 7.0 to 8.3 and the temperature is at least about 30° C. for short probes (for example, 10 to 50 nucleotides) and at least 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one, non-limiting example, stringent hybridization conditions are hybridization at 6× sodium chloride/sodium citrate (SSC) at about 45° C., followed by one or more washes in 0.1×SSC, 0.2% SDS at about 68 ° C. In one, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.).

As used herein, the term “IL-9 polypeptide” refers to IL-9, an analog, derivative or a fragment thereof, including mature and immature forms of IL-9 (see, Van

Snick et al., 1989, J Exp. Med. 169:363-68 and Yang et al., 1989, Blood 74:1880-84, which are both incorporated by reference herein in their entireties), or a fusion protein comprising IL-9, an analog, derivative or a fragment thereof. The IL-9 polypeptide may be from any species. The nucleotide and/or amino acid sequences of IL-9 polypeptides can be found in the literature or public databases, or the nucleotide and/or amino acid sequences can be determined using cloning and sequencing techniques known to one of skill in the art. For example, the nucleotide sequence of human IL-9 can be found in the GenBank database (see, e.g., Accession No. NM_{—}000590; FIG. 12). The amino acid sequence of human IL-9 can be found in the GenBank database (see, e.g., Accession Nos. A60480, NP_{—}000584 and AAC17735; FIG. 13) and in U.S. patent application Ser. No. 10/412,703, filed Apr. 11, 2003 and published Nov. 27, 2003, as US 2003/0219439 A1, entitled, “Recombinant Anti-Interleukin-9 Antibodies,” (the amino acid sequence of human IL-9 on page 5 is specifically incorporated herein by reference). In one embodiment, an IL-9 polypeptide is human IL-9, an analog, derivative or a fragment thereof.

As used herein, the terms “IL-9 receptor” and “IL-9R” refer to an IL-9 receptor or an analog, derivative, or fragment thereof, or a fusion protein comprising an IL-9 receptor, an analog, derivative, or a fragment thereof. As used herein, the terms “one or more subunits” and “a subunit” in the context of an IL-9R refer to the IL-9R ligand-specific alpha subunit (“IL-9Rα”) and/or common γ_{c} chain (also present in IL-2R, IL-4R, IL-7R, and IL-15R complexes) of the functional IL-9R or an analog, derivative, or fragment thereof. In one embodiment, a functional IL-9R mediates a proliferative response in T cells treated with IL-9 as determined by any cell proliferation assay known to those skilled in the art (e.g., a [³H]-thymidine incorporation assay or a hexosaminidase assay) (see, e.g., Renauld et al., 1992, Proc. Natl. Acad. Sci. USA, 89:5690-94 and Bauer et al., 1998, J Biol. Chem. 273:9255-60, which are both incorporated by reference herein in their entireties). Preferebly, treating a T cell line expressing a functional IL-9R (e.g., TS1 RA3 cells (R&D Systems) expressing both human and murine IL-9Rα) with IL-9, results in a dose-dependent increase in T cell proliferation, as measured by any cell proliferation assay known to those skilled in the art (see, Renauld et al., 1992, Proc. Natl. Acad. Sci. USA, 89:5690-94 and Bauer et al., 1998, J Biol. Chem. 273:9255-60). In another preferred embodiment, a functional IL-9R, comprising the γ_{c} and IL-9Rα chains, initiates a signaling cascade through the Janus kinases JAK1 and JAK3, thereby activating homo- and heterodimers of the signal transducer and activator transcription (STAT) factors STAT-1, STAT-3 and STAT-5 (see, Bauer et al., 1998, J Biol. Chem. 273:9255-60). In another preferred embodiment, a functional IL-9R may prevent apoptosis through a mechanism involving STAT-3 and STAT-5, as determined by apoptosis assays known to those skilled in the art (see, Bauer et al., 1998, J Biol. Chem. 273:9255-60). The IL-9R or one or more subunits thereof may be from any species. The nucleotide and/or amino acid sequences of the IL-9R and the subunits thereof can be found in the literature or in public databases, or the nucleotide and/or amino acid sequences can be determined using cloning and sequencing techniques known to one of skill in the art. For example, the nucleotide sequence of human IL-9R can be found in the GenBank database (see, e.g., Accession Nos. NM_{—}002186, NM_{—}176786, and NM_{—}000206; FIG. 14). The amino acid sequence of human IL-9R can be found in the GenBank database (see, e.g., Accession Nos. NP_{—}002177; NP_{—}789743, and NP_{—}000197; FIG. 15) and in U.S. patent application Ser. No. 10/412,703, filed Apr. 11, 2003 and published Nov. 27, 2003, as US 2003/0219439 A1, entitled, “Recombinant Anti-Interleukin-9 Antibodies,” (the amino acid sequence of human IL-9R on pages 6-7 specifically incorporated herein by reference). In one embodiment, an IL-9R or one or more subunits thereof is a human IL-9R or one or more subunits thereof, an analog, derivative, or a fragment thereof.

As used herein, the term “immunomodulatory agent” and variations thereof including, but not limited to, immunomodulatory agents, immunomodulants or immunomodulatory drugs, refer to an agent that modulates a host's immune system. In a specific embodiment, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an antibody of the invention. Immunomodulatory agents include, but are not limited to, small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

The term “in combination” as used herein refers to the use of more than one therapies (e.g., prophylactic and/or therapeutic agents). The use of the term “in combination” does not restrict the order in which therapies (e.g., prophylactic and/or therapeutic agents) are administered to a subject with a disease or disorder (e.g., a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof). A first therapy (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disease or disorder (e.g., disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof).

As used herein, the term “immunospecifically binds to an antigen” and analogous terms refer to peptides, polypeptides, proteins, fusion proteins and antibodies (including antibody fragments thereof) that specifically bind to an antigen or a fragment and do not specifically bind to other antigens. A peptide, polypeptide, protein, or antibody that immunospecifically binds to an antigen may bind to other peptides, polypeptides, or proteins with lower affinity as determined by, e.g., immunoassays, BIAcore, or other assays known in the art. Antibodies (including antibody fragments thereof) that immunospecifically bind to an antigen may be cross-reactive with related antigens. Preferably, antibodies (including antibody fragments thereof) that immunospecifically bind to an antigen do not significantly cross-react with other antigens (i.e., is not detectable in routine immunological assays). An antibody binds specifically to an antigen when it binds to the antigen with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2nd ed., Raven Press, New York at pages 332-336 for a discussion regarding antibody specificity.

As used herein, the term “in combination” refers to the use of more than one therapy (e.g., more than one prophylactic agent and/or therapeutic agent). The use of the term “in combination” does not restrict the order in which therapies (e.g., prophylactic and/or therapeutic agents) are administered to a subject with a respiratory condition. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject with a respiratory condition.

The term “inorganic salt” as used herein refers to any compounds containing no carbon that result from replacement of part or all of the acid hydrogen or an acid by a metal or a group acting like a metal and are often used as tonicity adjusting compounds in pharmaceutical compositions and preparations of biological materials. The most common inorganic salts are NaCl, KCl, NaH₂PO₄, etc.

As used herein, the term “isolated” in the context of an organic or inorganic molecule (whether it be a small or large molecule), other than a proteinaceous agent or nucleic acid molecule, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In one embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it may also be substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated. In one embodiment, an antibody of the invention is isolated. In a specific embodiment, an “isolated” antibody is purified by a multi-step purification process that comprises three chromatography steps (cation exchange, protein A and anion exchange), a nanofiltration step, and a low pH treatment step (for a detailed description, see Section 6, infra).

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, nucleic acid molecules are isolated; however, “isolated” excludes members of a population of a library of clones such as a cDNA library.

The phrase “low to undetectable levels of aggregation” as used herein refers to samples containing no more than 5%, no more than 4%, no more than 3%, no more than 2%, no more than 1% and no more than 0.5% aggregation by weight of protein as measured by high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques.

The term “low to undetectable levels of fragmentation” as used herein refers to samples containing equal to or more than 80%, 85%, 90%, 95%, 98% or 99% of the total protein, for example, in a single peak as determined by HPSEC, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an antibody.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the term “mast cell modulator” refers to an agent which modulates the activation of a mast cell, mast cell degranulation, and/or expression of a particular protein such as a cytokine Such an agent may directly or indirectly modulate the activation of a mast cell, degranulation of the mast cell, and/or the expression of a particular protein such as a cytokine Non-limiting examples of mast cell modulators include, but are not limited to, small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides, or peptides), fusion proteins, antibodies, synthetic or natural inorganic molecules, synthetic or natural organic molecule, or mimetic agents which inhibit and/or reduce the expression, function, and/or activity of a stem cell factor, a mast cell protease, a cytokine (such as IL-3, IL-4, and IL-9), a cytokine receptor (such as IL-3R, IL-4R, and IL-9R), and a stem cell receptor. Other non-limiting examples of mast cell modulators include, but are not limited to small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides, or peptides), fusion proteins, antibodies, synthetic or natural inorganic molecules, synthetic or natural organic molecule, or mimetic agents which inhibit and/or reduce the expression, function and/or activity of IgE. In certain embodiments, a mast cell modulator is an agent that prevents or reduces the activation of additional mast cells following degranulation of mast cells. In other embodiments, a mast cell modulator is an agent that inhibits or reduces mast cell degranulation.

As used herein, the terms “non-responsive” and refractory” describe patients treated with a currently available therapy (e.g., prophylactic or therapeutic agent) for a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof which is not clinically adequate to relieve one or more symptoms associated with the disorder. Typically, such patients suffer from severe, persistently active disease and require additional therapy to ameliorate the symptoms associated with the disorder.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The term “polyol” as used herein refers to a sugar that contains many -OH groups compared to a normal saccharide.

As used herein, the terms “prevent,” “preventing,” and “prevention” refer to the inhibition of the development or onset of disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, or the prevention of the recurrence, onset, or development of one or more symptoms of a respiratory condition in a subject resulting from the administration of a therapy (e.g., a prophylactic or therapeutic agent), or the administration of a combination of therapies (e.g., a combination of prophylactic or therapeutic agents).

As used herein, the terms “prophylactic agent” and “prophylactic agents” refer to any agent(s) which can be used in the prevention of the onset, recurrence or development of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In certain embodiments, the term “prophylactic agent” refers to an antibody that immunospecifically binds to an IL-9 polypeptide. In certain other embodiments, the term “prophylactic agent” refers to an agent other than an antibody that immunospecifically binds to an IL-9 polypeptide. Preferably, a prophylactic agent is an agent which is known to be useful to or has been or is currently being used to the prevent or impede the onset, development, progression and/or severity of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. Prophylactic agents may be characterized as different agents based upon one or more effects that the agents have in vitro and/or in vivo. For example, a mast cell modulator may also be characterized as an immunomodulatory agent.

As used herein, the term “prophylactically effective amount” refers to the amount of a therapy (e.g., prophylactic agent) which is sufficient to result in the prevention of the development, recurrence, or onset of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, or to enhance or improve the prophylactic effect(s) of another therapy (e.g., a prophylactic agent).

As used herein, a “prophylactic protocol” refers to a regimen for dosing and timing the administration of one or more therapies (e.g., one or more prophylactic agents) that has a prophylactic effect.

A used herein, a “protocol” includes dosing schedules and dosing regimens. The protocols herein are methods of use and include prophylactic and therapeutic protocols.

The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

As used herein, the phrase “side effects” encompasses unwanted and adverse effects of a prophylactic or therapeutic agent. Side effects are always unwanted, but unwanted effects are not necessarily adverse. An adverse effect from a therapy (e.g., a prophylactic or therapeutic agent) might be harmful, uncomfortable, or risky. Undesired effects typically experienced by patients are numerous and known in the art. Many are described in the Physicians' Desk Reference (60th ed., 2006).

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such agents.

The terms “stability” and “stable” as used herein in the context of a liquid formulation comprising an antibody (including antibody fragment thereof) that immunospecifically binds to an antigen of interest (e.g., an IL-9 polypeptide) refer to the resistance of the antibody or (including antibody fragment thereof) in the formulation to aggregation, degradation or fragmentation under given manufacture, preparation, transportation and storage conditions. The “stable” formulations of the invention retain biological activity under given manufacture, preparation, transportation and storage conditions. The stability of said antibody (including antibody fragment thereof) can be assessed by degrees of aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody (including antibody fragment thereof) (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) in phosphate buffer, pH 6.0-6.5 that contains 150 mM NaCl, which reference formulation regularly gives a single monomer peak (≧97% area) by HPSEC. Alternatively, a reference formulation may be a reference standard frozen at −70° C. consisting of 10 mg/ml of an antibody (including antibody fragment thereof) (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) in phosphate buffer at pH 6.0-6.5, which reference formulation regularly gives a single monomer peak (≧97% area) by HPSEC. The overall stability of a formulation comprising an antibody (including antibody fragment thereof) can be assessed by various immunological assays including, for example, ELISA and radioimmunoassay using isolated antigen molecules or cells expressing the same.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgous monkey, chimpanzee, and a human), and more preferably a human. In a certain embodiment, the subject is a mammal, preferably a human, with a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In another embodiment, the subject is a farm animal (e.g., a horse, pig, or cow) or a pet (e.g., a dog or cat) with a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In another embodiment, the subject is a mammal, preferably a human, at risk of developing a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof (e.g., an immunocompromised or immunosuppressed mammal). In another embodiment, the subject is not an immunocompromised or immunosuppressed mammal, preferably a human. In another embodiment, the subject is a mammal, preferably a human, with a lymphocyte count that is not under approximately 500 cells/mm³. In another embodiment, the subject is a human infant or a human infant born prematurely. In another embodiment, the subject is a human child or a human adult. In another embodiment, the subject is a human child with bronchopulmonary dysplasia, congenital heart diseases, or cystic fibrosis. In another embodiment, the subject is an elderly human. In yet another embodiment, the subject is a human in an institution or group home, such as, but not limited to, a nursing home.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a respiratory condition. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a respiratory condition. In addition, a synergistic effect can result in improved efficacy of therapies (e.g., prophylactic or therapeutic agents) in the prevention or treatment of a respiratory condition. Finally, the synergistic effect of a combination of therapies (e.g., prophylactic or therapeutic agents) may avoid or reduce adverse or unwanted side effects associated with the use of any single therapy.

As used herein, the term “T cell receptor modulator” refers to an agent which modulates the phosphorylation of a T cell receptor, the activation of a signal transduction pathway associated with a T cell receptor and/or the expression of a particular protein associated with T cell receptor activity such as a cytokine Such an agent may directly or indirectly modulate the phosphorylation of a T cell receptor, the activation of a signal transduction pathway associated with a T cell receptor, and/or the expression of a particular protein associated with T cell receptor activity such as a cytokine Examples of T cell receptor modulators include, but are not limited to, peptides, polypeptides, proteins, fusion proteins and antibodies which immunospecifically bind to a T cell receptor or a fragment thereof. Further, examples of T cell receptor modulators include, but are not limited to, proteins, peptides, polypeptides (e.g., soluble T cell receptors), fusion proteins and antibodies that immunospecifically bind to a ligand for a T cell receptor or fragments thereof

As used herein, the terms “therapeutic agent” and “therapeutic agents” refer to any agent(s) which can be used in the prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In certain embodiments, the term “therapeutic agent” refers to an antibody that binds to an IL-9 polypeptide. In certain other embodiments, the term “therapeutic agent” refers an agent other than an antibody that immunospecifically binds to an IL-9 polypeptide. Preferably, a therapeutic agent is an agent that is known to be useful for, or has been or is currently being used for the prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. Therapeutic agents may be characterized as different agents based upon one or more effects the agents have in vivo and/or in vitro, for example, an anti-inflammatory agent may also be characterized as an immunomodulatory agent.

As used herein, the term “therapeutically effective amount” refers to the amount of a therapy (e.g., an antibody that immunospecifically binds to an IL-9 polypeptide), that is sufficient to reduce the severity of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, reduce the duration of a respiratory condition, ameliorate one or more symptoms of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, autoimmune diseases, inflammatory diseases, proliferative diseases, or infections (e.g., respiratory infections), or one or more symptoms thereof, cause regression of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, or enhance or improve the therapeutic effect(s) of another therapy.

The terms “therapies” and “therapy” can refer to any protocol(s), method(s), and/or agent(s) that can be used in the prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In certain embodiments, the terms “therapy” and “therapy” refer to anti-viral therapy, anti-bacterial therapy, anti-fungal therapy, biological therapy, supportive therapy, and/or other therapies useful in prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof known to skilled medical personnel.

As used herein, the term “therapeutic protocol” refers to a regimen for dosing and timing the administration of one or more therapies (e.g., therapeutic agents) that has a therapeutic effective.

As used herein, the terms “treat,” “treatment,” and “treating” refer to the reduction or amelioration of the progression, severity, and/or duration of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof resulting from the administration of one or more therapies (including, but not limited to, the administration of one or more prophylactic or therapeutic agents). In certain embodiments, such terms refer to a reduction in the swelling of organs or tissues, or a reduction in the pain associated with a respiratory condition. In other embodiments, such terms refer to a reduction in the inflammation or constriction of an airway(s) associated with asthma. In other embodiments, such terms refer to a reduction in the replication of an infectious agent, or a reduction in the spread of an infectious agent to other organs or tissues in a subject or to other subjects. In other embodiments, such terms refer to the reduction of the release of inflammatory agents by mast cells, or the reduction of the biological effect of such inflammatory agents. In other embodiments, such terms refer to a reduction of the growth, formation and/or increase in the number of hyperproliferative cells (e.g., cancerous cells). In yet other embodiments, such terms refer to the eradication, removal or control of primary, regional or metastatic cancer (e.g., the minimization or delay of the spread of cancer).

The term “very little to no loss of the biological activities” as used herein refers to antibody activities, including but not limited to, specific binding abilities of antibodies (including antibody fragments thereof) to an antigen of interest (e.g., an IL-9 polypeptide) as measured by various immunological assays, including, but not limited to ELISAs and radioimmunoassays. In one embodiment, the antibodies (including antibody fragments thereof) of the formulations of the invention retain approximately 50%, preferably 55%, 60%, 65%, 70%, 75%, 80%, 85%, 90%, 95% or 98% of the ability to immunospecifically bind to an antigen polypeptide as compared to a reference antibody (including antibody fragment thereof) (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) as measured by an immunological assay known to one of skill in the art or described herein. For example, an ELISA based assay may be used to compare the ability of an antibody (including antibody fragment thereof) to immunospecifically bind to an IL-9 polypeptide to a 4D4, 4D4 H2-1 D11, 4D4com-XF-9, 4D4com-2F9, 7F3, 71A10, 7F3 22D3, 7F3com-2H2, 7F3com-3H5, or 7F3com-3D4 reference standard. In this assay, referred to as the IL-9 Binding ELISA, plates are coated with an isolated IL-9 and the binding signal of a set concentration of a 4D4, 4D4 H2-1 D11, 4D4com-XF-9, 4D4com-2F9, 7F3, 71A10, 7F3 22D3, 7F3com-2H2, 7F3com-3H5, or 7F3com-3D4 reference standard is compared to the binding signal of the same concentration of a test antibody (including antibody fragment thereof). A “reference standard” as used herein refers to an antibody (including antibody fragment thereof) (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) that is frozen at −70° C. consisting of 10 mg/ml of an antibody (including antibody fragment thereof) (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) in phosphate buffer, pH 6.0-6.5, and containing 150 mM NaCl, which reference formulation regularly gives a single monomer peak (≧97% area) by HPSEC. In another embodiment, the term “very little to no loss of biological activities” as used herein refers to antibody activities, including other effector activities of the antibody.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. BRIEF DESCRIPTION OF THE FIGURES

FIGS. 1A-B show the amino acid sequences of the (A) variable heavy domain (SEQ ID NO.:7) of 4D4 with the VH CDR1 (SEQ ID NO.:1), the VH CDR2 (SEQ ID NO.:61), and the VH CDR3 (SEQ ID NO.:3) underlined, starting in order from VH CDR1 at the far left; and (B) variable light domain (SEQ ID. NO.:8) of 4D4, with the VL CDR1 (SEQ ID NO.:4), the VL CDR2 (SEQ ID NO.:5), and the VL CDR3 (SEQ ID NO.:6) underlined, starting in order from VL CDR1 at the far left.

FIGS. 2A-B show the amino acid sequences of the (A) variable heavy domain (SEQ ID NO.:9) of 4D4 H2-1 D11, with the VH CDR1 (SEQ ID NO.:1), the VH CDR2 (SEQ ID NO.:10), and the VH CDR3 (SEQ ID NO.:3) underlined, starting in order from VH CDR1 at the far left; and (B) variable light domain (SEQ ID. NO.:8) of 4D4 H2-1 D11, the VL CDR1 (SEQ ID NO.:4), the VL CDR2 (SEQ ID NO.:5), and the VL CDR3 (SEQ ID NO.:6) underlined, starting in order from VL CDR1 at the far left.

FIGS. 3A-B show the amino acid sequences of the (A) variable heavy domain (SEQ ID NO.:15) of 4D4com-XF-9, with the VH CDR1 (SEQ ID NO.:11), the VH CDR2 (SEQ ID NO.:10), and the VH CDR3 (SEQ ID NO.:12) underlined, starting in order from VH CDR1 at the far left; and (B) variable light domain (SEQ ID. NO.:16) of 4D4com-XF-9, the VL CDR1 (SEQ ID NO.:13), the VL CDR2 (SEQ ID NO.:14), and the VL CDR3 (SEQ ID NO.:63) underlined, starting in order from VL CDR1 at the far left.

FIGS. 4A-B show the amino acid sequences of the (A) variable heavy domain (SEQ ID NO.:17) of 4D4com-2F9, with the VH CDR1 (SEQ ID NO.:1), the VH CDR2 (SEQ ID NO.:10), and the VH CDR3 (SEQ ID NO.:12) underlined, starting in order from VH CDR1 at the far left; and (B) variable light domain (SEQ ID. NO.:18) of 4D4com-2F9, with the VL CDR1 (SEQ ID NO.:4), the VL CDR2 (SEQ ID NO.:14), and the VL CDR3 (SEQ ID NO.:64) underlined, starting in order from VL CDR1 at the far left.

FIGS. 5A-B show the amino acid sequences of the (A) variable heavy domain (SEQ ID NO.:21) of 7F3, with the VH CDR1 (SEQ ID NO.:19), the VH CDR2 (SEQ ID NO.:61), and the VH CDR3 (SEQ ID NO.:3) underlined, starting in order from VH CDR1 at the far left; and (B) variable light domain (SEQ ID. NO.:22) of 7F3, with the VL CDR1 (SEQ ID NO.:4), the VL CDR2 (SEQ ID NO.:5), and the VL CDR3 (SEQ ID NO.:20) underlined, starting in order from VL CDR1 at the far left.

FIGS. 6A-B show the amino acid sequences of the (A) variable heavy domain (SEQ ID NO.:23) of 71A10, with the VH CDR1 (SEQ ID NO.:19), the VH CDR2 (SEQ ID NO.:2), and the VH CDR3 (SEQ ID NO.:3) underlined, starting in order from VH CDR1 at the far left; and (B) variable light domain (SEQ ID. NO.:24) of 71A10, the VL CDR1 (SEQ ID NO.:4), the VL CDR2 (SEQ ID NO.:5), and the VL CDR3 (SEQ ID NO.:20) underlined, starting in order from VL CDR1 at the far left.

FIGS. 7A-B show the amino acid sequences of the (A) variable heavy domain (SEQ ID NO.:21) of 7F3 22D3, with the VH CDR1 (SEQ ID NO.:19), the VH CDR2 (SEQ ID NO.:61), and the VH CDR3 (SEQ ID NO.:3) underlined, starting in order from VH CDR1 at the far left; and (B) variable light domain (SEQ ID. NO.:25) of 7F3 22D3, with the VL CDR1 (SEQ ID NO.:4), the VL CDR2 (SEQ ID NO.:14), and the VL CDR3 (SEQ ID NO.:20) underlined, starting in order from VL CDR1 at the far left.

FIGS. 8A-B show the amino acid sequences of the (A) variable heavy domain (SEQ ID NO.:27) of 7F3com-2H2, the VH CDR1 (SEQ ID NO.:26), with the VH CDR2 (SEQ ID NO.:2), and the VH CDR3 (SEQ ID NO.:3) are underlined, starting in order from VH CDR1 at the far left; and (B) variable light domain (SEQ ID. NO.:28) of 7F3com-2H2, the VL CDR1 (SEQ ID NO.:62), the VL CDR2 (SEQ ID NO.:65), and the VL CDR3 (SEQ ID NO.:20) underlined, starting in order from VL CDR1 at the far left.

FIGS. 9A-B show the nucleotide sequences of the (A) variable heavy domain (SEQ ID NO.:43) of 7F3com-2H2 with the VH CDR1 (SEQ ID NO.:44), the VH CDR2 (SEQ ID NO.:45) and the VH CDR3 (SEQ ID NO.:46) underlined, starting in order from VH CDR1 at the far left; and (B) variable light domain (SEQ ID NO.:47) of 7F3com-2H2 with the VL CDR1 (SEQ ID NO.:48), the VL CDR2 (SEQ ID NO.:49), and the VL CDR3 (SEQ ID NO.:50) underlined, starting in order from VL CDR1 at the far left.

FIGS. 10A-B show the amino acid sequences of the (A) variable heavy domain (SEQ ID NO.:29) of 7F3com-3H5, with the VH CDR1 (SEQ ID NO.:19), the VH CDR2 (SEQ ID NO.:2), and the VH CDR3 (SEQ ID NO.:3) underlined, starting in order from VH CDR1 at the far left and (B) variable light domain (SEQ ID. NO.:30) of 7F3com-3H5, with the VL CDR1 (SEQ ID NO.:4), the VL CDR2 (SEQ ID NO.:14), and the VL CDR3 (SEQ ID NO.:20) underlined, starting in order from VL CDR1 at the far left.

FIGS. 11A-B show the amino acid sequences of the (A) variable heavy domain (SEQ ID NO.:31) of 7F3com-3D4, with the VH CDR1 (SEQ ID NO.:26), the VH CDR2 (SEQ ID NO.:2), and the VH CDR3 (SEQ ID NO.:3) underlined, starting in order from VH CDR1 at the far left and (B) variable light domain (SEQ ID. NO.:32) of 7F3com-3D4, with the VL CDR1 (SEQ ID NO.:62), the VL CDR2 (SEQ ID NO.:14), and the VL CDR3 (SEQ ID NO.:20) underlined, starting in order from VL CDR1 at the far left.

FIG. 12 shows the nucleotide sequence of human IL-9 (SEQ ID NO.:51) located in the GenBank database (Accession Nos. NM_{—}000590).

FIG. 13 shows the amino acid sequence for human IL-9 located in the GenBank database (Accession Nos. A60480 (SEQ ID NO.: 52), NP_{—}000584 (SEQ ID NO.:53) and AAC17735 (SEQ ID NO.:54)).

FIGS. 14A-C shows the nucleotide sequence of human IL-9R subunits found in the GenBank database (Accession Nos. NM_{—}002186 (SEQ ID NO.:55), NM_{—}176786 (SEQ ID NO.:56), and NM_{—}000206 (SEQ ID NO.:57)). (A) Accession No. NM_{—}002186 and (B) Accession No. NM_{—}176786 are the nucleotide sequences of human IL-9R alpha subunit isoform precursors. (C) Accession No. NM_{—}000206 is the nucleotide sequence of the human IL-9R gamma chain.

FIG. 15 shows the amino acid sequence of human IL-9R found in the GenBank database (Accession Nos. NP_{—}002177 (SEQ ID NO.:58); NP_{—}789743 (SEQ ID NO.:59), and NP_{—}000197 (SEQ ID NO.:60)). Accession Nos. NP_{—}002177 and NP_{—}789743 are the amino acid sequences of human IL-9R alpha subunit isoform precursors. NP_{—}000197 is the amino acid sequence of the human IL-9R gamma chain.

FIG. 16 is a schematic diagram showing the outline for preparing purified antibodies that immunospecifically bind to an IL-9 polypeptide.

FIGS. 17A-17B. Urea induced unfolding of 7F3com-2H2 in the absence of salt and in the presence of 150 mM NaCl demonstrates that in presence of histidine alone, the unfolding of the antibody is a simple 2-step process which is indicative of cooperative unfolding of all domains, whereas with the addition of salt, the unfolding of 7F3com-2H2 domains is a sequential process through the formation of a intermediate population. Graph of (A) 10 mM histidine, pH 6.0, and (B) 10 mM phosphate buffer pH 6.0.

FIGS. 18A-18B. Differential Scanning Calorimetry (DSC) profile of 7F3com-2H2 in (A) 10 mM histidine, pH 6.0 with no salt, 25 mM NaCl, and 150 mM NaCl; and in (B) 10 mM phosphate buffer, pH 6.0 in no salt, and 150 mM NaCl.

FIGS. 19A-19B. KI (postassium iodide) quenching studies of the 7F3com-2H2 antibody. (A) Stern-Volmer plot of Native and Intermediate 7F3com-2H2 in 10 mM histidine, 150 mM NaCl, ph 6.0. (B) Stern-Volmer plot of Native and unfolded 7F3com-2H2 in 10 mM phosphate, 150 mM NaCl, pH 6.0.

FIGS. 20A-20B. (A) Urea induced unfolding of 7F3com-2H2 at pH 8.1 in 10 mM Histidine, as followed by tryptophan fluorescence and ANS binding experiments. The error bar shown is obtained from 3 different sets of experiments done on different days. (B) Urea induced unfolding of 7F3com-2H2 at pH 8.1 in 10 mM phosphate, as followed by tryptophan fluorescence and ANS binding experiments.

FIG. 21. Results of a Fourier Transform Infrared Spectroscopy (FTIR) experiment performed to an alyze the structural non-idealities of the antibody 7F3com-2H2. The results indicate that pH alone is not responsible for 7F3com-2H2 (pI=8.1) associations in solution; it is strongly dependent on the buffer type.

FIG. 22. Urea induced unfolding of full-length, F_{ab}, and F_{c} fragments of 7F3com-2H2 in 10 mM histidine, pH 6 in absence of salt (left panel) and in presence of 150 mM NaCl (right panel). Unfolding of 7F3com-2H2 was measured by changes in the center of spectral mass (CSM) as a function of urea concentration. The solid lines are the curve obtained using either simple 2-state or non-2 state fitting. The inset panel shows the unfolding of full-length of 7F3com-2H2 in 10 mM phosphate buffer pH 6.0.

FIGS. 23a and b. (a) DCS studies with full-length, F_{ab}, and F_{c} fragments of 7F3com-2H2 indicates that in presence of histidine and salt there is a decreased domain-domain interaction in full-length mAb and destabilization of CH₂ domain of F_{c} region. (b) DSC studies with full-length, and isolated F_{ab} and F_{c} fragments of 7F3com-2H2 in phosphate buffer did not show any significant changes either in presence or absence of salt.

FIG. 24. Cartoon representation of charge shielding effect of NaCl. At pH 6 the 7F3com-2H2 antibody is positively charged and the imidazole side chain of histidine has more than 50% probability to be positively charged, causing charge-charge repulsion between histidine and the Ab.

FIG. 25. Viscosity of the 7F3com-2H2 antibody at pH 6 in two buffer systems. The solutions with and without NaCl have ionic strengths of 153 and 2.5 mM, respectively. All viscosities less than 20 cP were measured at a shear rate of 600 s⁻¹. Curves represent a fit to the third order polynomial η=η₀(1+k₁c+k₂c²+k₃c³ . . . ).

FIG. 26. Nephelometric Turbidity (light scattering at)90° as a measure of opalescence of 7F3com-2H2 in the identical conditions used for viscometry. Opalescence and viscosity are inversely affected by ionic strength for this Ab. Lines are to guide the eye.

FIG. 27. Osmotic pressure measurements via membrane osmometry of 7F3com-2H2 solutions at pH 6 in two buffer systems with ionic strengths matching those of FIG. 25. The higher ionic strength solutions result in negative second virial coefficients as well as larger apparent molecular weights than the low ionic strength buffer systems. Lines represent a linear regression.

FIG. 28a-c. (a) calculation of critical second virial coefficient from osmotic pressure data. (b) Table showing critical virial coefficients as reported in literature are in the −5 to −6 range. (c) Table providing linear regression of osmotic pressure data in FIG. 27 to equation in (a, left) and normalized to the protein volume as in the equation (a, right). The two conditions that are opalescent also exhibit second virial coefficients near the critical value suggesting that opalescence may be related to a phase separation (liquid-liquid or liquid-solid).

FIG. 29. Stability of anti-IL-9 antibody formulations at 40°. Various antibody formulations comprising 5 g/l anti-IL-9 antibody were incubated at 40° C. for up to 75 days. Antibody fragmentation was assessed by size exclusion chromatography at regular time intervals. Monomer concentration and antibody fragment concentration measured is plotted as a function of time.

FIG. 30. Representative chromatograms obtained with the antibody formulation comprising 2.2 mM Sodium Phosphate over the course of the experiment. The UV absorbance curves displayed correspond to the left axis. Elution data points obtained using a commercially available molecular weight marker correspond to the right axis.

### 5. DETAILED DESCRIPTION OF THE INVENTION

The stable liquid formulations of the present invention provide a ready-to-use preparation of a therapeutic or prophylactic antibody of interest (including antibody fragments thereof), for example, an antibody that immunospecifically binds to an IL-9 polypeptide (e.g., 7F3com-2H2), for administering to a subject (e.g., a human subject) without having to reconstitute the preparation using accurate and aseptical techniques, and waiting for a period of time until the solution clarifies before administering the formulation to the subject. In addition, such reconstituted solutions must be used within a certain period, leading to very costly waste. It simplifies the procedure of administering the formulation to a subject for a healthcare professional.

Furthermore, due to its high stability during storage, the formulations of the present invention can contain a therapeutic or prophylactic antibody of interest (including antibody fragment thereof), for example, an antibody that immunospecifically binds to an IL-9 polypeptide (e.g., 7F3com-2H2), at concentrations in the range of about 10 mg/ml to about 300 mg/ml. Such stability not only ensures the efficacy of the antibodies but also reduces possible risks of adverse effects in a subject. Furthermore, the use of fewer components in the formulation reduces the risk of contamination. In addition, the manufacturing process of the liquid formulations of the present invention is simplified and more efficient than the manufacturing process for the lyophilized version because all stages of the manufacturing of the liquid formulations are carried out in an aqueous solution, involving no drying process, such as lyophilization and freeze-drying. Accordingly, it is more cost effective as well.

Characteristics of antibodies that can be formulated in accordance with the methods of the invention include certain phase behaviors, such as formation of unfolded intermediates, colloidal instability, soluable association of the antibody molecules, and precipitation of the antibody molecules, as measured by sensitive analytical techniques, including but not limited to, high performance size exclusion chromatography (HPSEC), tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and 1-anilino-8-naphthalenesulfonic acid (ANS) protein binding techniques when the antibodies are formulated in the presence of zwitterionic buffers containing, for example, histidine, which can interact with the antibody by decreasing domain-domain interactions, possibly by disassembling salt bridges, due to the zwitterionic nature of histidine. This interaction can result in reduced colloidal stability, soluble association, and precipitation, all of which may lead to increased protein aggregation, an undesirable result contributing to the instability of the antibody formulations. Thus, the present invention provides methods of determining whether certain buffers, e.g., non-zwitterionic buffers (e.g., phosphate, tris, citrate, succinate, and acetate buffers) promote a reduction or decrease in certain phase behaviors (e.g., colloidal instability, soluble association, and precipitation) of particular proteins (e.g., monoclonal antibodies) that make them more suitable for formulation using the methods of the present invention.

5.1. Antibody Formulations

The stable liquid formulations of the present invention provide antibody formulations which exhibit little to no aggregation and high stability during long periods of storage. In a specific embodiment, such antibody formulations are homogeneous. In one embodiment, the formulations of the invention are sterile. The formulations of the present invention comprise an aqueous carrier (e.g., distilled water), phosphate or other non-zwitterionic buffers and, optionally, salt such as NaCl and a therapeutic or prophylactic antibody of interest (including antibody fragment thereof), for example, an antibody that immunospecifically binds to an IL-9 polypeptide, provided that the antibody is more stable in a non-zwitterionic buffer than in a zwitterionic buffer at a pH below the pI, at concentrations of about 10 mg/ml to about 300 mg/ml. In one embodiment, the formulations of the invention do not comprise other ingredients except for water or suitable solvents. In another preferred embodiment, the water is distilled. In a specific embodiment, the antibody that immunospecifically binds to an IL-9 polypeptide which is included in the liquid formulations of the invention is 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof In another embodiment, the antibody that immunospecifically binds to an IL-9 polypeptide which is included in the liquid formulations of the invention is not 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof In one embodiment, the antibody that immunospecifically binds to an IL-9 polypeptide which is included in the liquid formulation of the invention is an antibody (including antibody fragment thereof) comprising one or more of the VH CDRs and/or one or more of the VL CDRs listed in Table 1, supra. In another embodiment, the antibody (including antibody fragment thereof) that immunospecifically binds to an IL-9 polypeptide which is included in the liquid formulations of the invention is an antibody (including antibody fragment thereof) conjugated to another moiety, including but not limited to, a heterologous polypeptide, another antibody (including antibody fragment thereof), a marker sequence, a diagnostic agent, a therapeutic agent, a radioactive metal ion, a polymer, albumin, and a solid support. In yet another embodiment, liquid formulations of the invention comprise two or more antibodies or (including antibody fragments thereof) that immunospecifically binds to an IL-9 polypeptide, wherein at least one of the antibodies (including antibody fragments thereof) is 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof.

The concentration of an antibody of interest (including antibody fragment thereof), for example, an antibody that immunospecifically binds to an IL-9 polypeptide, which is included in the liquid formulations of the invention is at least 10 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 175 mg/ml, at least 200 mg/ml, at least 250 mg/ml, at least 275 mg/ml, or at least 300 mg/ml. The concentration of an antibody of interest (including antibody fragment thereof), for example, an antibody that immunospecifically binds to an IL-9 polypeptide (e.g., 7F3com-2H2), which is included in the liquid formulations of the invention is 10 mg/ml or higher, at least 15 mg/ml or higher, at least 20 mg/ml or higher, at least 25 mg/ml or higher, at least 30 mg/ml or higher, at least 35 mg/ml or higher, at least 40 mg/ml or higher, at least 45 mg/ml or higher, at least 50 mg/ml or higher, at least 55 mg/ml or higher, at least 60 mg/ml or higher, at least 65 mg/ml or higher, at least 70 mg/ml or higher, at least 75 mg/ml or higher, at least 80 mg/ml or higher, at least 85 mg/ml or higher, at least 90 mg/ml or higher, at least 95 mg/ml or higher, at least 100 mg/ml or higher, at least 105 mg/ml or higher, at least 110 mg/ml or higher, at least 115 mg/ml or higher, at least 120 mg/ml or higher, at least 125 mg/ml or higher, at least 130 mg/ml or higher, at least 135 mg/ml or higher, at least 140 mg/ml or higher, at least 150 mg/ml or higher, at least 175 mg/ml or higher, at least 200 mg/ml or higher, at least 250 mg/ml or higher, at least 275 mg/ml or higher, or at least 300 mg/ml or higher. In a specific embodiment, the concentration of an antibody of interest (including antibody fragment thereof), for example, an antibody that immunospecifically binds to an IL-9 polypeptide (e.g., 7F3com-2H2), which is included in the liquid formulation of the invention is about 50 mg/ml, 75 mg/ml, about 100 mg/ml, about 125 mg/ml, about 150 mg/ml, about 175 mg/ml, about 200 mg/ml, about 225 mg/ml, about 250 mg/ml, about 275 mg/ml, or about 300 mg/ml. In another embodiment, the concentration of an antibody of interest (including antibody fragment thereof), for example, an antibody that immunospecifically binds to an IL-9 polypeptide (e.g., 7F3com-2H2), which is included in the liquid formulation of the invention is between 10-50 mg/ml, between 15-500 mg/ml, between 50-300 mg/ml, between 50-250 mg/ml, between 50-200 mg/ml, between 50-150 mg/ml, between 100-200 mg/ml, between 50-175 mg/ml, between 50-150 mg/ml, between 50-125 mg/ml, or between 50-100 mg/ml. In a specific embodiment, a formulation of the invention comprises about 100 mg/ml of an anti-IL-9 antibody (e.g., 7F3com-2H2).

The formulation may be buffered by phosphate (although other appropriate buffers may be used such as tris, citrate, succinate, and acetate buffers). The concentration of phosphate which is included in the liquid formulations of the invention ranges from 1 mM to 5mM, 1 mM to 100 mM, 10 mM to 30mM, 25 mM to 75 mM, or 10 mM to 100 mM. In a specific embodiment, the concentration of phosphate which is included in the liquid formulations of the invention is 2 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. Phosphate may be used any form suitable for formulation and parenteral administration. The purity of phosphate should be at least 98%, at least 99%, or at least 99.5%. As used herein, the term “purity” in the context of phosphate refers to chemical purity of phosphate as understood in the art, e.g., as described in The Merck Index, 13^{th} ed., O'Neil et al. ed. (Merck & Co., 2001). In a specific embodiment, a formulation of the invention comprises 25 mM phosphate buffer. In addition to phosphate, other buffers, particularly non-zwitterionic buffers, may be used in the formulations of the present invention (e.g., tris, citrate, succinate and acetate buffers) at concentration ranges from 1 mM to 100 mM, 25 mM to 75 mM, or 10 mM to 100 mM, or at a concentration of 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In certain embodiments, a formulation of the invention comprises a salt. In one embodiment, a formulation of the invention comprises a salt selected from the group consisting of: NaCl, KCl, CaCl₂, and MgCl₂. In a specific embodiment, the salt may be NaCl. The concentration of NaCl that may be included in the liquid formulations of the invention may range from 10 mM to 300 mM, 50 mM to 200 mM, 100 mM to 200 mM, or 125 mM to 175 mM. In a specific embodiment, the concentration of NaCl which may be included in the liquid formulations of the invention may be about 10 mM, about 25 mM, about 50 mM, about 75 mM, about 100 mM, about 125 mM, about 150 mM, about 175 mM, about 200 mM, about 225 mM, about 250 mM, or about 300 mM . The purity of NaCl may be at least 98%, at least 99%, or at least 99.5%. In a specific embodiment, the concentration of NaCl included in the liquid formulations of the invention may be about 150 mM. As used herein, the term “purity” in the context of NaCl refers to chemical purity of NaCl as understood in the art, e.g., as described in The Merck Index, 13^{th} ed., O'Neil et al. ed. (Merck & Co., 2001).

The pH of the formulation generally should not be equal to the isoelectric point of the particular antibody (including antibody fragment thereof) to be used in the formulation (e.g., the isoelectric point of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 ranges from 8.65 to 8.89) and may range from about 4.0 to about 8.0, or may range from about 6.0 to about 6.5. In a specific embodiment, a formulation of the invention comprises an anti-IL-9 antibody (e.g., 7F3com-2H2) and may have a pH of about 6.0.

Optionally, the formulations of the present invention may further comprise other excipients, such as saccharides (e.g., sucrose, mannose, trehalose, etc.), polyols (e.g., mannitol, sorbitol, etc.) and surfactants (e.g., Tween-20 or Tween-80). In one embodiment, the other excipient is a saccharide (e.g., a sugar). In a specific embodiment, the saccharide is sucrose, which is at a concentration ranging from between about 0% to about 10%, 1% to about 20%, about 5% to about 15%, or about 8% to 10% of the formulation. In another embodiment, the saccharide is trehalose, which is at a concentration of about 0% to about 10%, 1% to about 20%, or about 5% to about 15%, or about 8% to 10% of the formulation. In another embodiment, an excipient is a polyol. In a specific embodiment, the polyol is at a concentration of 0.001%, 0.005%, 0.01%, 0.02%, 0.05%, 0.08%, 0.1%, 0.5%, or 1% of the formulation. In a specific embodiment, the polyol is sorbitol, which is at a concentration ranging from between about 0% to about 0.1%, 0.001% to about 1%, or about 0.01% to about 0.1% of the formulation. The liquid formulations of the present invention may or may not contain mannitol. In another embodiment, an excipient is a surfactant. In specific embodiments, the surfactant is Tween-20 or Tween-80, which is at a concentration ranging from between about 0% to about 0.1%, 0.001% to about 1%, or about 0.01% to about 0.1% of the formulation. In a specific embodiment, the surfactant is Tween-20 or Tween-80, which is at a concentration of 0.001%, 0.005%, 0.01%, 0.02%, 0.05%, 0.08%, 0.1%, 0.5%, or 1% of the formulation.

It will be understood by one skilled in the art that the formulations of the invention may be isotonic with human blood, that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this invention include, but are not limited to, saccharides, salts and amino acids.

In certain embodiments, a formulation of the invention may comprise between about 51 mg/ml and about 150 mg/ml anti-IL-9 antibody (e.g., 7F3com-2H2), between about 10 mM and about 50 mM sodium phosphate, between about 100 mM and about 200 mM NaCl and has a pH of between about 5.5 and about 7.0. In a further embodiment, a formulation of the invention may comprise about 100 mg/ml anti-IL-9 antibody (e.g., 7F3com-2H2), about 25 mM sodium phosphate, about 150 mM NaCl and has a pH of about 6.0. In a specific embodiment, a formulaitn of the invention consists of about 100 mg/ml 7F3com-2H2 anti-IL-9 antibody, about 25 mM sodium phosphate, about 150 mM NaCl and has a pH of about 6.0.

The liquid formulations of the present invention exhibit stability at the temperature range of 38° C.-42° C. for at least 15 days and, in some embodiments, not more than 25 days, at the temperature range of 20° C.-24° C. for at least 6 months, at the temperature range of 2° C.-8° C. (in particular, at 4° C.) for at least 6 months, at least 1 year, at least 1.5 years, at least 2 years, at least 2.5 years, at least 3 years or at least 4 years, and at the temperature of −20° C. for at least 2 years, at least 3 years, at least 4 years, or at least 5 years, as assessed by high performance size exclusion chromatography (HPSEC). The techniques of tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and/or 1-anilino-8-naphthalenesulfonic acid (ANS) protein binding are also used to assess the phase behaviors, other physical properties and stability of the molecule. Namely, the liquid formulations of the present invention have low to undetectable levels of aggregation and/or fragmentation, as defined herein, after the storage for the defined periods as set forth above. For example, no more than 5%, no more than 4%, no more than 3%, no more than 2%, no more than 1%, and most preferably no more than 0.5% of the antibody (including antibody fragment thereof) forms an aggregate as measured by HPSEC after the storage for the defined periods as set forth above.

Furthermore, liquid formulations of the present invention exhibit almost no loss in biological activities of the antibody (including antibody fragment thereof) during the prolonged storage under the condition described above, as assessed by various immunological assays including, for example, enzyme-linked immunosorbent assay (ELISA) and radioimmunoassay to measure the ability of the antibody (including antibody fragment thereof) to immunospecifically bind to an antigen. The liquid formulations of the present invention retain after the storage for the above-defined periods more than 80%, more than 85%, more than 90%, more than 95%, more than 98%, more than 99%, or more than 99.5% of the initial biological activities (e.g., the ability to bind to an IL-9 polypeptide) of the formulation prior to the storage. In some embodiments, the liquid formulations of the present invention retain after the storage for the above-defined periods at least 80%, at least 85%, at least 90%, at least 95%, at least 98%, at least 99%, or at least 99.5% of the biological activity (e.g., the ability to bind to an IL-9 polypeptide) compared to a reference antibody representing the antibody prior to the storage.

The liquid formulations of the present invention can be prepared as unit dosage forms. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody or antibody fragment ranging from about 15 mg/ml to about 300 mg/ml, about 50 mg/ml to about 300 mg/ml, about 50 mg/ml to about 150 mg/ml, about 75 mg/ml to about 300 mg/ml, about 95 mg/ml to about 300 mg/ml, about 100 mg/ml to about 300 mg/ml, about 150 mg/ml to about 300 mg/ml, about 200 mg/ml to about 300 mg/ml, about 100 mg/ml to about 200 mg/ml, about 100 mg/ml to about 150 mg/ml, or about 100 mg/ml to about 175 mg/ml. If necessary, these preparations can be adjusted to a desired concentration by adding a sterile diluent to each vial.

The invention encompasses stable liquid formulations comprising a single antibody of interest (including antibody fragment thereof), for example, an antibody that immunospecifically binds to an IL-9 polypeptide. The invention also encompasses stable liquid formulations comprising two or more antibodies of interest (including antibody fragments thereof), for example, antibodies that immunospecifically bind to an IL-9 polypeptide(s). In a specific embodiment, a stable liquid formulation of the invention comprises 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or a fragment thereof that immunospecifically binds to an IL-9 polypeptide. In another embodiment, a stable liquid formulation of the invention comprises two or more antibodies (including antibody fragments thereof) that immunospecifically bind to an IL-9 polypeptide, wherein one of the antibodies (including antibody fragments thereof) is 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof. In an alternative embodiment, a stable liquid formulation of the invention comprises two or more antibodies (including antibody fragments thereof) that immunospecifically bind to an IL-9 polypeptide, with the proviso that the antibodies (including antibody fragments thereof) do not include 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof. 5.1.1. Antibodies Useful in the Formulations of the Invention

The antibodies for use in accordance with the methods of the present invention include, but are not limited to, antibodies that display certain phase behaviors such as, for example, formation of unfolded intermediates, reduced colloidal stability, soluble association and precipitation when formulated in the presence of a zwitterionic buffer (e.g., histidine buffer) at a pH below the pI of the antibodies in the presence of salt (e.g., NaCl), as compared to the antibodies when formulated in the presence of a non-zwitterionic buffer (e.g., phosphate buffer) at the same pH and in the presence (or absence) of salt at the same concentration, as measured by, for example, high performance size exclusion chromatography (HPSEC), tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and 1-anilino-8-naphthalenesulfonic acid (ANS) protein binding techniques. In specific embodiments, the antibodies (or fragments thereof) for use in accordance with the methods of the invention include, but are not limited to, 4D4, 4D4 H2-1 D11, 4D4com-XF-9, 4D4com-2F9, 7F3, 71A10, 7F3 22D3, 7F3com-2H2, 7F3com-3H5, or 7F3com-3D4. Preferably, the antibody (or fragments thereof) for use in accordance with the methods of the present invention is 7F3com-2H2. Other therapeutic or prophylactic antibodies for use in accordance with the methods of the invention are disclosed, for example in Sections 5.1.1.1 to 5.1.1.17, infra.

The antibodies useful in the present invention include, but are not limited to, monoclonal antibodies, synthetic antibodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies of the present invention include immunoglobulin molecules and immunologically active portions of immunoglobulin molecules, i.e., molecules that contain an antigen binding site that immunospecifically binds to an antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies useful in the present invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice or other animal that express antibodies from human genes.

The antibodies useful in the present invention may be monospecific, bispecific, trispecific or of greater multispecificity. Multispecific antibodies may immunospecifically bind to different epitopes of a polypeptide or may immunospecifically bind to both a polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies useful in the present invention include derivatives of the antibodies. Standard techniques known to those of skill in the art can be used to introduce mutations in the nucleotide sequence encoding an antibody to be used with the methods of the invention, including, for example, site-directed mutagenesis and PCR-mediated mutagenesis which result in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In one embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies useful in the present invention include derivatives that are modified, i. e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

Antibodies useful in the present invention or fragments thereof can also comprise a framework region known to those of skill in the art. In certain embodiments, one or more framework regions, preferably, all of the framework regions, of an antibody to be used in the methods of the invention or fragment thereof are human. In certain other embodiments of the invention, the fragment region of an antibody of the invention or fragment thereof is humanized. In certain embodiments, the antibody to be used with the methods of the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments of the invention, the antibodies useful in the present invention have half-lives in a mammal, preferably a human, of greater than 12 hours, greater than 1 day, greater than 3 days, greater than 6 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. Antibodies or antigen-binding fragments thereof having increased in vivo half-lives can be generated by techniques known to those of skill in the art. For example, antibodies or antigen-binding fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and U.S. Pat. No. 7,083,784, entitled “Molecules with Extended Half-Lives, Compositions and Uses Thereof”, filed Dec. 12, 2001, by Johnson et al., which are incorporated herein by reference in their entireties). Such antibodies or antigen-binding fragments thereof can be tested for binding activity to RSV antigens as well as for in vivo efficacy using methods known to those skilled in the art, for example, by immunoassays described herein.

Further, antibodies or antigen-binding fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatizated antibodies or antigen-binding fragments thereof can be tested for binding activity to RSV antigens as well as for in vivo efficacy using methods known to those skilled in the art, for example, by immunoassays described herein.

The antibodies useful in the present invention can be single-chain antibodies. The design and construction of a single-chain antibody is described in Marasco et al, 1993, Proc Natl Acad Sci 90:7889-7893, which is incorporated herein by reference in its entirety.

In certain embodiments, the antibodies useful in the present invention bind to an intracellular epitope, i.e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). sFv are antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

In a further embodiment, the intrabody preferably does not encode an operable secretory sequence and thus remains within the cell (see generally Marasco, Wash., 1998, “Intrabodies: Basic Research and Clinical Gene Therapy Applications” Springer: New York).

5.1.1.1. IL-9 Antibodies

In specific embodiments, the present invention provides formulations of antibodies that immunospecifically bind to an IL-9 polypeptide (preferably, a human IL-9 polypeptide). In particular, the invention provides for the formulations of the following antibodies that immunospecifically bind to an IL-9 polypeptide: 4D4 or an antigen-binding fragment thereof, 4D4 H2-1 D11 or an antigen-binding fragment thereof, 4D4com-XF-9 or an antigen-binding fragment thereof, 4D4com-2F9 or an antigen-binding fragment thereof, 7F3 or an antigen-binding fragment thereof, 71A10 or an antigen-binding fragment thereof, 7F3 22D3 or an antigen-binding fragment thereof, 7F3com-2H2 or an antigen-binding fragment thereof, 7F3com-3H5 or an antigen-binding fragment thereof, and 7F3com-3D4 or an antigen-binding fragment thereof. In one embodiment, an antibody that immunospecifically binds to an IL-9 polypeptide is 7F3com-2H2 or an antigen-binding fragment thereof (e.g., one or more CDRs of 7F3com-2H2). The constant regions for 4D4, 4D4 H2-1 D11, 4D4com-XF-9, 4D4com-2F9, 71A10, 7F3 22D3, 7F3com, 7F3com-2H2, 7F3com-3H5, and 7F3com-3D4 are identical to the constant regions of palizvizumab (MedImmune, Inc.) IgG₁ (see U.S. Pat. No. 5,824,307, issued Oct. 20, 1998).

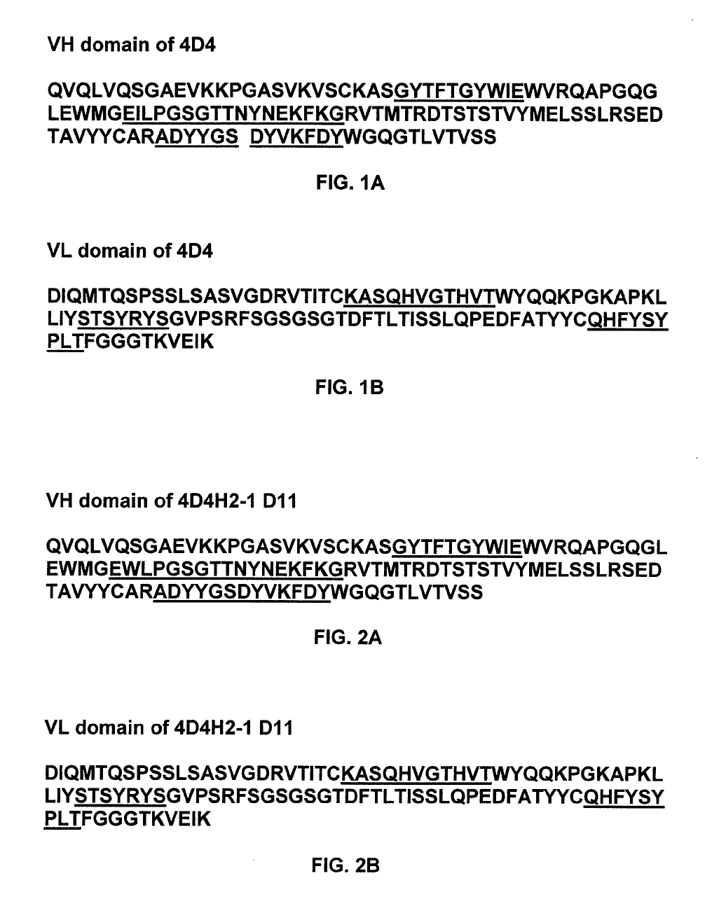

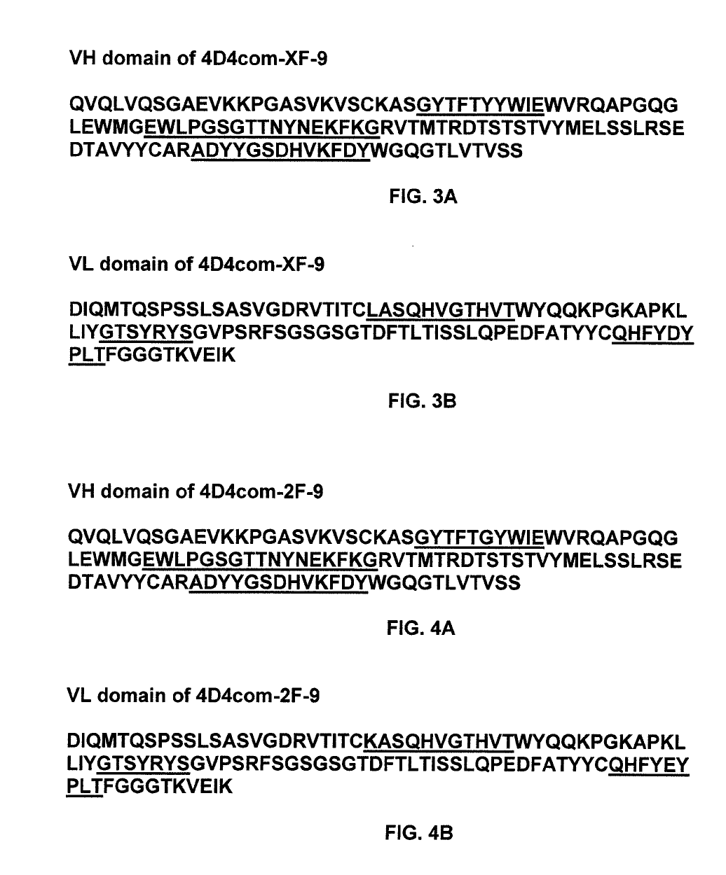

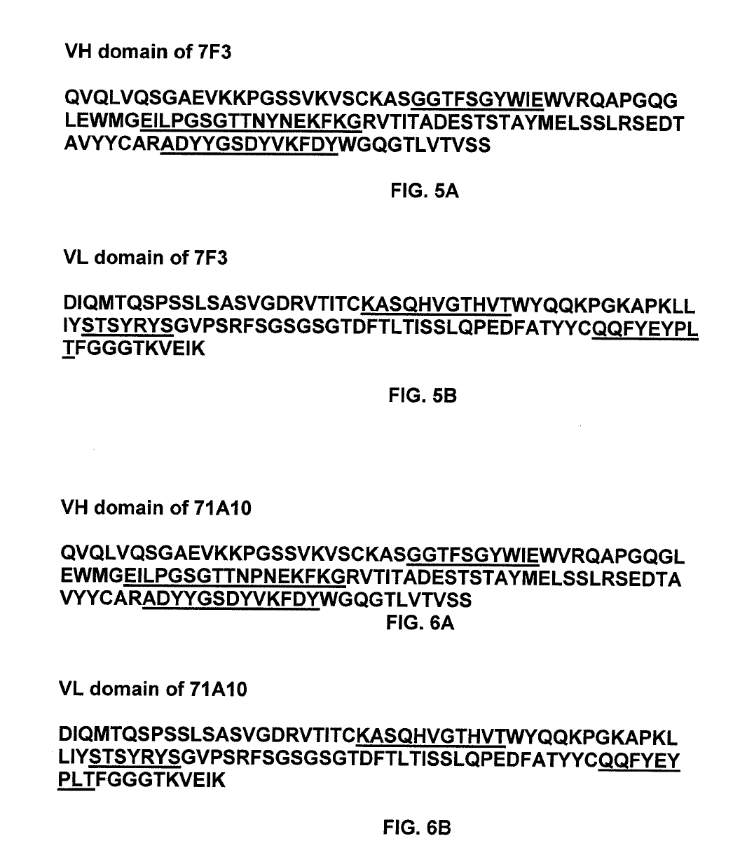

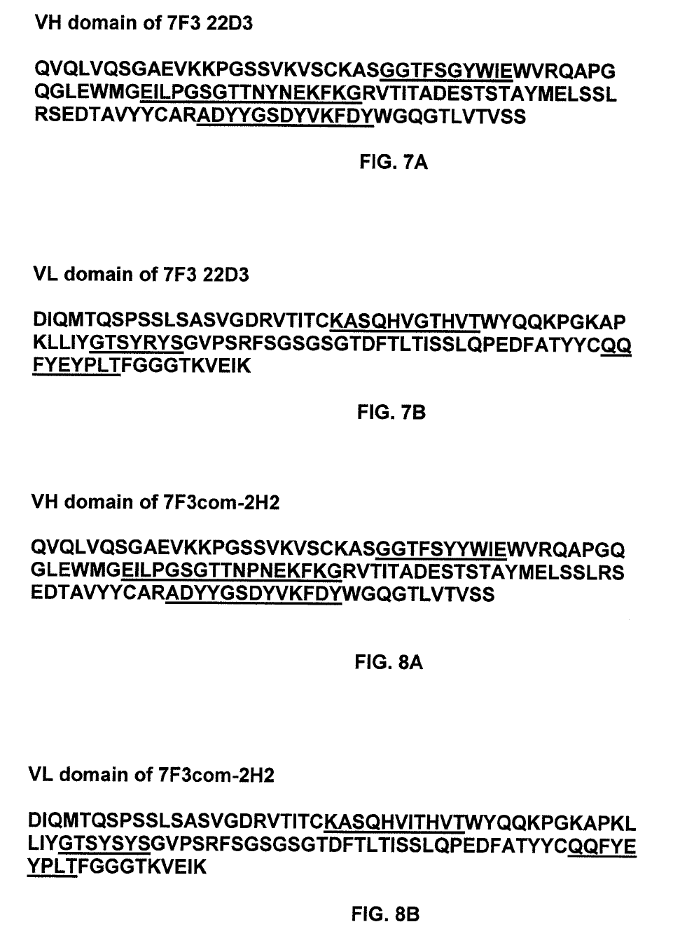

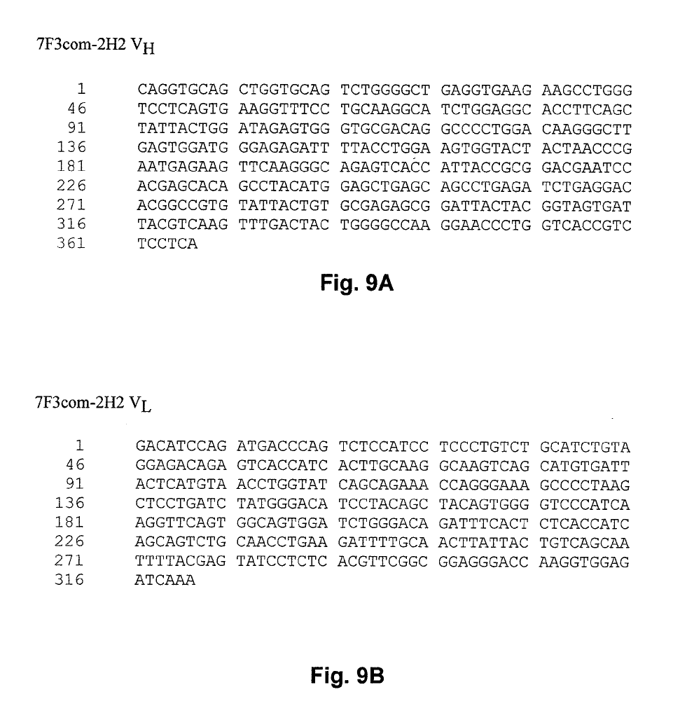

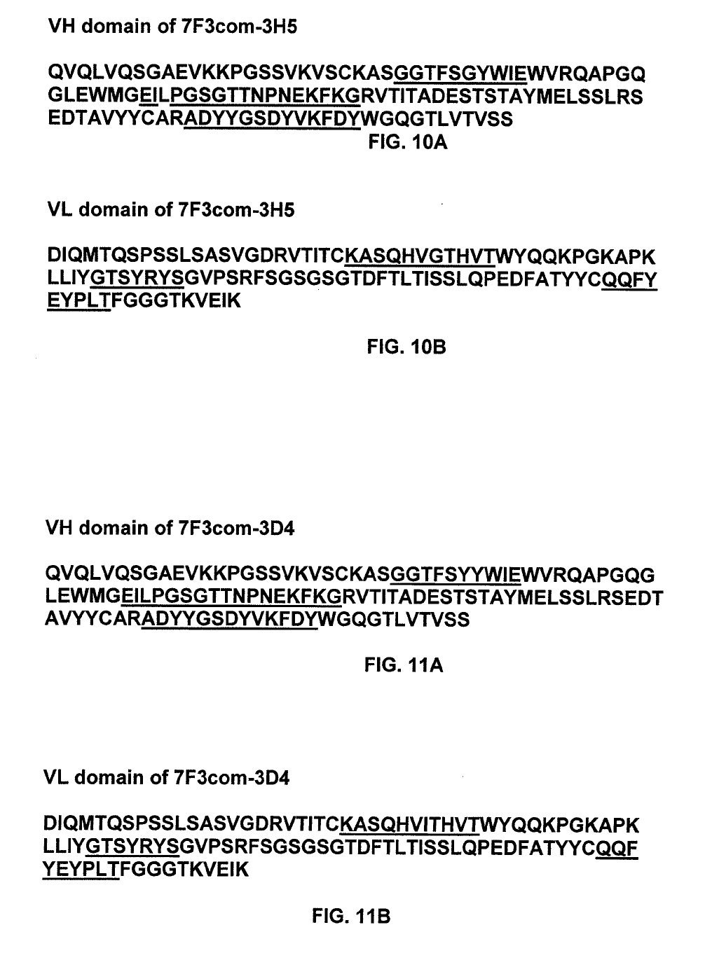

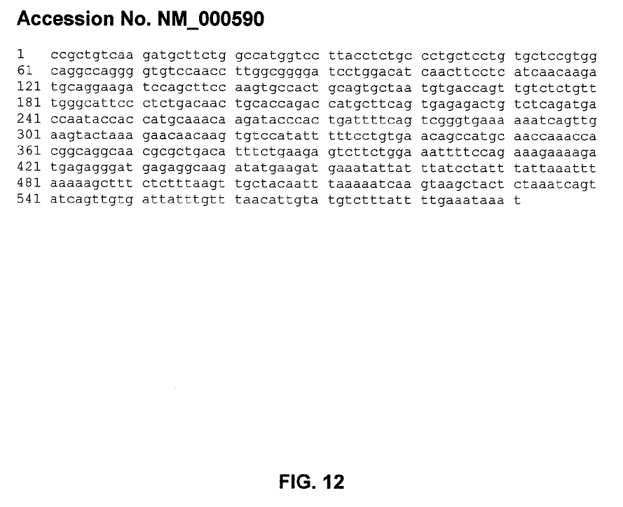

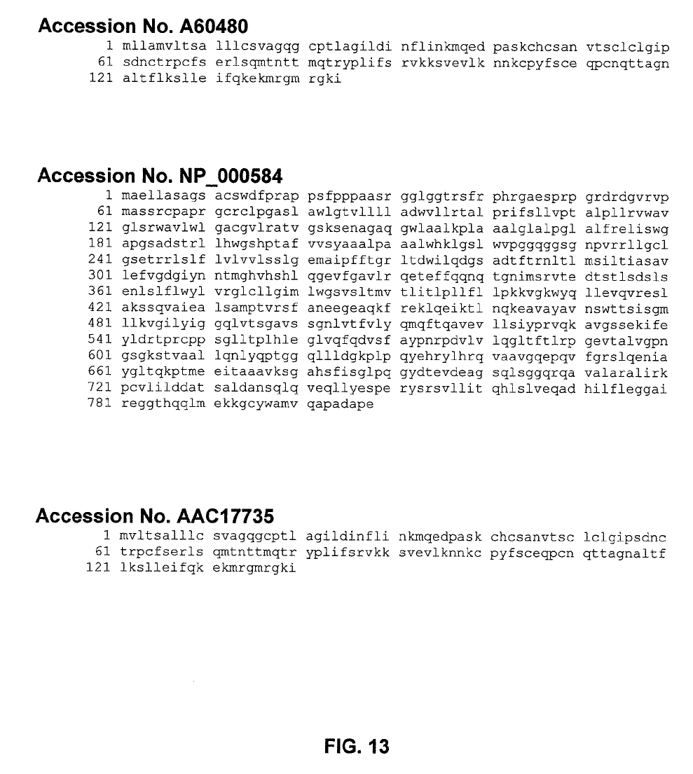

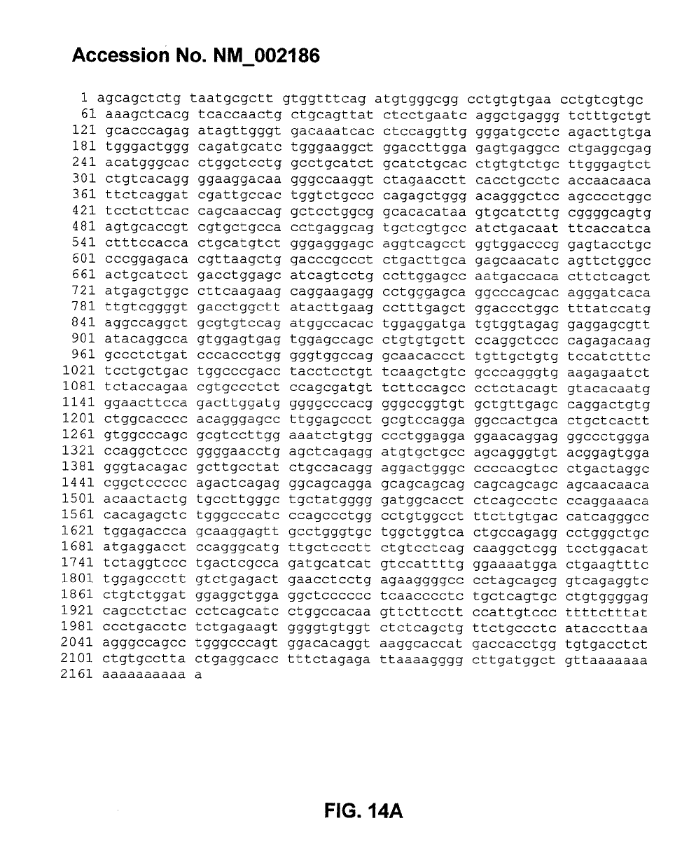

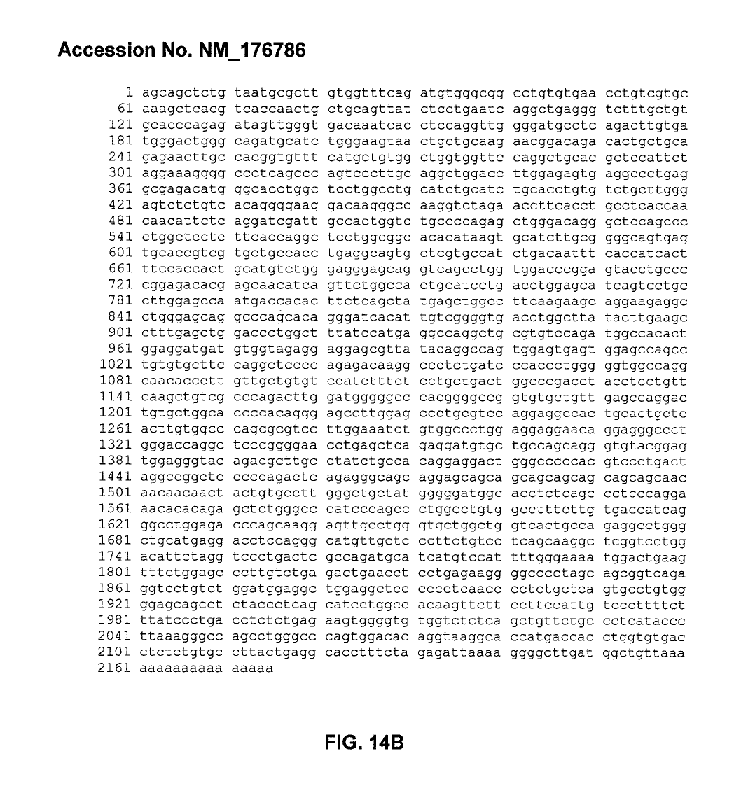

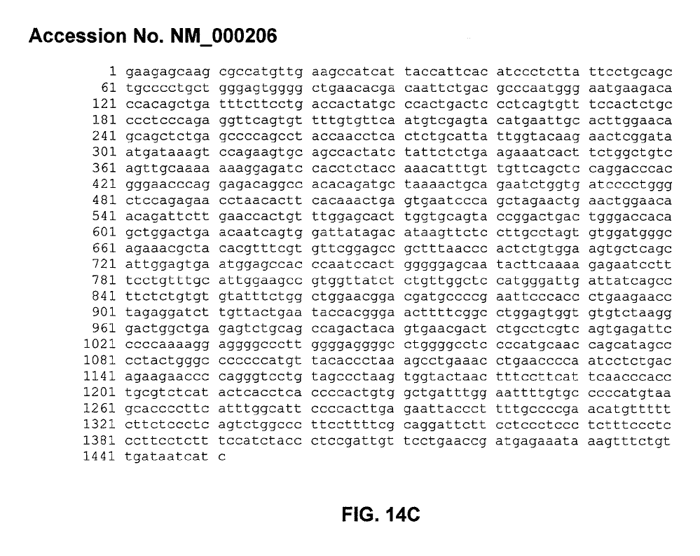

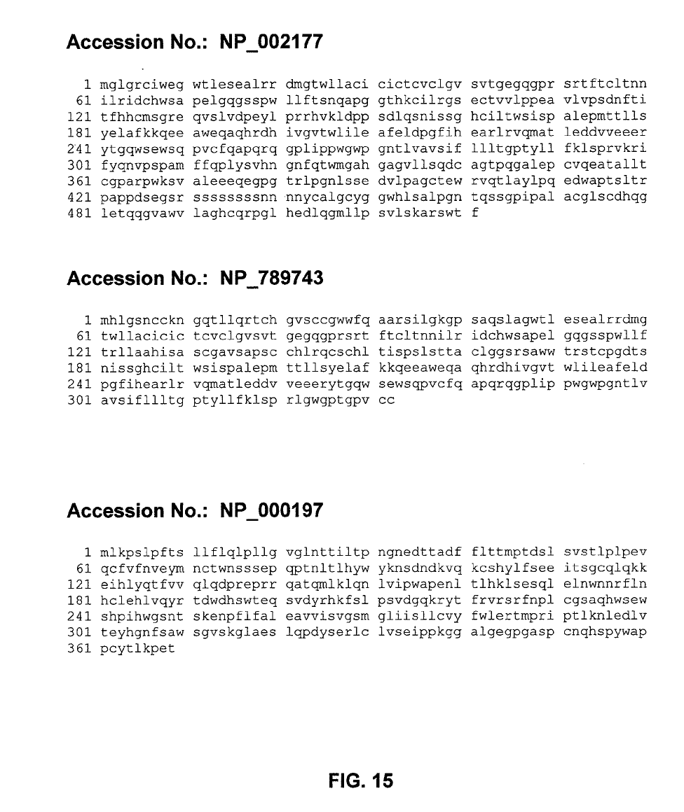

The present invention provides formulations of antibodies that immunospecifically bind an IL-9 polypeptide, said antibodies comprising a VH domain having an amino acid sequence of the VH domain of 4D4 (FIG. 1A; SEQ ID NO.:7), 4D4 H2-1 D11 (FIG. 2A; SEQ ID NO.:9), 4D4com-XF-9 (FIG. 3A; SEQ ID NO.:15), 4D4com-2F9 (FIG. 4A; SEQ ID NO.:17), 7F3 (FIG. 5A; SEQ ID NO.:21), 71A10 (FIG. 6A; SEQ ID NO.:23), 7F3 22D3 (FIG. 7A; SEQ ID NO.:21), 7F3com-2H2 (FIG. 8A; SEQ ID NO.:27), 7F3com-3H5 (FIG. 10A; SEQ ID NO.:29), or 7F3com-3D4 (FIG. 11A; SEQ ID NO.:31). In one embodiment, an antibody that immunospecifically binds to an IL-9 polypeptide comprises a VH domain having an amino acid sequence of the VH domain of 7F3com-2H2 (FIG. 8A; SEQ ID NO:27).

The present invention provides formulations of antibodies that immunospecifically bind to an IL-9 polypeptide, said antibodies comprising a VH CDR having an amino acid sequence of any one of the VH CDRs listed in Table 1, supra. In particular, the invention provides antibodies that immunospecifically bind to an IL-9 polypeptide, said antibodies comprising (or alternatively, consisting of) one, two, three, four, five or more VH CDRs having an amino acid sequence of any of the VH CDRs listed in Table 1, supra. In one embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26. In another embodiment, an antibody that immunospecifically binds to an IL-9 polypeptide comprises a VH CDR2 having the amino acid sequence of SEQ ID NO.:2, SEQ ID NO.:10 or SEQ ID NO.:61. In another embodiment, an antibody that immunospecifically binds to an IL-9 polypeptide comprises a VH CDR3 having the amino acid sequence of SEQ ID NO.:3 or SEQ ID NO.:12. In another embodiment, an antibody that immunospecifically binds to an IL-9 polypeptide comprises a VH CDR1 having the amino acid sequence of SEQ ID NO.: 1, SEQ ID NO.:11, SEQ ID NO.:19, or SEQ ID NO.:26 and a VH CDR2 having the amino acid sequence of SEQ ID NO.:2, SEQ ID NO.:10 or SEQ ID NO.:61. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VH CDR3 having the amino acid sequence of SEQ ID NO.:3 or SEQ ID NO.:12. In another embodiment, an antibody that immunospecifically binds to an IL-9 polypeptide comprises a VH CDR2 having the amino acid sequence of SEQ ID NO.:2, SEQ ID NO.:10 or SEQ ID NO.:61 and a VH CDR3 having the amino acid sequence of SEQ ID NO.:3 or SEQ ID NO.:12. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26, a VH CDR2 having the amino acid sequence of SEQ ID NO.:2, SEQ ID NO.:10 or SEQ ID NO.:61, and a VH CDR3 having the amino acid sequence of SEQ ID NO.:3 or SEQ ID NO.:12.

The present invention provides formulations of antibodies that immunospecifically bind to an IL-9 polypeptide, said antibodies comprising a VL domain having an amino acid sequence of the VL domain for 4D4 (FIG. 1B; SEQ ID NO.:8), 4D4 H2-1 D11 (FIG. 2B; SEQ ID NO.:8), 4D4com-XF-9 (FIG. 3B; SEQ ID NO.:16), 4D4com-2F9 (FIG. 4B; SEQ ID NO.:18), 7F3 (FIG. 5B; SEQ ID NO.:22), 71A10 (FIG. 6B; SEQ ID NO.:24), 7F3 22D3 (FIG. 7B; SEQ ID NO.:25), 7F3com-2H2 (FIG. 8B; SEQ ID NO.:28), 7F3com-3H5 (FIG. 10B; SEQ ID NO.:30), or 7F3com-3D4 (FIG. 11B; SEQ ID NO.:32). In one embodiment, an antibody that immunospecifically binds to an IL-9 polypeptide comprises a VL domain having an amino acid sequence of the VL domain for 7F3com-2H2 (FIG. 8B; SEQ ID NO.:28).

The present invention also provides formulations of antibodies that immunospecifically bind to an IL-9 polypeptide, said antibodies comprising a VL CDR having an amino acid sequence of any one of the VL CDRs listed in Table 1, supra. In particular, the invention provides antibodies that immunospecifically bind to an IL-9 polypeptide, said antibodies comprising (or alternatively, consisting of) one, two, three or more VL CDRs having an amino acid sequence of any of the VL CDRs listed in Table 1, supra. In one embodiment, an antibody that immunospecifically binds to an IL-9 polypeptide comprises a VL CDR1 having the amino acid sequence of SEQ ID NO.:4, SEQ ID NO.:13 or SEQ ID NO.:62. In another embodiment, an antibody that immunospecifically binds to an IL-9 polypeptide comprises a VL CDR2 having the amino acid sequence of SEQ ID NO.:5, SEQ ID NO.:14 or SEQ ID NO.:65. In another embodiment, an antibody that immunospecifically binds to an IL-9 polypeptide comprises a VL CDR3 having the amino acid sequence of SEQ ID NO.:6, SEQ ID NO.:20, SEQ ID NO.:63 or SEQ ID NO.:64. In another embodiment, an antibody of that immunospecifically binds to an IL-9 polypeptide comprises a VL CDR1 having the amino acid sequence of SEQ ID NO.:4, SEQ ID NO.:13 or SEQ ID NO.:62 and a VL CDR2 having the amino acid sequence of SEQ ID NO.:5, SEQ ID NO.:14 or SEQ ID NO.:65. In another embodiment of an antibody that immunospecifically binds to an IL-9 polypeptide comprises a VL CDR1 having the amino acid sequence of SEQ ID NO.:4, SEQ ID NO.:13 or SEQ ID NO.:62 and a VL CDR3 having the amino acid sequence of SEQ ID NO.:6, SEQ ID NO.:20, SEQ ID NO.:63 or SEQ ID NO.:64. In another embodiment, an antibody that immunospecifically binds to an IL-9 polypeptide comprises a VL CDR2 having the amino acid sequence of SEQ ID NO.:5, SEQ ID NO.:14 or SEQ ID NO.:65 and a VL CDR3 having the amino acid sequence of SEQ ID NO.:6, SEQ ID NO.:20, SEQ ID NO.:63 or SEQ ID NO.:64. In another embodiment, an antibody that immunospecifically binds to an IL-9 polypeptide comprises a VL CDR1 having the amino acid sequence of SEQ ID NO.:4, SEQ ID NO.:13 or SEQ ID NO.:62, a VL CDR2 having the amino acid sequence of SEQ ID NO.:5, SEQ ID NO.:14 or SEQ ID NO.:65, and a VL CDR3 having the amino acid sequence of SEQ ID NO.:6, SEQ ID NO.:20, SEQ ID NO.:63 or SEQ ID NO.:64, being a part of the antibody.

The present invention provides formulations of antibodies that immunospecifically bind to an IL-9 polypeptide, said antibodies comprising a VH domain disclosed herein combined with a VL domain disclosed herein, or other VL domain (e.g., a VL domain disclosed in U.S. patent application Ser. No. 10/412,703, filed Apr. 12, 2002, and published as U.S. Pat. Pub. No. US 2003/0219439 A1, which is incorporated herein by reference in its entirety). The present invention also provides antibodies that immunospecifically bind to an IL-9 polypeptide, said antibodies comprising a VL domain disclosed herein combined with a VH domain disclosed herein, or other VH domain (e.g., a VH domain disclosed in U.S. patent application Ser. No. 10/412,703, filed Apr. 12, 2002, and published as U.S. Pat. Pub. No. US 2003/0219439 A1).

The present invention provides formulations of antibodies that immunospecifically bind to an IL-9 polypeptide, said antibodies comprising (or alternatively, consisting of) a said antibodies comprising (or alternatively, consisting of) a VH CDR listed in Table 1, supra and a VL CDR disclosed in U.S. patent application Ser. No. 10/412,703, filed Apr. 12, 2002, and published as U.S. Pat. Pub. No. US 2003/0219439 A1. The present invention also provides antibodies that immunospecifically bind to an IL-9 polypeptide, said antibodies comprising (or alternatively, consisting of) a VL CDR listed in Table 1, supra and a VH CDR disclosed in U.S. patent application Ser. No. 10/412,703, filed Apr. 12, 2002, and published as U.S. Pat. Pub. No. US 2003/0219439 A1. The invention further provides antibodies that immunospecifically bind to an IL-9 polypeptide, said antibodies comprising combinations of VH CDRs and VL CDRs described herein and disclosed in U.S. patent application Ser. No. 10/412,703, filed Apr. 12, 2002, and published as U.S. Pat. Pub. No. US 2003/0219439 A1.

The present invention provides formulations of antibodies that immunospecifically bind to an IL-9 polypeptide, said antibodies comprising one or more VH CDRs and one or more VL CDRs listed in Table 1, supra. In particular, the invention provides an antibody that immunospecifically binds to an IL-9 polypeptide, said antibody comprising (or alternatively, consisting of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 1, supra.

In one embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR1 having the amino acid sequence of SEQ ID NO.:4, SEQ ID NO.:13 or SEQ ID NO.:62. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR2 having the amino acid sequence of SEQ ID NO.:5, SEQ ID NO.:14 or SEQ ID NO.:65. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR3 having an amino acid sequence of SEQ ID NO.:6, SEQ ID NO.:20, SEQ ID NO.:63 or SEQ ID NO.:64.

In one embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR1 having the amino acid sequence of SEQ ID NO.:4, SEQ ID NO.:13 or SEQ ID NO.:62. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR2 having the amino acid sequence of SEQ ID NO.:5, SEQ ID NO.:14 or SEQ ID NO.:65. In another embodiment, an antibody that immunospecifically binds to an IL-9 polypeptide comprises a VH CDR1 having the amino acid sequence of SEQ ID NO.:1, SEQ ID NO.:11, SEQ ID NO.:19, or SEQ ID NO.:26 and a VL CDR3 having an amino acid sequence of SEQ ID NO.:6, SEQ ID NO.:20, SEQ ID NO.:63 or SEQ ID NO.:64.

In one embodiment, an antibody that immunospecifically binds to an IL-9 polypeptide comprises a VH CDR3 having the amino acid sequence of SEQ ID NO.:3 or SEQ ID NO.:12 and a VL CDR1 having the amino acid sequence of SEQ ID NO.:4, SEQ ID NO.:13 or SEQ ID NO.:62. In another embodiment, an antibody that immunospecifically binds to an IL-9 polypeptide comprises a VH CDR3 having the amino acid sequence of SEQ ID NO.:3 or SEQ ID NO.:12 and a VL CDR2 having the amino acid sequence of SEQ ID NO.:5, SEQ ID NO.:14 or SEQ ID NO.:65. In another embodiment, an antibody that immunospecifically binds to an IL-9 polypeptide comprises a VH CDR3 having the amino acid sequence of SEQ ID NO.:3 or SEQ ID NO.:12 and a VL CDR3 having an amino acid sequence of SEQ ID NO.:6, SEQ ID NO.:20, SEQ ID NO.:3 or SEQ ID NO.:64.

The present invention provides formulations of antibodies that immunospecifically bind to an IL-9 polypeptide, said antibodies comprising derivatives of the VH domains, VH CDRs, VL domains, or VL CDRs described herein that immunospecifically bind to an IL-9 polypeptide. Standard techniques known to those of skill in the art can be used to introduce mutations (e.g., deletions, additions, and/or substitutions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In one embodiment, the derivatives have conservative amino acid substitutions are made at one or more predicted non-essential amino acid residues (i.e., amino acid residues which are not critical for the antibody to immunospecifically bind to an IL-9 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

In specific embodiments, the present invention provides for formulations of antibodies that immunospecifically bind to an IL-9 polypeptide, said antibodies comprising the amino acid sequence of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 with one or more amino acid residue substitutions in the variable light (VL) domain and/or variable heavy (VH) domain. The present invention also provides for antibodies that immunospecifically bind to an IL-9 polypeptide, said antibodies comprising the amino acid sequence of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 with one or more amino acid residue substitutions in one or more VL CDRs and/or one or more VH CDRs. The present invention also provides for antibodies that immunospecifically bind to an IL-9 polypeptide, said antibodies comprising the amino acid sequence of 4D4, 4D4 H2-1 D11, 4D4com-XF-9, 4D4com-2F9, 7F3, 71A10, 7F3 22D3, 7F3com-2H2, 7F3com-3H5, or 7F3com-3D4, or a VH and/or VL domain thereof with one or more amino acid residue substitutions in one or more VH frameworks and/or one or more VL frameworks. The antibody generated by introducing substitutions in the VH domain, VH CDRs, VL domain VL CDRs and/or frameworks of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 can be tested in vitro and/or in vivo, for example, for its ability to bind to an IL-9 polypeptide, or for its ability to inhibit or reduce IL-9 mediated cell proliferation, or for its ability to prevent, treat and/or manage an autoimmune disorder, an inflammatory disorder, a proliferative disorder or a respiratory infection, or a symptom thereof

In one embodiment, an antibody that immunospecifically binds to an IL-9 polypeptide comprises a nucleotide sequence that hybridizes to the nucleotide sequence encoding 4D4, 4D4 H2-1 D11, 4D4com-XF-9, 4D4com-2F9, 7F3, 71A10, 7F3 22D3, 7F3com-2H2, 7F3com-3H5 or 7F3com-3D4, or an antigen-binding fragment thereof under stringent conditions, e.g., hybridization to filter-bound DNA in 6x sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In another embodiment, an antibody that immunospecifically binds to an IL-9 polypeptide comprises an amino acid sequence of a VH domain or an amino acid sequence a VL domain encoded by a nucleotide sequence that hybridizes to the nucleotide sequence encoding the VH or VL domains of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 under stringent conditions described herein or under other stringent hybridization conditions which are known to those of skill in the art. In another embodiment, an antibody that immunospecifically binds to an IL-9 polypeptide comprises an amino acid sequence of a VH domain and an amino acid sequence of a VL domain encoded by a nucleotide sequence that hybridizes to the nucleotide sequence encoding the VH and VL domains of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 under stringent conditions described herein or under other stringent hybridization conditions which are known to those of skill in the art. In another embodiment, an antibody that immunospecifically binds to an IL-9 polypeptide comprises an amino acid sequence of a VH CDR or an amino acid sequence of a VL CDR encoded by a nucleotide sequence that hybridizes to the nucleotide sequence encoding any one of the VH CDRs or VL CDRs listed in Table 1, supra under stringent conditions described herein or under other stringent hybridization conditions which are known to those of skill in the art. In another embodiment, an antibody that immunospecifically binds to an IL-9 polypeptide comprises an amino acid sequence of a VH CDR and an amino acid sequence of a VL CDR encoded by nucleotide sequences that hybridize to the nucleotide sequences encoding any one of the VH CDRs listed in Table 1, supra, and any one of the VL CDRs listed Table 1, supra, under stringent conditions described herein or under other stringent hybridization conditions which are known to those of skill in the art.

In a specific embodiment, an antibody that immunospecifically binds to an IL-9 polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, or an antigen-binding fragment thereof. In another embodiment, an antibody that immunospecifically binds to an IL-9 polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4. In another embodiment, an antibody that immunospecifically binds to an IL-9 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4.

In another embodiment, an antibody that immunospecifically binds to an IL-9 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1, supra. In another embodiment, an antibody that immunospecifically binds to an IL-9 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VL CDRs listed in Table 1, supra.

The present invention encompasses formulations of antibodies that compete with an antibody described herein for binding to an IL-9 polypeptide. In particular, the present invention encompasses antibodies that compete with 4D4, 4D4 H2-1 D11, 4D4com-XF-9, 4D4com-2F9, 7F3, 71A10, 7F3 22D3, 7F3com-2H2, 7F3com-3H5 or 7F3com-3D4 or an antigen-binding fragment thereof for binding to the IL-9 polypeptide. In a specific embodiment, the invention encompasses an antibody that reduces the binding of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 to an IL-9 polypeptide by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 5 to 15%, 10 to 25%, 25% to 50%, 45 to 75%, or 75 to 99% relative to a control such as PBS in the competition assay described herein or competition assays well known in the art. In another embodiment, the invention encompasses formulations of an antibody that reduces binding of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 to an IL-9 polypeptide by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 5 to 15%, 10 to 25%, 25% to 50%, 45 to 75%, or 75 to 99% relative to a control such as PBS in an ELISA competition assay. In one embodiment, an ELISA competition assay may be performed in the following manner: recombinant IL-9 is prepared in PBS at a concentration of 10 μg/ml. 100 μl of this solution is added to each well of an ELISA 98-well microtiter plate and incubated overnight at 4-8° C. The ELISA plate is washed with PBS supplemented with 0.1% Tween to remove excess recombinant IL-9. Non-specific protein-protein interactions are blocked by adding 100 μl of bovine serum albumin (BSA) prepared in PBS to a final concentration of 1%. After one hour at room temperature, the ELISA plate is washed. Unlabeled competing antibodies are prepared in blocking solution at concentrations ranging from 1 μg/ml to 0.01 μg/ml. Control wells contain either blocking solution only or control antibodies at concentrations ranging from 1 μg/ml to 0.01 μg/ml. Test antibody (e.g., 7F3com-2H2) labeled with horseradish peroxidase is added to competing antibody dilutions at a fixed final concentration of 1 μg/ml. 100 μl of test and competing antibody mixtures are added to the ELISA wells in triplicate and the plate is incubated for 1 hour at room temperature. Residual unbound antibody is washed away. Bound test antibody is detected by adding 100 μl of horseradish peroxidase substrate to each well. The plate is incubated for 30 min. at room temperature, and absorbance is read using an automated plate reader. The average of triplicate wells is calculated. Antibodies which compete well with the test antibody reduce the measured absorbance compared with control wells. In another preferred embodiment, the invention encompasses an antibody that reduces the binding of 7F3com-2H2 to an IL-9 polypeptide by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 5 to 15%, 10 to 25%, 25% to 50%, 45 to 75%, or 75 to 99% relative to a control such as PBS in an ELISA competition assay (described above).

In another embodiment, the invention encompasses formulations of an antibody that reduces the binding of an antibody comprising (alternatively, consisting of) an antigen-binding fragment (e.g., a VH domain, a VH CDR, a VL domain or a VL CDR) of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 to an IL-9 polypeptide by at least 2%, at least 5%, at least 10%, at least 15%, at least 20%, at least 25% , at least 30%, at least 35%, at least 40%, at least 45%, at least 50%, at least 55%, at least 60%, at least 65%, at least 70%, at least 75%, at least 80%, at least 85%, at least 90%, at least 95% or more, or 5 to 15%, 10 to 25%, 25% to 50%, 45 to 75%, or 75 to 99% relative to a control such as PBS in a competition assay described herein or well-known to one of skill in the art. In another embodiment, the invention encompasses an antibody that reduces the binding of an antibody comprising (alternatively, consisting of) an antigen-binding fragment (e.g., a VH domain, VL domain, a VH CDR, or a VL CDR) of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 to an IL-9 polypeptide by at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 5% to 15%, 10% to 25%, 25% to 50%, 45% to 75%, or 75% to 99% relative to a control such as PBS in an ELISA competition assay. In one embodiment, the invention encompasses an antibody that reduces the binding of an antibody comprising (alternatively, consisting of) an antigen-binding fragment of 7F3com-2H2 to an IL-9 polypeptide by at least 2%, at least 5%, at least 10%, at least 15%, at least 20%, at least 25% , at least 30%, at least 35%, at least 40%, at least 45%, at least 50%, at least 55%, at least 60%, at least 65%, at least 70%, at least 75%, at least 80%, at least 85%, at least 90%, at least 95% or more, or 5 to 15%, 10 to 25%, 25% to 50%, 45 to 75%, or 75 to 99% relative to a control such as PBS in an ELISA competition assay.

The present invention encompasses formulations of polypeptides or proteins comprising (alternatively, consisting of) VH domains that compete with the VH domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 for binding to an IL-9 polypeptide. The present invention also encompasses formulations of polypeptides or proteins comprising (alternatively, consisting of) VL domains that compete with a VL domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 for binding to an IL-9 polypeptide.

The present invention encompasses formulations of polypeptides or proteins comprising (alternatively, consisting of) VH CDRs that compete with a VH CDR listed in Table 1, supra, for binding to an IL-9 polypeptide. The present invention also encompasses polypeptides or proteins comprising (alternatively consisting of) VL CDRs that compete with a VL CDR listed in Table 1, supra, for binding to an IL-9 polypeptide.

The antibodies that immunospecifically bind to an IL-9 polypeptide include derivatives that are modified, i.e., by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides formulations of antibodies that immunospecifically bind to an IL-9 polypeptide, said antibodies comprising a framework region known to those of skill in the art (e.g., a human or non-human framework). The framework regions may be naturally occurring or consensus framework regions. The fragment region of an antibody of the invention may be human (see, e.g., Chothia et al., 1998, J. Mol. Biol. 278:457-479 for a listing of human framework regions, which is incorporated herein by reference in its entirety).

The present invention encompasses formulations of antibodies that immunospecifically bind to an IL-9 polypeptide, said antibodies comprising the amino acid sequence of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 with mutations (e.g., one or more amino acid substitutions) in the framework regions. In certain embodiments, antibodies that immunospecifically bind to an IL-9 polypeptide comprise the amino acid sequence of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 with one or more amino acid residue substitutions in the framework regions of the VH and/or VL domains. The amino acid substitutions in the framework region may improve binding of the antibody to an IL-9 polypeptide.

In a specific embodiment, formulations of antibodies that immunospecifically bind to an IL-9 polypeptide comprise the amino acid sequence of one or more of the CDRs of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, a VH framework region 1 having the amino acid sequence of QVQLVQSGAEVKKPGASVKVSCKAS (SEQ ID NO.: 33) or QVQLVQSGAEVKKPGSSVKVSCKAS (SEQ ID NO.: 37), a VH framework region 2 having the amino acid sequence of WVRQAPGQGLEWMG (SEQ ID NO.: 34), a VH framework region 3 region having the amino acid sequence of RVTMTRDTSTSTVYMELSSLRSEDTAVYYCAR (SEQ ID NO.: 35) or RVTITADESTSTAYMELSSLRSEDTAVYYCAR (SEQ ID NO.: 38), and a VH framework region 4 having the amino acid sequence of WGQGTLVTVSS (SEQ ID NO.: 36). In another embodiment, antibodies that immunospecifically bind to an IL-9 polypeptide comprise the amino acid sequence of one or more of the CDRs of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, a VL framework region 1 having the amino acid sequence of DIQMTQSPSSLSASVGDRVTITC (SEQ ID NO.: 39), a VL framework region 2 having the amino acid sequence of WYQQKPGKAPKLLIY (SEQ ID NO.: 40), a VL framework region 3 region having the amino acid sequence of GVPSRFSGSGSGTDFTLTISSLQ PEDFATYYC (SEQ ID NO.: 41), and a VL framework region 4 region having the amino acid sequence of FGGGTKVEIK (SEQ ID NO.: 42). In yet another embodiment, antibodies that immunospecifically bind to an IL-9 polypeptide comprise the amino acid sequence of one or more of the CDRs of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, a VH framework region 1 having the amino acid sequence of SEQ ID NO.: 33 or SEQ ID NO.: 37, a VH framework region 2 having the amino acid sequence of SEQ ID NO.: 34, a VH framework region 3 having the amino acid sequence of SEQ ID NO.: 35 or SEQ ID NO.: 38, a VH framework region 4 having the amino acid sequence of SEQ ID NO.: 36, a VL framework region 1 having the amino acid sequence of SEQ ID NO.: 39, a VL framework region 2 having the amino acid sequence of SEQ ID NO.: 40, a VL framework region 3 having the amino acid sequence of SEQ ID NO.: 41, and a VL framework region 4 having the amino acid sequence of SEQ ID NO.: 42.

The present invention also encompasses formulations of antibodies that immunospecifically bind to an IL-9 polypeptide, said antibodies comprising the amino acid sequence of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 with mutations (e.g., one or more amino acid residue substitutions) in the variable and framework regions. The amino acid substitutions in the variable and framework regions may improve binding of the antibody to an IL-9 polypeptide.

The present invention also provides formulations of antibodies of the invention that comprise constant regions known to those of skill in the art. The constant regions of an antibody of the invention or fragment thereof may be human.

The invention encompasses formulations of antibodies that immunospecifically bind to an IL-9 polypeptide expressed by an immune cell such as an activated T cell or a mast cell. The invention also encompasses antibodies that immunospecifically bind to an IL-9 polypeptide and modulate an activity or function of T cells, B cells, mast cells, neutrophils, and/or eosinophils. The invention further encompasses antibodies that immunospecifically bind to an IL-9 polypeptide and inhibit or reduce the infiltration of inflammatory cells into a tissue, joint, or organ of a subject and/or inhibit or reduce epithelial cell hyperplasia.

The invention encompasses formulations of antibodies that immunospecifically bind to an IL-9 polypeptide found in the milieu, i.e., not bound to an IL-9R or a subunit thereof. The invention also encompasses antibodies that immunospecifically bind to an IL-9 polypeptide bound to a soluble IL-9Rα subunit. The invention further encompasses antibodies that immunospecifically bind to an IL-9 polypeptide bound to a cellular membrane-bound IL-9R or a subunit thereof

In one embodiment, an antibody that immunospecifically binds to an IL-9 polypeptide inhibits and/or reduces the interaction between the IL-9 polypeptide and the IL-9R or a subunit thereof by approximately 25%, approximately 30%, approximately 35%, approximately 45%, approximately 50%, approximately 55%, approximately 60%, approximately 65%, approximately 70%, approximately 75%, approximately 80%, approximately 85%, approximately 90%, approximately 95%, or approximately 98% relative to a control such as PBS or an IgG control antibody in an in vivo and/or in vitro assay described herein or well-known to one of skill in the art (e.g., an immunoassay such as an ELISA). In an alternative embodiment, an antibody that immunospecifically binds to an IL-9 polypeptide does not inhibit the interaction between an IL-9 polypeptide and the IL-9R or a subunit thereof relative to a control such as PBS or an IgG control antibody in an in vivo and/or in vitro assay described herein or well-known to one of skill in the art (e.g., an immunoassay such as an ELISA). In another embodiment, an antibody that immunospecifically binds to an IL-9 polypeptide inhibits the interaction between the IL-9 polypeptide and the IL-9R by less than 20%, less than 15%, less than 10%, or less than 5% relative to a control such as PBS or an IgG control antibody using, for example, an immunoassay such as an ELISA.

In one embodiment, antibodies that immunospecifically bind to an IL-9 polypeptide inhibit or reduce the interaction between the IL-9 polypeptide and the IL-9R or one or more subunits thereof by at least 25%, at least 30%, at least 35%, at least 45%, at least 50%, at least 55%, at least 60%, at least 65%, at least 70%, at least 75%, at least 80%, at least 85%, at least 90%, at least 95%, or at least 98% relative to a control such as phosphate buffered saline (“PBS”) or an IgG control antibody in an in vivo and/or in vitro assay described herein or well-known to one of skill in the art (e.g., a cell proliferation assay using an IL-9 dependent cell line such as an IL-9 dependent mouse T cell line expressing the human IL-9R). In an alternative embodiment, antibodies that immunospecifically bind to an IL-9 polypeptide do not inhibit the interaction between an IL-9 polypeptide and the IL-9R or one or more subunits thereof relative to a control such as PBS or an IgG control antibody in an in vivo and/or in vitro assay described herein or well-known to one of skill in the art (e.g., a cell proliferation assay using an IL-9 dependent cell line such as an IL-9 dependent mouse T cell line expressing the human IL-9R). In another embodiment, antibodies that immunospecifically bind to an IL-9 polypeptide inhibit the interaction between the IL-9 polypeptide and the IL-9R or one or more subunits thereof by less than 20%, less than 15%, less than 10%, or less than 5% relative to a control such as PBS or an IgG control antibody in vivo and/or in vitro assay described herein or well-known to one of skill in the art, (e.g., a cell proliferation assay using an IL-9 dependent cell line such as an IL-9 dependent mouse T cell line expressing the human IL-9R).

The present invention encompasses formulations of antibodies that immunospecifically bind to an IL-9 polypeptide and do not induce or reduce cytokine expression and/or release relative to a control such as PBS or an IgG control antibody in an in vivo and/or in vitro assay described herein or well-known to one of skill in the art. In one embodiment, antibodies that immunospecifically bind to an IL-9 polypeptide and do not induce an increase in the concentration cytokines such as, e.g., IFN-γ, IL-2, IL-4, IL-5, IL-6, IL-7, IL-10, IL-12, IL-15, and IL-23 in the serum of a subject administered such an antibody relative to the concentration of such cytokines in the serum of a subject administered a ctrol such as PBS or an IgG control antibody. In an alternative embodiment, antibodies that immunospecifically bind to an IL-9 polypeptide induce cytokine expression and/or release relative to a control such as PBS or an IgG control antibody in an in vitro and/or in vivo assay described herein or well-known to one of skill in the art. In a specific embodiment, an antibody that immunospecifically binds to an IL-9 polypeptide induces an increase in the concentration of cytokines such as, e.g., IFN-γ, IL-2, IL-12, and IL-15 in the serum of a subject administered such an antibody relative to the concentration of such cytokines in the serum of a subject administered a control such as PBS or an IgG control antibody. In another specific embodiment, an antibody that immunospecifically binds to an IL-9 polypeptide induces an increase in the concentration of cytokines produced by Th1 cells, such as IFN-γ and IL-12, in a subject administered such an antibody relative to the concentration of such cytokines in the serum of a subject administered a control such as PBS or an IgG control antibody. In another specific embodiment, an antibody that immunospecifically binds to an IL-9 polypeptide induces a decrease in the concentration of cytokines such as, e.g., IL-4, IL-5, IL-10, IL-13, and IL-23 in the serum of a subject administered such an antibody relative to the concentration of such cytokines in the serum of a subject administered a control such as PBS or an IgG control antibody.. In another specific embodiment, an antibody that immunospecifically binds to an IL-9 polypeptide induces a decrease in the concentration of cytokines produced by mast cells, such as TNF-α, IL-4, and IL-13, in the serum of a subject administered such an antibody relative to the concentration of such cytokines in the serum of a subject administered a control such as PBS or an IgG control antibody. In yet another specific embodiment, an antibody that immunospecifically binds to an IL-9 polypeptide induces a decrease in the concentration of cytokines produced by Th2 cells, such as IL-4, IL-5, IL-13, and IL-10, in the serum of a subject administered such an antibody relative to the concentration of such cytokines in the serum of a subject administered a control such as PBS or an IgG control antibody. Serum concentrations of a cytokine can be measured by any technique well-known to one of skill in the art such as, e.g., ELISA or Western blot assay.

In one embodiment, antibodies that immunospecifically bind to an IL-9 polypeptide reduce and/or inhibit proliferation of inflammatory cells (e.g., mast cells, T cells, B cells, macrophages, neutrophils, basophils, and/or eosinophils) by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art (e.g., a trypan blue assay or ³H-thymidine assay). In another embodiment, antibodies that immunospecifically bind to an IL-9 polypeptide reduce and/or inhibit infiltration of inflammatory cells into the upper and/or lower respiratory tracts by at least at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art. In yet another embodiment, antibodies that immunospecifically bind to an IL-9 polypeptide reduce and/or inhibit infiltration of inflammatory cells into the upper and/or respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well known in the art and reduce and/or inhibit proliferation of inflammatory cells by at least by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art (e.g., a trypan blue assay or ³H-thymidine assay).

In certain embodiments, antibodies that immunospecifically bind to an IL-9 polypeptide reduce mast cell degranulation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art (see, e.g., Windmiller and Backer, 2003, J. Biol. Chem. 278:11874-78 for examples of mast cell degranulation assays). In other embodiments, antibodies that immunospecifically bind to an IL-9 polypeptide inhibit and/or reduce mast cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art. In other embodiments, antibodies that immunospecifically bind to an IL-9 polypeptide inhibit and/or reduce the expression and/or release of products of mast cell activation and/or degranulation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or an IgG control antibody in an in vivo and/or in vitro assay described herein or well-known to one of skill in the art.

In a specific embodiment, antibodies that immunospecifically bind to an IL-9 polypeptide inhibit and/or reduce the expression, activity, serum concentration, and/or release of mast cell proteases, such as chymase and tryptase, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art. In one embodiment, mast cell activity may be measured by culturing primary mast cells or a mast cell line in vitro in the presence of 10 ng/ml of IL-9. Baseline levels of protease (e.g., chymase and tryptase) and leukotriene are determined in the supernatant by commercially available ELISA kits. The ability of antibodies to modulate protease or leukotriene levels is assessed by adding an IL-9-reactive antibody or control antibody directly to cell cultures at a concentration of 1 μg/ml. Protease and leukotriene levels are assessed at 24 and 36 hour timepoints. In another specific embodiment, antibodies that immunospecifically bind to an IL-9 polypeptide inhibit and/or reduce the expression, activity, serum concentration, and/or release of mast cell leukotrienes, such as C4, D4, and E4 by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. In another specific embodiment, antibodies that immunospecifically bind to an IL-9 polypeptide inhibit and/or reduce the expression, activity, serum concentration, and/or release of mast cell cytokines, such as TNF-α, IL-4, and IL-13 by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., an ELISA or Western blot assay).

In other embodiments, antibodies that immunospecifically bind to an IL-9 polypeptide inhibit and/or reduce mast cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known in the art. In other embodiments, antibodies that immunospecifically bind to an IL-9 polypeptide inhibit and/or reduce mast cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In yet other embodiments, antibodies that immunospecifically bind to an IL-9 polypeptide inhibit and/or reduce mast cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vitro and/or in vivo assay described herein or well-known in the art and inhibit and/or reduce mast cell proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In one embodiment, reductions in mast cell infiltration may be measured in vivo by sensitizing animals to ovalbumin. Briefly, 100 μg of ovalbumin complexed with aluminum adjuvant is administered subcutaneously on days 1 and 21. Throughout the three-week sensitization procedure, animals are administered an IL-9 reactive antibody or a control antibody at a 10 mg/kg dose every 5 to 7 days. On days 29, 30 and 31, animals are exposed to ovalbumin without adjuvant by aerosol delivery, or alternatively, by intrasal instillation of 100 μl of a 1 μg/ml solution prepared in PBS. On day 31, 6 hours after the last ovalbumin challenge, animals are euthanized and lung tissue is fixed by perfusion with formalin. Mast cell infiltration is assessed histologically by counting mast cells per field in lung epithelial tissue sections. Using this experimental design, mast cell precursors may be differentiated from mast cells in lung epithelium by assessing (for example) whether metachromatic granules are present, and/or by immunohistochemistry using differentiation-dependent cell surface markers (e.g., FcepsilonRI).

In other embodiments, antibodies that immunospecifically bind to an IL-9 polypeptide inhibit and/or reduce infiltration of mast cell precursors in the upper and/or lower respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known in the art. In other embodiments, antibodies that immunospecifically bind to an IL-9 polypeptide inhibit and/or reduce proliferation of mast cell precursors by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In yet other embodiments, antibodies that immunospecifically bind to an IL-9 polypeptide inhibit and/or reduce infiltration of mast cell precursors into the upper and/or lower respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known in the art and inhibit and/or reduce proliferation of mast cell precursors at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In one embodiment, mast cell precursor infiltration may be measured in vivo by the mast cell infiltration assay described supra.

In certain embodiments, antibodies that immunospecifically bind to an IL-9 polypeptide mediate depletion of peripheral blood T-cells by inducing an increase in apoptosis of T-cells, particularly Th2 cells. In one embodiment, Th2 T lymphocyte depletion may be measured in vivo by sensitizing animals with ovalbumin. Briefly, 100 μg of ovalbumin complexed with aluminum adjuvant is administered subcutaneously on days 1 and 21. Throughout the three-week sensitization procedure, animals are administered an IL-9 reactive antibody or a control antibody at a 10 mg/kg dose every 5 to 7 days. On day 28, animals receive a 100 μg boost of ovalbumin protein without adjuvant intravenously. Two days following the intravenous boost, the animals are euthanized. Spleen cells are recovered and analyzed by flow cytometry. Splenic Th2 T lymphocytes, identifiable by cytoplasmic staining for IL-4, should be reduced in animals receiving an IL-9 neutralizing antibody compared with the control antibody recipients. In another embodiment, antibodies that immunospecifically bind to an IL-9 polypeptide mediate inhibit and/or reduce Thl and Th2 differentiation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., FACS). In certain embodiments, antibodies that immunospecifically bind to an IL-9 polypeptide inhibit and/or reduce T cell infiltration, particularly Th2 cell infiltration, in the upper and/or lower respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay well-known to one of skill in the art. In other embodiments, antibodies that immunospecifically bind to an IL-9 polypeptide inhibits and/or reduce T cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In yet other embodiments, antibodies that immunospecifically bind to an IL-9 polypeptide inhibit and/or reduce T cell infiltration, particularly Th2 cell infiltration, in the upper and/or lower respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98%, inhibit and/or reduce T cell proliferation, particularly Th2 cell proliferation, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98%, and/or increases apoptosis of T cells relative to a control such as PBS or a control IgG antibody in an in vivo and/or in vitro assay described herein or well-known to one of skill in the art.

In certain embodiments, antibodies that immunospecifically bind to an IL-9 polypeptide reduce macrophage infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay well-known to one of skill in the art. In one embodiment, reductions in macrophage infiltration may be measured in vivo by sensitizing animals to ovalbumin. Briefly, 100 μg of ovalbumin complexed with aluminum adjuvant is administered subcutaneously on days 1 and 21. Throughout the three-week sensitization procedure, animals are administered IL-9 reactive antibody or control antibody at 10 mg/kg dose every 5 to 7 days. On days 29, 30 and 31, animals are exposed to ovalbumin without adjuvant by aerosol delivery, or alternatively, by intrasal instillation of 100 μl of a 1 μg/ml solution prepared in PBS. On day 31, 6 hours after the last ovalbumin challenge, animals are euthanized and lung tissue is fixed by perfusion with formalin. Macrophage infiltration is assessed by immunocytochemistry by counting CD14 positive cells per field in lung tissue sections. In other embodiments, antibodies that immunospecifically bind to an IL-9 polypeptide inhibit and/or reduce macrophage proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art (e.g., a trypan blue assay, FACS or ³H thymidine assay). In yet other embodiments, antibodies that immunospecifically bind to an IL-9 polypeptide inhibit and/or reduce macrophage infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art and inhibit and/or reduce macrophage proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art.

In certain embodiments, antibodies that immunospecifically bind to an IL-9 polypeptide reduce B cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art. In one embodiment, reductions in B lymphocyte infiltration may be measured in vivo by systemically sensitizing animals to ovalbumin. Briefly, 100 μg of ovalbumin complexed with aluminum adjuvant is administered subcutaneously on days 1 and 21. Throughout the three-week sensitization procedure, animals are administered an IL-9 reactive antibody or a control antibody at a 10 mg/kg dose every 5 to 7 days. On days 29, 30 and 31, animals are exposed to ovalbumin without adjuvant by aerosol delivery, or alternatively, by intrasal instillation of 100 μl of a 1 μg/ml solution prepared in PBS. On day 31, 6 hours after the last ovalbumin challenge, animals are euthanized and lung tissue is fixed by perfusion with formalin. B lymphocyte infiltration is assessed by immunocytochemistry by counting CD19 positive cells per field in lung tissue sections. In other embodiments, antibodies that immunospecifically bind to an IL-9 polypeptide inhibit and/or reduce B cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well- known to one of skill in the art (e.g., a trypan blue assay, FACS or ³H thymidine assay). In yet other embodiments, antibodies that immunospecifically bind to an IL-9 polypeptide inhibit and/or reduce B cell infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art and inhibits and/or reduces B cell proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art.

In certain embodiments, antibodies that immunospecifically bind to an IL-9 polypeptide reduce eosinophil infiltration in the upper and/or lower respiratory tracts by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art (see, e.g., Li et al., 2000, Am. J. Respir. Cell Mol. Biol. 25:644-51). In other embodiments, antibodies that immunospecifically bind to an IL-9 polypeptide inhibit and/or reduce eosinophil proliferation, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein (see Section 5.6) or well known to one of skill in the art (e.g., a trypan blue assay, FACS or ³H thymidine assay). In yet other embodiments, antibodies that immunospecifically bind to an IL-9 polypeptide inhibit and/or reduce eosinophil infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art and inhibits and/or reduces eosinophil proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art.

In other embodiments, antibodies that immunospecifically bind to an IL-9 polypeptide reduce neutrophil infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well known to one of skill in the art. In other embodiments, antibodies that immunospecifically bind to an IL-9 polypeptide inhibit and/or reduce neutrophil proliferation, by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assays described herein or well-known to one of skill in the art (e.g., a trypan blue assay, FACS or ³H thymidine assay). In yet other embodiments, antibodies that immunospecifically bind to an IL-9 polypeptide inhibit and/or reduce neutrophil infiltration by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art and inhibits and/or reduces neutrophil proliferation at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art.

In one embodiment, an antibody that immunospecifically binds to an IL-9 polypeptide neutralizes or inhibits IL-9 mediated biological effects including, but not limited to inflammatory cell recruitment, epithelia hyperplasia, mucin production of epithelial cells, and mast cell activation, degranulation, proliferation, and/or infiltration.

In a specific embodiment, an antibody that immunospecifically binds to an IL-9 polypeptide acts synergistically with a proteinaceous agent (e.g., a peptide, polypeptide, or protein (including an antibody)) and/or a non-proteinaceous agent that antagonizes the expression, function, and/or activity of IgE to reduce or inhibit the activation, degranulation, proliferation, and/or infiltration of mast cells by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assays described herein or well known to one of skill in the art.

In another embodiment, an antibody that immunospecifically binds to an IL-9 polypeptide acts synergistically with a proteinaceous agent (e.g., a peptide, polypeptide, protein (including an antibody)) and/or a non-proteinaceous agent that antagonizes the expression, function, and/or activity of a mast cell protease to reduce or inhibit the activation, degranulation, proliferation, and/or infiltration of mast cells by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art.

In another embodiment, an antibody that immunospecifically binds to an IL-9 polypeptide acts synergistically with a proteinaceous agent (e.g., a peptide, polypeptide, and protein (including an antibody)) or a non-proteinaceous agent that antagonizes the expression, function, and/or activity of a stem cell factor to reduce or inhibit to reduce or inhibit the activation, degranulation, proliferation, and/or infiltration of mast cells by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay described herein or well-known to one of skill in the art. In one embodiment, primary mast cells or a mast cell line is cultured in vitro in the presence of 1 ng/ml IL-9 plus 1 ng/ml stem cell factor. Baseline levels of protease (e.g., chymase and tryptase) and leukotriene are determined in the supernatant by commercially available ELISA kits. The ability of antibodies to modulate protease or leukotriene levels is assessed by adding IL-9 reactive antibody or control antibody directly to cell cultures at a concentration of 1 μg/ml. Protease and leukotriene levels are assessed at 24 and 36 hour time points.

The formulations of antibodies of the present invention that immunospecifically bind to an IL-9 polypeptide may be monospecific, bispecific, trispecific or of greater multispecificity. Multispecific antibodies may be specific for different epitopes of an IL-9 polypeptide or may be specific for both an IL-9 polypeptide as well as for a heterologous epitope, such as a heterologous polypeptide or solid support material. See, e.g., International publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that have a high binding affinity for an IL-9 polypeptide. In a specific embodiment, an antibody that immunospecifically binds to an IL-9 polypeptide has an association rate constant or kₒₙ rate (antibody (Ab)+antigen

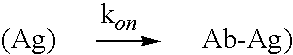

of at least 10⁵M⁻¹s⁻¹, at least 1.5×10⁵ M⁻¹s⁻1, at least 2×10⁵M⁻¹s⁻¹, at least 2.5×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹, or 10⁵-10⁸M⁻¹s⁻¹, 1.5×10⁵ M⁻¹s⁻¹-1×10⁷M⁻¹s⁻¹, 2×10⁵-1×10⁶M⁻¹s⁻¹, or 4.5×10⁵×10⁷M⁻¹s⁻¹. In one embodiment, an antibody that immunospecifically binds to an IL-9 polypeptide has a kₒₙ of at least 2×10⁵M⁻¹s⁻¹, at least 2.5×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻ⁱs⁻¹, at least 5×10⁷M⁻¹s⁻¹, or at least 10⁸M⁻¹s⁻¹ as determined by a BIAcore assay and the antibody neutralizes human IL-9 in the microneutralization assay as described herein. In one embodiment, an antibody that immunospecifically binds to an IL-9 polypeptide has a kₒₙ of at most 10⁸M⁻¹s⁻¹, at most 10⁹ M⁻¹s⁻¹, at most 10¹⁰M⁻¹s⁻¹, at most 10¹¹ M⁻¹s⁻¹, or at most 10¹²M⁻¹s⁻¹ as determined by a BIAcore assay and the antibody neutralizes human IL-9 in the microneutralization assay as described herein. In accordance with these embodiments, such antibodies may comprise a VH domain and/or a VL domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4 or a VH CDR and/or a VL CDR of 4D4, 4D4 H2-1 D11, 4D4com-XF-9, 4D4com-2F9, 7F3, 71A10, 7F3 22D3, 7F3com-2H2, 7F3com-3H5 or 7F3com-3D4.

In another embodiment, an antibody that immunospecifically binds to an IL-9 polypeptide has a k_{off} rate (antibody (Ab)+antigen

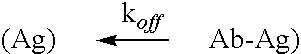

of less than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or 10⁻³-10⁻¹⁰ s⁻¹, 10⁻⁴-10⁻⁸ s⁻¹ or 10⁻⁵-10⁻⁸ s⁻¹. In one embodiment, an antibody that immunospecifically binds to an IL-9 polypeptide has a k_{off} of 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹ as determined by a BIAcore assay and the antibody neutralizes human IL-9 in the microneutralization assay described herein. In another preferred embodiment, an antibody that immunospecifically binds to an IL-9 polypeptide has a k_{off} of greater than 10⁻¹³ s⁻¹, greater than 10⁻¹² s⁻¹, greater than 10⁻¹¹s⁻¹, greater than 10⁻¹⁰ s⁻¹, greater than 10⁻⁹ s⁻¹, or greater than 10⁻⁸ s⁻¹. In accordance with these embodiments, such antibodies may comprise a VH domain and/or a VL domain of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, or a VH CDR and/or a VL CDR of 4D4, 4D4 H2-1 D11, 4D4com-XF-9, 4D4com-2F9, 7F3, 71A10, 7F3 22D3, 7F3com-2H2, 7F3com-3H5 or 7F3com-3D4.

In another embodiment, an antibody that immunospecifically binds to an IL-9 polypeptide has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or 10²-5×10⁵ M⁻¹, 10⁴-1×10¹⁰ M⁻¹, or 10⁵-1×10⁸ M⁻¹. In another embodiment, an antibody that immunospecificaly binds to an IL-9 polypeptide has a Kₐ of at most 10¹¹ M⁻¹, at most 5×10¹¹ M⁻¹, at most 10¹² M⁻¹, at most 5×10¹² M⁻¹, at most 10¹³ M⁻¹, at most 5×10¹³ M⁻¹, at most 10¹⁴ M⁻¹, or at most 5×10¹⁴ M⁻¹. In another embodiment, an antibody that immunospecifically binds to an IL-₉ polypeptide has a dissociation constant or K_{d} (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³M, less than 5×10⁻¹³ M, less than 10⁻¹⁴ M, less than 5×10⁻¹⁴ M, less than 10⁻¹⁵ M, or less than 5×10⁻¹⁵ M or 10⁻² M-5×10⁻⁵ M, 10⁻⁶-10⁻¹⁵ M, or 10⁻⁸-10⁻¹⁴ M. In one embodiment, an antibody that immunospecifically binds to an IL-9 polypeptide has a K_{d} of less than 10⁻⁹ M, less than 5×10⁻⁹ M, less than 10⁻¹⁰ M, less than 5×10⁻¹⁰ M, less than 1×10⁻¹¹ M, less than 5×10⁻¹¹ M, less than 1×10⁻¹² M, less than 5×10⁻¹² M, less than 10⁻¹³ M, less than 5×10⁻¹³ M or less than 1×10⁻¹⁴ M, or 10⁻⁹ M-10⁻¹⁴ M as determined by a BIAcore assay and the antibody neutralizes human IL-9 in the microneutralization assay described herein. In another preferred embodiment, an antibody that immunospecifically binds to an IL-9 polypeptide has a K_{d} of greater than 10⁻⁹ M, greater than 5×10⁻⁹ M, greater than 10⁻¹⁰ M^{,} greater than 5×10⁻¹⁰ M, greater than 10⁻¹¹ M, greater than 5×10⁻¹¹ M, greater than 10⁻¹² M, greater than 5×10⁻¹² M, greater than 6×10⁻¹² M, greater than 10⁻¹³ M, greater than 5×10⁻¹³ M, greater than 10⁻¹⁴ M, greater than 5×10⁻¹⁴ M or greater than 10⁻⁹ M-10⁻¹⁴ M. In accordance with these embodiments, such antibodies may comprise a VH domain and/or a VL domain of of 4D4, 4D4 H2-1 D11, 4D4com-XF-9, 4D4com-2F9, 7F3, 71A10, 7F3 22D3, 7F3com-2H2, 7F3com-3H5 or 7F3com-3D4, or a VH CDR and/or a VL CDR of 4D4, 4D4 H2-1 D11, 4D4com-XF-9, 4D4com-2F9, 7F3, 71A10, 7F3 22D3, 7F3com-2H2, 7F3com-3H5 or 7F3com-3D4.

In certain embodiments, formulations of the antibodies of the invention do not include antibodies known in the art that immunospecifically bind to an IL-9 polypeptide. Non-limiting examples of known antibodies that immunospecifically bind to an IL-9 polypeptide include 4D4, 4D4 H2-1 D11, 4D4com-XF-9, 4D4com-2F9, 7F3, 71A10, 7F3 22D3, 7F3com-2H2, 7F3com-3H5 or 7F3com-3D4.

In specific embodiments, formulations of antibodies of the invention bind antigenic epitope-bearing peptides and polypeptides of IL-9, and said antigenic epitope-bearing peptides and polypeptides comprise or consist of an amino acid sequence of at least 4, at least 5, at least 6, at least 7, at least 8, at least 9, at least 10, at least 11, at least 12, at least 13, at least 14, at least 15, at least 20, at least 25, at least 30, at least 40, at least 50 contiguous amino acid residues, or between about 15 to about 30 contiguous amino acids of IL-9 found in any species. Polypeptides comprising immunogenic or antigenic epitopes may be at least 8, at least 10, at least 15, at least 20, at least 25, at least at least 30, or at least 35 amino acid residues in length.

IL-9 epitope-bearing peptides, polypeptides, and fragments thereof may be produced by any conventional means. See, e.g., Houghten, R. A. (1985) “General method for the rapid solid-phase synthesis of large numbers of peptides: specificity of antigen-antibody interaction at the level of individual amino acids,” Proc. Natl. Acad. Sci. USA 82:5 13 1-5 135; this “Simultaneous Multiple. Peptide Synthesis (SMPS)” process is further described in U.S. Pat. No. 4,631,211 to Houghten et al. (1986).

The present invention provides formulations of peptides, polypeptides and/or proteins comprising one or more variable or hypervariable regions of the antibodies described herein. eptides, polypeptides or proteins comprising one or more variable or hypervariable regions of antibodies of the invention further comprise a heterologous amino acid sequence. In certain embodiments, such a heterologous amino acid sequence may comprise at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 75 contiguous amino acid residues, at least 100 contiguous amino acid residues or more contiguous amino acid residues. Such peptides, polypeptides and/or proteins may be referred to as fusion proteins.

In a specific embodiment, formulations of peptides, polypeptides or proteins comprising one or more variable or hypervariable regions of the antibodies of the invention are 10 amino acid residues, 15 amino acid residues, 20 amino acid residues, 25 amino acid residues, 30 amino acid residues, 35 amino acid residues, 40 amino acid residues, 45 amino acid residues, 50 amino acid residues, 75 amino acid residues, 100 amino acid residues, 125 amino acid residues, 150 amino acid residues or more amino acid residues in length. In certain embodiments, peptides, polypeptides, or proteins comprising one or more variable or hypervariable regions of an antibody of the invention immunospecifically bind to an IL-9 polypeptide. In other embodiments, peptides, polypeptides, or proteins comprising one or more variable or hypervariable regions of an antibody of the invention do not immunospecifically bind to an IL-9 polypeptide.

In a specific embodiment, the present invention provides formulations of peptides, polypeptides and/or proteins comprising a VH domain and/or VL domain of one of the antibodies described herein (see Table 1, supra). In one embodiment, the present invention provides peptides, polypeptides and/or proteins comprising one or more CDRs having the amino acid sequence of any of the CDRs listed in Table 1, supra. In accordance with these embodiments, the peptides, polypeptides or proteins may further comprise a heterologous amino acid sequence.

Peptides, polypeptides or proteins comprising one or more variable or hypervariable regions have utility, e.g., in the production of anti-idiotypic antibodies which in turn may be used to prevent, treat and/or manage one or more symptoms associated with a disease or disorder (e.g., an autoimmune disorder, an inflammatory disorder, a proliferative disorder or an infection (e.g., a respiratory infection)). The anti-idiotypic antibodies produced can also be utilized in immunoassays, such as, e.g., ELISAs, for the detection of antibodies which comprise a variable or hypervariable region contained in the peptide, polypeptide or protein used in the production of the anti-idiotypic antibodies.

5.1.1.2. Antibodies that Immunospecifically Bind to an RSV Antigen

The formulations of the present invention comprise an isolated antibody that immunospecifically binds to an RSV antigen. The antibodies can be monclonal antibodies, human antibodies, humanized antibodies or chimeric antibodies. In certain embodiments, the anti-RSV antigen antibody is palivizumab. Palivizumab is a humanized monoclonal antibody presently used for the prevention of RSV infection in pediatric patients. The present invention provides formulations of antibodies that immunospecifically bind to one or more RSV antigens. The antibodies useful in the invention may immunospecifically bind to one or more RSV antigens regardless of the strain of RSV. The present invention also provides antibodies that differentially or preferentially bind to RSV antigens from one strain of RSV versus another RSV strain. In one embodiment, the formulations comprise antibodies that immunospecifically bind to the RSV F glycoprotein, G glycoprotein or SH protein. In another embodiment, the formulations comprise antibodies that immunospecifically bind to the RSV F glycoprotein. In another embodiment, the formulations comprise antibodies that bind to the A, B, or C antigenic sites of the RSV F glycoprotein. Other RSV antibodies that can be formulated using the methods of the present invention are also disclosed in U.S. application Ser. No. 11/473,537, filed Jun. 23, 2006, entitled “Antibody Formulations Having Optimized Aggregation and Fragmentation Profiles,” which is herein incorporated by reference in its entirety.

5.1.1.3. Antibodies that Immunospecifically Bind to Human Metapneumovirus (hMPV)

The formulations of the present invention may comprise an isolated antibody that specifically binds to an antigen of human metapneumovirus (hMPV) and compositions comprising this antibody. The term “anti-hMPV-antigen antibody” refers to an antibody or antibody fragment thereof that binds immunospecifically to a hMPV antigen. A hMPV antigen refers to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein. A hMPV antigen also refers to a polypeptide that has a similar amino acid sequence compared to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein.

The anti-hMPV-antigen antibodies used in this invention can be monoclonal antibodies, human antibodies, humanized antibodies or chimeric antibodies. In some preferred embodiments, the anti-hMPV antibody of the invention is the antibody disclosed in U.S. patent application Ser. No. 10/628,088, filed Jul. 25, 2003 and published May 20, 2004, as U.S. Pat. Pub. No. US 2004/0096451 A1.

The anti-hMPV-antigen antibodies of this section can be made, formulated, administered, used therapeutically or used prophylactically as described in U.S. patent application Ser. No. 10/628,088, filed Jul. 25, 2003 and published May 20, 2004, as U.S. Pat. Pub. No. US 2004/0096451 A1, the contents of which are hereby incorporated by reference in their entirety.

5.1.1.4. Antibodies that Immunospecifically Bind to Integrin αᵥβ₃

The formulations of the present invention may also comprise an isolated antibody that specifically binds to integrin αᵥβ₃ and compositions comprising this antibody. The antibodies can be monoclonal antibodies, human antibodies, humanized antibodies or chimeric antibodies. In some preferred embodiments, the anti-integrin αᵥβ₃ antibody of the invention is MEDI-522 (Vitaxin®). Vitaxin® and compositions or formulations comprising Vitaxin® are disclosed, e.g., in International Publication Nos. WO 98/33919, WO 00/78815, and WO 02/070007; U.S. application Ser. No. 10/091,236, filed Mar. 4, 2002 and published Nov. 12, 2002, as U.S. Pat. Pub. No. US 2002/0168360, each of which is incorporated herein by reference in its entirety.

In further embodiments, the antibody that immunospecifically binds to integrin αᵥβ₃ is not Vitaxin® or an antigen-binding fragment of Vitaxin®. Examples of known antibodies that immunospecifically bind to integrin αᵥβ₃ include, but are not limited to, 11D2 (Searle), the murine monoclonal LM609 (Scripps, International Publication Nos. WO 89/05155 and U.S. Pat. No. 5,753,230, which is incorporated herein by reference in its entirety), International Publication Nos WO 98/33919 and WO 00/78815, each of which is incorporated herein by reference in its entirety), 17661-37E and 17661-37E 1-5 (USBiological), MON 2032 and 2033 (CalTag), ab7166 (BV3) and ab 7167 (BV4) (Abcam), and WOW-1 (Kiosses et al., Nature Cell Biology, 3:316-320).

The αᵥβ₃ integrin has been found on new blood vessels as well as surface of many solid tumors, activated macrophages, monocytes, and osteoclasts. As such, the anti-integrin αᵥβ₃ antibodies of this section can be used, for example, as an investigational antibody, or in the prevention or treatment of several destructive diseases.

The anti-integrin αᵥβ₃ antibodies of this section can be made, formulated, administered, used therapeutically or used prophylactically as described in U.S. patent application Ser. No. 10/091,236, filed Mar. 4, 2002 and published Nov. 12, 2002, as U.S. Pat. Pub. No. US 2002/0168360 A1; U.S. patent application Ser. No. 10/769,712, filed Jan. 30, 2004, and published as U.S. Pat. Pub. No. US 2004/0208870 A1; U.S. patent application No. 10/769,720, filed Jan. 30, 2004 and published Sep. 9, 2004, as U.S. Pat. Pub. No. US 2004/0176272; U.S. patent application Ser. No. 10/379,145, filed Mar. 4, 2003, and published as U.S. Pat. Pub. No. US 2005/0084489 A1; U.S. patent application Ser. No. 10/379,189, filed Mar. 4, 2003 and published as U.S. Pat. Pub. No. US 2004/0001835; PCT Application No. PCT/US04/02701, filed Jan. 30, 2004; International Application Publication No. WO 00/78815 A1, entitled “Anti-αᵥβ₃ recombinant human antibodies, nucleic acids encoding same and methods”, by Huse et al.; and International Application Publication No.: WO 98/33919 A1, entitled “Anti-alpha-V-veta-3 recombinant humanized antibodies, nucleic acids encoding same and methods of use”, by Huse et al.; International Publication No. WO 89/05155, the contents of which are hereby incorporated by reference in their entirety.

5.1.1.5. Antibodies that Immunospecifically Bind to CD2

The formulations of the present invention may comprise an isolated antibody that immunospecifically binds to CD2 and compositions comprising this antibody. The antibodies can be monoclonal antibodies, human antibodies, humanized antibodies or chimeric antibodies. In some preferred embodiments, the anti-CD2 antibody of the invention is siplizumab (MEDI-507). Siplizumab can selectively binds to cells expressing the CD2 antigen (specifically T cells, natural killer cells and thymocytes) and can be used, for example, in the prophylaxis and treatment of T cell lymphoma or other related conditions. MEDI-507 is disclosed, e.g., in International Publication No. WO 99/03502, International Application Nos. PCT/US02/22273 and PCT/US02/06761, and U.S. Pat. No. 6,849,258, U.S. application Ser. No. 10/091,268, published as US 2003/0068320 A1, and U.S. Ser. No. 10/091,313, published as US 2003/0044406 A1 each of which is incorporated herein by reference in its entirety. MEDI-507 is a humanized IgGlic class monoclonal antibody that immunospecifically binds to human CD2 polypeptide. MEDI-507 was constructed using molecular techniques to insert the CDRs from the rat monoclonal antibody LO-CD2a/BTI-322 into a human IgG1 framework. LO-CD2a/BTI-322 has the amino acid sequence disclosed, e.g., in U.S. Pat. Nos. 5,730,979, 5,817,311, and 5,951,983; and U.S. application Ser. No. 09/056,072 and U.S. Pat. No. 6,849,258 (each of which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 on Jul. 28, 1993 as Accession Number HB 11423.

The anti-CD2 antibodies of this section can be made, formulated, administered, used therapeutically or prophylactically, or in other context as described in U.S. patent application Ser. No. 10/091,268, filed Mar. 4, 2002, and published Apr. 15, 2003, as U.S. Pat. Pub. No. US 2003/0068320; U.S. patent application Ser. No. 10/091,313, filed Mar. 4, 2002, and published Mar. 6, 2003, as U.S. Pat. Pub. No. US 2003/0044406; and U.S. patent application Ser. No. 10/657,006, filed Sep. 5, 2003, and published Dec. 30, 2004, as U.S. Pat. Pub. No. US 2004/0265315, the contents of which are hereby incorporated by reference in their entirety.

5.1.1.6. Antibodies that Immunospecifically Bind to CD19

The formulations of the present invention may comprise an isolated antibody that immunospecifically binds to CD19 and a composition comprising this antibody. The antibodies can be monoclonal antibodies, human antibodies, humanized antibodies or chimeric antibodies. In some preferred embodiments, the anti-CD19 antibody of the invention is MT-103. MT-103 is the most-advanced clinical representative of a novel class of antibody derivatives called Bi-Specific T Cell Engagers (BiTE™). The BiTE compound MT-103 directs and activates the patient's own immune system against the cancer cells, stimulating T cells (a very potent type of white blood cell) to destroy B tumor cells (cancerous white blood cells). MT-103 specifically targets a particular protein (the CD19 antigen), which is present on cancerous B cells but not on other types of blood cells or healthy tissues, therefore avoiding the side effects of traditional chemotherapy

The anti-CD19 antibodies of this section can be made, formulated, administered, used therapeutically or prophylactically, or in other context as described in U.S. Pat. No. 6,723,538, and U.S. Pat. Pub. No. 2004/0162411.

The human CD19 molecule is a structurally distinct cell surface receptor that is expressed on the surface of human B cells. The invention relates to immunotherapeutic compositions and methods for the prophylaxis and treatment of GVHD, humoral rejection, and post-transplantation lymphoproliferative disorder in human subjects; autoimmune diseases and disorders; and cancers, using therapeutic antibodies that bind to the human CD19 antigen.

Hybridomas producing HB12a and HB12b anti-CD19 antibodies have been deposited under ATCC deposit nos. PTA-6580 and PTA-6581. See, also, U.S. application No. to be assigned (Attorney Docket No.: 11605-006-999) and U.S. application Ser. No. 11/355,905, filed Feb. 15, 2006, each of which is incorporated herein by reference in its entirety.

5.1.1.7. Antibodies that Immunospecifically Bind to EphA2

The formulations of the present invention may comprise an isolated antibody that immunospecifically binds to EphA2 and a compositions comprising this antibody. The antibodies of the invention can be monoclonal antibodies, human antibodies, humanized antibodies or chimeric antibodies. In some embodiments, the anti-EphA2 antibody of the invention is EA2. In some preferred embodiments, the EA2 antibody is human or humanized. In other embodiments, the is EA5. In some preferred embodiments, the EA5 antibody is human or humanized. Hybridomas producing the anti-EphA2 antibodies of the invention have been deposited with the American Type Culture Collection (ATCC, P.O. Box 1549, Manassas, Va. 20108) under the provisions of the Budapest Treaty on the International Recognition of the Deposit of Microorganisms for the Purposes of Patent Procedures, and assigned accession numbers, which are incorporated by reference, as shown in Table 2.

TABLE 2
Anti-EphA2 Antibodies.

| EphA2 Antibodies | Deposit No. | Date of Deposit |
|---|---|---|
| EA2.31 | PTA-4380 | May 22, 2002 |
| EA5.12 | PTA-4381 | May 22, 2002 |
| Eph099B-102.147 | PTA-4572 | Aug. 7, 2002 |
| Eph099B-208.261 | PTA-4573 | Aug. 7, 2002 |
| Eph099B-210.248 | PTA-4574 | Aug. 7, 2002 |
| Eph099B-233.152 | PTA-5194 | May 12, 2003 |
| Eph101.530.241 | PTA-4724 | Sep. 26, 2002 |

EphA2 is a 130 kDa receptor tyrosine kinase that is expressed in adult epithelia, where it is found at low levels and is enriched within sites of cell-cell adhesion (Zantek, et al, Cell Growth & Differentiation 10:629, 1999; Lindberg, et al., Molecular & Cellular Biology 10: 6316, 1990). EphA2 is upregulated on a large number of aggressive carcinoma cells. The anti-EphA2 antibodies of this invention can be used, for example, in the treatment of a variety of tumors, including breast, colon, prostate, lung and skin cancers, as well as to prevent metastasis.

The anti-EphA2 antibodies of this section can be made, formulated, administered, used therapeutically or used prophylactically as described in U.S. patent application Ser. No. 10/823,259, filed Apr. 12, 2004, and published Mar. 3, 2005 as U.S. Pat. Pub. No. US 2005/0049176 A1; U.S. patent application Ser. No. 10/823,254, filed on Apr. 12, 2004, and published Mar. 17, 2005 as U.S. Pat. Pub. No. US 2005/0059592 A1; U.S. patent application Ser. No. 10/436,782, filed on May 12, 2003 and published Feb. 12, 2004 as U.S. Pat. Pub. No. 2004/0028685; U.S. patent application Ser. No. 10/436,783, filed on May 12, 2003 and published May 13, 2004 as U.S. Pat. Pub. No. 2004/0091486; U.S. patent application Ser. No. 11/004,794, filed on Dec. 3, 2004 and published Jul. 14, 2005 as U.S. Pat. Pub. No. US 2005/0153923 A1; U.S. patent application Ser. No. 10/994,129, filed on Nov. 19, 2004 and published on Jul. 14, 2005 as U.S. Pat. Pub. No. US 2005/0152899 A1; U.S. patent application Ser. No. 11/004,795, filed on Dec. 3, 2004 and published Jul. 7, 2005 as U.S. Pat. Pub. No. US 2005/0147593 A1; and U.S. Provisional Application Nos. 60/662,517, 60/622,711, 60/622,489, filed Oct. 27, 2004, the contents of which are hereby incorporated by reference in their entirety.

5.1.1.8. Antibodies that Immunospecifically Bind to EphA4

The formulations of the present invention may comprise an isolated antibody that immunospecifically binds to an antigen of EphA4 and a composition comprising this antibody. The antibodies of the invention can be monoclonal antibodies, human antibodies, humanized antibodies or chimeric antibodies. The hybridoma producing the anti-EphA4 antibodies for use in connection with the methods of the invention has been deposited with the American Type Culture Collection (ATCC, P.O. Box 1549, Manassas, Va. 20108) on Jun. 4, 2004 under the provisions of the Budapest Treaty on the International Recognition of the Deposit of Microorganisms for the Purposes of Patent Procedures, and is assigned accession number PTA-6044, and is incorporated by reference.

EphA4 is a receptor tyrosine kinase that is expressed in brain, heart, lung, muscle, kidney, placenta, pancreas (Fox, et al, Oncogene 10:897, 1995) and melanocytes (Easty, et al., Int. J. Cancer 71:1061, 1997). EphA4 is overexpressed in a number of cancers. The anti-EphA4 antibodies of this section can be used, for example, to decrease the expression of EphA4 in the treatment of pancreatic cancers etc.

The anti-EphA4 antibodies of this section can be made, formulated, administered, used therapeutically or used prophylactically as described in U.S. patent application Ser. No. 10/863,729, filed Jun. 7, 2004 and published Jan. 20, 2005 as U.S. Pat. Pub. No. US 2005/0013819 A1; U.S. patent application Ser. No. 11/004,794, filed on Dec. 3, 2004 and published Jul. 14, 2005 as U.S. Pat. Pub. No. US 2005/0153923 A1; U.S. patent application Ser. Nos. 11/004,794 and 11/004,795, filed on Dec. 3, 2004 and published Jul. 7, 2005 as U.S. Pat. Pub. No. US 2005/0147593 A1 the contents of which are hereby incorporated by reference in their entirety.

5.1.1.9. Antibodies that Immunospecifically Bind to HMG1

The formulations of the present invention can comprise an antibody that immunospecifically binds to HMG1 and a composition comprising this antibody. The antibodies of the invention can be monoclonal antibodies, human antibodies, humanized antibodies or chimeric antibodies.

The early proinflammatory cytokines (e.g., TNF, IL-1, etc.) mediate inflammation, and induce the late release of high mobility group protein 1 (HMG1) (also known as HMG-1, HMG1, and HMGB1), a protein that accumulates in serum and mediates delayed lethality and further induction of early proinflammatory cytokines.

It has also been shown that HMG1 can be actively secreted by stimulated macrophages or monocytes in a process requiring acetylation of the molecule, which enables translocation from the nucleus to secretory lysosomes and results in the secretion of an acetylated form of HMG1. See, PCT/IB2003/005718. Thus, HMG1 passively released from necrotic cells and HMGB1 actively secreted by inflammatory cells are molecularly different.

Further, HMG1 has been implicated as a cytokine mediator of delayed lethality in endotoxemia. See, e.g., U.S. Pat. Nos. 6,468,533 and 6,448,223. More specifically, it has been demonstrated that bacterial endotoxin (lipopolysaccharide (LPS)) activates monocytes/macrophages to release HMG1 as a late response to activation, resulting in elevated serum HMG1 levels that are toxic. Antibodies against HMG1 have been shown to prevent lethality of endotoxin even when antibody administration is delayed until after the early cytokine response. Like other proinflammatory cytokines, HMG1 is a potent activator of monocytes. Intratracheal application of HMG1 causes acute lung injury, and anti-HMG1 antibodies protect against endotoxin-induced lung edema. In addition, serum HMG1 levels are elevated in critically ill patients with sepsis or hemorrhagic shock, and levels are significantly higher in non-survivors as compared to survivors.

The anti-HMG1 antibodies of this section can be made, formulated, administered, used therapeutically or used prophylactically as described in U.S. Patent Publication No. 2006-0099207 A1 filed Oct. 21, 2005, which is incorporated herein by reference in its entirety. Three clones, S6, S16 and G4 have been deposited with the American Type Culture Collection (10801 University Boulevard, Manassas, Va. 20110-2209) and assigned ATCC Deposit Nos. PTA-6143 (Deposited Aug. 4, 2004), PTA-6259 (Deposited Oct. 19, 2004) and PTA-6258 (Deposited Oct. 19, 2004) (also referred to herein as “S6”, “S16”, and “G4”, respectively) as described in U.S. Patent Publication No. 2006-0099207 A1 filed Oct. 21, 2005, which is incorporated herein by reference in its entirety.

5.1.1.10. Antibodies that Immunospecifically Bind to ALK

The formulations of the present invention can comprise an antibody that immunospecifically binds to ALK and a composition comprising this antibody. The antibodies of the invention can be monoclonal antibodies, human antibodies, humanized antibodies or chimeric antibodies.

Monoclonal antibodies against ALK as well as hybridoma cell lines producing ALK monoclonal antibodies 8B 10, 16G2-3 and 9C10-5 (deposited with the American Type Culture Collection (10801 University Boulevard, Manassas, Va. 20110-2209) and assigned ATCC Deposit Nos. to be assigned, respectively) as described in U.S. patent application Ser. No. 09/880,097, filed Jun. 14, 2001 and published Mar. 21, 2002, as U.S. Pat. Pub. No. 2002/0034768, which is incorporated herein by reference in its entirety.

Pleiotrophin (PTN) is a 136-amino acid, secreted, heparin-binding cytokine that has diverse functions including a role in angiogenesis. PTN has been shown to specifically bind to a receptor tyrosine kinase, Anaplastic Lymphoma Kinase (ALK), and such binding leads to auto-phosphorylation of the receptor and subsequent phosphorylation of a number of signal transduction molecules such as IRS-1, PLC-gamma, PI3 kinase, and Shc, and activates a cell survival pathway. See PCT Pat. App. Pub. No. WO 01/96364. Accordingly, agents and therapeutic treatments that regulate ALK-mediated signal transduction pathways can affect one or more ALK-regulated functions, including, for example, angiogenesis. ALK participates in various disease states, including cancers and diseases related to unwanted or excessive angiogenesis. Additionally, ALK participates in a desirable way in certain processes, such as wound healing. ALK and/or PTN are expressed, often at high levels, in a variety of tumors. Therefore, agents that downregulate ALK and/or PTN function may affect tumors by a direct effect on the tumor cells, an indirect effect on the angiogenic processes recruited by the tumor, or a combination of direct and indirect effects.

5.1.1.11. Antibodies that Immunospecifically Bind to CD20

The formulations of the present invention can comprise an antibody that immunospecifically binds to CD20 and a composition comprising this antibody. The antibodies of the invention can be monoclonal antibodies, human antibodies, humanized antibodies or chimeric antibodies.

CD20 is only expressed by B lymphocytes (Stashenko et al. (1980) J Immunol 125:1678-1685; Tedder et al., 1988a). CD20 forms a homo- or hetero-tetrameric complex that is functionally important for regulating cell cycle progression and signal transduction in B lymphocytes (Tedder and Engel, 1994). CD20 additionally regulates transmembrane Ca⁺⁺ conductance, possibly as a functional component of a Ca⁺⁺-permeable cation channel (Bubien et al. J Cell Biol 121:1121-1132; Kanzaki et al. (1997a) J Biol Chem 272:14733-14739; Kanzaki et al. (1997b) J Biol Chem 272:4964-4969; Kanzaki et al. (1995) J Biol Chem 270:13099-13104). Antibodies against CD20 are effective in treating non-Hodgkin's lymphoma (McLaughlin et al. (1998) Oncology 12:1763-1769; Onrust et al. (1989) J Biol Chem 264:15323-15327; Weiner (1999) Semin Oncol 26:43-51).

See, also, U.S. patent application Ser. No. 10/433,287, filed Sep. 30, 2003, published as US 20040137566 on Jul. 15, 2004, which is incorporated herein by reference in its entirety. 5.1.1.12. Antibodies that Immunospecifically Bind to CD22

The formulations of the present invention can comprise an antibody that immunospecifically binds to CD22 and a composition comprising this antibody. The antibodies of the invention can be monoclonal antibodies, human antibodies, humanized antibodies or chimeric antibodies.

Anti-CD22 antibodies have been described, for example, in U.S. Pat. Nos. 5,484,892; 6,183,744; 6,187,287; 6,254,868; 6,306,393, and in Tuscano et al., Blood 94(4):1382-92 (1999) (each of which is incorporated herein in its entirety by reference). The use of monoclonal antibodies, including anti-CD22 antibodies, in the treatment of non-Hodgkin's lymphoma is reviewed, for example, by Renner et al., Leukemia 11(Suppl. 2):S5509 (1997).

The use of humanized CD22 antibodies has been described for the treatment of autoimmune disorders (see, Tedder U.S. Patent Application Publication No. US2003/0202975) and for the treatment of B cell malignancies, such as lymphomas and leukemias (see, Tuscano U.S. Patent Application Publication No. U.S. 2004/0001828). Humanized CD22 antibodies that target specific epitopes on CD22 have been described for use in immunoconjugates for therapeutic uses in cancer (see U.S. Pat. Nos. 5,789,554 and 6,187,287 to Leung).

Exemplary VH and VK antibody regions of the invention were deposited with the American Type Culture Collection (ATCC). In particular, a plasmid encoding the humanized anti-CD22 VH sequence of the invention designated RHOv2 was deposited under ATCC deposit no. PTA-7372, on Feb. 9, 2006. A plasmid encoding the humanized anti-CD22 VH sequence of the invention designated RHOv2ACD was deposited under ATCC deposit no. PTA-7373, on Feb. 9, 2006. A plasmid encoding the humanized anti-CD22 VK sequence of the invention, RKA was deposited under ATCC deposit no. PTA-7370, on Feb. 9, 2006. A plasmid encoding the humanized anti-CD22 VK sequence of the invention, RKC, was deposited under ATCC deposit no. PTA-7371, on Feb. 9, 2006.

See, also, U.S. Provisional Application No. TBA, filed Mar. 6, 2006, attorney docket no. BC320P1, which is incorporated herein by reference in its entirety.

5.1.1.13. Antibodies that Immunospecifically Bind to Chitinase

The formulations of the present invention can comprise an antibody that immunospecifically binds to Chitinase and a composition comprising this antibody. The antibodies of the invention can be monoclonal antibodies, human antibodies, humanized antibodies or chimeric antibodies.

It is described that blocking a chitinase/chitinase-like protein, in vivo results in protection of bone and cartilage as well as a reduction in weight loss in a mouse RA model. These results support the role of chitinase/chitinase-like proteins in chronic inflammatory diseases and more specifically the role of chitinase/chitinase-like proteins in OCL-related diseases including bone metabolism and connective tissue disorders and diseases. Furthermore, these results validate human chitinase/chitinase-like proteins as potential therapeutic targets for the prevention and treatment of OCL-related diseases.

See, also, U.S. application Ser. No. 10/202,436, filed Jul. 23, 2002, published as US 20030049261 on Mar. 13, 2003, which is incorporated herein by reference in its entirety.

5.1.1.14. Antibodies that Immunospecifically Bind to Interferon Alpha

The formulations of the present invention can comprise an antibody that immunospecifically binds to interferon alpha and a composition comprising this antibody. The antibodies of the invention can be monoclonal antibodies, human antibodies, humanized antibodies or chimeric antibodies.

The invention provides a method of treating an interferon alpha-mediated disease or disorder in a subject, comprising administering to the subject an anti-IFN alpha antibody of the invention, such that the interferon-alpha mediated disease in the subject is treated. Examples of diseases that can be treated include autoimmune diseases (e.g., systemic lupus erythematosus, multiple sclerosis, insulin dependent diabetes mellitus, inflammatory bowel disease, psoriasis, autoimmune thyroiditis, rheumatoid arthritis and glomerulonephritis), transplant rejection and graft versus host disease.

Anti-interferon alpha monoclonal antibody has also been described in U.S. application Ser. No. 11/009,410, filed Dec. 10, 2004, which is incorporated herein by reference in its entirety.

5.1.1.15. Antibodies that Immunospecifically Bind to Interferon Alpha Receptor

The formulations of the present invention can comprise an antibody that immunospecifically binds to interferon alpha receptor and a composition comprising this antibody. The antibodies of the invention can be monoclonal antibodies, human antibodies, humanized antibodies or chimeric antibodies.

The invention also provides a method for inhibiting biological activity of a type I interferon on a cell expressing interferon alpha receptor 1 comprising contacting the cell with the antibody of the invention, such that the biological activity of the type I interferon is inhibited. The invention also provides a method of treating a type I interferon-mediated disease or disorder in a subject in need of treatment comprising administering to the subject the antibody, or antigen-binding portion thereof, of the invention, such that the type-I interferon mediated disease in the subject is treated. The type I interferon-mediated disease can be, for example, an interferon alpha-mediated disease.

Examples of disease or disorders that can be treated using the methods of the invention include systemic lupus erythematosus, insulin dependent diabetes mellitus, inflammatory bowel disease, multiple sclerosis, psoriasis, autoimmune thyroiditis, rheumatoid arthritis, glomerulonephritis, HIV infection, AIDS, transplant rejection and graft versus host disease.

Anti-interferon receptor monoclonal antibody has been described in U.S. Patent Publication No. 2006-0029601 A1, published Feb. 9, 2006, filed June 20, 2005, which is incorporated by reference herein by reference in its entirety.

5.1.1.16. Antibodies that have Therapeutic Utility

The formulations of the present invention comprise antibodies that have therapeutic utility, including but not limited to antibodies listed in Table 3.

TABLE 3
Therapeutic Antibodies That Can Be Used in Connection with the Present
Invention.

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | metastatic | tumor antigen MUC1 |
| | | cancers | |
| Antisoma | Theragyn | ovarian cancer | PEM antigen |
| | (pemtumomabytrrium- | | |
| | 90) | | |
| | Therex | breast cancer | PEM antigen |
| Boehringer | Blvatuzumab | head & neck | CD44 |
| Ingelheim | | cancer | |
| Centocor/J&J | Panorex | Colorectal | 17-1A |
| | | cancer | |
| | ReoPro | PTCA | Gp IIIb/IIIa |
| | ReoPro | Acute MI | Gp IIIb/IIIa |
| | ReoPro | Ischemic stroke | Gp IIIb/IIIa |
| Corixa | Bexocar | NHL | CD20 |
| CRC Technology | MAb, idiotypic 105AD7 | colorectal cancer | Gp72 |
| | | vaccine | |
| Crucell | Anti-EpCAM | cancer | Ep-CAM |
| Cytoclonal | MAb, lung cancer | non-small cell | NA |
| | | lung cancer | |
| Genentech | Herceptin | metastatic breast | HER-2 |
| | | cancer | |
| | Herceptin | early stage | HER-2 |
| | | breast cancer | |
| | Rituxan | Relapsed/refractory | CD20 |
| | | low-grade or | |
| | | follicular NHL | |
| | Rituxan | intermediate & | CD20 |
| | | high-grade NHL | |
| | MAb-VEGF | NSCLC, | VEGF |
| | | metastatic | |
| | MAb-VEGF | Colorectal | VEGF |
| | | cancer, | |
| | | metastatic | |
| | AMD Fab | age-related | CD18 |
| | | macular | |
| | | degeneration | |
| | E-26 (2^{nd} gen. IgE) | allergic asthma | IgE |
| | | & rhinitis | |
| IDEC | Zevalin (Rituxan + | low grade of | CD20 |
| | yttrium-90) | follicular, | |
| | | relapsed or | |
| | | refractory, | |
| | | CD20-positive, | |
| | | B-cell NHL and | |
| | | Rituximab- | |
| | | refractory NHL | |
| ImClone | Cetuximab + innotecan | refractory | EGF receptor |
| | | colorectal | |
| | | carcinoma | |
| | Cetuximab + cisplatin & | newly diagnosed | EGF receptor |
| | radiation | or recurrent head | |
| | | & neck cancer | |
| | Cetuximab + | newly diagnosed | EGF receptor |
| | gemcitabine | metastatic | |
| | | pancreatic | |
| | | carcinoma | |
| | Cetuximab + cisplatin + | recurrent or | EGF receptor |
| | 5FU or Taxol | metastatic head | |
| | | & neck cancer | |
| | Cetuximab + | newly diagnosed | EGF receptor |
| | carboplatin + paclitaxel | non-small cell | |
| | | lung carcinoma | |
| | Cetuximab + cisplatin | head & neck | EGF receptor |
| | | cancer | |
| | | (extensive | |
| | | incurable local- | |
| | | regional disease | |
| | | & distant | |
| | | metasteses) | |
| | Cetuximab + radiation | locally advanced | EGF receptor |
| | | head & neck | |
| | | carcinoma | |
| | BEC2 + Bacillus | small cell lung | mimics ganglioside |
| | Calmette Guerin | carcinoma | GD3 |
| | BEC2 + Bacillus | melanoma | mimics ganglioside |
| | Calmette Guerin | | GD3 |
| | IMC-1C11 | colorectal cancer | VEGF-receptor |
| | | with liver | |
| | | metasteses | |
| ImmonoGen | nuC242-DM1 | Colorectal, | nuC242 |
| | | gastric, and | |
| | | pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | intraoperative | CEA |
| | labeled arcitumomab) | tumors (radio | |
| | | imaging) | |
| | LeukoScan (Tc-99m- | soft tissue | CEA |
| | labeled sulesomab) | infection | |
| | | (radioimaging) | |
| | LymphoScan (Tc-99m- | lymphomas | CD22 |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN (+ | head & neck | NA |
| | yttrium-90) | cancer | |
| | HumaSPECT | colorectal | NA |
| | | imaging | |
| Medarex | MDX-101 (CTLA-4) | Prostate and | CTLA-4 |
| | | other cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath | chronic | CD52 |
| | (alemtuzumab) | lymphocytic | |
| | | leukemia | |
| NeoRx | CD20-streptavidin (+ | Non-Hodgkins | CD20 |
| | biotin-yttrium 90) | lymphoma | |
| | Avidicin (albumin + | metastatic | NA |
| | NRLU13) | cancer | |
| Peregrine | Oncolym (+ iodine-131) | Non-Hodgkins | HLA-DR 10 beta |
| | | lymphoma | |
| | Cotara (+ iodine-131) | unresectable | DNA-associated |
| | | malignant | proteins |
| | | glioma | |
| Pharmacia | C215 (+ staphylococcal | pancreatic | NA |
| Corporation | enterotoxin) | cancer | |
| | MAb, lung/kidney | lung & kidney | NA |
| | cancer | cancer | |
| | nacolomab tafenatox | colon & | NA |
| | (C242 + staphylococcal | pancreatic | |
| | enterotoxin) | cancer | |
| Protein Design | Nuvion | T cell | CD3 |
| Labs | | malignancies | |
| | SMART M195 | AML | CD33 |
| | SMART 1D10 | NHL | HLA-DR antigen |
| Titan | CEAVac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Trilex | CEAVac | colorectal | CEA |
| | | cancer, | |
| | | advanced | |
| | TriGem | metastatic | GD2-ganglioside |
| | | melanoma & | |
| | | small cell lung | |
| | | cancer | |
| | TriAb | metastatic breast | MUC-1 |
| | | cancer | |
| Viventia Biotech | NovoMAb-G2 | Non-Hodgkins | NA |
| | radiolabeled | lymphoma | |
| | Monopharm C | colorectal & | SK-1 antigen |
| | | pancreatic | |
| | | carcinoma | |
| | GlioMAb-H (+ gelonin | gliorna, | NA |
| | toxin) | melanoma & | |
| | | neuroblastoma | |
| Xoma | Rituxan | Relapsed/refractory | CD20 |
| | | low-grade or | |
| | | follicular NHL | |
| | Rituxan | intermediate & | CD20 |
| | | high-grade NHL | |
| | ING-1 | adenomcarcinoma | Ep-CAM |

5.1.1.17. Antibodies that can be Used for Inflammatory Disorders or Autoimmune Diseases

The formulations of the present invention further comprise any of the antibodies known in the art for the treatment and/or prevention of autoimmune disease or inflammatory disease. A non-limiting example of the antibodies that are used for the treatment or prevention of inflammatory disorders which can be engineered according to the invention is presented in Table 4A, and a non-limiting example of the antibodies that are used for the treatment or prevention of autoimmune disorder is presented in Table 4B.

TABLE 4A
Antibodies for Inflammatory Diseases and Autoimmune Diseases That Can
Be Used in Accordance with the Invention.

| Antibody | Target | | | | |
|---|---|---|---|---|---|
| Name | Antigen | Product Type | Isotype | Sponsors | Indication |
| 5G1.1 | Complement | Humanized | IgG | Alexion | Rheumatoid |
| | (C5) | | | Pharm Inc | Arthritis |
| 5G1.1 | Complement | Humanized | IgG | Alexion | SLE |
| | (C5) | | | Pharm Inc | |
| 5G1.1 | Complement | Humanized | IgG | Alexion | Nephritis |
| | (C5) | | | Pharm Inc | |
| 5G1.1-SC | Complement | Humanized | ScFv | Alexion | Cardiopulmonary |
| | (C5) | | | Pharm Inc | Bypass |
| 5G1.1-SC | Complement | Humanized | ScFv | Alexion | Myocardial |
| | (C5) | | | Pharm Inc | Infarction |
| 5G1.1-SC | Complement | Humanized | ScFv | Alexion | Angioplasty |
| | (C5) | | | Pharm Inc | |
| ABX-CBL | CBL | Human | | Abgenix Inc | GvHD |
| ABX-CBL | CD147 | Murine | IgG | Abgenix Inc | Allograft rejection |
| ABX-IL8 | IL-8 | Human | IgG2 | Abgenix Inc | Psoriasis |
| Antegren | VLA-4 | Humanized | IgG | Athena/Elan | Multiple Sclerosis |
| Anti- | CD11a | Humanized | IgG1 | Genentech | Psoriasis |
| CD11a | | | | Inc/Xoma | |
| Anti-CD18 | CD18 | Humanized | Fab′2 | Genentech Inc | Myocardial |
| | | | | | infarction |
| Anti-LFA1 | CD18 | Murine | Fab′2 | Pasteur- | Allograft rejection |
| | | | | Merieux/ | |
| | | | | Immunotech | |
| Antova | CD40L | Humanized | IgG | Biogen | Allograft rejection |
| Antova | CD40L | Humanized | IgG | Biogen | SLE |
| BTI-322 | CD2 | Rat | IgG | Medimmune | GvHD, Psoriasis |
| | | | | Inc | |
| CDP571 | TNF-alpha | Humanized | IgG4 | Celltech | Crohn's |
| CDP571 | TNF-alpha | Humanized | IgG4 | Celltech | Rheumatoid |
| | | | | | Arthritis |
| CDP850 | E-selectin | Humanized | | Celltech | Psoriasis |
| Corsevin M | Fact VII | Chimeric | | Centocor | Anticoagulant |
| D2E7 | TNF-alpha | Human | | CAT/BASF | Rheumatoid |
| | | | | | Arthritis |
| Hu23F2G | CD11/18 | Humanized | | ICOS Pharm | Multiple Sclerosis |
| | | | | Inc | |
| Hu23F2G | CD11/18 | Humanized | IgG | ICOS Pharm | Stroke |
| | | | | Inc | |
| IC14 | CD14 | | | ICOS Pharm | Toxic shock |
| | | | | Inc | |
| ICM3 | ICAM-3 | Humanized | | ICOS Pharm | Psoriasis |
| | | | | Inc | |
| IDEC-114 | CD80 | Primatised | | IDEC | Psoriasis |
| | | | | Pharm/Mitsubishi | |
| IDEC-131 | CD40L | Humanized | | IDEC | SLE |
| | | | | Pharm/Eisai | |
| IDEC-131 | CD40L | Humanized | | IDEC | Multiple Sclerosis |
| | | | | Pharm/Eisai | |
| IDEC-151 | CD4 | Primatised | IgG1 | IDEC | Rheumatoid |
| | | | | Pharm/Glaxo | Arthritis |
| | | | | SmithKline | |
| IDEC-152 | CD23 | Primatised | | IDEC Pharm | Asthma/Allergy |
| Infliximab | TNF-alpha | Chimeric | IgG1 | Centocor | Rheumatoid |
| | | | | | Arthritis |
| Infliximab | TNF-alpha | Chimeric | IgG1 | Centocor | Crohn's |
| LDP-01 | beta2- | Humanized | IgG | Millennium | Stroke |
| | integrin | | | Inc | |
| | | | | (LeukoSite | |
| | | | | Inc.) | |
| LDP-01 | beta2- | Humanized | IgG | Millennium | Allograft rejection |
| | integrin | | | Inc | |
| | | | | (LeukoSite | |
| | | | | Inc.) | |
| LDP-02 | alpha4beta7 | Humanized | | Millennium | Ulcerative Colitis |
| | | | | Inc | |
| | | | | (LeukoSite | |
| | | | | Inc.) | |
| MAK- | TNF alpha | Murine | Fab′2 | Knoll Pharm, | Toxic shock |
| 195F | | | | BASF | |
| MDX-33 | CD64 (FcR) | Human | | Medarex/Centeon | Autoimmune |
| | | | | | haematogical |
| | | | | | disorders |
| MDX-CD4 | CD4 | Human | IgG | Medarex/Eisai/ | Rheumatoid |
| | | | | Genmab | Arthritis |
| MEDI-507 | CD2 | Humanized | | Medimmune | Psoriasis |
| | | | | Inc | |
| MEDI-507 | CD2 | Humanized | | Medimmune | GvHD |
| | | | | Inc | |
| OKT4A | CD4 | Humanized | IgG | Ortho Biotech | Allograft rejection |
| OrthoClone | CD4 | Humanized | IgG | Ortho Biotech | Autoimmune |
| OKT4A | | | | | disease |
| Orthoclone/ | CD3 | Murine | mIgG2a | Ortho Biotech | Allograft rejection |
| anti-CD3 | | | | | |
| OKT3 | | | | | |
| RepPro/ | gpIIbIIIa | Chimeric | Fab | Centocor/Lilly | Complications of |
| Abciximab | | | | | coronary |
| | | | | | angioplasty |
| rhuMab- | IgE | Humanized | IgG1 | Genentech/Novartis/ | Asthma/Allergy |
| E25 | | | | Tanox | |
| | | | | Biosystems | |
| SB-240563 | IL5 | Humanized | | GlaxoSmithKline | Asthma/Allergy |
| SB-240683 | IL-4 | Humanized | | GlaxoSmithKline | Asthma/Allergy |
| SCH55700 | IL-5 | Humanized | | Celltech/Schering | Asthma/Allergy |
| Simulect | CD25 | Chimeric | IgG1 | Novartis | Allograft rejection |
| | | | | Pharm | |
| SMART | CD3 | Humanized | | Protein | Autoimmune |
| a-CD3 | | | | Design Lab | disease |
| SMART | CD3 | Humanized | | Protein | Allograft rejection |
| a-CD3 | | | | Design Lab | |
| SMART | CD3 | Humanized | IgG | Protein | Psoriasis |
| a-CD3 | | | | Design Lab | |
| Zenapax | CD25 | Humanized | IgG1 | Protein | Allograft rejection |
| | | | | Design | |
| | | | | Lab/Hoffman- | |
| | | | | La Roche | |

TABLE 4B
Antibodies for Autoimmune Disorders That Can Be Used In Accordance
with the Invention.

| Antibody | Indication | Target Antigen |
|---|---|---|
| ABX-RB2 | | antibody to CBL antigen on T cells, |
| | | B cells and NK cells |
| | | fully human antibody from the |
| | | Xenomouse |
| 5c8 (Anti CD-40 | Phase II trials were halted in October | CD-40 |
| ligand antibody) | 1999 examine “adverse events” | |
| IDEC 131 | systemic lupus erythyematous | anti CD40 |
| | (SLE) | humanized |
| IDEC 151 | rheumatoid arthritis | primatized; anti-CD4 |
| IDEC 152 | Asthma | primatized; anti-CD23 |
| IDEC 114 | Psoriasis | primatized anti-CD80 |
| MEDI-507 | rheumatoid arthritis; multiple | anti-CD2 |
| | sclerosis | |
| | Crohn's disease | |
| | Psoriasis | |
| LDP-02 (anti-b7 | inflammatory bowel disease | a4b7 integrin receptor on white |
| mAb) | Chron's disease | blood cells (leukocytes) |
| | ulcerative colitis | |
| SMART Anti- | autoimmune disorders | Anti-Gamma Interferon |
| Gamma Interferon | | |
| antibody | | |
| Verteportin | rheumatoid arthritis | |
| MDX-33 | blood disorders caused by | monoclonal antibody against FcRI |
| | autoimmune reactions | receptors |
| | Idiopathic Thrombocytopenia | |
| | Purpurea (ITP) | |
| | autoimmune hemolytic anemia | |
| MDX-CD4 | treat rheumatoid arthritis and other | monoclonal antibody against CD4 |
| | autoimmunity | receptor molecule |
| VX-497 | autoimmune disorders | inhibitor of inosine monophosphate |
| | multiple sclerosis | dehydrogenase |
| | rheumatoid arthritis | (enzyme needed to make new RNA |
| | inflammatory bowel disease | and DNA |
| | lupus | used in production of nucleotides |
| | psoriasis | needed for lymphocyte |
| | | proliferation) |
| VX-740 | rheumatoid arthritis | inhibitor of ICE |
| | | interleukin-1 beta (converting |
| | | enzyme |
| | | controls pathways leading to |
| | | aggressive immune response) |
| VX-745 | specific to inflammation | inhibitor of P38MAP kinase |
| | involved in chemical signalling of | mitogen activated protein kinase |
| | immune response | |
| | onset and progression of | |
| | inflammation | |
| Enbrel (etanercept) | | targets TNF (tumor necrosis factor) |
| IL-8 | | fully human monoclonal antibody |
| | | against IL-8 (interleukin 8) |
| Apogen MP4 | | recombinant antigen |
| | | selectively destroys disease |
| | | associated T-cells |
| | | induces apoptosis |
| | | T-cells eliminated by programmed |
| | | cell death |
| | | no longer attack body's own cells |
| | | specific apogens target specific T- |
| | | cells |

5.1.1.18. Antibodies Having Increased Half-Lives

The present invention provides for formulations of antibodies and antibody fragments that immunospecifically bind to an antigen of interest (e.g., an IL-9 polypeptide) which have an extended half-life in vivo. In particular, the present invention provides formulations of antibodies and antibody fragments that immunospecifically bind to an antigen of interest (e.g., an IL-9 polypeptide) which have a half-life in an animal, preferably a mammal (e.g., a human), of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies and single chain antibodies) or antibody fragments (e.g., Fab fragments) in vivo, for example, inert polymer molecules such as high molecular weight polyethyleneglycol (PEG) can be attached to the antibodies (including antibody fragments thereof) with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies (including antibody fragments thereof) can be tested for binding activity as well as for in vivo efficacy using methods known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No. 6,277,375, each of which is incorporated herein by reference in its entirety.

Further, antibodies (including antibody fragments thereof) can be conjugated to albumin in order to make the antibody (including antibody fragment thereof) more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

5.1.1.19. Antibody Conjugates

The present invention provides formulations of antibodies (including antibody fragments thereof) that immunospecifically binds to an antigen of interest (e.g., an IL-9 polypeptide) recombinantly fused or chemically conjugated (including both covalent and non-covalent conjugations) to a heterologous protein or polypeptide (or fragment of a polypeptide of at least 10, at least 20, at least 30, at least 40, at least 50, at least 60, at least 70, at least 80, at least 90 or at least 100 amino acids) to generate fusion proteins. In particular, the invention provides formulations of fusion proteins comprising an antigen-binding fragment of an antibody described herein (e.g., a Fab fragment, Fd fragment, Fv fragment, F(ab)₂ fragment, a VH domain, a VH CDR, a VL domain or a VL CDR) and a heterologous protein, polypeptide, or peptide. The heterologous protein, polypeptide, or peptide that the antibody (including antibody fragments thereof) may be fused to is useful for targeting the antibody to respiratory epithelial cells, mast cells, neutrophils, eosinophils, B cells, macrophages, or activated T cells. For example, an antibody that immunospecifically binds to a cell surface receptor expressed by a particular cell type (e.g., a respiratory epithelial cell, a mast cell, a neutrophil, an eosinophil, a B cell, a macrophage, or an activated T cell) may be fused or conjugated to an antibody (including antibody fragment thereof) of the invention. In a specific embodiment, an antibody that immunospecifically binds to an IL-9 polypeptide is fused or conjugated to an anti-stem cell factor or an anti-kit ligand. Methods for fusing or conjugating proteins, polypeptides, or peptides to an antibody (including antibody fragment thereof) are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies (including antibody fragments thereof), or the encoded antibodies or fragments thereof, may be altered by being subjected to random mutagenesis by error-prone PCR, random nucleotide insertion or other methods prior to recombination. A polynucleotide encoding an antibody (including antibody fragment thereof) thereof may be recombined with one or more components, motifs, sections, parts, domains, fragments, etc. of one or more heterologous molecules.

Moreover, the antibodies (including antibody fragments thereof) can be fused to marker sequences, such as a peptide to facilitate purification. The marker amino acid sequence may be a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of a disease or disorder (e.g., an autoimmune disorder, an inflammatory disorder, a proliferative disorder, or an infection (e.g., a respiratory infection)) as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Sc, ¹¹³Sn, and ¹¹⁷Sn; and positron emitting metals using various positron emission tomographies, and noradioactive paramagnetic metal ions.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); anthracyclines (e.g., daunorubicin (formerly daunomycin) and doxorubicin); antibiotics (e.g., dactinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos.: 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709); adenosine deaminase inhibitors (e.g., Fludarabine phosphate and 2-Chlorodeoxyadenosine); ibritumomab tiuxetan (ZEVALIN®); tositumomab (BEXXAR®)) and pharmaceutically acceptable salts, solvates, clathrates, and prodrugs thereof.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), an anti-angiogenic agent, e.g., angiostatin, endostatin or a component of the coagulation pathway (e.g., tissue factor); or, a biological response modifier such as, for example, a lymphokine (e.g., interferon gamma (“IFN-γ”), interleukin-1 (“IL-1”), interleukin-2 (“IL-2”), interleukin-5 (“IL-5”), interleukin-6 (“IL-6”), interleuking-7 (“IL-7”), interleukin-10 (“IL-10”), interleukin-12 (“IL-12”), interleukin-15 (“IL-15”), interleukin-23 (“IL-23”), granulocyte macrophage colony stimulating factor (“GM-CSF”), and granulocyte colony stimulating factor (“G-CSF”)), or a growth factor (e.g., growth hormone (“GH”)), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid. fibrinopeptides A and B from the α and β chains of fibrinogen, fibrin monomer). In a specific embodiment, an antibody that immunospecifically binds to an IL-9 polypeptide is conjugated with a leukotriene antagonist (e.g., montelukast, zafirlukast, pranlukast, and zyleuton).

Moreover, an antibody can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹L, ¹³¹Y, ¹³¹Ho, ¹³¹Sm, to polypeptides or any of those listed supra. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in

Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The therapeutic moiety or drug conjugated to an antigen of interest (e.g., an IL-9 polypeptide) or fragment thereof should be chosen to achieve the desired prophylactic or therapeutic effect(s) for a particular disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, in a subject. A clinician or other medical personnel should consider the following when deciding on which therapeutic moiety or drug to conjugate to an antibody of interest, for example, an antibody that immunospecifically binds to an IL-9 polypeptide or fragment thereof: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The therapeutic moiety or drug conjugated to an antibody of interest, (including antibody fragment thereof), for example, an antibody that immunospecifically binds to an IL-9 polypeptide should be chosen to achieve the desired prophylactic or therapeutic effect(s) for a particular disorder in a subject. A clinician or other medical personnel should consider the following when deciding on which therapeutic moiety or drug to conjugate to an antibody of interest (including antibody fragment thereof), for example, an antibody that immunospecifically binds to an IL-9 polypeptide: the nature of the disease, the severity of the disease, and the condition of the subject.

5.2. Method of Preparing the Antibody Formulations

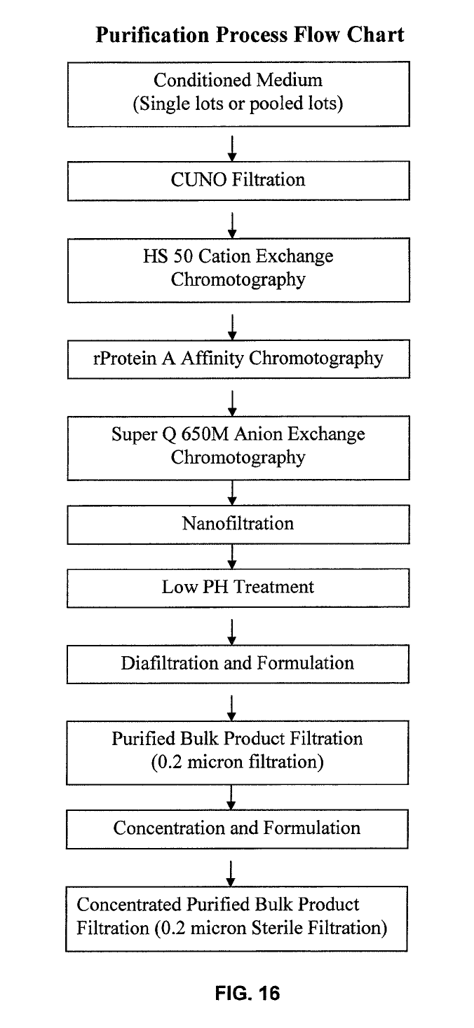

The present invention provides methods for preparing liquid formulations of antibodies or derivatives, analogues, or fragments thereof that immunospecifically bind to an an antigen of interest (e.g., an IL-9 polypeptide). FIG. 16 is a schematic diagram showing the outline for preparing purified anti-IL-9 antibodies. The methods for preparing liquid formulations of the present invention may comprise: purifying the antibody (including antibody fragment thereof) from conditioned medium (either single lots or pooled lots of medium) and concentrating a fraction of the purified antibody (including antibody fragment thereof) to a final concentration of from about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml. Conditioned medium containing the antibody (including antibody fragment thereof), for example, an antibody that immunospecifically binds to an IL-9 polypeptide may be subjected to CUNO filtration and the filtered antibody is subjected to HS50 cation exchange chromatography. The fraction from the HS50 cation exchange chromatography is then subjected to rProtein A affinity chromatography followed by low pH treatment. Following low pH treatment, the antibody (including antibody fragment thereof) fraction is subject to super Q 650 anion exchange chromatography and then nanofiltration. The fraction of the antibody (including antibody fragment thereof) obtained after nanofiltration is then subjected to diafiltration and ultrafiltration to buffer exchange and concentrate the antibody (including antibody fragment thereof) fraction into the formulation buffer using the same membrane. For a detailed description for preparation of the antibody formulations, see Section 6, infra.

The formulation buffer of the present invention comprises phosphate (or other non-zwitterions such as tris, citrate, succinate, and acetate) at a concentration ranging from about 1 mM to about 100 mM, about 5 mM to about 50 mM, about 10 mM to about 30 mM, about 10 mM to about 25 mM, about 25 mM to about 75 mM, or about 10 mM to about 100 mM. In a specific embodiment, the formulation buffer of the present invention comprises phosphate (or other non-zwitterions such as tris, citrate, succinate, and acetate)at a concentration of about 10 mM, about 12 mM, about 15 mM, about 20 mM, about 25 mM, about 50 mM, about 55 mM, about 60 mM, about 65 mM, about 70 mM, about 75 mM, about 80 mM, about 90 mM, about 95 mM, or about 100 mM. The pH of the formulation may range from about 4.0 to about 8.0, e.g., about 6.0 to about 6.5.

The liquid formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody (including antibody fragment thereof) that immunospecifically binds to an IL-9 polypeptide ranging from about 10 mg/ml to about 300 mg/ml. If necessary, these preparations can be adjusted to a desired concentration by adding a sterile diluent to each vial. In a specific embodiment, the liquid formulations of the present invention are formulated into single dose vials as a sterile liquid that contains 50 mM phosphate buffer at pH 6.2 and 150 mM sodium chloride. Each 1.0 mL of solution contains 100 mg of the antibody (including antibody fragment thereof), 50 mg and 1 mg of sodium chloride in water. In one embodiment, the antibody (including antibody fragment thereof) of the invention is supplied at 100 mg/ml in 3 cc USP Type I borosilicate amber vials (West Pharmaceutical Services—Part No. 6800-0675). The target fill volume is 1.2 mL.

The liquid formulations of the present invention may be sterilized by various sterilization methods, including sterile filtration, radiation, etc. In a most preferred embodiment, the difiltrated antibody formulation is filter-sterilized with a presterilized 0.2 micron filter. Sterilized liquid formulations of the present invention may be administered to a subject to prevent, treat and/or manage a disease or disorder (e.g., a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection)) or one or more symptoms thereof.

Although the invention is directed to liquid non-lyophilized formulations, it should be noted for the purpose of equivalents that the formulations of the invention may be lyophilized if desired. Thus, the invention encompasses lyophilized forms of the formulations of the invention.

5.3. Methods of Preparing Antibodies

The antibodies (including antibody fragments thereof) that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), and Harlow et al., Using Antibodies: A laboratory Manual, Cold Spring Harbor Laboratory Press (1999) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a

RIMMS (repetitive immunization multiple sites) technique can be used to immunize an animal (Kilpatrack et al., 1997, Hybridoma 16:381-9, incorporated herein by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, 5,969,108, 6,33,187, 5,824,520, and 5,702,892; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. The vectors for expressing the VH or VL domains may comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, W098/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,8 16397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and lgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morca et al., Methods 20(3):267-79 (2000), Baca et al., J. Biol. Chem. 272(16):10678-84 (1997), Roguska et al., Protein Eng. 9(10):895-904 (1996), Couto et al., Cancer Res. 55 (23 Supp):5973s -5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature 332:323, which are incorporated herein by reference in their entireties).

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to an antigen (e.g., an IL-9 polypeptide) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

5.3.1. Recombinant Expression of an Antibody

Recombinant expression of an antibody contained in a formulation of the invention (e.g., a heavy or light chain of an antibody of the invention or a fragment thereof or a single chain antibody of the invention) may require construction of an expression vector containing a polynucleotide that encodes the antibody. Once a polynucleotide encoding an antibody molecule, heavy or light chain of an antibody, or fragment thereof (preferably, but not necessarily, containing the heavy or light chain variable domain) has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody (including antibody fragment thereof), or a heavy or light chain CDR, operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, and eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention, derivative, analog, or fragment thereof is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.4. Methods of Monitoring the Stability and Aggregation of Antibody Formulations

There are various methods available for assessing the stability of protein formulations, including antibody formulations, based on the physical and chemical structures of the proteins as well as on their biological activities. For example, to study denaturation of proteins, methods such as charge-transfer absorption, thermal analysis, fluorescence spectroscopy, circular dichroism (CD), NMR, and HPSEC, tangential flow filtration (TFF), static light scattering (SLS), Fourier Transform Infrared Spectroscopy (FTIR), urea-induced protein unfolding techniques, intrinsic tryptophan fluorescence, differential scanning calorimetry, and 1-anilino-8-naphthalenesulfonic acid (ANS) protein binding techniques are available. See, for example, Wang et al., 1988, J. of Parenteral Science & Technology 42(Suppl):S4-S26.

The rCGE and HPSEC are the most common and simplest methods to assess the formation of protein aggregates, protein degradation, and protein fragmentation. Accordingly, the stability of the liquid formulations of the present invention may be assessed by these methods.

For example, the stability of the liquid formulations of the present invention may be evaluated by HPSEC or rCGE, wherein the percent area of the peaks represents the non-degraded antibody or non-degraded antibody fragments. In particular, approximately 250 μg of the antibody (including antibody fragment thereof) (approximately 25 μl of a liquid formulation comprising 10 mg/ml said antibody or antibody fragment) is injected onto a TosoH Biosep TSK G30005 W_{XL} column (7.8 mm×30 cm) fitted with a TSK SW x1 guard column (6.0 mm CX 4.0 cm). The antibody (including antibody fragment thereof) is eluted isocratically with 0.1 M disodium phosphate containing 0.1 M sodium sulfate and 0.05% sodium azide, at a flow rate of 0.8 to 1.0 ml/min. Eluted protein is detected using UV absorbance at 280 nm. Reference standards are run in the assay as controls, and the results are reported as the area percent of the product monomer peak compared to all other peaks excluding the included volume peak observed at approximately 12 to 14 minutes. Peaks eluting earlier than the monomer peak are recorded as percent aggregate.

The liquid formulations of the present invention exhibit low to undetectable levels of aggregation as measured by any of the methods described above, that is, no more than 5%, no more than 4%, no more than 3%, no more than 2%, no more than 1%, and no more than 0.5% aggregate by weight protein, and low to undetectable levels of fragmentation, that is, 80% or higher, 85% or higher, 90% or higher, 95% or higher, 98% or higher, or 99% or higher, or 99.5% or higher of the total peak area in the peak(s) representing intact antibodies (including antibody fragments thereof). In the case of SDS-PAGE, the density or the radioactivity of each band stained or labeled with radioisotope can be measured and the % density or % radioactivity of the band representing non-degraded antibodies (including antibody fragments thereof) can be obtained.

The stability of the liquid formulations of the present invention can be also assessed by any assays which measure the biological activity of the antibody in the formulation. The biological activities of antibodies include, but are not limited to, antigen-binding activity, complement-activation activity, Fc-receptor binding activity, and so forth. Antigen-binding activity of the antibodies (including antibody fragments thereof) can be measured by any method known to those skilled in the art, including but not limited to ELISA, radioimmunoassay, Western blot, and the like. Complement-activation activity can be measured by a C3a/C4a assay in the system where the antibody is reacted in the presence of the complement components with the cells expressing the antigen to which the antibody immunospecifically binds. Also see Harlow et al., Antibodies: A Laboratory Manual, (Cold Spring Harbor Laboratory Press, 2nd ed. 1988) (incorporated by reference herein in its entirety). An ELISA based assay, e.g., may be used to compare the ability of an antibody (including antibody fragments thereof) to immunospecifically bind to an IL-9 polypeptide to 4D4, 4D4 H2-1 D11, 4D4com-XF-9, 4D4com-2F9, 7F3, 71A10, 7F3 22D3, 7F3com-2H2, 7F3com-3H5, or 7F3com-3D4 (or any other antibody that is in a formulation of the invention) reference standards. In this assay, referred to as the VnR Binding ELISA, plates are coated with an isolated IL-9 polypeptide and the binding signal of a set concentration of 4D4, 4D4 H2-1 D11, 4D4com-XF-9, 4D4com-2F9, 7F3, 71A10, 7F3 22D3, 7F3com-2H2, 7F3com-3H5, or 7F3com-3D4 reference standards is compared to the binding signal of the same concentration of a test antibody (including antibody fragment thereof).

The purity of the liquid antibody formulations of the invention may be measured by any method well-known to one of skill in the art such as, e.g., HPSEC. The sterility of the liquid antibody formulations may be assessed as follows: sterile soybean-casein digest medium and fluid thioglycollate medium are inoculated with a test liquid antibody formulation by filtering the liquid antibody formulation through a sterile filter having a nominal porosity of 0.45 μm. When using the Sterisure™ or Steritest™ method, each filter device is aseptically filled with approximately 100 ml of sterile soybean-casein digest medium or fluid thioglycollate medium. When using the conventional method, the challenged filter is aseptically transferred to 100 ml of sterile soybean-casein digest medium or fluid thioglycollate medium. The media are incubated at appropriate temperatures and observed three times over a 14 day period for evidence of bacterial or fungal growth.

5.5. Prophylactic and Therapeutic Utility of the Antibody Formulations

The present invention is also directed to antibody-based therapies which involve administering to a subject, preferably a human, the liquid antibody formulations (or “antibody formulations” or “liquid formulations”) of the present invention for preventing, treating and/or managing a disease or disorder, for example, a disease or disordre associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof (see U.S. Provisional Appn. No. 60/477,801, filed Jun. 10, 2003 and published as U.S. Pat. Pub. No. US 2006/0029601 A1 entitled “Methods of Preventing or Treating Respiratory Conditions,” U.S. application Ser. No. 10/823810, filed Apr. 12, 2004 and published as U.S. Pat. Pub. No. US 2005/0147607 A1 entitled “Methods of Preventing or Treating Respiratory Conditions” and a U.S. Provisional Appn. (identified by Attorney Docket No. 10271-113-999) filed concurrently herewith on Apr. 12, 2004, entitled “Methods of Preventing or Treating Respiratory Conditions,” which are all incorporated by reference herein in their entireties). In specific embodiments, the liquid formulations of the invention comprise an antibody (including antibody fragment thereof) at concentrations of from about 10 mg/ml to about 300 mg/ml in a solution containing phosphate, which antibody (including antibody fragment thereof) immunospecifically binds to an IL-9 polypeptide. The liquid formulations of the invention may comprise a single antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3com-2H2, 7F3com-3H5, or 7F3com-3D4). The liquid formulations of the invention may also comprise two or more antibodies (including antibody fragments thereof) that immunospecifically bind to an IL-9 polypeptide. In a specific embodiment, antibodies (including antibody fragments thereof) included in such liquid formulations are 4D4, 4D4 H2-1 D11, 4D4com-XF-9, 4D4com-2F9, 7F3, 71A10, 7F3 22D3, 7F3com-2, 7F3com-3H5, or 7F3com-3D4 or fragments thereof. In an alternative embodiment, antibodies (including antibody fragments thereof) included in such liquid formulations are not 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or fragments thereof. In yet another embodiment, the liquid formulations of the invention comprise an antibody (including antibody fragment thereof) that immunospecifically binds to an IL-9 polypeptide, and the antibody (including antibody fragment thereof) is also conjugated to another moiety, including but not limited to, a heterologous protein, peptide or polypeptide, another antibody (including antibody fragment thereof), a marker sequence, a diagnostic agent, a therapeutic agent, a radioactive metal ion, and a solid support.

The liquid formulations of the present invention may be used locally or systemically in the body as a therapeutic. Particularly, the liquid formulations of the invention may be used in the prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. The formulations of the invention can be used to regulate the activity of cells expressing an IL-9R. In a specific embodiment, the formulations of the invention are used to regulate various activities of a body, including but not limited to, immune functions. The formulations of the present invention may also be advantageously utilized in combination with one or more other therapies (e.g., one or more other prophylactic or therapeutic agents), preferably therapies useful in the prevention, treatment and/or management of a disease or disorder, for example, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. When one or more other therapies (e.g., prophylactic or therapeutic agents) are used, they can be administered separately, in any appropriate form and by any suitable route. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

Any therapy (e.g., prophylactic or therapeutic agents) which is known to be useful, or which has been used or is currently being used for the prevention, treatment and/or management of one or more symptoms associated with a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), can be used in combination with the liquid antibody formulations of the present invention in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th

Ed., Merck Sharp & Dohme Research Laboratories, Rahway, N.J., 1999; and Cecil Textbook of Medicine, 20th Ed., Bennett and Plum (eds.), W. B. Saunders, Philadelphia, 1996 for information regarding therapies, in particular prophylactic or therapeutic agents, which have been or are currently being used for preventing, treating and/or managing diseases or disorders associated with or characterized by aberrant expression and/or activity of an IL-9 polypeptide, diseases or disorders associated with or characterized by aberrant expression and/or activity of the IL-9R or one or more subunits thereof, autoimmune diseases, inflammatory diseases, proliferative diseases, or infections (e.g., respiratory infections), or one or more symptoms thereof. Examples of prophylactic and therapeutic agents include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

A liquid formulation of the invention may be administered to a mammal, preferably a human, concurrently with one or more other therapies (e.g., one or more other prophylactic or therapeutic agents), preferably therapies useful for the prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. The term “concurrently” is not limited to the administration of prophylactic or therapeutic agents/therapies at exactly the same time, but rather it is meant that a liquid formulation of the invention and the other agent/therapy are administered to a mammal in a sequence and within a time interval such that the antibody (including antibody fragment thereof) that immunospecifically binds to an IL-9 polypeptide contained in the liquid formulation can act together with the other agent/therapy to provide an increased benefit than if they were administered otherwise. For example, a liquid formulation of the invention and one or more other prophylactic or therapeutic agents useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, may be administered at the same time or sequentially in any order at different points in time; however, if not administered at the same time, they should be administered sufficiently close in time so as to provide the desired therapeutic or prophylactic effect.

In various embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered within the same patient visit. In other embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered at about 2 to 4 days apart, at about 4 to 6 days apart, at about 1 week part, at about 1 to 2 weeks apart, or more than 2 weeks apart. In preferred embodiments, a liquid formulation of the invention and one or more other therapies (e.g., prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered in a time frame where both agents are still active. One skilled in the art would be able to determine such a time frame by determining the half-life of the administered agents.

In certain embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, liquid formulation of the invention and one or more other therapies (e.g., prophylactic or therapeutic agents), preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, are administered in metronomic dosing regimens, either by continuous infusion or frequent administration without extended rest periods. Such metronomic administration can involve dosing at constant intervals without rest periods. Typically the prophylactic or therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the prophylactic and therapeutic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months.

In one embodiment, a liquid formulation of the invention is administered in a dosing regimen that maintains the plasma concentration of the antibody (including antibody fragment thereof) immunospecific for an IL-9 polypeptide at a desirable level (e.g., about 0.1 to about 100 μg/ml), which continuously blocks the an IL-9R activity. In a specific embodiment, the plasma concentration of the antibody (including antibody fragment thereof) is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment and/or management of a chronic disease or disorder.

In one embodiment, a liquid formulation of the invention is administered to a subject with a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof using a dosing regimen that maintains the plasma concentration of the an antibody (including antibody fragment thereof) that immunospecifically binds to an IL-9 polypeptide at a level that blocks at least 40%, at least 50%, at least 55%, at least 60%, at least 65%, at least 70%, at least 75%, at least 80%, at least 85%, at least 90% or at least 95% of IL-9R binding to an IL-9 polypeptide. In a specific embodiment, the plasma concentration of the an antibody (including antibody fragment thereof) that immunospecifically binds to an IL-9 polypeptide is maintained at about 0.1 μg/ml to about 100 μg/ml in a subject with a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof.

In some embodiments, a liquid formulation of the invention is administered intermittently to a subject, wherein the liquid formulation comprises an antibody (including antibody fragment thereof) conjugated to a moiety (e.g., a therapeutic agent or a toxin).

When used in combination with other therapies (e.g., prophylactic and/or therapeutic agents) useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, the liquid formulations of the invention and the other therapy can act additively or synergistically. The invention contemplates administration of a liquid formulation of the invention in combination with other therapies (e.g., prophylactic or therapeutic agents) preferably therapies useful for prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof by the same or different routes of administration, e.g., oral and parenteral. In certain embodiments, when a liquid formulation of the invention is administered concurrently with one or more therapies (e.g., prophylactic or therapeutic agents) that potentially produce adverse side effects (including, but not limited to, toxicity), the therapies (e.g., prophylactic or therapeutic agents) can advantageously be administered at a dose that falls below the threshold that the adverse side effect is elicited.

5.5.1. Cancer Treatment

The liquid formulations of the invention may be administered to a subject in need thereof to prevent, treat and/or manage a cancer or one or more symptoms thereof. The liquid formulations of the invention may also be administered in combination with one or more other therapies, preferably therapies useful for the prevention, management or treatment of cancer (including, but not limited to the prophylactic or therapeutic agents listed in Section 5.5.1.1, infra) to a subject in need thereof to prevent, treat and/or manage a cancer or one or more symptoms thereof. In a specific embodiment, the invention provides a method of preventing, treating and/or managing cancer or one or more symptoms thereof, said method comprising administering to a subject in need thereof a dose of a prophylactically or therapeutically effective amount of a liquid formulation of the invention. In another embodiment, the invention provides a method of preventing, treating and/or managing cancer or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents other than antibodies (including antibody fragments thereof) that immunospecifically bind to an IL-9 polypeptide).

The liquid formulations of the invention may be used as a first, second, third or fourth line cancer treatment. The invention provides methods for preventing, treating and/or managing one or more symptoms of a cancer in a subject refractory to conventional therapies for such a cancer, said methods comprising administering to said subject a dose of a prophylactically or therapeutically effective amount of a liquid formulation of the invention. A cancer may be determined to be refractory to a therapy means when at least some significant portion of the cancer cells are not killed or their cell division arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

The invention provides methods for preventing, treating and/or managing cancer or one or more symptoms thereof in a subject refractory to existing single agent therapies for such a cancer, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that immunospecifically bind to an IL-9 polypeptide. The invention also provides methods for preventing, treating and/or managing cancer by administering a liquid formulation of the invention in combination with any other treatment (e.g., radiation therapy, chemotherapy or surgery) to patients who have proven refractory to other treatments but are no longer on these treatments. The invention also provides methods for the management or treatment of a patient having cancer and immunosuppressed by reason of having previously undergone other cancer therapies. The invention also provides alternative methods for the prevention, treatment and/or management of cancer or one or more symptoms thereof, where chemotherapy, radiation therapy, hormonal therapy, and/or biological therapy/immunotherapy has proven or may prove too toxic, i.e., results in unacceptable or unbearable side effects, for the subject being treated. Further, the invention provides methods for preventing the recurrence of cancer in patients that have been treated and have no disease activity by administering a liquid formulation of the invention.

Cancers that can be treated by the methods encompassed by the invention include, but are not limited to, neoplasms, tumors, metastases, or any disease or disorder characterized by uncontrolled cell growth. The cancer may be a primary or metastatic cancer. The cancer may or may not express an IL-9R. Specific examples of cancers that can be treated by the methods encompassed by the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone cancer and connective tissue sarcomas such as but not limited to bone sarcoma, myeloma bone disease, osteosarcoma, chondrosarcoma, Ewing's sarcoma, Paget's disease of bone, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease), and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

5.5.1.1. Anti-Cancer Therapies

The present invention provides methods of preventing, treating and/or managing cancer or one or more symptoms thereof, said methods comprising administering to a subject in need thereof a liquid formulation of the invention and one or more therapies (e.g. prophylactic or therapeutic agents) other than antibodies (including antibody fragments thereof) that immunospecifically bind to an IL-9 polypeptide. Therapeutic or prophylactic agents include, but are not limited to, peptides, polypeptides, proteins, fusion proteins, nucleic acid molecules, small molecules, mimetic agents, synthetic drugs, inorganic molecules, and organic molecules. Any agent or therapy (e.g., chemotherapies, radiation therapies, hormonal therapies, and/or biological therapies/immunotherapies) which is known to be useful, or which has been used or is currently being used for the prevention, treatment and/or management of cancer or one or more symptoms thereof can be used in combination with a liquid formulation of the invention in accordance with the invention described herein.

In certain embodiments, the anti-cancer agent is an immunomodulatory agent, such as a chemotherapeutic agent. In certain other embodiments, the anti-cancer agent is an immunomodulatory agent other than a chemotherapeutic agent. In other embodiments, the anti-cancer agent is not an immunomodulatory agent. In specific embodiments, the anti-cancer agent is an anti-angiogenic agent. In other embodiments, the anti-cancer agent is not an anti-angiogenic agent. In specific embodiments, the anti-cancer agent is an anti-inflammatory agent. In other embodiments, the anti-cancer agent is not an anti-inflammatory agent.

In particular embodiments, the anti-cancer agent is, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors (e.g., anti-EphA2 antibodies that result in the phosphorylation of EphA2 and the degration of EphA2 (see, U.S. Patent Application No. 60/418,213, which is incorporated herein by reference in its entirety); elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine;

carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip;

naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; VITAXIN™ (see U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents”); vorozole; zanoterone; zeniplatin; zilascorb; and zinostatin stimalamer. In another preferred embodiment, an antibody derivative such as MT103, part of a class of antibody derivatives known as Bi-Specific T Cell Engagers (BiTE™; MedImmune, Inc.), may also be used in combination with one or more liquid formulations of the present invention.

Other examples of anti-cancer agents include, but are not limited to, angiogenesis inhibitors, topoisomerase inhibitors and immunomodulatory agents (such as chemotherapeutic agents and non-therapeutic immunomodulatory agents, including but not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies), and antibodies that immunospecifically bind to tumor-associated antigens (e.g., HERCEPTIN®).

The invention also encompasses administration of a liquid formulation of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radiaoactive source is placed inside the body close to cancer cells or a tumor mass.

In specific embodiments, patients with breast cancer are administered a prophylactically or therapeutically effective amount of a liquid formulation of the invention in combination with the administration of a prophylactically or therapeutically effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), Herceptin®, tamoxifen, the combination of tamoxifen and cytotoxic chemotherapy. In certain embodiments, patients with metastatic breast cancer are administered a prophylactically or therapeutically effective amount of one or more liquid formulations of the invention in combination with the administration of an effective amount of taxanes such as docetaxel and paclitaxel. In other embodiments, a prophylactically or therapeutically effective amount of a liquid formulation of the invention is administered in combination with the administration of a prophylactically or therapeutically effective amount of taxanes plus standard doxorubicin and cyclophosphamide for adjuvant treatment of node-positive, localized breast cancer.

In specific embodiments, patients with prostate cancer are administered a prophylactically or therapeutically effective amount of a liquid formulation of the invention in combination with the administration of a prophylactically or therapeutically effective amount of one or more other agents useful for prostate cancer therapy including but not limited to: external-beam radiation therapy, interstitial implantation of radioisotopes (i.e., I¹²⁵, palladium, Iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel. In specific embodiments, patients with ovarian cancer are administered a prophylactically or therapeutically effective amount of a liquid formulation of the invention in combination with a prophylactically or therapeutically effective amount of one or more other agents useful for ovarian cancer therapy including but not limited to: intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that a prophylactically or therapeutically effective amount of a liquid formulation of the invention is administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors. In specific embodiments, patients with bone sarcomas are administered a prophylactically or therapeutically effective amount of a liquid formulation of the invention in combination with a prophylactically or therapeutically effective amount of one or more other agents useful for bone sarcoma therapy including but not limited to: doxorubicin, ifosfamide, cisplatin, high-dose methotrexate, cyclophosphamide, etoposide, vincristine, dactinomycin, and surgery.

In specific embodiments, patients with tumor metastatic to bone are administered a prophylactically or therapeutically effective amount of a liquid formulation of the invention in combination with a prophylactically or therapeutically effective amount of one or more other agents useful for bone metastatic tumor therapy including but not limited to: agents or therapies used in treatment of underlying malignancy (non-limiting examples are hormone inhibitors for prostate or breast cancer metastasized to bone and surgery), radiotherapy (non-limiting examples are strontium 89 and samarium 153, which are bone-seeking radionuclides that can exert antitumor effects and relieve symptoms), and bisphosponates.

Cancer therapies and their dosages, routes of administration and recommended usage are known in the art and have been described in such literature as the Physicians' Desk Reference (60th ed., 2006).

5.5.2. Proliferative Disorders

The liquid antibody formulations of the invention can be used to prevent, treat and/or manage a proliferative disorder or one or more symptoms thereof. In a specific embodiment, the proliferative disorder is characterized by aberrant proliferation (e.g. uncontrolled proliferation or lack of proliferation) of cells that IL-9 mediates the growth of, including, but not limited to T cells, erythroid progenitors, B cells, mast cells, eosinophils, neutrophils, and fetal thymocytes.

The present invention provides methods for preventing, treating and/or managing one or more symptoms of a non-cancerous disorder (i.e., a disorder that does not have the potential to metasasize) associated with IL-9 mediated cellular hyperproliferation, particularly of epithelial cells (e.g., as in asthma, COPD, lung fibrosis, bronchial hyperresponsiveness, psoriasis, lymphoproliferative disorder, and seborrheic dermatitis) and endothelial cells (e.g., as in restenosis, hyperproliferative vascular disease, Behcet's Syndrome, atherosclerosis, and macular degeneration), said methods comprising administering to a subject in need thereof an effective amount of one or more antibodies of the invention. The present invention also provides methods for preventing, treating and/or managing a non-cancerous disorder associated with IL-9 mediated cellular hyperproliferation, said methods comprising of administering to a subject in need thereof an effective amount of one or more antibodies of the invention and an effective amount of one or more other therapies (e.g., one or more prophylactic or therapeutic agents) other than antibodies of the invention useful for the prevention, treatment and/or management of said disorder.

The invention provides methods for preventing, treating and/or managing one or more symptoms of a non-cancerous disorder associated with IL-9 mediated cellular hyperproliferation in a subject refractory to conventional therapies for such disorder, said methods comprising administering to subject an effective amount of one or more antibodies, compositions, or combination therapies of the invention. In certain embodiments, a patient with a non-cancerous disorder associated with IL-9 mediated cellular hyperproliferation is refractory to a therapy when the hyperproliferation has not been eradicated and/or the symptoms have not been alleviated. The determination of whether a patient is refractory can be made either in vivo or in vitro by any method known in the art for assaying the effectiveness of a treatment of non-cancerous hyperproliferation disorders, using art-accepted meanings of “refractory” such a context. In various embodiments, a patient with a non-cancerous disorder associated with IL-9 mediated cellular hyperproliferation is refractory when the patient's levels of IL-9 remain abnormal and/or if cellular proliferation has not been decreased. The present invention also provides methods for preventing, treating and/or managing a non-cancerous disorder associated with IL-9 mediated cellular hyperproliferation in a subject refractory to conventional therapies for such disorder, said methods comprising of administering to a subject in need thereof an effective amount of one or more antibody formulations of the invention and an effective amount of one or more other therapies (e.g., one or more prophylactic or therapeutic agents) other than antibody formulations of the invention useful for the prevention, treatment and/or management of said disorder.

In a specific embodiment, an effective amount of one or more antibodies of the invention is administered in combination with an effective amount of a liquid formulation of the invention containing an antibody (including antibody fragment thereof), (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4) to a subject at risk of or with a proliferative disorder. The liquid antibody formulations of the invention or combination therapies of the invention may be used as the first, second, third, fourth, or fifth therapy to prevent, treat and/or manage a proliferative disorder or one or more symptom thereof. The invention also includes methods of preventing, treating and/or managing a proliferative disorder or one or more symptoms thereof in a patient undergoing therapies for other disease or disorders. The invention encompasses methods of preventing, treating and/or managing a proliferative disorder or one or more symptoms thereof in a patient before any adverse effects or intolerance to therapies other than antibodies of the invention develops. The invention also encompasses methods of preventing, treating and/or managing a proliferative disorder or a symptom thereof in patients who are susceptible to adverse reactions to conventional therapies.

The invention encompasses methods for preventing, treating and/or managing a proliferative disorder or a symptom thereof in a patient who has proven refractory to therapies other than antibodies, compositions, or combination therapies of the invention. In certain embodiments, a patient with a proliferative disorder is refractory to a therapy when proliferation disorders has not been eradicated and/or the symptoms have not been alleviated. The determination of whether a patient is refractory can be made either in vivo or in vitro by any method known in the art for assaying the effectiveness of a treatment of proliferative disorders, using art-accepted meanings of “refractory” such a context. In various embodiments, a patient with a proliferative disorder is refractory when the patient's levels of IL-9 remain abnormal and/or if cellular proliferation has not been decreased.

The present invention provides methods for preventing, treating and/or managing a proliferative disorder or one or more symptoms thereof as an alternative to other conventional therapies. In specific embodiments, the patient being managed or treated in accordance with the methods of the invention is to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, persons with neuropsychiatric disorders or those who take psychotropic drugs, persons with histories of seizures, or persons on medication that would negatively interact with conventional agents used to manage or treat a proliferative disorder.

Therapies and dosages, routes of administration, and recommended usage of therapies for preventing, treating and/or managing proliferative disorders or one or more symptoms thereof are known in the art and have been described in such literature as the Physicians' Desk Reference (60th ed., 2006).

5.5.3. Inflammatory Disorder Treatment

The liquid formulations of the invention may be administered to a subject in need thereof to prevent, treat and/or manage an inflammatory disorder (e.g., asthma) or one or more symptoms thereof. The liquid formulations of the invention may also be administered in combination with one or more other therapies, preferably therapies useful for the prevention, treatment and/or management of an inflammatory disorder (including, but not limited to the prophylactic or therapeutic agents listed in Section 5.5.3.1, infra) to a subject in need thereof to prevent, treat and/or manage an inflammatory disorder or one or more symptoms thereof. In a specific embodiment, the invention provides a method of preventing, treating and/or managing an inflammatory disorder or one or more symptoms thereof, said method comprising administering to a subject in need thereof a dose of a prophylactically or therapeutically effective amount of a liquid formulation of the invention. In another embodiment, the invention provides a method of preventing, treating and/or managing an inflammatory disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that immunospecifically bind to an IL-9 polypeptide.

The invention provides methods for preventing, treating and/or managing one or more symptoms of an inflammatory disorder in a subject refractory to conventional therapies (e.g., methotrexate and a TNF-α antagonist (e.g., REMICADE™ or ENBREL™)) for such an inflammatory disorder, said methods comprising administering to said subject a dose of a prophylactically or therapeutically effective amount of a liquid formulation of the invention. The invention also provides methods for preventing, treating and/or managing one or more symptoms of an inflammatory disorder in a subject refractory to existing single agent therapies for such an inflammatory disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that immunospecifically bind to an IL-9 polypeptide. The invention also provides methods for managing or treating an inflammatory disorder by administering a liquid formulation of the invention in combination with any other treatment to patients who have proven refractory to other treatments but are no longer on these treatments. The invention also provides alternative methods for the treatment of an inflammatory disorder where another therapy has proven or may prove too toxic, i.e., results in unacceptable or unbearable side effects, for the subject being treated. For example, the liquid formulations of the invention may be administered to a subject, wherein the subject is refractory to a TNF antagonist or methotrexate. Further, the invention provides methods for preventing the recurrence of an inflammatory disorder in patients that have been treated and have no disease activity by administering a liquid formulation of the invention.

Inflammatory disorders that can be treated by the methods encompassed by the invention include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, osteoarthritis, spondyloarthropathies (e.g., psoriatic arthritis, ankylosing spondylitis, Reiter's Syndrome (reactive arthritis), inflammatory osteolysis, Wilson's disease and chronic inflammation resulting from chronic viral or bacteria infections. As described herein in Section 5.5.4.1, some autoimmune disorders are associated with an inflammatory condition.

Anti-inflammatory therapies and their dosages, routes of administration and recommended usage are known in the art and have been described in such literature as the Physicians' Desk Reference (60th ed., 2006).

5.5.3.1. Anti-Inflammatory Therapies

The present invention provides methods of preventing, treating and/or managing an inflammatory disorder or one or more symptoms thereof, said methods comprising administering to a subject in need thereof a liquid formulation of the invention and one or more therapies (e.g., prophylactic or therapeutic agents other than antibodies (including antibody fragments thereof) that immunospecifically bind to an IL-9 polypeptide. Any agent or therapy which is known to be useful, or which has been used or is currently being used for the prevention, treatment and/or management of an inflammatory disorder or one or more symptoms thereof can be used in combination with a liquid formulation of the invention in accordance with the invention described herein.

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (see Table 2, infra, for non-limiting examples of leukotriene and typical dosages of such agents)).

In a specific embodiment, an effective amount of one or more antibodies of the invention is administered in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety) to a subject to prevent, treat and/or manage an inflammatory disorder or one or more symptoms thereof. In another embodiment, an effective amount of one or more antibodies of the invention is administered in combination with an effective amount of siplizumab (MedImmune, Inc., International Publication No. WO 02/069904) to a subject to prevent, treat and/or manage an inflammatory disorder or one or more symptoms thereof. In another embodiment, an effective amount of one or more antibodies of the invention is administered in combination with an effective amount of one or more EphA2 inhibitors (e.g., one or more anti-EphA2 antibodies (MedImmune, Inc.; International Publication No. WO 02/102974 A4, dated Dec. 27, 2002, entitled “Mutant Proteins, High Potency Inhibitory Antibodies and FIMCH Crystal Structure,” International Publication No. 03/094859 A2, dated Nov. 20, 2003, entitled “EphA2 Monoclonal Antibodies and Methods of Use Thereof,” U.S. application Ser. No. 10/436,783 and published as U.S. Pat. Pub. No. US 2004/0091486 A1; and U.S. application Ser. No. 10/994,129 and published as U.S. Pat. Pub. No. US 2005/0152899 A1, each of which is incorporated herewith by reference)) to a subject to prevent, treat and/or manage an inflammatory disorder or one or more symptoms thereof. In yet another preferred embodiment, an effective amount of one or more antibodies of the invention is administered in combination with an effective amount of VITAXIN™, siplizumab, and/or EphA2 inhibitor to a subject to prevent, treat and/or manage an inflammatory disorder or one or more symptoms thereof.

In one embodiment, an effective amount of one or more antibody formulations of the invention is administered in combination with a mast cell protease inhibitor to a subject at risk of or with an inflammatory disorder. In another embodiment, the mast cell protease inhibitor is a tryptase kinase inhibitor, such as, but not limited to GW-45, GW-58, and genisteine. In a specific embodiment, the mast cell protease inhibitor is phosphatidylinositide-3′ (PI3)-kinase inhibitors, such as, but not limited to calphostin C. In another embodiment, the mast cell protease inhibitor is a protein kinase inhibitor such as, but not limited to staurosporine. In accordance with this embodiments, the mast cell protease inhibitor is preferably administered locally to the affected area.

Specific examples of immunomodulatory agents which can be administered in combination with a liquid formulation of the invention to a subject with an inflammatory disorder include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies)); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (e.g., anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies).

Any TNF-α antagonist well-known to one of skill in the art can be used in the compositions and methods of the invention. Non-limiting examples of TNF-α antagonists which can be administered in combination with a liquid formulation of the invention to a subject with an inflammatory disorder include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that blocks, reduces, inhibits or neutralizes the function, activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS). Examples of antibodies that immunospecifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that immunospecifically bind to TNF-α disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192).

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Non-limiting examples of anti-inflammatory agents which can be administered in combination with a liquid formulation of the invention to a subject with an inflammatory disorder include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In specific embodiments, patients with osteoarthritis are administered a prophylactically or therapeutically effective amount of a liquid formulation of the invention in combination with other agents or therapies useful for osteoarthritis prevention, treatment and/or management including but not limited to: analgesics (non-limiting examples are acetaminophen, in a dose up to 4000mg/d; phenacetin; and tramadol, in a daily dose in the range of 200 to 300 mg); NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin. Low dose NSAIDs are preferred, e.g., ibuprofen at 1200 mg/d, naproxen at 500 mg/d. A gastroprotective agent, e.g., misoprostol, famotidine or omeprazole, is preferred to use concurrently with a NSAID); nonacetylated salicylates including but not limited to salsalate; cyclooxygenase (Cox)-2-specific inhibitors (CSIs), including but not limited to, celecoxib and rofecoxib; intra- or periarticular injection of a depot glucocorticoid preparation; intra-articular injection of hyaluronic acid; capsaicin cream; copious irrigation of the osteroarthritis knee to flush out fibrin, cartilage shards and other debris; and joint replacement surgery. The liquid formulations of the invention can also be used in combination with other nonpharmacologic measures in prevention, treatment and/or management of osteoarthritis including but not limited to: reduction of joint loading (non-limiting examples are correction of poor posture, support for excessive lumbar lordosis, avoid excessive loading of the involved joint, avoid prolonged standing, kneeling and squatting); application of heat to the affected joint; aerobic exercise and other physical therapies.

In specific embodiments, patients with rheumatoid arthritis are administered a prophylactically or therapeutically effective amount of a liquid formulation of the invention in combination with other agents or therapies useful in prevention, treatment and/or management of rheumatoid arthritis including but not limited to: NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin.); analgesics (non-limiting examples are acetaminophen, phenacetin and tramadol); CSIs including but not limited to, celecoxib and rofecoxib; glucocorticoids (preferably low-dose oral glucocorticoids, e.g., <7.5 mg/d prednisone, or monthly pulses with high-dose glucocorticoids, or intraarticular glucocorticoids); disease-modifying antirheumatic drugs (DMARDs) including but not limited to, methotrexate (preferably given intermittent low dose, e.g., 7.5-30 mg once weekly), gold compounds (e.g., gold salts), D-penicillamine, the antimalarials (e.g., chloroquine), and sulfasalazine; TNF-α neutralizing agents including but not limited to, etanercept and infliximab; immunosuppressive and cytotoxic agents (examples include but not limited to, azathioprine, leflunomide, cyclosporine, and cyclophosphamide), and surgery (examples include but not limited to, arthroplasties, total joint replacement, reconstructive hand surgery, open or arthroscopic synovectomy, and early tenosynovectomy of the wrist). The liquid formulations of the invention may also be used in combination with other measures in prevention, treatment and/or management of the rheumatoid arthritis including but not limited to: rest, splinting to reduce unwanted motion of inflamed joint, exercise, used of a variety of orthotic and assistive devices, and other physical therapies. The liquid formulations of the invention may also be used in combination with some nontraditional approaches in prevention, treatment and/or management of rheumatoid arthritis including but not limited to, diets (e.g., substituting omega-3 fatty acids such as eicosapentaenoic acid found in certain fish oils for dietary omega-6 essential fatty acids found in meat), vaccines, hormones and topical preparations.

In specific embodiments, patients with chronic obstructive pulmonary disease (COPD) are administered a prophylactically or therapeutically effective amount of a liquid formulation of the invention in combination with other agents or therapies useful in prevention, treatment and/or management of COPD including but not limited to: bronchodilators including but not limited to, short- and long-acting β₂-adrenergic agonists (examples of short-acting β₂ agonist include but not limited to, albuterol, pirbuterol, terbutaline, and metaproterenol; examples of long-acting β₂ agonist include but not limited to, oral sustained-release albuterol and inhaled salmeterol), anticholinergics (examples include but not limited to ipratropium bromide), and theophylline and its derivatives (therapeutic range for theophylline is preferably 10-20 μg/mL); glucocorticoids; exogenous α₁AT (e.g., α₁AT derived from pooled human plasma administered intravenously in a weekly dose of 60 mg/kg); oxygen; lung transplantation; lung volume reduction surgery; endotracheal intubation, ventilation support; yearly influenza vaccine and pneumococcal vaccination with 23-valent polysaccharide; exercise; and smoking cessation.

In specific embodiments, patients with pulmonary fibrosis are administered a prophylactically or therapeutically effective amount of a liquid formulation of the invention in combination with an effective amount of one or more other agents useful for pulmonary fibrosis therapy including but not limited to: oxygen; corticosteroids (a non-limiting example is to administer daily prednisone beginning at 1-1.5 mg/kg/d (up to 100 mg/d) for six weeks and tapering slowly over 3-6 months to a minimum maintenance dose of 0.25 mg/kg/d); cytotoxic drugs (non-limiting examples are cyclophosphamide at 100-120 mg orally once daily, and azathioprine at 3 mg/kg up to 200 mg orally once daily); bronchodilators (non-limiting examples are short- and long-acting β₂-adrenergic agonists, anticholinergics, and theophylline and its derivatives); and antihistamines (non-limiting examples are diphenhydramine and doxylamine).

In specific embodiments, patients with asthma are administered a prophylactically or therapeutically effective amount of a liquid formulation of the invention in combination with an effective amount of one or more other agents useful for asthma therapy. Non-limiting examples of such agents include adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-Kappa-β inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)). In a specific embodiment, the anti-inflammatory agent is a leukotriene inhibitor (e.g., montelukast (SINGULAIR™), zafirlukast (ACCOLATE™), pranlukast (ONON™), or zileuton (ZYFLO™) (see Table 5)).

TABLE 5
Leukotriene Inhibitors for Asthma Therapy.

| Leukotriene Modifier | Usual Daily Dosage |
|---|---|
| Montelukast (SINGULAIR ™) | 4 mg for 2-5 years old |
| | 5 mg for 6 to 15 years old |
| | 10 mg for 15 years and older |
| Zafirlukast (ACCOLATE ™) | 10 mg b.i.d. for 5 to 12 years old |
| | twice daily |
| | 20 mg b.i.d. for 12 years or older |
| | twice daily |
| Pranlukast (ONON ™) | Only avialable in Asia |
| Zyleuton (ZYFLO ™) | 600 mg four times a day for 12 years |
| | and older |

In specific embodiments, patients with allergy are administered a prophylactically or therapeutically effective amount of a liquid formulation of the invention in combination with an effective amount of one or more other agents useful for allergy therapy. Non-limiting examples of such agents include antimediator drugs (e.g., antihistamine, see Table 6, infra, for non-limiting examples of antihistamine and typical dosages of such agents), corticosteroids, decongestants, sympathomimetic drugs (e.g., α-adrenergic and β-adrenergic drugs), TNX901 (Leung et al., 2003, N Engl J Med 348(11):986-993), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), theophylline and its derivatives, glucocorticoids, and immunotherapies (e.g., repeated long-term injection of allergen, short course desensitization, and venom immunotherapy).

TABLE 6
H₁ Antihistamines.

| Chemical class and representative drugs | Usual daily dosage |
|---|---|
| Ethanolamine | |
| Diphehydramine | 25-50 mg every 4-6 hours |
| Clemastine | 0.34-2.68 mg every 12 hours |
| Ethylenediamine | |
| Tripelennamine | 25-50 mg every 4-6 hours |
| Alkylamine | |
| Brompheniramine | 4 mg every 4-6 hours; or |
| | 8-12 mg of SR form every |
| | 8-12 hour |
| Chlorpheniramine | 4 mg every 4-6 hours; or |
| | 8-12 mg of SR form every |
| | 8-12 hour |
| Triprolidine (1.25 mg/5 ml) | 2.5 mg every 4-6 hours |
| Phenothiazine | |
| Promethazine | 25 mg at bedtime |
| Piperazine | |
| Hydroxyzine | 25 mg every 6-8 hours |
| Piperidines | |
| Astemizole (nonsedating) | 10 mg/d |
| Azatadine | 1-2 mg every 12 hours |
| Cetirzine | 10 mg/d |
| Cyproheptadine | 4 mg every 6-8 hour |
| Fexofenadine (nonsedating) | 60 mg every 12 hours |
| Loratidine (nonsedating) | 10 mg every 24 hours |

5.5.4. Autoimmune Disorder Treatment

The liquid formulations of the invention may be administered to a subject in need thereof to prevent, treat and/or manage an autoimmune disorder or one or more symptoms thereof. The liquid formulations of the invention may also be administered in combination with one or more other therapies, preferably therapies useful for the prevention, management or treatment of an autoimmune disorder (including, but not limited to the prophylactic or therapeutic agents listed in Section 5.5.4.1 hereinbelow) to a subject in need thereof to prevent, treat and/or manage an autoimmune disorder or one or more symptoms thereof. In a specific embodiment, the invention provides a method of preventing, treating and/or managing an autoimmune disorder or one or more symptoms thereof, said method comprising administering to a subject in need thereof a dose of a prophylactically or therapeutically effective amount of a liquid formulation of the invention. In another embodiment, the invention provides a method of preventing, treating and/or managing an autoimmune disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that immunospecifically bind to an IL-9 polypeptide.

The invention provides methods for preventing, treating and/or managing an autoimmune disorder or one or more symptoms thereof in a subject refractory to conventional therapies for such an autoimmune disorder, said methods comprising administering to said subject a dose of a prophylactically or therapeutically effective amount of a liquid formulation of the invention. The invention also provides methods for preventing, treating and/or managing an autoimmune disorder or one or more symptoms thereof in a subject refractory to existing single agent therapies for such an autoimmune disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that immunospecifically bind to an IL-9 polypeptide. The invention also provides methods for preventing, treating and/or managing an autoimmune disorder or one or more symptoms thereof by administering a liquid formulation of the invention in combination with any other treatment to patients who have proven refractory to other treatments but are no longer on these treatments. The invention also provides alternative methods for the management or treatment of an autoimmune disorder where another therapy has proven or may prove too toxic, i.e., results in unacceptable or unbearable side effects, for the subject being treated. Particularly, the invention provides alternative methods for the management or treatment of an autoimmune disorder where the patient is refractory to other therapies. Further, the invention provides methods for preventing the recurrence of an autoimmune disorder in patients that have been treated and have no disease activity by administering a liquid formulation of the invention.

In autoimmune disorders, the immune system triggers an immune response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders that can be treated by the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

Autoimmune therapies and their dosages, routes of administration and recommended usage are known in the art and have been described in such literature as the Physicians' Desk Reference (60th ed., 2006).

5.5.4.1. Autoimmune Disorder Therapies

The present invention provides methods of preventing, treating and/or managing an autoimmune disorder or one or more symptoms thereof, said methods comprising administering to a subject in need thereof a liquid formulation of the invention and one or more therapies (e.g., prophylactic or therapeutic agents) other than antibodies (including antibody fragments thereof) that immunospecifically bind to an IL-9 polypeptide. Any agent or therapy which is known to be useful, or which has been used or is currently being used for the prevention, treatment and/or management of an autoimmune disorder or one or more symptoms thereof can be used in combination with a liquid formulation of the invention in accordance with the invention described herein. Examples of such agents include, but are not limited to, immunomodulatory agents, anti-inflammatory agents and TNF-α antagonists. Specific examples of immunomodulatory agents, anti-inflammatory agents and TNF-α antagonists which can be used in combination with a liquid formulation of the invention for the prevention, treatment and/or management of an autoimmune disorder are disclosed herein above.

In specific embodiments, patients with multiple sclerosis (MS) are administered a prophylactically or therapeutically effective amount of a liquid formulation of the invention in combination with other agents or therapies useful in prevention, treatment and/or management of MS including but not limited to: IFN-β1b (Betaseron) (e.g., 8.0 million international unites (MIU) is administered by subcutaneous injection every other day); IFN-β1a (Avonex) (e.g., 6.0 MIU is administered by intramuscular injection once every week); glatiramer acetate (Copaxone) (e.g., 20 mg is administered by subcutaneous injection every day); mitoxantrone (e.g., 12 mg/m² is administered by intravenous infusion every third month); azathioprine (e.g., 2-3 mg/kg body weight is administered orally each day); methotrexate (e.g., 7.5 mg is administered orally once each week); cyclophosphamide; intravenous immunoglobulin (e.g., 0.15-0.2 g/kg body weight administered monthly for up to 2 years); glucocorticoids; methylprednisolone (e.g., administered in bimonthly cycles at high doses); 2-chlorodeoxyadenosine (cladribine); baclofen (e.g., 15 to 80 mg/d in divided doses, or orally in higher doses up to 240 mg/d, or intrathecally via an indwelling catheter); cycloenzaprine hydrochloride (e.g., 5-10 mg bid or tid); clonazepam (e.g., 0.5 to 1.0 mg tid, including bedtime dose); clonidine hydrochloride (e.g., 0.1 to 0.2 mg tid, including a bedtime dose); carbamazepine (e.g., 100-1200 mg/d in divided, escalating doses); gabapentin (e.g., 300-3600 mg/d); dilantin (e.g., 300-400 mg/d); amitriptyline (e.g., 25-150 mg/d); baclofen (e.g., 10-80 mg/d); primidone (e.g., 125-250 mg bid or tid); ondansetron (e.g., 4 to 8 mg bid or tid); isoniazid (e.g., up to 1200 mg in divided doses); oxybutynin (e.g., 5 mg bid or tid); tolterodine (e.g., 1-2 mg bid); propantheline (e.g., 7.5 to 15 mg qid); bethanecol (e.g., 10-50 mg tid or qid); terazosin hydrochloride (e.g., 1-5 mg at bedtime); sildenafil citrate (e.g., 50-100 mg po prn); amantading (e.g., 100 mg bid); pemoline (e.g., 37.5 mg bid); high dose vitamins; calcium orotate; gancyclovir; antibiotic; and plasma exchange.

In specific embodiments, patients with psoriasis are administered a prophylactically or therapeutically effective amount of a liquid formulation of the invention in combination with other agents or therapies useful in prevention, treatment and/or management of psoriasis including but not limited to: topical steroid cream or ointment; tar (examples including but not limited to, Estar, Psorigel, Fototar cream, and LCD 10% in Nutraderm lotion or mixed directly with triamcinolone 0.1% cream); occlusion; topical vitamin D analogue (a non-limiting example is calcipotriene ointment); ultraviolet light; PUVA (psoralen plus ultraviolet A); methotrexate (e.g., up to 25 mg once weekly or in divided doses every 12 hours for three doses once a week); synthetic retinoid (a non-limiting examples is etretinate, e.g., in dosage of 0.5-1 mg/kg/d); immunomodulatory therapy (a non-limiting example is cyclosporine); sulfasalazine (e.g., in dosages of 1 g three times daily).

In specific embodiments, patients with Crohn's disease are administered a prophylactically or therapeutically effective amount of a liquid formulation of the invention in combination with other agents or therapies useful in prevention, treatment and/or management of Crohn's disease including but not limited to: antidiarrheals (e.g., loperamide 2-4 mg up to 4 times a day, diphenoxylate with atropine 1 tablet up to 4 times a day, tincture of opium 8-15 drops up to 4 times a day, cholestyramine 2-4 g or colestipol 5 g once or twice daily), antispasmodics (e.g., propantheline 15 mg, dicyclomine 10-20 mg, or hyoscyamine 0.125 mg given before meals), 5-aminosalicylic acid agents (e.g., sulfasalazine 1.5-2 g twice daily, mesalamine (ASACOL®) and its slow release form (PENTASA®), especially at high dosages, e.g., PENTASA® 1 g four times daily and ASACOL® 0.8-1.2 g four times daily), corticosteroids, immunomodulatory drugs (e.g., azathioprine (1-2 mg/kg), mercaptopurine (50-100 mg), cyclosporine, and methotrexate), antibiotics, TNF inhibitors (e.g., inflixmab (REMICADE®)), immunosuppressive agents (e.g., tacrolimus, mycophenolate mofetil, and thalidomide), anti-inflammatory cytokines (e.g., IL-10 and IL-11), nutritional therapies, enteral therapy with elemental diets (e.g., Vivonex for 4 weeks), and total parenteral nutrition.

In specific embodiments, patients with lupus erythematosus are administered a prophylactically or therapeutically effective amount of a liquid formulation of the invention in combination with other agents or therapies useful in prevention, treatment and/or management of lupus erythematosus including but not limited to: antimalarials (including but not limited to, hydroxychloroquine); glucocorticoids (e.g., low dose, high dose, or high-dose intravenous pulse therapy can be used); immunosuppressive agents (including but not limited to, cyclophosphamide, chlorambucil, and azanthioprine); cytotoxic agents (including but not limited to methotrexate and mycophenolate mofetil); androgenic steroids (including but not limited to danazol); and anticoagulants (including but not limited to warfarin).

In a specific embodiment, an prophylactically or therapeutically effective amount of one or more liquid antibody formulations of the invention is administered in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or

Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety) to a subject to prevent, treat and/or manage an autoimmune disorder or one or more symptoms thereof. In another preferred embodiment, an effective amount of one or more antibodies of the invention is administered in combination with an effective amount of siplizumab (MedImmune, Inc., International Publication No. WO 02/069904) to a subject to prevent, treat and/or manage an autoimmune disorder or one or more symptoms thereof. In another embodiment, an effective amount of one or more antibodies of the invention is administered in combination with an effective amount of one or more EphA2 inhibitors (e.g., one or more anti-EphA2 antibodies (MedImmune, Inc.; International Publication No. WO 02/102974 A4, dated Dec. 27, 2002, entitled “Mutant Proteins, High Potency Inhibitory Antibodies and FIMCH Crystal Structure,” International Publication No. 03/094859 A2, dated Nov. 20, 2003, entitled “EphA2 Monoclonal Antibodies and Methods of Use Thereof,” U.S. application Ser. No. 10/436,783 and published as U.S. Pat. Pub. No. US 2004/0091486 A1; and U.S. application Ser. No. 10/994,129 and published as U.S. Pat. Pub. No. US 2005/0152899 A1, each of which is incorporated herewith by reference)) to a subject to prevent, treat and/or manage an autoimmune disorder or one or more symptoms thereof. In yet another embodiment, an effective amount of one or more antibodies of the invention is administered in combination with an effective amount of VITAXIN™, siplizumab, and/or EphA2 inhibitor to a subject to prevent, treat and/or manage an autoimmune disorder or one or more symptoms thereof.

The antibody formulations of the invention or combination therapies of the invention may be used as the first, second, third, fourth, or fifth therapy to prevent, treat and/or manage an autoimmune disorder or one or more symptom thereof. The invention also includes methods of preventing, treating and/or managing an autoimmune disorder or one or more symptoms thereof in a patient undergoing therapies for other disease or disorders. The invention encompasses methods of preventing, treating and/or managing an autoimmune disorder or one or more symptoms thereof in a patient before any adverse effects or intolerance to therapies other than antibodies of the invention develops. The invention also encompasses methods of preventing, treating and/or managing an autoimmune disorder or a symptom thereof in refractory patients. The invention encompasses methods for preventing, treating and/or managing a proliferative disorder or a symptom thereof in a patient who has proven refractory to therapies other than antibodies, compositions, or combination therapies of the invention. The determination of whether a patient is refractory can be made either in vivo or in vitro by any method known in the art for assaying the effectiveness of a treatment of autoimmune disorders, using art-accepted meanings of “refractory” such a context. In certain embodiments, a patent with an autoimmune disorder is refractory to a therapy when one or more symptoms of an autoimmune disorder is not prevented, managed, and/or alleviated. The invention also encompasses methods of preventing, treating and/or managing an autoimmune disorder or a symptom thereof in patients who are susceptible to adverse reactions to conventional therapies.

The present invention encompasses methods for preventing, treating and/or managing an autoimmune disorder or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease, patients with broncho-pulmonary dysplasia, patients with congenital heart disease, patients with cystic fibrosis, patients with acquired or congenital heart disease, and patients suffering from an infection), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat and/or manage a viral respiratory infection or one or more symptoms thereof.

Autoimmune therapies and their dosages, routes of administration and recommended usage are known in the art and have been described in such literature as the Physicians' Desk Reference (60th ed., 2006).

5.5.5. Viral Infections

One or more antibody formulations of the invention can be administered to a subject to prevent, treat and/or manage a viral infection or one or more symptoms thereof. One or more antibody formulations of the invention may be administered in combination with one or more other therapies (e.g., one or more prophylactic or therapeutic agents) other than antibody formulations of the invention useful for the prevention, treatment and/or management of a viral infection to a subject predisposed to or with a viral infection, preferably a respiratory viral infection. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons and other interferons, and AZT.

In specific embodiments, the anti-viral agent is an immunomodulatory agent that is immunospecific for a viral antigen. As used herein, the term “viral antigen” includes, but is not limited to, any viral peptide, polypeptide and protein (e.g., HIV gp120, HIV nef, RSV F glycoprotein, RSV G glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response. Antibodies useful in this invention for treatment of a viral infectious disease include, but are not limited to, antibodies against antigens of pathogenic viruses, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporiipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory synctial virus), and metapneumovirus (e.g., avian pneumovirus and human metapneumovirus)), picornaviridae (e.g., enterovirus, rhinovirus, hepatovirus (e.g., human hepatits A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Specific examples of antibodies available useful for the treatment of a viral infectious disease include, but are not limited to, PRO542 (Progenics) which is a CD4 fusion antibody useful for the treatment of HIV infection; Ostavir (Protein Design Labs, Inc., CA) which is a human antibody useful for the treatment of hepatitis B virus; and Protovir (Protein Design Labs, Inc., CA) which is a humanized IgG1 antibody useful for the treatment of cytomegalovirus (CMV); and palivizumab (SYNAGIS®; MedImmune, Inc.; International Publication No. WO 02/43660) which is a humanized antibody useful for treatment of RSV.

In a specific embodiment, the anti-viral agents used in the compositions and methods of the invention inhibit or reduce a pulmonary or respiratory virus infection, inhibit or reduce the replication of a virus that causes a pulmonary or respiratory infection, or inhibit or reduce the spread of a virus that causes a pulmonary or respiratory infection to other cells or subjects. In another preferred embodiment, the anti-viral agents used in the compositions and methods of the invention inhibit or reduce infection by RSV, hMPV, or PIV, inhibit or reduce the replication of RSV, hMPV, or PIV, or inhibit or reduce the spread of RSV, hMPV, or PIV to other cells or subjects. Examples of such agents and methods of treatment of RSV, hMPV, and/or PIV infections include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons. See U.S. patent application Ser. No. 10/628,088, filed Jul. 25, 2003 and published as U.S. Pat. Pub. No. US 2004/0096451 A1, entitled “Methods of Treating and Preventing RSV, HMPV, and PIV Using Anti-RSV, Anti-HMPV, and Anti-PIV Antibodies,” and U.S. patent application Ser. No. 10/371,122 filed Feb. 21, 2003 and published as U.S. Pat. Pub. No. US 2004/0005544 A1, which are incorporated herein by reference in its entirety.

In preferred embodiments, the viral infection is RSV and the anti-viral antigen is an antibody that immunospecifically binds to an antigen of RSV. In certain embodiments, the anti-RSV-antigen antibody binds immunospecifically to an RSV antigen of the Group A of RSV. In other embodiments, the anti-RSV-antigen antibody binds immunospecifically to an RSV antigen of the Group B of RSV. In other embodiments, an antibody binds to an antigen of RSV of one Group and cross reacts with the analogous antigen of the other Group. In particular embodiments, the anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein. In additional specific embodiments, the anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to a RSV antigen are known in the art. For example, SYNAGIS® (Palivizumab) is a humanized monoclonal antibody presently used for the prevention of RSV infection in pediatric patients. In a specific embodiment, an antibody to be used with the methods of the present invention is palivizumab or an antibody-binding fragment thereof (e.g., a fragment containing one or more complementarity determining regions (CDRs) and preferably, the variable domain of palivizumab). The amino acid sequence of palivizumab is disclosed, e.g., in Johnson et al., 1997, J. Infectious Disease 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

One or more antibodies or antigen-binding fragments thereof that bind immunospecifically to a RSV antigen comprise a Fc domain with a higher affinity for the FcRn receptor than the Fc domain of palivizumab can also be used in accordance with the invention. Such antibodies are described in U.S. patent application Ser. No. 10/020,354, filed Dec. 12, 2001, which is incorporated herein by reference in its entireties. Further, the anti-RSV-antigen antibody A4B4; P12f2 P12f4; P11d4; A1e9; A12a6; A13c4; A17d4; A4B4; 1X-493L1; FR H3-3F4; M3H9; Y10H6; DG; AFFF; AFFF(1); 6H8; L1-7E5; L2-15B10; A13a11; A1h5; A4B4(1);A4B4-F52S; or A4B4L1FR-S28R can be used in accordance with the invention. These antibodies are disclosed in International Application Publication No.: WO 02/43660, entitled “Methods of Administering/Dosing Anti-RSV Antibodies for Prophylaxis and Treatment”, by Young et al., and U.S. patent application Ser. No. 10/628,088 filed Jul. 25, 2003 and published as U.S. Pat. Pub. No. US 2004/0096451 A1, entitled “Methods of Treating and Preventing RSV, HMPV, and PIV Using Anti-RSV, Anti-HMPV, and Anti-PIV Antibodies” which are incorporated herein by reference in their

In certain embodiments, the anti-RSV-antigen antibodies are the anti-RSV-antigen antibodies of or are prepared by the methods of U.S. application Ser. No. 09/724,531, filed Nov. 28, 2000; Ser. No. 09/996,288, filed Nov. 28, 2001; and Ser. No. 09/996,265, filed Nov. 28, 2001 and published as U.S. Pat. Pub. No. US 2003/0091584 A1, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application No. 60/388,921, filed Jun. 14, 2002, and U.S. patent application Ser. No. 10/461,863, filed Mar. 7, 2003 and published as U.S. Pat. Pub. No. US 2004/0018200 A1, which are incorporated by reference herein in their entireties.

Anti-viral therapies and their dosages, routes of administration and recommended usage are known in the art and have been described in such literature as the Physicians' Desk Reference (60th ed., 2006). Additional information on respiratory viral infections is available in Cecil Textbook of Medicine (18th ed., 1988).

5.5.5.1. Therapies for Viral Infections

In a specific embodiment, the invention provides methods of preventing, treating and/or managing a viral respiratory infection or one or more symptoms thereof, said method comprising administering to a subject in need thereof an effective amount of one or more antibody formulations of the invention. In another embodiment, the invention provides a method of preventing, treating and/or managing a viral respiratory infection or one or more symptoms thereof, said method comprising administering to a subject in need thereof an effective amount of one or more antibody formulations of the invention and an effective amount of one or more therapies (e.g., one or more prophylactic or therapeutic agents) other the than antibody formulations of the invention.

In certain embodiments, an effective amount of one or more antibody formulations of the invention is administered in combination with an effective amount of one or more therapies (e.g., one or more prophylactic or therapeutic agents) currently being used, have been used, or are known to be useful in the prevention, treatment and/or management of a viral infection, e.g., a viral respiratory infection, or one or more symptoms thereof to a subject in need thereof. Therapies for a viral infection, e.g., a viral respiratory infection include, but are not limited to, anti-viral agents such as amantadine, oseltamivir, ribaviran, palivizumab (SYNAGIS™), and anamivir. In certain embodiments, an effective amount of one or more antibody formulations of the invention is administered in combination with one or more supportive measures to a subject in need thereof to prevent, treat and/or manage a viral infection or one or more symptoms thereof. Non-limiting examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial infections).

Any type of viral infection or condition resulting from or associated with a viral infection (e.g., a respiratory condition) can be prevented, treated and/or managed in accordance with the methods of the invention, said methods comprising administering an effective amount of one or more antibody formulations of the invention alone or in combination with an effective amount of another therapy (e.g., a prophylactic or therapeutic agent other than antibody formulations of the invention). Examples of viruses which cause viral infections include, but are not limited to, retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, hMPV, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and Ply), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Biological responses to a viral infection include, but not limited to, elevated levels of IgE antibodies, increased proliferation and/or infiltration of T cells, increased proliferation and/or infiltration of B cells, epithelial hyperplasia, and mucin production. In a specific embodiment, the invention also provides methods of preventing, treating and/or managing viral respiratory infections that are associated with or cause the common cold, viral pharyngitis, viral laryngitis, viral croup, viral bronchitis, influenza, parainfluenza viral diseases (“PIV”) diseases (e.g., croup, bronchiolitis, bronchitis, pneumonia), respiratory syncytial virus (“RSV”) diseases, metapneumavirus diseases, and adenovirus diseases (e.g., febrile respiratory disease, croup, bronchitis, pneumonia), said method comprising administering an effective amount of one or more antibody formulations of the invention alone or in combination with an effective amount of another therapy.

In a specific embodiment, influenza virus infections, PIV infections, hMPV infections, adenovirus infections, and/or RSV infections, or one or more of symptoms thereof are prevented, treated and/or managed in accordance with the methods of the invention. In a specific embodiment, the invention provides methods for preventing, treating and/or managing a RSV respiratory infection or one or more symptoms thereof, said methods comprising administering to a subject in need thereof an effective amount of one or more antibody formulations of the invention alone or in combination with one or more anti-viral agents such as, but not limited to, amantadine, rimantadine, oseltamivir, znamivir, ribaviran, RSV-IVIG (i.e., intravenous immune globulin infusion) (RESPIGAM™), and palivizumab (SYNAGIS™). In a specific embodiment, the invention provides methods for preventing, treating and/or managing a PIV infection or one or more symptoms thereof, said methods comprising administering to a subject in need thereof an effective amount of one or more antibody formulations of the invention alone or in combination with an effective amount of one or more anti-viral agents such as, but not limited to, amantadine, rimantadine, oseltamivir, znamivir, ribaviran, and palivizumab (SYNAGIS™). In another specific embodiment, the invention provides methods for preventing, treating and/or managing a hMPV infection or one or more symptoms thereof, said methods comprising of administering an effective amount of one or more antibody formulations of the invention alone or in combination with an effective amount of one or more anti-viral agents, such as, but not limited to, amantadine, rimantadine, oseltamivir, znamivir, ribaviran, and palivizumab (SYNAGIS™) to a subject in need thereof. In a specific embodiment, the invention provides methods for preventing, treating and/or managing influenza, said methods comprising administering an effective amount of one or more antibody formulations of the invention alone or in combination with an effective amount of an anti-viral agent such as, but not limited to zanamivir (RELENZA®), oseltamivir (TAMIFLU®), rimantadine, and amantadine (SYMADINE®; SYMMETREL®) to a subject in need thereof.

The invention provides methods for preventing the development of asthma in a subject who suffers from or had suffered from a viral respiratory infection, said methods comprising adminsitering an effective amount of one or more antibody formulations of the invention alone or in combination with an effective amount of another therapy. In a specific embodiment, the subject is an infant born prematurely, an infant, or a child. In another specific embodiment, the subject suffered from or suffers from RSV infection.

In a specific embodiment, the invention provides methods for preventing, treating and/or managing one or more secondary responses to a primary viral infection, said methods comprising of administering an effective amount of one or more antibody formulations of the invention alone or in combination with an effective amount of other therapies (e.g., other prophylactic or therapeutic agents). Examples of secondary responses to a primary viral infection, particularly a primary viral respiratory infection, include, but are not limited to, asthma-like responsiveness to mucosal stimula, elevated total respiratory resistance, increased susceptibility to secondary viral, bacterial, and fungal infections, and development of such conditions such as, but not limited to, pneumonia, croup, and febrile bronchitis.

In a specific embodiment, the invention provides methods of preventing, treating and/or managing a viral infection or one or more symptoms thereof, said methods comprising administering to a subject in need thereof an effective amount of one or more antibody formulations of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety). In another specific embodiment, the invention provides methods for preventing, treating and/or managing a viral infection or one or more symptoms thereof, said methods comprising administering to a subject in need thereof an effective amount of one or more antibodies of the invention in combination with an effective amount of siplizumab (MedImmune, Inc., International Pub. No. WO 02/069904). In another embodiment, the invention provides methods for preventing, treating and/or managing a viral infection or one or more symptoms thereof, said methods comprising administering to a subject in need thereof an effective amount of one or more antibodies of the invention in combination with an effective amount of one or more EphA2 inhibitors (e.g., one or more anti-EphA2 antibodies (MedImmune, Inc.; International Publication No. WO 02/102974 A4, dated Dec. 27, 2002, entitled “Mutant Proteins, High Potency Inhibitory Antibodies and FIMCH Crystal Structure,” International Publication No. 03/094859 A2, dated Nov. 20, 2003, entitled “EphA2 Monoclonal Antibodies and Methods of Use Thereof,” U.S. application Ser. No. 10/436,783 and published as U.S. Pat. Pub. No. US 2004/0091486 A1; and U.S. patent application Ser. No. 10/994,129 and published as U.S. Pat. Pub. No. US 2005/0152899 A1, each of which is incorporated herewith by reference)). In yet another embodiment, the invention provides methods for preventing, treating and/or managing a viral infection or one or more symptoms thereof, said methods comprising administering to a subject in need thereof an effective amount of one or more antibodies of the invention in combination with an effective amount of VITAXIN™, siplizumab, and/or EphA2.

In one embodiment, an effective amount of one or more antibody formulations of the invention is administered in combination with an effective amount of one or more anti-IgE antibodies to a subject to prevent, treat and/or manage a viral infection or one or more symptoms thereof. In a specific embodiment, an effective amount of one or more antibody formulations of the invention is administered in combination with an effective amount of anti-IgE antibody TNX901 to a subject to prevent, treat and/or manage a viral infection or one or more symptoms thereof. In a specific embodiment, an effective amount of one or more antibody formulations of the invention is administered in combination with an effective amount of anti-IgE antibody rhuMAb-E25 omalizumab to a subject to prevent, treat and/or manage a viral infection or one or more symptoms thereof.

In another embodiment, an effective amount of one or more antibody formulations of the invention is administered in combination with an effective amount of anti-IgE antibody HMK-12 to a subject to prevent, treat and/or manage a viral infection or one or more symptoms thereof. In a specific embodiment, an effective amount of one or more antibody formulations of the invention is administered in combination with an effective amount of anti-IgE antibody 6HD5 to a subject to prevent, treat and/or manage a viral infection or one or more symptoms thereof. In another embodiment, an effective amount of one or more antibody formulations of the invention is administered in combination with an effective amount of anti-IgE antibody MAb Hu-901 to a subject to prevent, treat and/or manage a viral infection or one or more symptoms thereof

The invention encompasses methods for preventing the development of viral infections, e.g., viral respiratory infections, in a patient expected to suffer from a viral infection or at increased risk of such an infection, e.g., patients with suppressed immune systems (e.g., organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from a respiratory infection). The patients may or may not have been previously treated for a respiratory infection.

The antibody formulations of the invention or combination therapies of the invention may be used as the first, second, third, fourth, or fifth therapy to prevent, treat and/or manage a viral infection, e.g., a viral respiratory infection, or one or more symptom thereof. The invention also includes methods of preventing, treating and/or managing a viral infection, e.g., a viral respiratory infection, or one or more symptoms thereof in a patient undergoing therapies for other diseases or disorders associated with or characterized by aberrant expression and/or activity of an IL-9 polypeptide, diseases or disorders associated with or characterized by aberrant expression and/or activity of the IL-9R or one or more subunits thereof, autoimmune diseases, inflammatory diseases, proliferative diseases, or infections (e.g., respiratory infections), or one or more symptoms thereof. The invention encompasses methods of preventing, treating and/or managing a viral infection, e.g., a viral respiratory infection, or one or more symptoms thereof in a patient before any adverse effects or intolerance to therapies other than antibody formulations of the invention develops. The invention also encompasses methods of preventing, treating and/or managing a viral infection, e.g., a viral respiratory infection, or a symptom thereof in refractory patients. In certain embodiments, a patient with a viral infection, e.g., a viral respiratory infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a viral respiratory infection is refractory when viral replication has not decreased or has increased. The invention also encompasses methods of preventing the onset or reoccurrence of viral respiratory infections in patients at risk of developing such infections. The invention also encompasses methods of preventing, treating and/or managing a viral infection, e.g., a viral respiratory infection, or a symptom thereof in patients who are susceptible to adverse reactions to conventional therapies. The invention further encompasses methods for preventing, treating and/or managing a viral infection, e.g., a viral respiratory infection, for which no anti-viral therapy is available.

The invention encompasses methods for preventing, treating and/or managing a viral infection, e.g., a viral respiratory infection, or a symptom thereof in a patient who has proven refractory to therapies other than antibody formulations of the invention but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring viral infections despite management or treatment with existing therapies.

The present invention encompasses methods for preventing, treating and/or managing a viral infection, e.g., a viral respiratory infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat and/or manage a viral infection or one or more symptoms thereof.

Viral infection therapies and their dosages, routes of administration and recommended usage are known in the art and have been described in such literature as the Physicians' Desk Reference (60th ed., 2006).

5.5.6. Bacterial Infections

The invention provides a method of preventing, treating and/or managing a bacterial infection or one or more symptoms thereof, said method comprising administering to a subject in need thereof an effective amount of one or more antibody formulations of the invention. In another embodiment, the invention provides a method of preventing, treating and/or managing a bacterial infection or one or more symptoms thereof, said method comprising administering to a subject in need thereof an effective amount of a one or more antibody formulations of the invention and an effective amount of one or more therapies (e.g., one or more prophylactic or therapeutic agents), other than antibody formulations of the invention. Anti-bacterial agents and therapies well known to one of skill in the art for the prevention, treatment and/or management of bacterial infections can be used in the compositions and methods of the invention. Non-limiting examples of anti-bacterial agents include proteins, polypeptides, peptides, fusion proteins, antibodies, nucleic acid molecules, organic molecules, inorganic molecules, and small molecules that inhibit or reduce a bacterial infection, inhibit or reduce the replication of bacteria, or inhibit or reduce the spread of bacteria to other subjects. In particular, examples of anti-bacterial agents include, but are not limited to, penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In one embodiment, the anti-bacterial agent is an agent that inhibits or reduces a pulmonary or respiratory bacterial infection, inhibits or reduces the replication of a bacteria that causes a pulmonary or respiratory infection, or inhibits or reduces the spread of a bacteria that causes a pulmonary or respiratory infection to other subjects. In cases in which the pulmonary or respiratory bacterial infection is a mycoplasma infection (e.g., pharyngitis, tracheobronchitis, and pneumonia), the anti-bacterial agent is e.g., a tetracycline, erythromycin, or spectinomycin. In cases in which the pulmonary or respiratory bacterial infection is pneumonia caused by an aerobic gram negative bacilli (GNB), the anti-bacterial agent is preferably penicillin, first second, or third generation cephalosporin (e.g., cefaclor, cefadroxil, cephalexin, or cephazolin), erythomycin, clindamycin, an aminoglycoside (e.g., gentamicin, tobramycin, or amikacine), or a monolactam (e.g., aztreonam). In cases in which the pulmonary or respiratory bacterial infection is tuberculosis, the anti-bacterial agent is preferably, rifampcin, isonaizid, pyranzinamide, ethambutol, and streptomycin. In cases in which the respiratory infection is recurrent aspiration pneumonia, the anti-bacterial agent is preferably penicillin, an aminoglycoside, or a second or third generation cephalosporin.

5.5.6.1. Therapies for Bacterial Infections

Any type of bacterial infection or condition resulting from or associated with a bacterial infection (e.g., a respiratory infection) can be prevented, treated and/or managed in accordance with the methods of invention. Examples of bacteria which cause bacterial infections include, but not limited to, the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Erwinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcus (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, and Vampirovibrio family.

In a specific embodiment, the invention provides methods for preventing, treating and/or managing a bacterial respiratory infection or one or more symptoms thereof, said method comprising administering to a subject in need thereof an effective amount of one or more antibody formulations of the invention. In another embodiment, the invention provides a method of preventing, treating and/or managing a bacterial respiratory infection or one or more symptoms thereof, said method comprising administering to a subject in need thereof an effective amount of a one or more antibody formulations of the invention and an effective amount of one or more therapies (e.g., prophylactic or therapeutic agents), other than antibody formulations of the invention.

In certain embodiments, the invention provides methods to prevent, treat and/or manage a bacterial infection, e.g., a bacterial respiratory infection, or one or more of the symptoms, said methods comprising administering to a subject in need thereof one or more antibody formulations of the invention in combination with and effective amount of one or more therapies (e.g., one or more prophylactic or therapeutic agents), other than antibody formulations of the invention, used to prevent, treat and/or manage bacterial infections. Therapies for bacterial infections, particularly, bacterial respiratory infections include, but are not limited to, anti-bacterial agents (e.g., aminoglycosides (e.g., gentamicin, tobramycin, amikacin, netilimicin) aztreonam, celphalosporins (e.g., cefaclor, cefadroxil, cephalexin, cephazolin), clindamycin, erythromycin, penicillin (e.g., penicillin V, crystalline penicillin G, procaine penicillin G), spectinomycin, and tetracycline (e.g., chlortetracycline, doxycycline, oxytetracycine)) and supportive respiratory therapy, such as supplemental and mechanical ventilation. In certain embodiments, one or more antibody formulations of the invention are administered in combination with one or more supportive measures to a subject in need thereof to prevent, treat and/or manage a bacterial infection or one or more symptoms thereof. Non-limiting examples of supportive measures include humidification of air by ultrasonic nebulizer, aerolized racemic epinephrine, oral dexamethasone, intravenous fluids, intubation, fever reducers (e.g., ibuprofen, acetometaphin), and more preferably, antibiotic or anti-viral therapy (i.e., to prevent or treat secondary infections).

The invention provides methods for preventing, treating and/or managing a biological response to a bacterial infection, e.g., a bacterial respiratory infection, such as, but not limited to, elevated levels of IgE antibodies, mast cell proliferation, degranulation, and/or infiltration, increased proliferation and/or infiltration of B cells, and increased proliferation and/or infiltration of T cells, said methods comprising administering to a subject in need thereof an effective amount of one or more antibody formulations of the invention alone or in combination with an effective amount one or more therapies (e.g. a prophylactic or therapeutic agent) other than antibody formulations of the invention. The invention also provides methods of preventing, treating and/or managing respiratory conditions caused by or associated with bacterial infections, e.g., bacterial respiratory infections, such as, but not limited to, pneumonococcal pneumonia, pneumonia caused by aerobic gram-negative bacilli, recurrent aspiration pneumonia, legionellosis, streptococcal disease, infections caused by Hemophilus, whooping cough, meningitis, or tuberculosis, said methods comprising administering to a subject in need thereof an effective amount of one or more antibody formulations of the invention alone or in combination with an effective amount of another therapy.

In a specific embodiment, the methods of the invention are utilized to prevent, treat and/or manage a bacterial respiratory infection caused by Pneumonococcus, Mycobacteria, aerobic gram-negative bacilli, Streptococcus, or Hemophilus or one or more symptoms thereof, said method comprising administering to a subject in need thereof of an effective amount of one or more antibody formulations of the invention alone or in combination with an effective amount of one or more other therapies (e.g., one or more prophylactic or therapeutic agents) other than antibody formulations of the invention.

In a specific embodiment, the invention provides methods for preventing, treating and/or managing one or more secondary conditions or responses to a primary bacterial infection, e.g., a primary bacterial respiratory infection, said method comprising administering to a subject in need thereof an effective amount of one or more antibody formulations of the invention alone or in combination with an effective amount of other therapies (e.g., other prophylactic or therapeutic agents). Examples of secondary conditions or responses to a primary bacterial infection, particularly a bacterial respiratory infection, include, but are not limited to, asthma-like responsiveness to mucosal stimula, elevated total respiratory resistance, increased susceptibility to secondary viral, bacterial, and fungal infections, and development of such conditions such as, but not limited to, pneumonia, croup, and febrile bronchitits.

In a specific embodiment, the methods of the invention are used to prevent, treat and/or manage a bacterial infection, e.g., a bacterial respiratory infection, or one or more symptoms thereof, said methods comprising administering to a subject in need thereof an effective amount of one or more antibodies of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety). In another specific embodiment, the methods of the invention are used to prevent, treat and/or manage a bacterial infection, e.g., a bacterial respiratory infection, or one or more symptoms thereof, said methods comprising administering to a subject in need thereof an effective amount of one or more antibodies of the invention in combination with an effective amount of siplizumab (MedImmune, Inc., International Pub. No. WO 02/069904). In another embodiment, the methods of the invention are used to prevent, treat and/or manage a bacterial infection, e.g., a bacterial respiratory infection, or one or more symptoms thereof, said methods comprising administering to a subject in need thereof an effective amount of one or more antibodies of the invention in combination with an effective amount of one or more EphA2 inhibitors (e.g., one or more anti-EphA2 antibodies (MedImmune, Inc.; International Publication No. WO 02/102974 A4, dated Dec. 27, 2002, entitled “Mutant Proteins, High Potency Inhibitory Antibodies and FIMCH Crystal Structure,” International Publication No. 03/094859 A2, dated Nov. 20, 2003, entitled “EphA2 Monoclonal Antibodies and Methods of Use Thereof,” U.S. application Ser. No. 10/436,783 and published as U.S. Pat. Pub. No. US 2004/0091486 A1; and U.S. application Ser. No. 10/994,129 and published as U.S. Pat. Pub. No. US 2005/0152899 A1, each of which is incorporated herewith by reference)). In yet another embodiment, the invention provides methods of preventing, treating and/or managing a bacterial infection, e.g., a bacterial respiratory infection, or one or more symptoms thereof, said methods comprising administering an effective amount of one or more antibodies of the invention in combination with an effective amount of VITAXIN™, siplizumab, and/or EphA2.

The invention encompasses methods for preventing the development of bacterial infections, e.g., bacterial respiratory infections, in a patient expected to suffer from a bacterial respiratory infection or at increased risk of such an infection, e.g., patients with suppressed immune systems (e.g., organ-transplant recipients, AIDS patients, patients undergoing chemotherapy, the elderly, infants born prematurely, infants, children, patients with carcinoma of the esophagus with obstruction, patients with tracheobronchial fistula, patients with neurological diseases (e.g., caused by stroke, amyotrophic lateral sclerosis, multiple sclerosis, and myopathies), and patients already suffering from an infection, particularly a respiratory infection). The patients may or may not have been previously treated for an infection.

The antibody formulations of the invention or combination therapies of the invention may be used as the first, second, third, fourth, or fifth therapy to prevent, treat and/or manage a bacterial infection, e.g., a bacterial respiratory infection, or one or more symptom thereof The invention also includes methods of preventing, treating and/or managing a bacterial infection, e.g., a bacterial respiratory infection, or one or more symptoms thereof in a patient undergoing therapies for other diseases or disorders. The invention encompasses methods of preventing, treating and/or managing a bacterial infection, e.g., a bacterial respiratory infection, or one or more symptoms thereof in a patient before any adverse effects or intolerance to therapies other than antibody formulations of the invention develops. The invention also encompasses methods of preventing, treating and/or managing a bacterial infection, e.g., a bacterial respiratory infection, or a symptom thereof in refractory patients. In certain embodiments, a patient with a bacterial respiratory infection is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a bacterial respiratory infection is refractory when bacterial replication has not decreased or has increased. The invention also encompasses methods of preventing the onset or reoccurrence of a bacterial infection, e.g., a bacterial respiratory infection, in patients at risk of developing such infection. The invention also encompasses methods of preventing, treating and/or managing a bacterial infection, e.g., a bacterial respiratory infection, or a symptom thereof in patients who are susceptible to adverse reactions to conventional therapies. The invention further encompasses methods for preventing, treating and/or managing bacterial infections, e.g., bacterial respiratory infections, for which no anti-bacterial therapy is available.

The invention encompasses methods for preventing, treating and/or managing a bacterial infection, e.g., a bacterial respiratory infection, or a symptom thereof in a patient who has proven refractory to therapies other than antibody formulations of the invention, but are no longer on these therapies. In certain embodiments, the patients being managed or treated in accordance with the methods of this invention are patients already being treated with anti-inflammatory agents, antibiotics, anti-virals, anti-fungals, or other biological therapy/immunotherapy. Among these patients are refractory patients, patients who are too young for conventional therapies, and patients with reoccurring bacterial infections despite management or treatment with existing therapies.

The present invention encompasses methods for preventing, treating and/or managing a bacterial infection, e.g., a bacterial respiratory infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat and/or manage a bacterial infection, e.g., a bacterial respiratory infection, or one or more symptoms thereof.

Bacterial infection therapies and their dosages, routes of administration and recommended usage are known in the art and have been described in such literature as the Physicians' Desk Reference (60th ed., 2006).

5.5.7. Fungal Infections

Anti-fungal agents and therapies well known to one of skill in the art for prevention, treatment and/or management of a fungal infection or one or more symptoms thereof (e.g., a fungal respiratory infection) can be used in the compositions and methods of the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONE®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEND®). See, e.g., Table 7, infra, for a list of specific anti-fungal agents and their recommended dosages.

TABLE 7
Anti-fungal Agents.

| Anti-fungal Agent | Dosage |
|---|---|
| Amphotericin B | |
| ABELCET ® (lipid complex | 5 mg/kg/day |
| injection) | |
| AMBISOME ® (liposome for | 3-5 mg/kg/day |
| injection) | |
| AMPHOTEC ® (complex for | 3-4 mg/kg/day |
| injection) | |
| Caspofungin acetate | 70 mg on day one followed by 50 mg/ |
| (CANCIDAS ®) | day |
| Fluconazole (DIFLUCAN ®) | up to 400 mg/day (adults) |
| | up to 12 mg/kg/day (children) |
| Itraconazole (SPORANOX ®) | 200-400 mg/day |
| Flucytosine (ANCOBON ®) | 50-150 mg/kg/day in divided dose |
| | every 6 hours |
| Liposomal nystatin | 1-4 mg/kg |
| Ketoconazole (NIZORAL ®) | 200 mg single daily dose up to |
| | 400 mg/day in two divided doses |
| | (adults) |
| | 3.3-6.6 mg/kg/day for children 2 |
| | years old and older |
| Voriconazole (VFEND ®) | 6 mg/kg i.v. loading dose every 12 |
| | hours for two doses, followed by |
| | maintenance dose of 4 mg/kg i.v. |
| | every 12 hours, then oral |
| | maintenance dose of 200-100 mg |
| | tablet |

In certain embodiments, the anti-fungal agent is an agent that inhibits or reduces a respiratory fungal infection, inhibits or reduces the replication of a fungus that causes a pulmonary or respiratory infection, or inhibits or reduces the spread of a fungus that causes a pulmonary or respiratory infection to other subjects. In cases in which the pulmonary or respiratory fungal infection is Blastomyces dermatitidis, the anti-fungal agent is preferably itraconazole, amphotericin B, fluconazole, or ketoconazole. In cases in which the pulmonary or respiratory fungal infection is pulmonary aspergilloma, the anti-fungal agent is preferably amphotericin B, liposomal amphotericin B, itraconazole, or fluconazole. In cases in which the pulmonary or respiratory fungal infection is histoplasmosis, the anti-fungal agent is preferably amphotericin B, itraconazole, fluconazole, or ketoconazole. In cases in which the pulmonary or respiratory fungal infection is coccidioidomycosis, the anti-fungal agent is preferably fluconazole or amphotericin B. In cases in which the pulmonary or respiratory fungal infection is cryptococcosis, the anti-fungal agent is preferably amphotericin B, fluconazole, or combination of the two agents. In cases in which the pulmonary or respiratory fungal infection is chromomycosis, the anti-fungal agent is preferably itraconazole, fluconazole, or flucytosine. In cases in which the pulmonary or respiratory fungal infection is mucormycosis, the anti-fungal agent is preferably amphotericin B or liposomal amphotericin B. In cases in which the pulmonary or respiratory fungal infection is pseudoallescheriasis, the anti-fungal agent is preferably itraconazole ore miconazole.

Anti-fungal therapies and their dosages, routes of administration, and recommended usage are known in the art and have been described in such literature as Dodds et al., 2000 Pharmacotherapy 20(11) 1335-1355, the Physicians' Desk Reference (60th ed., 2006) and the Merk Manual of Diagnosis and Therapy (17th ed., 1999).

5.5.7.1. Anti-Fungal Therapies

One or more antibody formulations of the invention can be administered according to methods of the invention to a subject to prevent, treat and/or manage a fungal infection or one or more symptoms thereof. One or more antibody formulations of the invention may be also administered to a subject to prevent, treat and/or manage a fungal infection and/or one or more symptoms thereof in combination with one or more other therapies (e.g., one or more prophylactic or therapeutic agents) other than antibody formulations of the invention which are useful for the prevention, treatment and/or management of a fungal infection or one or more symptoms thereof.

In a specific embodiment, the invention provides a method of preventing, treating and/or managing a fungal infection or one or more symptoms thereof, said method comprising administering to a subject in need thereof an effective amount of one or more antibody formulations of the invention. In another embodiment, the invention provides a method of preventing, treating and/or managing a fungal infection or one or more symptoms thereof, said method comprising administering to a subject in need thereof an effective amount of a one or more antibody formulations of the invention and an effective amount of one or more therapies (e.g., prophylactic or therapeutic agents), other than antibody formulations of the invention.

Any type of fungal infection or condition resulting from or associated with a fungal infection (e.g., a respiratory infection) can be prevented, treated and/or managed in accordance with the methods of invention. Examples of fungus which cause fungal infections include, but not limited to, Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

In a specific embodiment, the invention provides a method of preventing, treating and/or managing a fungal respiratory infection or one or more symptoms thereof, said method comprising administering to a subject in need thereof an effective amount of one or more antibody formulations of the invention. In another embodiment, the invention provides a method of preventing, treating and/or managing a fungal respiratory infection or one or more symptoms thereof, said method comprising administering to a subject in need thereof an effective amount of one or more antibody formulations of the invention and an effective amount of one or more therapies (e.g., one or more prophylactic or therapeutic agents) other than antibody formulations of the invention.

In certain embodiments, an effective amount of one or more antibody formulations is administered in combination with an effective amount of one or more therapies (e.g., one or more prophylactic or therapeutic agents), other than antibody formulations of the invention, which are currently being used, have been used, or are known to be useful in the prevention, treatment and/or management of a fungal infection, e.g., a fungal respiratory infection, to a subject in need thereof. Therapies for fungal infections include, but are not limited to, anti-fungal agents such as azole drugs e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B colloidal dispersion (“ABCD”)(AMPHOTEC®), liposomal amphotericin B (AMBISONE®)), postassium iodide (KI), pyrimidine (e.g., flucytosine (ANCOBON®)), and voriconazole (VFEND®). In certain embodiments, an effective amount of one or more antibody formulations of the invention are administered in combination with one or more supportive measures to a subject in need thereof to prevent, treat and/or manage a fungal infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

The invention also provides methods for preventing, treating and/or managing a biological response to a fungal respiratory infection such as, but not limited to, elevated levels of IgE antibodies, elevated nerve growth factor (NGF) levels, mast cell proliferation, degranulation, and/or infiltration, increased proliferation and/or infiltration of B cells, and increased proliferation and/or infiltration of T cells, said methods comprising administration of an effective amount of one or more antibody formulations that immunospecifically bind to an IL-9 polypeptide alone or in combination with one or more other therapies.

In a specific embodiment, the invention provides methods for preventing, treating and/or managing one or more secondary conditions or responses to a primary fungal infection, e.g., a primary fungal respiratory infection, said method comprising of administering to a subject in need thereof an effective amount of one or more antibody formulations of the invention alone or in combination with an effective amount of other therapies (e.g., other prophylactic or therapeutic agents) other than antibody formulations of the invention. Examples of secondary conditions or responses to a primary fungal infections, particularly primary fungal respiratory infection include, but are not limited to, asthma-like responsiveness to mucosal stimula, elevated total respiratory resistance, increased susceptibility to secondary viral, fungal, and fungal infections, and development of such conditions such as, but not limited to, pneumonia, croup, and febrile bronchitis.

In a specific embodiment, the invention provides methods to prevent, treat and/or manage a fungal infection, e.g., a fungal respiratory infection, or one or more symptoms thereof, said methods comprising administering to a subject in need thereof an effective amount of one or more antibodies of the invention in combination with an effective amount of VITAXIN™ (MedImmune, Inc., International Publication No. WO 00/78815, International Publication No. WO 02/070007 A1, dated Sep. 12, 2002, entitled “Methods of Preventing or Treating Inflammatory or Autoimmune Disorders by Administering Integrin AlphaV Beta3 Antagonists,” International Publication No. WO 03/075957 A1, dated Sep. 18, 2003, entitled “The Prevention or Treatment of Cancer Using Integrin AlphaVBeta3 Antagonists in Combination With Other Agents,” U.S. Patent Pub. No. US 2002/0168360 A1, dated Nov. 14, 2002, entitled “Methods of Preventing or Treating Inflammatory or Autoimmune Disorders by Administering Integrin αᵥβ3 Antagonists in Combination With Other Prophylactic or Therapeutic Agents,” and International Publication No. WO 03/075741 A2, dated Sep. 18, 2003, entitled, “Methods of Preventing or Treating Disorders by Administering an Integrin αvβ3 Antagonist in Combination With an HMG-CoA Reductase Inhibitor or a Bisphosphonate,” each of which is incorporated herewith by reference in its entirety) to a subject in need thereof. In another specific embodiment, the invention provides methods of preventing, treating and/or managing a fungal respiratory infection or one or more symptoms thereof, said methods comprising administering to a subject in need thereof an effective amount of one or more antibodies of the invention in combination with an effective amount of siplizumab (MedImmune, Inc., International Pub. No. WO 02/069904) to a subject in need thereof. In another embodiment, the invention provides methods of preventing, treating and/or managing a fungal respiratory infection or one or more symptoms thereof, said methods comprising administering an effective amount of one or more antibodies of the invention in combination with an effective amount of one or more EphA2 inhibitors (e.g., one or more anti-EphA2 antibodies (MedImmune, Inc.; International Publication No. WO 02/102974 A4, dated Dec. 27, 2002, entitled “Mutant Proteins, High Potency Inhibitory Antibodies and FIMCH Crystal Structure,” International Publication No. 03/094859 A2, dated Nov. 20, 2003, entitled “EphA2 Monoclonal Antibodies and Methods of Use Thereof,” U.S. application Ser. No. 10/436,783; and U.S. application Ser. No. 10/994,129 and published as U.S. 2005/0152899 A1, each of which is incorporated herewith by reference)) to a subject in need thereof. In yet another embodiment, the invention provides methods of preventing, treating and/or managing a fungal infection, e.g., a fungal respiratory infection, or one or more symptoms thereof, said methods comprising administering an effective amount of one or more antibodies of the invention in combination with an effective amount of VITAXIN™, siplizumab, and/or EphA2 to a subject in need thereof.

The invention encompasses methods for preventing the development of fungal respiratory infections in a patient expected to suffer from a fungal infection, e.g., a fungal respiratory infection, or at increased risk of such an infection. Such subjects include, but are not limited to, patients with suppressed immune systems (e.g., patients organ-transplant recipients, AIDS patients, patients undergoing chemotherapy, patients with carcinoma of the esophagus with obstruction, patients with tracheobronchial fistula, patients with neurological diseases (e.g., caused by stroke, amyotorphic lateral sclerosis, multiple sclerosis, and myopathies), and patients already suffering from a respiratory condition, particularly a respiratory infection). In a specific embodiment, the patient suffers from bronchopulmonary dysplasia, congenital heart disease, cystic fibrosis, and/or acquired or congenital immunodeficiency. In another specific embodiment, the patient is an infant born prematurely, an infant, a child, an elderly human, or a human in a group home, nursing home, or some other type of institution. The invention also encompasses methods of preventing, treating and/or managing a respiratory condition or one or more symptoms thereof in patients who are susceptible to adverse reactions to conventional therapies for respiratory conditions for which no therapies are available.

The antibody formulations of the invention or combination therapies of the invention may be used as the first, second, third, fourth, or fifth therapy to prevent, treat and/or manage a fungal infection, e.g., a fungal respiratory infection or one or more symptom thereof. The invention also includes methods of preventing, treating and/or managing a fungal infection, e.g., a fungal respiratory infection or one or more symptoms thereof in a patient undergoing therapies for other disease or disorders. The invention encompasses methods of preventing, treating and/or managing a fungal infection, e.g., a fungal respiratory infection or one or more symptoms thereof in a patient before any adverse effects or intolerance to therapies other antibody formulations of the invention develops. The invention also encompasses methods of preventing, treating and/or managing a fungal infection, e.g., a fungal respiratory infection or a symptom thereof in refractory patients. In certain embodiments, a patient with a fungal infection, e.g., a fungal respiratory infection, is refractory to a therapy when the infection has not significantly been eradicated and/or the symptoms have not been significantly alleviated. The determination of whether a patient is refractory can be made either in vivo or in vitro by any method known in the art for assaying the effectiveness of a treatment of infections, using art-accepted meanings of “refractory” in such a context. In various embodiments, a patient with a fungal infection, e.g., a fungal respiratory infection, is refractory when fungal replication has not decreased or has increased. The invention also encompasses methods of preventing the onset or reoccurrence of fungal infections, e.g., fungal respiratory infections, in patients at risk of developing such infections. The invention also encompasses methods of preventing, treating and/or managing a fungal infection, e.g., a fungal respiratory infection, or a symptom thereof in patients who are susceptible to adverse reactions to conventional therapies. The invention further encompasses methods for preventing, treating and/or managing fungal infections, e.g., fungal respiratory infections, for which no anti-fungal therapy is available.

The invention encompasses methods for preventing, treating and/or managing a fungal infection, e.g., a fungal respiratory infection, or a symptom thereof in a patient who has proven refractory to therapies other than antibody formulations of the invention but are no longer on these therapies. In certain embodiments, the patients being managed or treated in accordance with the methods of this invention are patients already being treated with antibiotics, anti-virals, anti-fungals, or other biological therapy/immunotherapy. Among these patients are refractory patients, patients who are too young for conventional therapies, and patients with reoccurring fungal infections despite management or treatment with existing therapies.

The present invention provides methods for preventing, treating and/or managing a fungal infection, e.g., a fungal respiratory infection, or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (e.g., post-operative patients, chemotherapy patients, and patients with immunodeficiency disease), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat and/or manage a fungal infection, e.g., a fungal respiratory infection, or one or more symptoms thereof.

Fungal infection therapies and their dosages, routes of administration and recommended usage are known in the art and have been described in such literature as the Physicians' Desk Reference (60th ed., 2006).

5.6. Methods of Administering the Antibody Formulations

The invention provides methods of prevention, treatment and/or management of a disorder, for example, a disorder associated with or characterized by aberrant expression and/or activity of the IL-9R or one or more subunits thereof, autoimmune diseases, inflammatory diseases, proliferative diseases, or infections (e.g., respiratory infections), or one or more symptoms thereof by administrating to a subject of an effective amount of liquid formulations of the invention. Various delivery systems are known and can be used to administer a liquid formulation of the present invention or a prophylactic or therapeutic agent. Methods of administering antibody liquid formulations of the present invention or a therapy (e.g., a prophylactic or therapeutic agent) include, but are not limited to, parenteral administration (e.g., intradermal, intramuscular, intraperitoneal, intravenous and, preferably, subcutaneous), epidural administration, topical administration, and mucosal administration (e.g., intranasal and oral routes). In a specific embodiment, liquid formulations of the present invention are administered intramuscularly, intravenously, or subcutaneously. In one embodiment, the liquid formulations of the invention are administered subcutaneously. The formulations may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In a specific embodiment, the liquid formulations of the invention are administered intratumorally or at the site of inflammation.

In a specific embodiment, the antibody formulations of the invention comprise a pharmaceutically acceptable carrier. In one embodiment, the pharmaceutically acceptable carrier is water for injection, USP, 5% dextrose in water (D5W) or saline.

Generally, the antibodies (including antibody fragments thereof) that immunospecifically bind to an IL-9 polypeptide contained in the liquid formulations of the invention are derived from a subject that is of the same species origin or species reactivity as recipient of the liquid formulations of the invention. Thus, in one embodiment, liquid formulations of the invention comprising human or humanized antibodies that immunospecifically bind to an IL-9 polypeptide contained in the liquid formulations of the invention are administered to a human patient for therapy or prophylaxis.

The invention also provides that a liquid formulation of the present invention is packaged in a hermetically sealed container such as an ampoule or sachette indicating the quantity of antibody (including antibody fragment thereof). Preferably, the liquid formulations of the present invention are in a hermetically sealed container indicating the quantity and concentration of the antibody (including antibody fragment thereof).

Preferably, the liquid formulation of the present invention is supplied in a hermetically sealed container and comprises at least 10 mg/ml, 15 mg/ml, 20 mg/ml, 30 mg/ml, 40 mg/ml, 50 mg/ml, 60 mg/ml, 70 mg/ml, 80 mg/ml, 90 mg/ml, 100 mg/ml, 150 mg/ml, 175 mg/ml, 200 mg/ml, 250 mg/ml, or 300 mg/ml of an antibody (including antibody fragment thereof) that immunospecifically binds to an IL-9 polypeptide, in a quantity of 1 ml, 2 ml, 3 ml, 4 ml, 5 ml, 6 ml, 7 ml, 8 ml, 9 ml, 10 ml, 15 ml, or 20 ml and, most preferably, 1.2 ml. In a specific embodiment of the invention, a liquid formulation of the invention is supplied in a hermetically sealed container and comprises at least 15 mg/ml, at least 20 mg/ml, at least 25 mg/ml, at least 50 mg/ml, at least 100 mg/ml, at least 150 mg/ml, at least 175 mg/ml, at least 200 mg/ml, at least 250 mg/ml or at least 300 mg/ml of an antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3, 71A10, 7F3 22D3, 7F3com-2H2, 7F3com-3H5, or 7F3com-3D4 or an antigen-binding fragment thereof) for intravenous injections, and at least 15 mg/ml, 20 mg/ml, 50 mg/ml, 80 mg/ml, 100 mg/ml, 150 mg/ml, 175 mg/ml, 200 mg/ml, 250 mg/ml or 300 mg/ml an antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or a fragment thereof) for repeated subcutaneous administration. In a specific embodiment, an antibody formulation of the present invention may be produced by lyophilizing the aqueous antibody formulation. In a specific embodiment, the lyophilized antibody aqueous antibody solution may be reconstituted with a pharmaceutically acceptable carrier. In a specific embodiment, a pharmaceutically acceptable carrier is water for injection, USP, 5% dextrose in water (D5W) or saline.

The amount of a liquid formulation of the present invention which will be effective in the prevention, treatment and/or management of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof can be determined by standard clinical techniques well-known in the art or described herein. The precise dose to be employed in the formulation will also depend on the route of administration, and the seriousness of the inflammatory disorder, autoimmune disorder or cancer, and should be decided according to the judgment of the practitioner and each patient's circumstances. Effective doses may be extrapolated from dose-response curves derived from in vitro or animal model test systems.

For formulations of the antibodies, proteins, polypeptides, peptides and fusion proteins encompassed by the invention, the dosage administered to a patient may be calculated using the patient's weight in kilograms (kg) multiplied by the dose to be administered in mg/kg. The required volume (in mL) to be given is then determined by taking the mg dose required divided by the concentration of the antibody formulation. The final calculated required volume will be obtained by pooling the contents of as many vials as are necessary into syringe(s) to administer the antibody formulation of the invention. The final calculated required volume will be obtained by pooling the contents of as many vials as are necessary into syringe(s) to administer the drug. A maximum volume of 2.0 mL of the antibody formulation can be injected per site. The dose (in mL) can be calculated using the following formula: Dose (mL)=[volunteer weight] (kg)×[dose] mg/kg÷100 mg/mL of the antibody formulation. Generally, human antibodies have a longer half-life within the human body than antibodies from other species due to the immune response to the foreign polypeptides. Thus, lower dosages of human antibodies and less frequent administration is often possible. Further, the dosage, volume and frequency of administration of liquid formulations of the present invention may be reduced by increasing the concentration of an antibody (including antibody fragment thereof) in the formulations, increasing affinity and/or avidity of the antibody (including antibody fragment thereof), and/or increasing the half-life of the antibody (including antibody fragment thereof).

In a specific embodiment, the dosage administered to a patient will be calculated using the patient's weight in kilograms (kg) multiplied by the dose to be administered in mg/kg. The required volume (in mL) to be given is then determined by taking the mg dose required divided by the concentration of the antibody (including antibody fragment thereof) in the formulations (100 mg/mL). The final calculated required volume will be obtained by pooling the contents of as many vials as are necessary into syringe(s) to administer the drug. A maximum volume of 2.0 mL of antibody (including antibody fragment thereof) in the formulations can be injected per site.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In a specific embodiment, 0.1 to 20 mg/kg/week, 1 to 15 mg/kg/week, 2 to 8 mg/week, 3 to 7 mg/kg/week, or 4 to 6 mg/kg/week of an antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or a fragment thereof) in a liquid formulation of the invention is administered to a subject with an inflammatory disorder, an autoimmune disorder or cancer. In another embodiment, a subject is administered one or more doses of a prophylactically or therapeutically effective amount of a liquid formulation of the invention, wherein the prophylactically or therapeutically effective amount is not the same for each dose.

In one embodiment, a liquid formulation of the invention is administered in a dosing regimen that maintains the plasma concentration of the antibody immunospecific for αᵥβ3 at a desirable level (e.g., about 0.1 to about 100 μg/ml), which continuously blocks the an IL-9 polypeptide activity. In a specific embodiment, the plasma concentration of the antibody is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment and/or management of a chronic disease or disorder.

In specific embodiments, a liquid formulation of the invention comprising a conjugated antibody (including antibody fragment thereof) immunospecific for an IL-9 polypeptide is administered intermittently. As used herein, “a conjugated antibody or antibody fragment” refers to an antibody (including antibody fragment thereof) that is conjugated or fused to another moiety, including but not limited to, a heterologous peptide, polypeptide, another antibody (including antibody fragment thereof), a marker sequence, a diagnostic agent, a therapeutic moiety, a therapeutic drug, a radioactive metal ion, a polymer, albumin, and a solid support.

In another embodiment, a subject, preferably a human, is administered one or more doses of a prophylactically or therapeutically effective amount of an antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3, 71A10, 7F3 22D3, 7F3com-2H2, 7F3com-3H5, or 7F3com-3D4 or a fragment thereof) in a liquid formulation of the invention, wherein the dose of a prophylactically or therapeutically effective amount of the antibody (including antibody fragment thereof) in the liquid formulation of the invention administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In another embodiment, a subject, preferably a human, is administered one or more doses of a prophylactically or therapeutically effective amount of an antibody (including antibody fragment thereof) that immunospecifically binds to an IL-9 polypeptide (e.g., 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or a fragment thereof) in a liquid formulation of the invention, wherein the dose of a prophylactically or therapeutically effective amount of the antibody (including antibody fragment thereof) in the liquid formulation of the invention administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg//kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

The dosages of prophylactic or therapeutically agents are described in the Physicians' Desk Reference (60th ed., 2006).

5.7. Biogical Assays

The antibodies (including antibody fragment thereof) of the liquid formulations of the invention may be characterized in a variety of ways well-known to one of skill in the art. For example, antibodies (including antibody fragments thereof) of the liquid formulations of the invention may be assayed for the ability to immunospecifically bind to antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Cwirla et al., 1990, Proc. Natl. Acad. Sci. USA 87:6378-6382; and Felici, 1991, J. Mol. Biol. 222:301-310) (each of these references is incorporated herein in its entirety by reference). For example, antibodies (including antibody fragments thereof) that have been identified to immunospecifically bind to an IL-9 polypeptide can then be assayed for their specificity and affinity for an IL-9 polypeptide.

The antibodies (including antibody fragments thereof) of the liquid formulations of the invention may be assayed for immunospecific binding to antigen and cross-reactivity with other antigens by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%- 20% SDS-PAGE depending on the molecular weight of the antigen), transferring the protein sample from the polyacrylamide gel to a membrane such as nitrocellulose, PVDF or nylon, incubating the membrane in blocking solution (e.g., PBS with 3% BSA or non-fat milk), washing the membrane in washing buffer (e.g., PBS-Tween 20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. In one embodiment, an ELISA may be performed by coating a high binding 96-well microtiter plate (Costar) with 2 μg/ml of rhu-IL-9 in PBS overnight. Following three washes with PBS, the plate is incubated with three-fold serial dilutions of Fab at 25° C. for 1 hour. Following another three washes of PBS, 1 μg/ml anti-human kappa-alkaline phosphatase-conjugate is added and the plate is incubated for 1 hour at 25° C. Following three washes with PBST, the alkaline phosphatase activity is determined in 50 ηl/AMP/PPMP substrate. The reactions are stopped and the absorbance at 560 nm is determined with a VMAX microplate reader. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of the contained in a liquid formulation of the present invention or a fragment thereof for a specific antigen and the binding off-rates can be determined from the data by scatchard plot analysis. Competition with a second antibody can also be determined using radioimmunoassays. In one example, an IL-9 polypeptide is incubated with an antibody conjugated to a labeled compound (e.g., ³H or ¹²⁵I) in the presence of increasing amounts of an unlabeled second antibody.

For example, in one embodiment, BlAcore kinetic analysis is used to determine the binding on and off rates of antibodies of the liquid formulations of the invention to an IL-9 polypeptide. BlAcore kinetic analysis comprises analyzing the binding and dissociation of an IL-9 polypeptide from chips with immobilized antibodies of the invention on their surface. A typical BlAcore kinetic study involves the injection of 250 μL of an antibody reagent (mAb, Fab) at varying concentration in HBS buffer containing 0.005% Tween-20 over a sensor chip surface, onto which has been immobilized the antigen. The flow rate is maintained constant at 75 μL/min. Dissociation data is collected for 15 min. or longer as necessary. Following each injection/dissociation cycle, the bound mAb is removed from the antigen surface using brief, 1 min. pulses of dilute acid, typically 10-100 mM HCl, though other regenerants are employed as the circumstances warrant. More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 nM solution of the antigen in 10 mM NaOAc, pH4 or pH5 is prepared and passed over the EDC/NHS-activated surface until approximately 30-50 RU's worth of antigen are immobilized. Following this, the unreacted active esters are “capped” off with an injection of 1M Et-NH2. A blank surface, containing no antigen, is prepared under identical immobilization conditions for reference purposes. Once an appropriate surface has been prepared, a suitable dilution series of each one of the antibody reagents is prepared in HBS/Tween-20, and passed over both the antigen and reference cell surfaces, which are connected in series. The range of antibody concentrations that are prepared varies, depending on what the equilibrium binding constant, K_{D}, is estimated to be. As described above, the bound antibody is removed after each injection/dissociation cycle using an appropriate regenerant.

If the antibodies (including antibody fragments thereof) of the liquid formulations of the invention are immunospecific for a receptor ligand, the antibodies can also be assayed for their ability to inhibit the binding of the ligand to its receptor using techniques known to those of skill in the art. For example, cells expressing IL-9 receptor can be contacted with IL-9 in the presence or absence of an antibody (including antibody fragment thereof) of liquid formulations and the ability of the antibody (including antibody fragment thereof) to inhibit IL-9's binding can measured by, for example, flow cytometry or a scintillation assay. IL-9 or the antibody (including antibody fragment thereof) contained in the liquid formulation can be labeled with a detectable compound such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, o-phthaldehyde and fluorescamine) to enable detection of an interaction between IL-9 and its host cell receptor. Alternatively, the ability of antibodies (including antibody fragment thereof) of the liquid formulations of the invention to inhibit ligand from binding to its receptor can be determined in cell-free assays. For example, an IL-9 polypeptide can be contacted with an antibody (including antibody fragment thereof) of the liquid formulations of the invention and the ability of the antibody (including antibody fragment thereof) to inhibit the IL-9 polypeptide from binding to its host cell receptor can be determined. Preferably, the antibody (including antibody fragment thereof) of the liquid formulations of the invention of the invention is immobilized on a solid support and an IL-9 polypeptide is labeled with a detectable compound. Alternatively, an IL-9 polypeptide is immobilized on a solid support and the antibody (including antibody fragment thereof) contained within a liquid formulation of the invention is labeled with a detectable compound. An IL-9 may be partially or completely purified (e.g., partially or completely free of other polypeptides) or part of a cell lysate. Further, an IL-9 polypeptide may be a fusion protein comprising IL-9, a derivative, analog or fragment thereof and a domain such as glutathionine-S-transferase. Alternatively, an IL-9 polypeptide can be biotinylated using techniques well known to those of skill in the art (e.g., biotinylation kit, Pierce Chemicals; Rockford, Ill.).

In a specific embodiment, the ability of antibodies (including antibody fragments thereof) of the liquid formulations of the invention to inhibit ligand binding to its host cell receptor can be measured by cell proliferation assays. As an example, the murine TS1-RA3 T cell line expressing both human and murine IL-9Ra may be grown continuously in growth medium (DMEM) containing rhulL-9 (25 ng/ml, R & D Systems). Upon withdrawal of rhulL-9, TS1-RA3 undergoes cell death in 18-24 hours. TS1-RA3 cells grown in RPMI 1640 (ATCC) medium supplemented with 10% FBS and 25 ng/ml rHu-IL9. Prior to the assay, the cells are washed with media containing no IL-9 and resuspended at 5×10⁵ cells/ml in media containing 2 ng/ml rhulL-9. The cells are distributed into a black clear bottom non-binding 96-well microtiter plate (100 μl cells/well) and 100 ml of serially diluted variant Fabs is then added to the plate. The plate is incubated at 72 hours at 37° C., 5% CO2. 20 μl/well of Alamar blue® is added, and the cells are incubated for an additional 4-5 hours. Cell metabolism is quantitated using a fluorimeter with excitation at 555 nm and emission at 590 nm.

5.7.1. In vitro Studies

The antibodies, compositions, or combination therapies of the invention can be tested in vitro and/or in vivo for their ability to modulate the biological activity of immune cells (e.g., T cells, neutrophils, and mast cells), endothelial cells, and epithelial cells. The ability of an antibody, composition, or combination therapy of the invention to modulate the biological activity of immune cells (e.g., T cells, B cells, mast cells, macrophages, neutrophils, and eosinophils), endothelial cells, and epithelial cells can be assessed by: detecting the expression of antigens (e.g., activation of genes by IL-9, such as, but not limited to, mucin genes (e.g., MUC2, MUC5AC, MUC5B, and MUC6) and genes involved in lymphocyte activation (e.g., Lgamma-6A/E)); detecting the proliferation of immune cells, endothelia cells and/or epithelial cells; detecting the activation of signaling molecules (e.g., the phosphorylation of Stat2, the phosphorylation of JAK3, or the phosphorylation of the IL-9R); detecting the effector function of immune cells (e.g., T cells, B cells, mast cells, macrophages, neutrophils, and eosinophils), endothelial cells, and/or epithelial cells; or detecting the differentiation of immune cells, endothelial cells, and/or epithelial cells. Techniques known to those of skill in the art can be used for measuring these activities. For example, cellular proliferation can be assayed by 3H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and FACS analysis. The activation of signaling molecules can be assayed, for example, by kinase assays and electrophoretic shift assays (EMSAs). Mast cell degranulation can be assayed, for example by measuring serotonin (5-HT) release or histamine release with high-performance liquid chromatogoraphy (see, e.g., Taylor et al. 1995 Immunology 86(3): 427-433 and Kurosawa et al., 1998 Clin Exp Allergy 28(8): 1007-1012).

The antibodies, compositions, or combination therapies of the invention are preferably tested in vitro and then in vivo for the desired therapeutic or prophylactic activity prior to use in humans. For example, assays which can be used to determine whether administration of a specific pharmaceutical composition is indicated include cell culture assays in which a patient tissue sample is grown in culture and exposed to, or otherwise contacted with, a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent) for each individual patient. In various specific embodiments, in vitro assays can be carried out with representative cells of cell types involved a disease or disorder associated with or characterized by aberrant expression and/or activity of an IL-9 polypeptide, a disease or disorder associated with or characterized by aberrant expression and/or activity of an IL-9R or one or more subunits thereof, an inflammatory disorder, an autoimmune disorder, a proliferative disorder, or an infection (e.g., a respiratory infection) to determine if a pharmaceutical composition of the invention has a desired effect upon such cell types.

The effect of an antibody, a composition, or a combination therapy of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen which is conjugated to FITC or phycoerythrin, and measuring the number of T-cells by FACS.

The methods of the invention for preventing, treating and/or managing a viral respiratory infection or one or more symptoms thereof can be tested for their ability to inhibit viral replication or reduce viral load in in vitro assays. For example, viral replication can be assayed by a plaque assay such as described, e.g., by Johnson et al., 1997, Journal of Infectious Diseases 176:1215-1224 176:1215-1224. The antibodies, compositions, or combination therapies administered according to the methods of the invention can also be assayed for their ability to inhibit or downregulate the expression of viral polypeptides. Techniques known to those of skill in the art, including, but not limited to, western blot analysis, northern blot analysis, and RT-PCR can be used to measure the expression of viral polypeptides.

The methods of the invention for preventing, treating and/or managing a respiratory infection or one or more symptoms thereof can be tested for activity against bacteria causing respiratory infections in in vitro assays well-known in the art. In vitro assays known in the art can also be used to test the existence or development of resistance of bacteria to a therapy (e.g., an antibody of the invention, other prophylactic or therapeutic agent, a combination thereof, or a composition thereof) of the invention. Such in vitro assays are described in Gales et al., 2002, Diag. Nicrobiol. Infect. Dis. 44(3):301-311; Hicks et al., 2002, Clin. Microbiol. Infect. 8(11): 753-757; and Nicholson et al., 2002, Diagn. Microbiol. Infect. Dis. 44(1): 101-107.

The therapies (e.g., an antibody of the liquid formulations of the invention alone or in combination with prophylactic or therapeutic agents, other than antibodies of the invention) of the invention for preventing, treating and/or managing a respiratory infection or one or more symptoms thereof can be tested for anti-fungal activity against different species of fungus. Any of the standard anti-fungal assays well-known in the art can be used to assess the anti-fungal activity of a therapy. The anti-fungal effect on different species of fungus can be tested. The tests recommended by the National Committee for Clinical Laboratories (NCCLS) (See National Committee for Clinical Laboratories Standards. 1995, Proposed Standard M27T. Villanova, Pa., all of which is incorporated herein by reference in its entirety) and other methods known to those skilled in the art (Pfaller et al., 1993, Infectious Dis. Clin. N. Am. 7: 435-444) can be used to assess the anti-fungal effect of a therapy. The antifungal properties of a therapy may also be determined from a fungal lysis assay, as well as by other methods, including, inter alia, growth inhibition assays, fluorescence-based fungal viability assays, flow cytometry analyses, and other standard assays known to those skilled in the art.

For example, the anti-fungal activity of a therapy can be tested using macrodilution methods and/or microdilution methods using protocols well-known to those skilled in the art (see, e.g., Clancy et al., 1997 Journal of Clinical Microbiology, 35(11): 2878-82; Ryder et al., 1998, Antimicrobial Agents and Chemotherapy, 42(5): 1057-61; U.S. Pat. No. 5,521,153; U.S. Pat. No. 5,883,120, U.S. Pat. No. 5,521,169, all of which are incorporated by reference in their entirety). Briefly, a fungal strain is cultured in an appropriate liquid media, and grown at an appropriate temperature, depending on the particular fungal strain used for a determined amount of time, which is also depends on the particular fungal strain used. An innoculum is then prepared photometrically and the turbidity of the suspension is matched to that of a standard, e.g., a McFarland standard. The effect of a therapy on the turbidity of the inoculum is determined visually or spectrophotometrically. The minimal inhibitory concentration (“MIC”) of the therapy is determined, which is defined as the lowest concentration of the lead compound which prevents visible growth of an inoculum as measured by determining the culture turbidity.

The anti-fungal activity of a therapy can also be determined utilizing colorimetric based assays well-known to one of skill in the art. One exemplary colorimetric assay that can be used to assess the anti-fungal activity of a therapy is described by Pfaller et al. (1994, Journal of Clinical Microbiology, 32(8): 1993-6, which is incorporated herein by reference in its entirety; also see Tiballi et al., 1995, Journal of Clinical Microbiology, 33(4): 915-7). This assay employs a colorimetric endpoint using an oxidation-reduction indicator (Alamar Biosciences, Inc., Sacramento Calif.).

The anti-fungal activity of a therapy can also be determined utilizing photometric assays well-known to one of skill in the art (see, e.g., Clancy et al., 1997 Journal of Clinical Microbiology, 35(11): 2878-82; Jahn et al., 1995, Journal of Clinical Microbiology, 33(3): 661-667, each of which is incorporated herein by reference in its entirety). This photometric assay is based on quantifying mitochondrial respiration by viable fungi through the reduction of 3-(4,5-dimethyl-2thiazolyl)-2,5,-diphenyl-2H-tetrazolium bromide (MTT) to formazan. MIC's determined by this assay are defined as the highest concentration of the test therapy associated with the first precipitous drop in optical density. In some embodiments, the therapy is assayed for anti-fungal activity using macrodilution, microdilution and MTT assays in parallel.

Further, any in vitro assays known to those skilled in the art can be used to evaluate the prophylactic and/or therapeutic utility of an antibody, a composition, a combination therapy disclosed herein for a respiratory infection or one or more symptoms thereof.

5.7.2. In vivo Assays

The antibodies, compositions, or combination therapies of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering the therapies (e.g., prophylactic and/or therapeutic agents), whether such therapies are administered separately or as an admixture, and the frequency of administration of the therapies.

Animal models for autoimmune disorders can also be used to assess the efficacy of an antibody, a composition, or a combination therapy of the invention. Animal models for autoimmune disorders such as type 1 diabetes, thyroid autoimmunity, systemic lupus erythematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Efficacy in preventing, treating and/or managing an autoimmune disorder may be demonstrated, e.g., by detecting the ability of an antibody, a composition, or a combination therapy of the invention to reduce one or more symptoms of the autoimmune disorder, to reduce mean absolute lymphocyte counts, to decrease T cell activation, to decrease T cell proliferation, to reduce cytokine production, or to modulate one or more particular cytokine profiles. Efficacy in preventing or treating psoriasis may be demonstrated, e.g., by detecting the ability of an antibody, fragment thereof, or composition of the invention to reduce one or more symptoms of psoriasis, to reduce mean absolute lymphocyte counts, to reduce cytokine production, to modulate one or more particular cytokine profiles, to decrease scaling, to decrease erythema, to decrease plaque elevation, to decrease T cell activation in the dermis or epidermis of an affected area, to decrease T cell infiltration to the dermis or epidermis of an affected area, to reduce PASI, to improve the physician's global assessment score, or to improve quality of life.

Animal models for cancer can be used to assess the efficacy of an antibody, a composition, or a combination therapy of the invention. Examples of animal models for lung cancer include, but are not limited to, lung cancer animal models described by Zhang & Roth (1994, In vivo 8(5):755-69) and a transgenic mouse model with disrupted p53 function (see, e.g., Morris et al., 1998, J La State Med Soc 150(4):179-85). An example of an animal model for breast cancer includes, but is not limited to, a transgenic mouse that overexpresses cyclin Dl (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res 61(6):2395-8). Examples of animal models for pancreatic cancer include, but are not limited to, a metastatic model of Panc02 murine pancreatic adenocarcinoma (see, e.g., Wang et al., 2001, Int J Pancreatol 29(1):37-46) and nu-nu mice generated in subcutaneous pancreatic tumours (see, e.g., Ghaneh et al., 2001, Gene Ther 8(3):199-208). Examples of animal models for non-Hodgkin's lymphoma include, but are not limited to, a severe combined immunodeficiency (“SCID”) mouse (see, e.g., Bryant et al., 2000, Lab Invest 80(4):553-73) and an IgHmu-HOX11 transgenic mouse (see, e.g., Hough et al., 1998, Proc Natl Acad Sci USA 95(23):13853-8). An example of an animal model for esophageal cancer includes, but is not limited to, a mouse transgenic for the human papillomavirus type 16 E7 oncogene (see, e.g., Herber et al., 1996, J Virol 70(3):1873-81). Examples of animal models for colorectal carcinomas include, but are not limited to, Apc mouse models (see, e.g., Fodde &

Smits, 2001, Trends Mol Med 7(8):369-73 and Kuraguchi et al., 2000, Oncogene 19(50):5755-63).

The anti-inflammatory activity of an antibody, a composition, or a combination therapy of the invention can be determined by using various experimental animal models of inflammatory arthritis known in the art and described in Crofford L. J. and Wilder R. L., “Arthritis and Autoimmunity in Animals,” in Arthritis and Allied Conditions: A Textbook of Rheumatology, McCarty (eds.), Chapter 30 (Lee and Febiger, 1993). Experimental and spontaneous animal models of inflammatory arthritis and autoimmune rheumatic diseases can also be used to assess the anti-inflammatory activity of the antibodies, compositions, or combination therapies of invention.

The anti-inflammatory activity of an antibody, a composition, or a combination therapy of invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test therapies (e.g., prophylactic or therapeutic agents) is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of an antibody, a composition, a combination therapy of the invention.

Animal models for allergies and asthma are known in the art, such as constant-flow inflation with end-inspiratory occlusion described in Ewart et al., 1995 J

Appl Physiol 79(2):560-566 and other assays described in, e.g., Komai et al., 2003 Br J Pharmacol 138(5): 912-920; Kenyon et al., 2003 Toxicol Appl Pharmacol 186(2): 90-100; Path et al., 2002 Am J Resp & Critical Care Med 166(6): 818-826; Martins et al., 1990 Crit Care Med 19:515-519; Nicolaides et al., 1997 Proc Natl Acad Sci USA 94:13175-13180; McLane et al., 1998 19:713-720; and Temann et al., 1998 J Exp Med 188(7): 1307-1320. For example, the murine adoptive transfer model is an animal model used to assess the efficacy an antibody, a composition, or a combination therapy of the invention for the prevention, treatment, management, and/or asthma include. In the murine adoptive transfer model, aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747). Airway hypersensitivity can be induced in mice by ovalbumin (Tomkinson et al., 2001, J. Immunol. 166:5792-5800) or Schistosoma mansoni egg antigen (Tesciuba et al., 2001, J. Immunol. 167:1996-2003).

Efficacy in preventing or treating an inflammatory disorder may be demonstrated, e.g., by detecting the ability of an antibody, a composition, or a combination therapy of the invention to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life.

Changes in inflammatory disease activity may also be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF.

The efficacy of an antibody, a composition, or a combination therapy of the invention in preventing, treating and/or managing Type I allergic reaction may be assessed by its ability to induce anti-IgE antibodies that inhibit IgE from binding to is receptor on mast cells or basophils in vitro. IgE levels can be assayed by immunoassays, gel electrophoresis followed by visualization, radioimmunosorbent test (RIST), radioallergosorbent test (RAST), or any other method known to those skilled in the art.

Animal models for viral infections can also be used to assess the efficacy of an antibody, a composition, or a combination therapy of the invention. Animal models for viral infections such as EBV-associated diseases, gammaherpesviruses, infectious mononucleosis, simian immunodeficiency virus (“SIV”), Borna disease virus infection, hepatitis, varicella virus infection, viral pneumonitis, Epstein-Barr virus pathogenesis, feline immunodeficiency virus (“FIV”), HTLV type 1 infection, human rotaviruses, and genital herpes have been developed (see, e.g., Hayashi et al., 2002, Histol Histopathol 17(4):1293-310; Arico et al., 2002, J Interferon Cytokine Res 22(11):1081-8; Flano et al., 2002, Immunol Res 25(3):201-17; Sauermann, 2001, Curr Mol Med 1(4):515-22; Pletnikov et al., 2002, Front Biosci 7:d593-607; Engler et al., 2001, Mol Immunol 38(6):457-65; White et al., 2001, Brain Pathol 11(4):475-9; Davis & Matalon, 2001, News Physiol Sci 16:185-90; Wang, 2001, Curr Top Microbiol Immunol. 258:201-19; Phillips et al., 2000, J Psychopharmacol. 14(3):244-50; Kazanji, 2000, AIDS Res Hum Retroviruses. 16(16):1741-6; Saif et al., 1996, Arch Virol Suppl. 12:153-61; and Hsiung et al., 1984, Rev Infect Dis. 6(1):33-50).

Animal models for viral respiratory infections such as, but not limited to,

PIV (see, e.g., Shephard et al., 2003 Res Vet Sci 74(2): 187-190; Ottolini et al., 2002 J Infect Dis 186(12): 1713-1717), RSV (see, e.g., Culley et al., 2002 J Exp Med 196(10): 1381-1386; and Curtis et al., 2002 Exp Biol Med 227(9): 799-802). In a specific embodiment, cotton rats are administered an antibody of the invention, a composition, or a combination therapy according to the methods of the invention, challenged with 10⁵ pfu of

RSV, and four or more days later the rats are sacrificed and RSV titer and anti-RSV antibody serum titer is determined. Accordingly, a dosage that results in a 2 log decrease or a 99% reduction in RSV titer in the cotton rat challenged with 10⁵ pfu of RSV relative to the cotton rat challenged with 10⁵ pfu of RSV but not administered the formulation is the dosage of the formulation that can be administered to a human for the prevention, treatment and/or management of one or more symptoms associated with RSV infection. Further, in accordance with this embodiment, the tissues (e.g., the lung tissues) from the sacrificed rats can be examined for histological changes.

The antibodies, compositions, or combination therapies of the invention can be tested for their ability to decrease the time course of viral infection. The antibodies, compositions, or combination therapies of the invention can also be tested for their ability to increase the survival period of humans suffering from a viral infection by at least 25%, at least 50%, at least 60%, at least 75%, at least 85%, at least 95%, or at least 99%. Further, antibodies, compositions, or combination therapies of the invention can be tested for their ability reduce the hospitalization period of humans suffering from viral infection by at least 60%, at least 75%, at least 85%, at least 95%, or at least 99%. Techniques known to those of skill in the art can be used to analyze the function of the antibodies, compositions, or combination therapies of the invention in vivo.

Animal models for bacterial infections can also be used to assess the efficacy of an antibody, a composition, or a combination therapy of the invention. Animal models for bacterial infections such as H. pylori-infection, genital mycoplasmosis, primary sclerosing cholangitis, cholera, chronic lung infection with Pseudomonas aeruginosa, Legionnaires' disease, gastroduodenal ulcer disease, bacterial meningitis, gastric Helicobacter infection, pneumococcal otitis media, experimental allergic neuritis, leprous neuropathy, mycobacterial infection, endocarditis, Aeromonas-associated enteritis, Bacteroides fragilis infection, syphilis, streptococcal endocarditis, acute hematogenous osteomyelitis, human scrub typhus, toxic shock syndrome, anaerobic infections, Escherichia coli infections, and Mycoplasma pneumoniae infections have been developed (see, e.g., Sugiyama et al., 2002, J Gastroenterol. 37 Suppl 13:6-9; Brown et al., 2001, Am J Reprod Immunol. 46(3):232-41; Vierling, 2001, Best Pract Res Clin Gastroenterol. 15(4):591-610; Klose, 2000, Trends Microbiol. 8(4):189-91; Stotland et al., 2000, Pediatr Pulmonol. 30(5):413-24; Brieland et al., 2000, Immunopharmacology 48(3):249-52; Lee, 2000, Baillieres Best Pract Res Clin Gastroenterol. 14(1):75-96; Koedel & Pfister, 1999, Infect Dis Clin North Am. 13(3):549-77; Nedrud, 1999, FEMS Immunol Med Microbiol. 24(2):243-50; Prellner et al., 1999, Microb Drug Resist. 5(1):73-82; Vriesendorp, 1997, J Infect Dis. 176 Suppl 2:S164-8; Shetty & Antia, 1996, Indian J Lepr. 68(1):95-104; Balasubramanian et al., 1994, Immunobiology 191(4-5):395-401; Carbon et al., 1994, Int J Biomed Comput. 36(1-2):59-67; Haberberger et al., 1991, Experientia. 47(5):426-9; Onderdonk et al., 1990, Rev Infect Dis. 12 Suppl 2:S169-77; Wicher & Wicher, 1989, Crit Rev Microbiol. 16(3):181-234; Scheld, 1987, J Antimicrob Chemother. 20 Suppl A:71-85; Emslie & Nade, 1986, Rev Infect Dis. 8(6):841-9; Ridgway et al., 1986, Lab Anim Sci. 36(5):481-5; Quimby & Nguyen, 1985, Crit Rev Microbiol. 12(1):1-44; Onderdonk et al., 1979, Rev Infect Dis. 1(2):291-301; Smith, 1976, Ciba Found Symp. (42):45-72, and Taylor-Robinson, 1976, Infection. 4(1 Suppl):4-8).

The antibodies, compositions, or combination therapies of the invention can be tested for their ability to decrease the time course of bacterial infection, e.g., a bacterial respiratory infection by at least 25%, at least 50%, at least 60%, at least 75%, at least 85%, at least 95%, or at least 99%. The antibodies, compositions, or combination therapies of the invention can also be tested for their ability to increase the survival period of humans suffering from a bacterial infection by at least 25%, at least 50%, at least 60%, at least 75%, at least 85%, at least 95%, or at least 99%. Further, the antibodies, compositions, or combination therapies administered according to the methods of the invention can be tested for their ability reduce the hospitalization period of humans suffering from bacterial infection, e.g., a bacterial respiratory infection, by at least 60%, at least 75%, at least 85%, at least 95%, or at least 99%. Techniques known to those of skill in the art can be used to analyze the function of the Antibodies of the invention, compositions, or combination therapies of the invention in vivo.

The efficacy of the antibodies, compositions, or combination therapies of the invention for the prevention, treatment and/or management of a fungal infection can be assessed in animal models for such infections. Animal models for fungal infections such as Candida infections, zygomycosis, Candida mastitis, progressive disseminated trichosporonosis with latent trichosporonemia, disseminated candidiasis, pulmonary paracoccidioidomycosis, pulmonary aspergillosis, Pneumocystis carinii pneumonia, cryptococcal meningitis, coccidioidal meningoencephalitis and cerebrospinal vasculitis, Aspergillus niger infection, Fusarium keratitis, paranasal sinus mycoses, Aspergillus fumigatus endocarditis, tibial dyschondroplasia, Candida glabrata vaginitis, oropharyngeal candidiasis, X-linked chronic granulomatous disease, tinea pedis, cutaneous candidiasis, mycotic placentitis, disseminated trichosporonosis, allergic bronchopulmonary aspergillosis, mycotic keratitis, Cryptococcus neoformans infection, fungal peritonitis, Curvularia geniculata infection, staphylococcal endophthalmitis, sporotrichosis, and dermatophytosis have been developed (see, e.g., Arendrup et al., 2002, Infection 30(5):286-91; Kamei, 2001, Mycopathologia 152(1):5-13; Guhad et al., 2000, FEMS Microbiol

Lett.192(1):27-31; Yamagata et al., 2000, J Clin Microbiol. 38(9):32606; Andrutis et al., 2000, J Clin Microbiol. 38(6):2317-23; Cock et al., 2000, Rev Inst Med Trop Sao Paulo 42(2):59-66; Shibuya et al., 1999, Microb Pathog. 27(3):123-31; Beers et al., 1999, J Lab Clin Med. 133(5):423-33; Najvar et al., 1999, Antimicrob Agents Chemother.43(2):413-4; Williams et al., 1988, J Infect Dis. 178(4):1217-21; Yoshida, 1988, Kansenshogaku Zasshi. 1998 June; 72(6):621-30; Alexandrakis et al., 1998, Br J Ophthalmol. 82(3):306-11; Chakrabarti et al., 1997, J Med Vet Mycol. 35(4):295-7; Martin et al., 1997, Antimicrob Agents Chemother. 41(1):13-6; Chu et al., 1996, Avian Dis. 40(3):715-9; Fidel et al., 1996, J Infect Dis. 173(2):425-31; Cole et al., 1995, FEMS Microbiol Lett. 15;126(2):177-80; Pollock et al., 1995, Nat Genet. 9(2):202-9; Uchida et al., 1994, Jpn J Antibiot. 47(10):1407-12; : Maebashi et al., 1994, J Med Vet Mycol. 32(5):349-59; Jensen & Schonheyder, 1993, J Exp Anim Sci. 35(4):155-60; Gokaslan & Anaissie, 1992, Infect Immun. 60(8):3339-44; Kurup et al., 1992, J Immunol. 148(12):3783-8; Singh et al., 1990, Mycopathologia. 112(3):127-37; Salkowski & Balish, 1990, Infect Immun. 58(10):3300-6; Ahmad et al., 1986, Am J Kidney Dis. 7(2):153-6; Alture-Werber E, Edberg S C, 1985, Mycopathologia. 89(2):69-73; Kane et al., 1981, Antimicrob Agents Chemother. 20(5):595-9; Barbee et al., 1977, Am J Pathol. 86(1):281-4; and Maestrone et al., 1973, Am J Vet Res. 34(6):833-6). Animal models for fungal respiratory infections such as Candida albicans, Aspergillus fumigatus, invasive pulmonary aspergillosis, Pneumocystis carinii, pulmonary cryptococcosis, Pseudomonas aeruginosa, Cunninghamella bertholletia (see, e.g., Aratani et al., 2002 Med Mycol 40(6):557-563; Bozza et al., 2002 Microbes Infect 4(13): 1281-1290; Kurup et al., 2002 Int Arch Allergy Immunol 129(2):129-137; Hori et al., 2002 Eur J Immuno 32(5): 1282-1291; Rivera et al., 2002 J Immuno 168(7): 3419-3427; Vassallo et al., 2001, Am J Respir Cell Mol Biol 25(2): 203-211; Wilder et al., 2002 Am J Respir Cell Mol Biol 26(3): 304-314; Yonezawa et al., 2000 J Infect Chemother 6(3): 155-161; Cacciapuoti et al., 2000 Antimicrob Agents Chemother 44(8): 2017-2022; and Honda et al., 1998 Mycopathologia 144(3):141-146).

The antibodies, compositions, or combination therapies of the invention can be tested for their ability to decrease the time course of fungal respiratory infection by at least 25%, at least 50%, at least 60%, at least 75%, at least 85%, at least 95%, or at least 99%. The antibodies, compositions, or combination therapies of the invention can also be tested for their ability to increase the survival period of humans suffering from a fungal respiratory infection by at least 25%, at least 50%, at least 60%, at least 75%, at least 85%, at least 95%, or at least 99%. Further, antibodies, compositions, or combination therapies administered according to the methods of the invention can be tested for their ability reduce the hospitalization period of humans suffering from fungal respiratory infection by at least 60%, at least 75%, at least 85%, at least 95%, or at least 99%. Techniques known to those of skill in the art can be used to analyze the function of the antibodies, compositions, or combination therapies of the invention in vivo.

Further, any assays known to those skilled in the art can be used to evaluate the prophylactic and/or therapeutic utility of an antibody, a composition, a combination therapy disclosed herein for prevention, treatment, management, and/or amelioration of disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof.

5.7.3. Toxicity Assays

The toxicity and/or efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED50 with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Further, any assays known to those skilled in the art can be used to evaluate the prophylactic and/or therapeutic utility of an antibody, a composition, a combination therapy disclosed herein for a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof.

5.8. Diagnostic Uses of Antibody Formulations

Antibodies (including molecules comprising, or alternatively consisting of, antibody fragments or variants thereof) of the liquid formulations of the invention that immunospecifically bind to an antigen of interest (e.g., an IL-9 polypeptide) can be used for diagnostic purposes to detect, diagnose, prognose, or monitor a disease or disorder, for example, a disease or disorder associated with or characterized by aberrant expression and/or activity of, e.g., an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. The invention provides for the detection of aberrant expression of IL-9 comprising: (a) assaying the expression of IL-9 in a biological sample from an individual using one or more antibodies of the liquid formulations of the invention that immunospecifically binds to an IL-9 polypeptide; and (b) comparing the level of IL-9 with a standard level of IL-9, e.g., in normal biological samples, whereby an increase or decrease in the assayed level of IL-9 compared to the standard level of IL-9 is indicative of a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof. In specific embodiments, aberrant expression level of IL-9 is indicative of an autoimmune disorder or a disease or condition associated therewith. In another specific embodiment, an aberrant expression level of IL-9 is indicative of an inflammatory disorder or a disease or condition associated therewith, such as asthma. In preferred embodiments, an aberrant expression level of IL-9 is indicative of a respiratory infection, such as, but not limited to RSV, PVI, or hMPV.

In certain embodiments, the labeled antibodies of the liquid formulations of the invention that immunospecifically bind to IL-9 are used for diagnostic purposes to detect, diagnose, prognose, or monitor a respiratory infection, e.g., RSV infection, PIV infection, or hMPV. The invention provides methods for the detection of a respiratory infection, comprising: (a) assaying the expression of IL-9 in cells or a tissue sample of a subject using one or more antibodies that immunospecifically bind to IL-9; and (b) comparing the level of IL-9 with a control level, e.g., levels in normal tissue samples not infected, whereby an increase in the assayed level of IL-9 compared to the control level of IL-9 is indicative of a respiratory infection.

Antibodies of the liquid formulations of the invention can be used to assay IL-9 levels in a biological sample using classical immunohistological methods as described herein or as known to those of skill in the art (e.g., see Jalkanen et al., 1985, J. Cell. Biol. 101:976-985; and Jalkanen et al., 1987, J. Cell . Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

One aspect of the invention is the detection and diagnosis of a disease or disorder associated with aberrant expression of IL-9 in an animal, preferably a mammal, and most preferably a human. In one embodiment, diagnosis comprises: a) administering (for example, parenterally, subcutaneously, or intraperitoneally) to a subject an effective amount of a labeled antibody (including molecules comprising, or alternatively consisting of, antibody fragments or variants thereof) of the liquid formulations of the invention that immunospecifically binds to an IL-9 polypeptide; b) waiting for a time interval following the administering for permitting the labeled antibody to preferentially concentrate at sites in the subject where IL-9 is expressed (and for unbound labeled molecule to be cleared to background level); c) determining background level; and d) detecting the labeled antibody in the subject, such that detection of labeled antibody (including antibody fragment thereof) above the background level and above or below the level observed in a person without the disease or disorder indicates that the subject has a particular disease or disorder associated with aberrant expression of IL-9. Background level can be determined by various methods including, comparing the amount of labeled molecule detected to a standard value previously determined for a particular system. Aberrant expression of IL-9 can occur particularly in lymphoid and myeloid cell types. A more definitive diagnosis of respiratory infection may allow health professionals to employ preventive measures or aggressive treatment earlier and thereby prevent the development or further progression of the infection.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S.W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds, Masson Publishing Inc. (1982). Depending on several variables, including the type of label used and the mode of administration, the time interval following the administration for permitting the labeled molecule to preferentially concentrate at sites in the subject and for unbound labeled molecule to be cleared to background level is 6 to 48 hours, 6 to 24 hours, or 6 to 12 hours. In another embodiment the time interval following administration is 5 to 20 days or 5 to 10 days.

In an embodiment, monitoring of the disease or disorder is carried out by repeating the method for diagnosing the disease or disorder, for example, one month after initial diagnosis, six months after initial diagnosis, one year after initial diagnosis, etc.

Presence of the labeled IL-9 antibody can be detected in the patient using methods known in the art for in vivo scanning These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the IL-9 antibody is labeled with a radioisotope and is detected in the patient using a radiation responsive surgical instrument (Thurston et al., U.S. Patent No. 5,441,050). In another embodiment, the IL-9 antibody is labeled with a fluorescent compound and is detected in the patient using a fluorescence responsive scanning instrument. In another embodiment, the IL-9 antibody is labeled with a positron emitting metal and is detected in the patient using positron emission-tomography. In yet another embodiment, the IL-9 antibody is labeled with a paramagnetic label and is detected in a patient using magnetic resonance imaging (MRI).

The antibodies of the invention (including molecules comprising, or alternatively consisting of, antibody fragments or variants thereof) may be utilized for immunophenotyping of cell lines and biological samples by their IL-9 expression or IL-9 receptor expression. Various techniques can be utilized using the antibodies, fragments, or variants of the liquid formulations of the invention to screen for cellular populations (that express IL-9 and/or IL-9 receptor, particularly immune cells, i.e., T and B lymphocytes, mast cells, eosinophils, macrophages, neutrophils and epithelial cells or IL-9 receptor, and include magnetic separation using antibody-coated magnetic beads, “panning” with antibody attached to a solid matrix (i.e., plate), and flow cytometry (see, e.g., U.S. Pat. No. 5,985,660; and Morrison et al., Cell, 96:737-49 (1999)).

These techniques allow for the screening of particular populations of cells, such as might be found with hematological malignancies (i.e., minimal residual disease (MRD) in acute leukemic patients) and “non-self' cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

5.9. Kits

The invention provides a pharmaceutical pack or kit comprising one or more containers filled with a liquid formulation of the invention. In a specific embodiment, the liquid formulations of the invention comprise antibodies (including antibody fragments thereof) recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. The invention also provides a pharmaceutical pack or kit comprising in one or more first containers a liquid formulation of the invention and in one or more second containers one or more other prophylactic or therapeutic agents useful for the prevention, management or treatment of a disease or disorder, for example, a disease or disorder associated with or characterized by aberrant expression and/or activity of,e.g., an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof In one embodiment, the liquid formulations of the invention are formulated in single dose vials as a sterile liquid containing 50 mM phosphate buffer at pH 6.2 and 150 mM sodium chloride. The formulations of the invention may be supplied in 3 cc USP Type I borosilicate amber vials (West Pharmaceutical Serices—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises a liquid formulation of the invention, in one or more containers. In another embodiment, a kit comprises a liquid formulation of the invention, in one or more containers, and one or more other prophylactic or therapeutic agents useful for the prevention, management or treatment of a disease or disorder. The disease or disorder may be associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (e.g., a respiratory infection), or one or more symptoms thereof, in one or more other containers. In a specific embodiment, the antibodies (including antibody fragments thereof) included in said liquid formulations is 4D4, 4D4 H2-1 D11, 4D4com-XF-9, 4D4com-2F9, 7F3, 71A10, 7F3com-2H2, 7F3com-3H5, or 7F3com-3D4 or an antigen-binding fragment. In an alternative embodiment, the antibody (including antibody fragment thereof) included in said liquid formulations is not 4D4, 4D4 H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof. Preferably, the kit further comprises instructions for preventing, treating and/or managing a disorder (e.g., using the liquid formulations of the invention alone or in combination with another prophylactic or therapeutic agent), as well as side effects and dosage information for method of administration.

5.10. Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., an antibody of the invention that immunospecifically binds to an antigen of interest (e.g., an IL-9 polypeptide), is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intranasal, or topical delivery.

In one embodiment, the unit dosage form is suitable for intravenous, intramuscular, intranasal, oral, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, total lymphocyte, mast cell counts, T cell counts, IgE production, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a liquid formulation containing an antibody. The packaging material includes instruction means which indicate that said antibody can be used to prevent, treat and/or manage one or more symptoms associated with a disorder associated with aberrant expression and/or activity of, e.g., an IL-9 polypeptide, a disorder associated with aberrant expression and/or activity of an IL-9R or one or more subunits thereof, an autoimmune disorder, an inflammatory disorder, a proliferative disorder, an infection (e.g., a respiratory infection), or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises a liquid formulation containing an antibody of interest, e.g., an antibody that immunospecifically binds to an IL-9 polypeptide and the other pharmaceutical agent comprises a second, different antibody that immunospecifically binds to an IL-9 polypeptide, and wherein said packaging material includes instruction means which indicate that said agents can be used to prevent, treat and/or manage a disorder associated with aberrant expression and/or activity of an IL-9 polypeptide, a disorder associated with aberrant expression and/or activity of an IL-9R or one or more subunits thereof, an autoimmune disorder, an inflammatory disorder, a proliferative disorder, an infection (e.g., a respiratory infection), or one or more symptoms thereof by administering specific doses and using specific dosing regimens as described herein.

The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material, wherein one pharmaceutical agent comprises a liquid formulation containing an antibody that immunospecifically binds to an IL-9 polypeptide and the other pharmaceutical agent comprises a prophylactic or therapeutic agent other than an antibody that immunospecifically binds to an IL-9 polypeptide, and wherein said packaging material includes instruction means which indicate that said agents can be used to prevent, treat and/or manage one or more symptoms associated with a disorder associated with aberrant expression and/or activity of an IL-9 polypeptide, a disorder associated with aberrant expression and/or activity of an IL-9R or one or more subunits thereof, an autoimmune disorder, an inflammatory disorder, a proliferative disorder, an infection (e.g., a respiratory infection), or one or more symptoms thereof by administering specific doses and using specific dosing regimens as described herein.

The present invention provides that the adverse effects that may be reduced or avoided by the methods of the invention are indicated in informational material enclosed in an article of manufacture for use in preventing, treating and/or managing one or more symptoms associated with an autoimmune disorder, an inflammatory disorder or an infection. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

Further, the information material enclosed in an article of manufacture for use in preventing, treating and/or managing disease or disorder, for example a disease or disorder characterized by aberrant expression and/or activity of an IL-9 polypeptide, a disorder characterized by aberrant expression and/or activity of an IL-9R or one or more subunits thereof, an inflammatory disorder, an autoimmune disorder, a proliferative disorder, or an infection (e.g., a respiratory infection) or one or more symptoms thereof can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### 6. SPECIFIC EMBODIMENTS

1. An antibody formulation comprising an aqueous carrier, phosphate, and 50 mg/ml or higher of an antibody or antibody fragment, said antibody formulation being formulated for administration to a human subject.

2. An antibody formulation formulated for administration to a human subject, said formulation comprising an aqueous carrier, phosphate, and 10 mg/ml or higher of an antibody or antibody fragment, wherein said antibody or antibody fragment displays a reduction in one or more of the following phase behaviors when formulated in a phosphate buffer at a pH below the pI of said antibody in the presence of salt, as compared to said antibody when formulated in a histidine buffer at said pH in the presence of salt at the same concentration:

a) formation of unfolded intermediates;

b) colloidal instability;

c) soluble association of the antibody molecules; or

d) precipitation of the antibody molecules;

wherein said at least one or more phase behaviors are measured by techniques selected from the group consisting of high performance size exclusion chromatography (HPSEC), tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and 1-anilino-8-naphthalenesulfonic acid (ANS) protein binding techniques.

3. The formulation of embodiment 1 or 2, wherein said antibody or antibody fragment immunospecifically binds to IL-9 polypeptide.

4. The formulation of embodiment 1 or 2, wherein the aqueous carrier is distilled water.

5. The formulation of embodiment 1 or 2, wherein the formulation has a pH in the range of between 4.0 and 8.0.

6. The formulation of embodiment 5, wherein the pH is in the range of about 6.0 and 6.5.

7. The formulation of embodiment 1 or 2, further comprising salt at a concentration of no more than about 200 mM.

8. The formulation of embodiment 7, wherein the salt is at a concentration ranging from about 100 mM to about 200 mM.

9. The formulation of embodiment 6, further comprising a salt at a concentration ranging from about 100 to about 200 mM.

10. The formulation of embodiment 1 or 2, further comprising a sugar.

11. The formulation of embodiment 6, further comprising a sugar.

12. The formulation of embodiment 10, wherein the sugar is sucrose.

13. The formulation of embodiment 11, wherein the sugar is sucrose.

14. The formulation of embodiment 10, wherein the sugar is trehalose.

15. The formulation of embodiment 11, wherein the sugar is trehalose.

16. The formulation of embodiment 12, wherein the sucrose is at a concentration of up to 10%.

17. The formulation of embodiment 13, wherein the sucrose is at a concentration of up to 10%.

18. The formulation of embodiment 14, wherein the trehalose is at a concentration of up to 10%.

19. The formulation of embodiment 15, wherein the trehalose is at a concentration of up to 10%.

20. The formulation of embodiment 1 or 2, further comprising a surfactant.

21. The formulation of embodiment 6, further comprising a surfactant.

22. The formulation of embodiment 9, further comprising a surfactant.

23. The formulation of embodiment 21, wherein the surfactant is Tween-20 or Tween-80.

24. The formulation of embodiment 22, wherein the surfactant is Tween-20 or Tween-80.

25. The formulation of embodiment 23, wherein the surfactant is at a concentration of up to 0.1%.

26. The formulation of embodiment 24, wherein the surfactant is at a concentration of up to 0.1%.

27. The formulation of embodiment 1 or 2, wherein the antibody or antibody fragment is at a concentration of at least 100 mg/ml.

28. The formulation of embodiment 1 or 2, wherein the antibody or antibody fragment is at a concentration in the range from about 50 mg/ml to about 150 mg/ml.

29. The formulation of embodiment 1 or 2, wherein phosphate is at a concentration in the range from about 10 mM to about 100 mM.

30. The formulation of embodiment 29, wherein phosphate is at a concentration in the range from about 25 mM to about 75 mM.

31. The formulation of embodiment 1 or 2, wherein the antibody or antibody fragment is stable during storage at 40° C. for at least 15 days as determined by high performance size exclusion chromatography (HPSEC).

32. The formulation of embodiment 1 or 2, wherein the antibody or antibody fragment is stable during storage at about ambient temperature for at least 6 months as determined by HPSEC.

33. The formulation of embodiment 1 or 2, wherein the antibody or antibody fragment is stable during storage at 4° C. for at least 1.5 years as determined by HPSEC.

34. The formulation of embodiment 1 or 2, wherein less than 5% of the antibody or antibody fragment forms an aggregate during storage as measured by HPSEC.

35. The formulation of embodiment 6, wherein less than 5% of the antibody or antibody fragment forms an aggregate during storage as measured by HPSEC.

36. The formulation of embodiment 9, wherein less than 5% of the antibody or antibody fragment forms an aggregate during storage as measured by HPSEC.

37. The formulation of embodiment 1 or 2, wherein less than 2% of the antibody or antibody fragment forms an aggregate during storage as measured by HPSEC.

38. The formulation of embodiment 6, wherein less than 2% of the antibody or antibody fragment forms an aggregate during storage as measured by HPSEC.

39. The formulation of embodiment 9, wherein less than 2% of the antibody or antibody fragment forms an aggregate during storage as measured by HPSEC.

40. The formulation of embodiment 1 or 2, wherein less than 1% of the antibody or antibody fragment forms an aggregate during storage as measured by HPSEC.

41. The formulation of embodiment 6, wherein less than 1% of the antibody or antibody fragment forms an aggregate during storage as measured by HPSEC.

42. The formulation of embodiment 9, wherein less than 1% of the antibody or antibody fragment forms an aggregate during storage as measured by HPSEC.

43. The formulation of embodiment 1 or 2, wherein the antibody or the fragment thereof retains at least 80% of binding ability compared to a reference antibody representing the antibody prior to storage.

44. The formulation of embodiment 6, wherein the antibody or the fragment thereof retains at least 80% of binding ability compared to a reference antibody representing the antibody prior to storage.

45. The formulation of embodiment 9, wherein the antibody or the fragment thereof retains at least 80% of binding ability compared to a reference antibody representing the antibody prior to storage.

46. The formulation of embodiment 1 or 2, wherein the antibody or the fragment thereof retains at least 85% of binding ability compared to a reference antibody.

47. The formulation of embodiment 6, wherein the antibody or the fragment thereof retains at least 85% of binding ability compared to a reference antibody.

48. The formulation of embodiment 9, wherein the antibody or the fragment thereof retains at least 85% of binding ability compared to a reference antibody.

49. The formulation of embodiment 1 or 2, wherein the antibody or the fragment thereof retains at least 90% of binding ability compared to a reference antibody.

50. The formulation of embodiment 6, wherein the antibody or the fragment thereof retains at least 90% of binding ability compared to a reference antibody.

51. The formulation of embodiment 9, wherein the antibody or the fragment thereof retains at least 90% of binding ability compared to a reference antibody.

52. The formulation of embodiment 1 or 2, wherein the antibody or the fragment thereof retains at least 95% of binding ability compared to a reference antibody.

53. The formulation of embodiment 6, wherein the antibody or the fragment thereof retains at least 95% of binding ability compared to a reference antibody.

54. The formulation of embodiment 9, wherein the antibody or the fragment thereof retains at least 95% of binding ability compared to a reference antibody.

55. The formulation of embodiment 1 or 2, wherein the antibody or antibody fragment is 4D4, 4D4H2-1 D11, 4D4com-XF-9, 4D4com-2F9, 7F3, 71A10, 7F3 22D3, 7F3com-2H2, 7F3com-3H5, or 7F3com-3D4 or an antigen binding fragment thereof.

56. The formulation of embodiment 6, wherein the antibody or antibody fragment is 4D4, 4D4H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof.

57. The formulation of embodiment 9, wherein the antibody or antibody fragment is 4D4, 4D4H2-1 D11, 4D4com-XF-9, 4D4com-2F9, 7F3, 71A10, 7F3 22D3, 7F3com-2H2, 7F3com-3H5, or 7F3com-3D4 or an antigen-binding fragment thereof.

58. The formulation of embodiment 57, wherein the antibody or antibody fragment is 7F3com-2H2.

59. A pharmaceutical unit dosage form suitable for parenteral administration to a human which comprises an antibody formulation of embodiment 1 or 2 in a suitable container.

60. The pharmaceutical unit dosage form of embodiment 59, wherein the antibody formulation is for intravenous, subcutaneous, or intramuscular injection.

61. A pharmaceutical unit dosage form suitable for aerosol administration to a human which comprises an antibody formulation of embodiment 1 or 2 in a suitable container.

62. The pharmaceutical unit dosage of embodiment 61, wherein the antibody formulation is administered intranasally.

63. An antibody formulation which is produced by lyophilizing the aqueous antibody formulation of embodiment 1 or 2.

64. A sealed container containing the formulation of embodiment 1 or 2.

65. A sealed container containing the formulation of embodiment 9.

66. The sealed container of embodiment 64, having sufficient volume for reconstitution with a pharmaceutically acceptable carrier.

67. The sealed container of embodiment 66, wherein said carrier is water for injection, USP, 5% dextrose in water (D5W) or saline.

68. The sealed container of embodiment 67, wherein said container maintains a sterile environment and allows reconstitution of the formulation without loss of sterility.

69. A kit comprising in one or more containers an antibody formulation comprising in an aqueous carrier, phosphate, and 50 mg/ml or higher of an antibody or fragment thereof, and instructions for use of the formulation, said antibody formulation being formulated for administration to a human subject.

70. The kit of embodiment 69, wherein the formulation is sterile.

71. The kit of embodiment 69, wherein the aqueous carrier is distilled water.

72. The kit of embodiment 70, wherein the antibody or antibody fragment is 4D4, 4D4H2-1 D11, 4D4com-XF-9, 4D4com-2F9, 7F3, 71A10, 7F3 22D3, 7F3com-2H2, 7F3com-3H5, or 7F3com-3D4 or an antigen binding fragment thereof.

73. The kit of embodiment 72, wherein the antibody or antibody fragment is 7F3com-2H2.

74. The kit of embodiment 70, 72, or 73, wherein the formulation is produced by lyophilizing the aqueous antibody formulation.

75. The kit of embodiment 70 or 73, wherein the antibody or antibody fragment is at a concentration of about 50 mg/ml to about 150 mg/ml.

76. A method of preventing, managing, treating or ameliorating an inflammatory disease, an autoimmune disease, a disorder associated with aberrant expression and/or activity of an IL-9 polypeptide, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (preferably, a respiratory infection), or one or more symptoms thereof, said method comprising administering to a subject in need thereof a prophylactically or therapeutically effective amount of an antibody formulation of embodiment 56.

77. A method of preventing, managing, treating or ameliorating a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (preferably, a respiratory infection), or one or more symptoms thereof, said method comprising administering to a subject in need thereof a prophylactically or therapeutically effective amount of an antibody formulation of embodiment 57.

78. A method of preventing, managing, treating or ameliorating a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (preferably, a respiratory infection), or one or more symptoms thereof, said method comprising administering to a subject in need thereof a prophylactically or therapeutically effective amount of an antibody formulation of embodiment 58.

79. The method of embodiment 76, wherein the antibody or antibody fragment thereof polypeptide is stable during storage at 40° C. for at least 15 days as determined by HPSEC.

80. The method of embodiment 77, wherein the antibody or antibody fragment thereof polypeptide is stable during storage at 40° C. for at least 15 days as determined by HPSEC.

81. The method of embodiment 78, wherein the antibody or antibody fragment thereof polypeptide is stable during storage at 40° C. for at least 15 days as determined by HPSEC.

82. The method of embodiment 76, wherein less than 5% of the antibody or antibody fragment forms an aggregate during storage as measured by HPSEC.

83. The method of embodiment 77, wherein less than 5% of the antibody or antibody fragment forms an aggregate during storage as measured by HPSEC.

84. The method of embodiment 78, wherein less than 5% of the antibody or antibody fragment forms an aggregate during storage as measured by HPSEC.

85. The method of embodiment 76, wherein the antibody or the fragment thereof retains at least 80% of binding ability to an IL-9 polypeptide compared to a reference antibody representing the antibody or antibody fragment prior to storage.

86. The method of embodiment 77, wherein the antibody or the fragment thereof retains at least 80% of binding ability to an IL-9 polypeptide compared to a reference antibody representing the antibody or antibody fragment prior to storage.

87. The method of embodiment 78, wherein the antibody or the fragment thereof retains at least 80% of binding ability to an IL-9 polypeptide compared to a reference antibody representing the antibody or antibody fragment prior to storage.

88. The method of embodiment 76, wherein the formulation is administered parenterally.

89. The method of embodiment 77, wherein the formulation is administered parenterally.

90. The method of embodiment 78, wherein the formulation is administered parenterally.

91. The method of embodiment 76, wherein the formulation is administered subcutaneously, orally or intranasally.

92. The method of embodiment 77, wherein the formulation is administered subcutaneously, orally or intranasally.

93. The method of embodiment 78, wherein the formulation is administered subcutaneously, orally or intranasally.

94. The method of embodiment 76, wherein the inflammatory disease is asthma or an allergy.

95. The method of embodiment 77, wherein the inflammatory disease is asthma or an allergy.

96. The method of embodiment 78, wherein the inflammatory disease is asthma or an allergy.

97. An antibody formulation for administration to a subject, said formulation comprising an aqueous carrier, phosphate, and 50 mg/ml or higher of 7F3com-2H2 or an antigen-binding fragment thereof.

98. The formulation of embodiment 97, wherein the formulation is sterile.

99. The formulation of embodiment 97, wherein the aqueous carrier is distilled water.

100. The formulation of embodiment 97 or 98, wherein the formulation has a pH in the range of between 4.0 and 8.0.

101. The formulation of embodiment 100, wherein the pH is in the range of about 6.0 and 6.5.

102. The formulation of embodiment 97 or 98, further comprising a salt at a concentration of no more than about 200 mM.

103. The formulation of embodiment 102, wherein the salt is at a concentration ranging from about 100 mM to about 200 mM.

104. The formulation of embodiment 101, wherein the salt is at a concentration ranging from about 100 mM to about 200 mM.

105. The formulation of embodiment 97 or 98, further comprising a sugar.

106. The formulation of embodiment 101, further comprising a sugar.

107. The formulation of embodiment 105, wherein the sugar is sucrose.

108. The formulation of embodiment 106, wherein the sugar is sucrose.

109. The formulation of embodiment 105, wherein the sugar is trehalose.

110. The formulation of embodiment 106, wherein the sugar is trehalose.

111. The formulation of embodiment 107, wherein the sucrose is at a concentration of up to 10%.

112. The formulation of embodiment 108, wherein the sucrose is at a concentration of up to 10%.

113. The formulation of embodiment 109, wherein the trehalose is at a concentration of up to 10%.

114. The formulation of embodiment 110, wherein the trehalose is at a concentration of up to 10%.

115. The formulation of embodiment 97 or 98, further comprising a surfactant.

116. The formulation of embodiment 101, further comprising a surfactant.

117. The formulation of embodiment 104, further comprising a surfactant.

118. The formulation of embodiment 116, wherein the surfactant is Tween-20 or Tween-80.

119. The formulation of embodiment 117, wherein the surfactant is Tween-20 or Tween-80.

120. The formulation of embodiment 118, wherein the Tween-20 or Tween-80 is at a concentration of up to 0.1%.

121. The formulation of embodiment 119, wehrein the Tween-20 or Tween-80 is at a concentration of up to 0.1%.

122. The formulation of embodiment 97 or 98, wherein the antibody or antibody fragment is at a concentration of at least 100 mg/ml.

123. The formulation of embodiment 97 or 98, wherein the antibody or antibody fragment is at a concentration in the range from about 50 mg/ml to about 150 mg/ml.

124. The formulation of embodiment 97 or 98, wherein the phosphate is at a concentration in the range from about 10 mM to about 100 mM.

125. The formulation of embodiment 123, wherein the phosphate is at a concnetration in the range from about 25 mM to about 75 mM.

126. The formualtion of embodiment 124, wherein the phosphate is at a concnetration in the range from about 25 mM to about 75 mM.

127. The formulation of embodiment 97 or 98, wherein the antibody or antibody fragment is stable during storage at 40° C. for at least 15 days as determined by high performance size exclusion chromatography (HPSEC).

128. The formulation of embodiment 97 or 98, wherein the antibody or antibody fragment is stable during storage at about ambient temperature for at least 6 months as determined by HPSEC.

129. The formulation of embodiment 97 or 98, wherein the antibody or antibody fragment is stable during storage at 4° C. for at least 1.5 years as determined by HPSEC.

130. The formulation of embodiment 97 or 98, wherein less than 5% of the antibody or antibody fragment forms an aggregate during storage as measured by HPSEC.

131. The formulation of embodiment 101, wherein less than 5% of the antibody or antibody fragment forms an aggregate during storage as measured by HPSEC.

132. The formulation of embodiment 104, wherein less than 5% of the antibody or antibody fragment forms an aggregate during storage as measured by HPSEC.

133. A method of preventing, treating or managing a disease or disorder comprising administering to a subject in need thereof a prophylactically or therapeutically effective amount of an antibody formulation of embodiment 1 or 2.

134. An antibody formulation for administration to a subject, said formulation comprising an aqueous carrier, between about 50 mg/ml and 150 mg/ml 7F3com-2H2 antibody, between about 10 mM and about 75 mM phosphate, between about 100 mM and 200 mM NaC1, wherein said formulation has a pH between about 5.5 and 6.5.

135. The formulation of embodiment 134, wherein said formulation comprises an aqueous carrier, about 100mg/m1 7F3com-2H2 antibody, about 25 mM phosphate, about 150 mM NaC1, wherein said formulation has a pH of about 6.0.

136. The formulation of embodiment 135, wherein the formulation is isotonic.

137. The formulation of embodiment 135, wherein the antibody or antibody fragment is stable during storage at 40° C. for at least 15 days as determined by high performance size exclusion chromatography (HPSEC).

138. The formulation of embodiment 135, wherein the antibody or antibody fragment is stable during storage at about ambient temperature for at least 6 months as determined by HPSEC.

139. The formulation of embodiment 135, wherein the antibody or antibody fragment is stable during storage at 4° C. for at least 1.5 years as determined by HPSEC.

140. The formulation of embodiment 135, wherein less than 5% of the antibody or antibody fragment forms an aggregate during storage as measured by HPSEC.

141. A method of preventing, managing, treating or ameliorating an inflammatory disease, an autoimmune disease, a disorder associated with aberrant expression and/or activity of an IL-9 polypeptide, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (preferably, a respiratory infection), or one or more symptoms thereof, said method comprising administering to a subject in need thereof a prophylactically or therapeutically effective amount of an antibody formulation of embodiment 135.

### 7. EXAMPLES

7.1. Antibody Purification and Antibody Formulations

The following section describes a method for purifying antibodies to be used in the formulations of the invention (see FIG. 16).

7.1.1. Buffer Components and Equipment

Buffers are tested for bioburden and endotoxin.

Buffers and Process Solutions

0.1 M citric acid 10 mM sodium citrate, 80 mM NaCl, pH 4.6

25 mM sodium phosphate, pH 6.5

20 mM Tris-HCl, 40 mM NaCl, pH 7.5

0.5 M sodium phosphate, pH 6.5

5 mM sodium phosphate, 40 mM NaCl, pH 6.5

50 mM Glycine-HCl, 30 mM NaCl, pH 2.5

50 mM Glycine-HC, pH 2.35

1.0 M Tris base

Cleaning and Storage Solutions

Water for Injection (WFI)

1.0 N NaOH

0.1 N NaOH

20% (v/v) ethanol

0.5 N NaOH, 400 ppm sodium hypochlorite

Formulation Buffers

≧10 mg/mL of antibody of interest

10-100 mM phosphate buffer, pH 4-8

0-200 mM NaCl or alternately 0-10% of sucrose or trehalose

0-0.1% Tween-20 or Tween-80

Equipment (Substitutions with Equivalent Performing Materials are Acceptable)

300 kg scale

Conductivity meter

Stir plate

pH meter

Vessels: Appropriately sized StedimTM bags, buffer tanks, PETG Bottles

Watson Marlow 1700 peristaltic pump

Wedgewood UV, pH, conductivity unit

Amersham Pharmacia chromatography controller

Packed POROS HS50 cation exchange gel

Packed Pharmacia rProtein A affinity gel

Packed POROS HQ anion exchange gel

Sterile, depyrogenated silicone tubing

Integritest Filter Integrity Tester II

Sterile Asahi Planova 20 N membrane viral removal filter

Millipore 0.2 micron Durapore filter

Millipore Multimedia filter

CUNO 60LP, 10/60 SP filter

CUNO filter housing

Class 100 hood

7.1.2. Purification and Formulation of the Antibodies

FIG. 16 outlines the process steps for the purification and formulation of the antibodies (including antibody fragments thereof) of the invention. The purification process comprises three chromatography steps, a nanofiltration step, a low pH treatment step, and formulation. These steps are designed to remove host cell proteins, DNA and cell culture components such as BSA and transferrin. In addition, the process includes steps to control bioburden and endotoxin and to remove and inactivate viruses.

7.1.2.1. Conditioned Medium (Steps 1 to 6)

Conditioned medium from a single cell culture lot or pooled from multiple cell culture lots is purified as a single lot. The combination of multiple cell culture lots into one purification lot is performed in order to utilize downstream processing steps sized for a single lot size and to decrease the number of purification lots. For example, because the working volumes of 130 L and 250 L cell culture bioreactors are approximately 100 L and 200 L, respectively, these two cell culture lots could be pooled and run as one 300 L purification lot. Process product samples are analyzed for DNA using a PicoGreen or a quantitative PCR assay to detect DNA. Protein concentration is determined either by a Protein A bindable HPLC assay or by UV absorbance at 280 nm. Product-containing process streams are monitored for endotoxin and bioburden. Column effluents are monitored for endotoxin. A description of each step is summarized below.

7.1.2.2. Conditioned Medium Adjustment and Filtration (Step 7)

The conditioned medium is adjusted to pH 4.6±0.2 with 0.1 M citric acid. The adjusted conditioned medium is then filtered using a CUNO filter in-line with a Millipore 0.2 micron Durapore filter.

7.1.2.3. Cation Exchange Chromatography Step (Step 8)

The adjusted and filtered conditioned medium is loaded onto a cation exchange column that has been equilibrated with 10 mM sodium phosphate, 80 mM sodium chloride, pH 4.6. The bound antibody is washed using the same buffer. The column is then washed with 25 mM sodium phosphate pH 6.5 to remove process impurities, especially BSA. The product is eluted using 20 mM Tris-HCl buffer, 40 mM NaCl, pH 7.5. Following elution of the product, the column is cleaned with 1.0 N NaOH and stored in 0.1 N NaOH at room temperature.

7.1.2.4. rProtein A Chromatography (Step 9)

The cation exchange product is loaded directly onto a rProtein A column equilibrated with 20 mM Tris-HCl buffer, 40 mM NaCl, pH 7.5. Following loading, the column is washed with the equilibration buffer, and the product is eluted with 50 mM glycine, 30 mM NaCl, pH 3.2. The rProtein A product is neutralized to pH 6.5±0.2 with 1.0 M Tris base. This chromatography step removes additional process-related impurities. At the end of the step, the column is washed with equilibration buffer, cleaned with 0.1 N NaOH, washed with equilibration buffer and stored in 20% (v/v) ethanol at room temperature.

7.1.2.5. Anion Exchange Chromatography (Step 10)

This chromatographic step is the final step designed to remove any trace levels of process-related impurities. The column is equilibrated with 0.5 M sodium phosphate, pH 6.5 followed by equilibration with 5 mM sodium phosphate, 40 mM sodium chloride, pH 6.5. Under these conditions, the neutralized rProtein A product is loaded onto the equilibrated anion exchange column, and under these conditions, the product is recovered in the non-bound fraction and the process-related impurities are retained in the column. The column is cleaned with 1.0 N NaOH and stored in 0.1 N NaOH at room temperature.

7.1.2.6. Nanofiltration (Step 11)

The anion exchange product is filtered through a sterile Planova™ 20 N membrane (pore size=20 nm) that is prepared by flushing first with WFI and then with 5 mM sodium phosphate, 40 mM sodium chloride pH 6.5. After the product is filtered, the filter is chased with a small volume of 5 mM sodium phosphate, 40 mM sodium chloride, pH 6.5 to maximize product recovery. After filtration the nanofilter is integrity tested.

7.1.2.7. Low pH Treatment (Step 12)

The pH of the nanofiltered product is adjusted to 3.4±0.1 with 50 mM glycine, pH 2.35 and held at this pH for 30±10 minutes. After low pH treatment, the product pH is adjusted to 6.5±0.2 with 1.0 M Tris base.

7.1.2.8. Formulation of Anti-IL-9 Antibodies

Sodium chloride (150 mM) is added to the Intermediate Drug Substance using a 4 M NaCl stock solution. It is then 0.2 micron filtered and concentrated to 20 g/L using tangential flow filtration with a 30 kD membrane. The product is then diafiltered into the formulation buffer), using a minimum of five buffer exchanges. The product is then concentrated to 100 g/L and filtered through a 1.2/0.2 micron membrane into sterile Stedim® storage bags. Sample purity is confirmed by Size Exclusion Chromotography (SEC).

7.2. Buffer Ion-Induced Formation of Intermediates

This example indicates that the interaction of histidine with full-length monoclonal antibodies results in reduction of domain-domain interactions and destabilization of some domains. Because of this effect of histidine, the protein molecule unfolds sequentially through the formation of intermediate species. By contrast, interaction of phosphate with full-length monoclonal antibodies does not result in formation of intermediate species under specified conditions.

7.2.1. Materials and Methods

### Urea-Induced Unfolding Experiments

Urea-induced unfolding of 7F3com-2H2 was conducted in the absence of salt or in presence of 150 mM NaCl, in either 10 mM histidine (pH 6.0) or 10 mM phosphate buffer (pH 6.0). Unfolding of 7F3com-2H2 was measured by intrinsic tryptophan fluorescence by monitoring changes in the center of spectral mass (CSM) as a function of urea concentration. The protein solution was excited at 293 nm and the emission spectra were recorded between 310 to 400 nm. The spectral bandwidth was set up at 4 nm for both excitation and emission. For unfolding and refolding experiments, the samples were incubated at 23° C. for 12 hours before measurements. This incubation time has been found optimal to allow the protein to reach the unfolding equilibrium. The data was normalized using (Y_{U}-Y_{obs})/(Y_{U}-Y_{F}), where Y_{obs} is the spectroscopic value observed at urea concentration and Y_{F} and Y_{U} are the spectroscopic values of folded and unfolded 7F3com-2H2, respectively. All experiments were carried out at 23° C. with a protein concentration of 0.67 μM.

### Differential Scanning Calorimetry (DSC)

DSC was conducted for 7F3com-2H2 under conditions of: (a)10 mM histidine, pH 6.0, and in the presence of no salt, 25 mM NaCl, or 150 mM NaCl and (b) 10 mM phosphate buffer, pH 6.0, and in the presence of no salt or 150 mM NaCl. DSC experiments were performed on a VP DSC Ultrasensitive Calorimeter from Microcal. Protein solutions were exhaustively dialyzed against corresponding buffer solutions for a minimum period of 12 hours. The final dialysate was used for reference cell. All the protein solutions and buffer were degassed with gentle stirring under vacuum before being loaded into the calorimeter to ensure no bubbles during heating process. Experiments were performed over a range of 30-100° C. at a scan rate of 1.5° C. per minute. Normalized heat capacity (Cp) data were corrected for buffer baseline. A protein concentration of 67 μM was used for DSC experiments.

### Potassium Iodide (KI) Quenching Experiments

Stern-Volmer plots were generated of native and intermediate 7F3com-2H2 in 10 mM histidine, 150 mM NaCl, pH 6.0 and of native and unfolded 7F3com-2H2 in 10 mM phosphate, 150 mM NaCl, pH 6.0. Increasing aliquots of KI from a 6M stock were added to protein at 0.67 μM in the respective buffer. The KI solution was prepared in 0.2 mM sodium thiosulfate to prevent formation of free iodine. Fluorescence intensity at the emission maximum (331 nm for native, 348 nm for intermediate, and 352 nm for unfolded) was measured after exciting 7F3com-2H2 at 293 nm.

Tryptophan fluorescence quenching induced by KI was analyzed according to the Stern-Volmer and Lehrer equations. The quenching process can be described by the classical Stern-Volmer relationship: F₀/F=1+K_{SV}[Q], where F₀ and F are the fluorescence intensities in the absence and presence of quencher, [Q] is the concentration of the quencher, and K_{SV} is the Stern-Volmer quenching constant. The fraction of total fluorophore accessible to the quencher can be calculated from the modified Stern-Volmer plot, also known as Lehrer plot: F₀/ΔF=1/K_{Q}fₐ[Q]+1/fₐ, where F₀ and [Q] have the same meaning as defined earlier, ΔF is the change in the fluorescence intensity due to quenching, K_{Q} is the Stern-Volmer quenching constant of the exposed tryptophan residues and fₐ is the fraction of the initial fluorescence which is accessible to the quencher. From this equation, a plot of F₀/ΔF=f(1/[Q]) yields a straight line whose extrapolation at 1/[Q]=0 gives the value of 1/fₐ on the axis F₀/ΔF.

1-Anilino-8-naphthalenesulfonate (ANS) Binding Experiment

Urea-induced unfolding of 7F3com-2H2 at pH 8.1 in either 10 mM histidine or 10 mM phosphate, was followed by tryptophan fluorescence and ANS binding assay. For all experiments, a protein concentration of 0.67 μM was used and the data was normalized using the (Y_{U}-Y_{obs})/(Y_{U}-Y_{F}) formula as mentioned previously. For ANS binding studies, 7F3com-2H2 was equilibrated overnight with different concentrations of urea at 23° C. The protein thus equilibrated with different concentration of denaturants was then treated with freshly prepared ANS. A protein-to-ANS ratio of 1:100 was maintained such that there was a large excess of ANS in solution. The mixture was incubated at 23° C. for 2 hours. The binding of the hydrophobic dye ANS to protein was monitored by measuring the fluorescence intensity between 400-600 nm after excitation at 350 nm.

7.2.2. Results

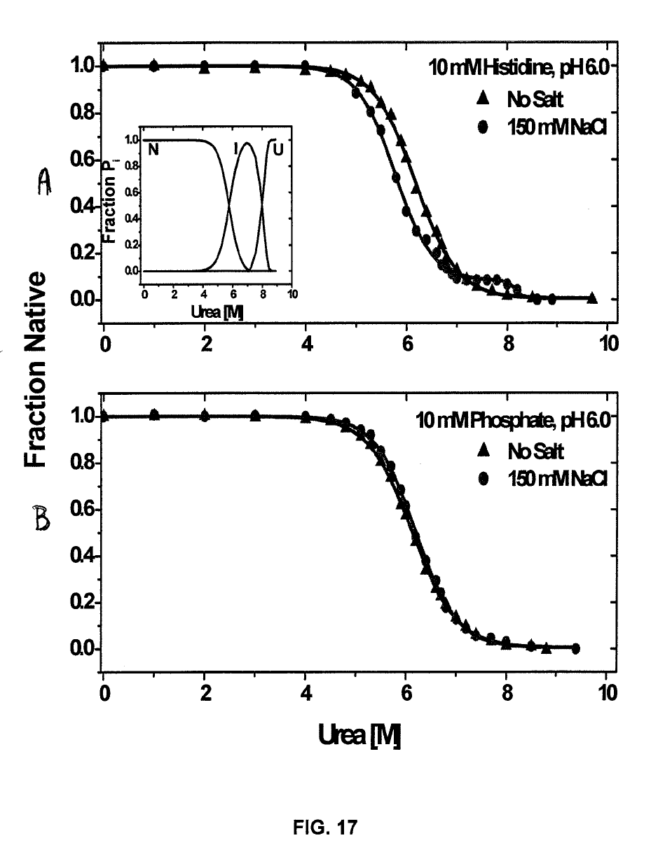

Urea-inducing folding experiments demonstrate that in the presence of histidine alone at pH 6.0, the unfolding of the antibody is a simple 2-step process which is indicative of cooperative unfolding of all domains, whereas with the addition of salt, the unfolding of 7F3com-2H2 domains is a sequential process through the formation of an intermediate population (see FIG. 17A). By contrast, in the presence of phosphate at pH 6.0, no such intermediate is formed with or without salt (see FIG. 17B).

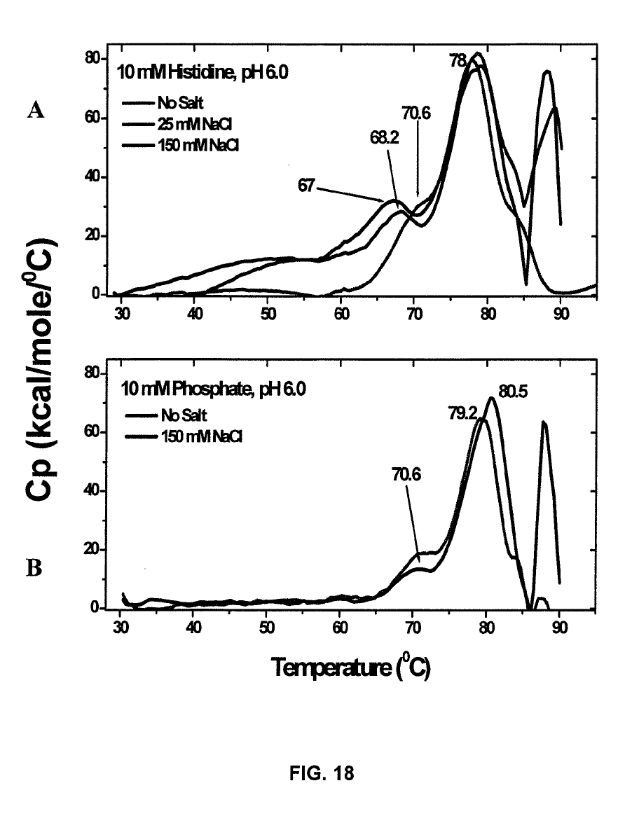

DSC experiments reveal that at pH 6 in the absence of salt, the DSC thermogram is complex with 3 partially overlapping transitions (see FIG. 18). The overlapping of the 3 transitions indicate strong domain-domain interactions. Addition of 150 mM NaCl destabilizes the first transition as demonstrated by the Tₘₗ being lowered by almost 3.5° C.

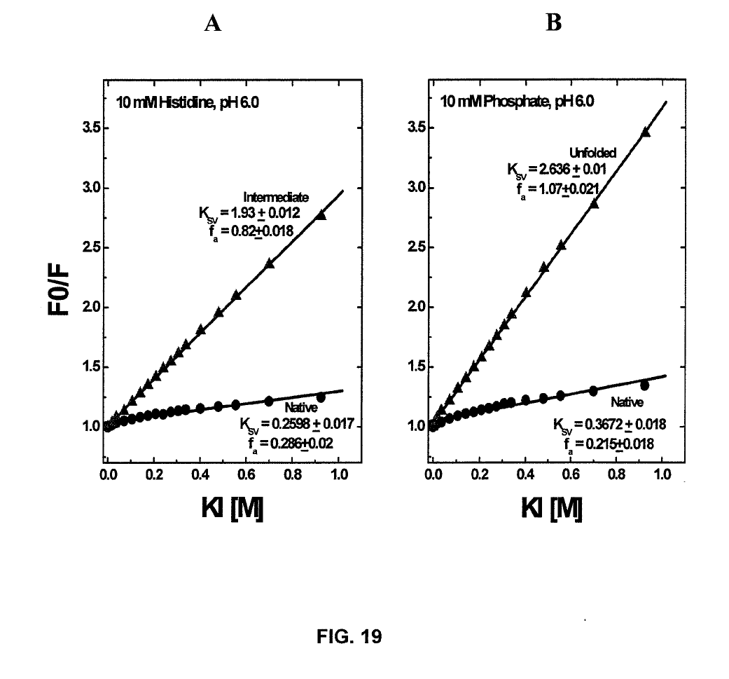

KI quelching studies indicate that at 7.5 M Urea, where the intermediate state was observed (see FIG. 17), a fₐ value of 0.82±0.02 was obtained (see FIG. 19A). This reveals that in the intermediate state, only 82% of tryptophan fluorescence emission is accessible to quenching by iodide, which is indicative of the partially folded structure of the intermediate (i.e., having buried trytophan residues). Conversely, the fₐ value obtained from Lehrer's plot at 7.5M urea in 10 mM phosphate is 1.07±0.02, which indicates that all tryptophan fluorescence emission is accessible to quenching by iodide and hence exposed to solvent, which is indicative of the complete unfolding of the protein molecule (see FIG. 19B).

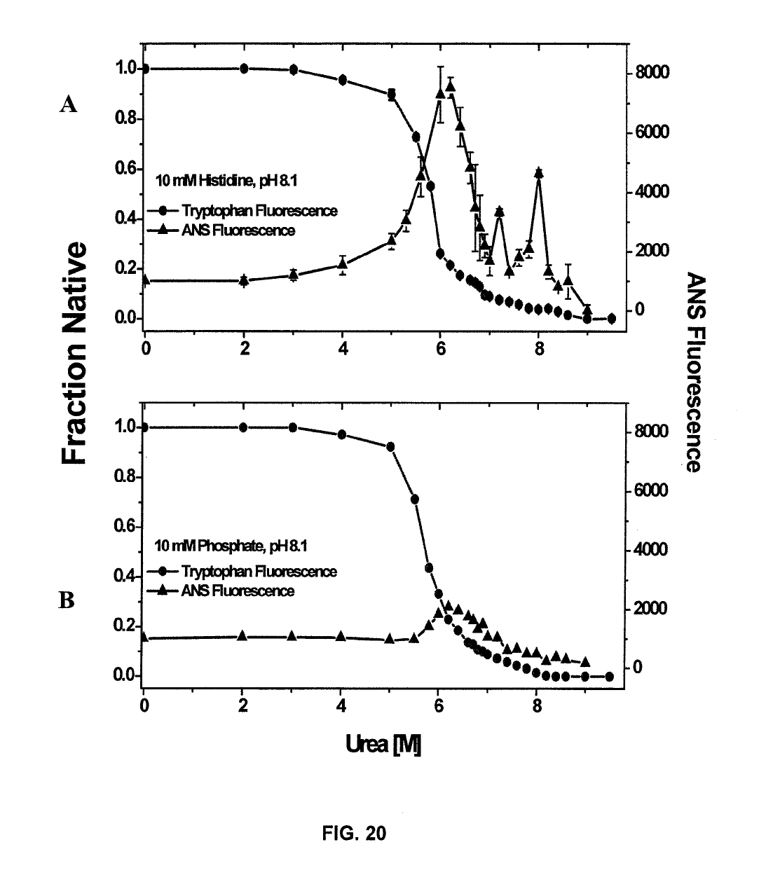

ANS binding studies show that at pH 8.1 (pI), even in the absence of salt, the unfolding transition in the case of histidine was a multi-step process through the formation of stable intermediate species (see FIG. 20A). However, under similar conditions in either tris or phosphate buffers at this pH, no significant population of intermediates was observed (see FIG. 20B). Furthermore, these studies show that there is increased binding of ANS in samples with significant intermediate species, indicating exposed hydrophobic regions.

7.3. Phase Behaviors of Antibodies: Effect of Histidine on Stability

This example shows that certain mAbs may exhibit different phase behaviors. These studies further demonstrate that certain buffer types, such as histidine buffers, under certain conditions, interact with 7F3com-2H2, thereby disrupting domain-domain interactions leading to intermolecular attraction and associations. This results in reduced solubility of 7F3com-2H2 during processing with histidine. 7.3.1. Materials and Methods

### Static Light Scattering

Static light scattering was used to determine second virial coefficients for promising precipitant conditions. An Electro-Optics laser model 1145AP (Hsintien City, Taiwan), a Brookhaven Instruments goniometer and cascade photodiode detector model BI-200SM and BI-APD (Holtsville, N.Y.) respectively, were used to determine the excess

Raleigh ratios at a 90° angle (scattering due to protein only) to the incident 633 nm light beam. In order to check for Raleigh scattering the intensity is also measured at 60° and 120° angles. If the particles being observed are Raleigh scatters (<λ/20) the intensity will not depend on the angle. The relationship used to determine the second virial coefficient is given here and is derived from the virial expansion of the ideal osmotic pressure equation.

$$\frac{Kc}{{R}_{90}} = \frac{1}{M} + 2 {B}_{22} c$$

where,
c=protein concentration (g/mL)
M=protein molecular weight, mass averaged (g/mol)
R₉₀=excess Raleigh Ratio at 90°

The optical constant K, is described by equation 2.

$$K = \frac{4 {π}^{2} {{n}_{0}^{2} ( dn / dc )}^{2}}{{N}_{A} {λ}^{4}}$$

where,
n₀=refractive index of the solvent
dn/dc=refractive index increment for the protein-solvent pair
N_{A}=Avogadro's number
λ=incident beam wavelength (in vacuum)

The Raleigh ratio is given by equation 3.

$$R = \frac{{I}_{θ} {r}^{2}}{{I}_{inc} {V}_{obs}} = {I}_{θ} c$$

where,
r=the distance from the observed volume to the detector
I_{inc}=the incident intensity of the laser beam
I_{θ}=the measured intensity of the scattered light
V_{obs}=the observed volume

The constant c can be determined from a system for which the Raleigh ratio is known, in this case toluene at 633 nm (14*10⁻⁶ cm⁻¹). Once the constant c is determined then raw intensity measurements can be converted to Raleigh ratios and the excess Raleigh ratio is simply the Raleigh ratio of the sample minus that of the pure solvent.

A plot of Kc/R₉₀ versus concentration will yield a slope equal to twice the second virial coefficient (SVC or B₂₂). The concentrations were measured using the absorbance at 280 nm using a sample of each solution for which light scattering was measured. All solutions were filtered using a 0.1 μm Anotop filter (Whatman Inc., England) to remove dust and protein concentrations were varied from 0.5 to 10 mg/mL as the SVC is a dilute solution parameter. All salts and buffers used for light scattering were HPLC grade from SigmaAldrich or Fischer. A positive SVC indicates a net pair-wise repulsion between protein molecules while a negative SVC indicates a net attraction between protein pairs. This pair-wise, or dilute solution parameter, has proven to be an effective aid for predictive crystallization and solubility estimates even for proteins with high solubilities.

### Fourier-Transform Infrared Spectroscopy (FTIR)

Fourier-Transform Infrared (FTIR) spectra were measured using a

BomemTM IR spectrometer (Quebec, Canada) and a dTGS detector. A fixed path length CaF₂ cell with a 7.5 μm Mylar spacer and variable path-length CaF₂ cell were used for liquid and solid measurements respectively. 128-scan interferogram was collected in single beam mode with a 4 cm⁻¹ resolution. A background water vapor scan was subtracted from both the solvent and protein samples. Then the solvent spectra were subtracted from each protein spectra and a 7-point Savitsky-Golay smoothing function was fit to allow for the second-derivative spectra to be obtained. The spectra are then baseline corrected so that the baseline is established at zero. The area of the curve is then integrated and normalized so that the area in amide I region (1705-1600 cm⁻¹) is equal to unity for all spectra. This allows for direct comparison of samples at different concentrations, such as solid and liquid. All subtraction and curve fitting in addition to baseline correction and area normalization was conducted using GRAMS™ software (Thermo Electron Corp., Waltham, Mass.). Protein concentrations between 10 and 12 mg/mL were used for all liquid IR scans.

The amide I region gives rise to peaks in the ˜1700-1600 cm⁻¹ from carbon-oxygen stretching and nitrogen-hydrogen stretching due to hydrogen bonding along the peptide backbone of protein molecules and thus gives a measure of secondary structure. General peak locations for particular types of protein secondary structures have been assigned empirically from proteins of known structure and thus, FTIR is primarily a qualitative technique.

7.3.2. Results

Mass averaged molecular weight (MW) determinations from SLS measurements show that 7F3com-2H2 has a lower extent of association with 10 mM histidine and 150 mM NaCl or in 10 mM phosphate at the pI (see Table 8 infra) than in histidine buffer alone.

TABLE 8
Mass Averaged Molecular Weight (MW) Determinations From Static Light
Scattering (SLS) Measurements.

| mAb sample (pI = 8.1) | B₂₂ | Up 95% | Low 95% | MW | Up 95% | Low 95% |
|---|---|---|---|---|---|---|
| 10 mM His, 150 mM NaCl, pH 6 | 3.5E−05 | 8.8E−05 | −1.8E−05 | 153 | 161 | 145 |
| (stock) | | | | | | |
| BX to 10 mM His pH 6 | −1.0E−04 | −5.2E−05 | −1.5E−04 | 127 | 131 | 122 |
| BX to 10 mM His pH 8.1 | −5.5E−05 | −2.5E−05 | −9.2E−05 | 282 | 299 | 262 |
| BX to 10 mM His, 150 mM NaCl, | 2.0E−05 | 6.3E−05 | −3.7E−05 | 192 | 208 | 175 |
| pH 8.1 | | | | | | |
| BX to 10 mM Tris, pH 8.1 | −8.9E−05 | −4.9E−05 | −1.3E−04 | 147 | 143 | 152 |
| BX to 10 mM Sodium Phosphate, pH | −1.5E−04 | −1.4E−04 | −1.7E−04 | 125 | 126 | 124 |
| 8.1 | | | | | | |

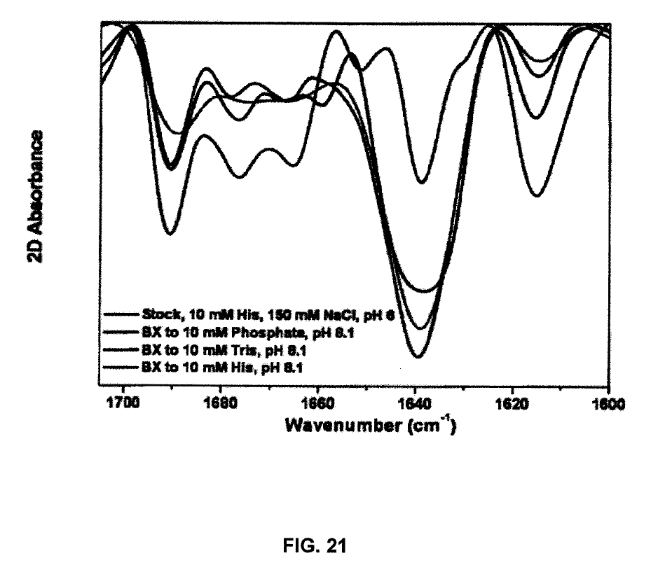

FTIR results indicate that pH alone is not responsible for 7F3com-2H2 associations in solution (pI=8.1) observed by SLS (see FIG. 21). Rather, whether 7F3com-2H2 associates or not is strongly dependent on the buffer type. Specifically, samples containing tris and phosphate buffers exhibit proportionally greater levels of monomeric protein than aggregated protein. Conversely, samples containing histidine buffer exhibit proportionally greater levels of protein in an aggregated state than in a monomeric state

6.4 Effect of Histidine on AB Domanin Stability and Domain-Domain Interactions

In this example, the effect of different buffer species on the domain stability and domain-domain interactions of antibody 7F3com-2H2 were examined using intrinsic tryptophan fluorescence spectroscopy and differential scanning calorimetry.

6.4.1 Materials and Methods

### Generation of F_{c} and F_{ab} Fragments

F_{c} and F_{ab} fragments were generated using papain digestion and were purified using chromatography.

### Urea Unfolding Studies

Unfolding of 7F3com-2H2 was measured by intrinsic tryptophan fluorescence. All experiments were carried out at 23° C. with a protein concentration of 0.67 μM.

### Differential Scanning Calorimetry

A protein concentration of 6.7 μM was used for DSC experiments; all the measurements were performed at a scan rate of 1.5° C./minute.

6.4.2 Results

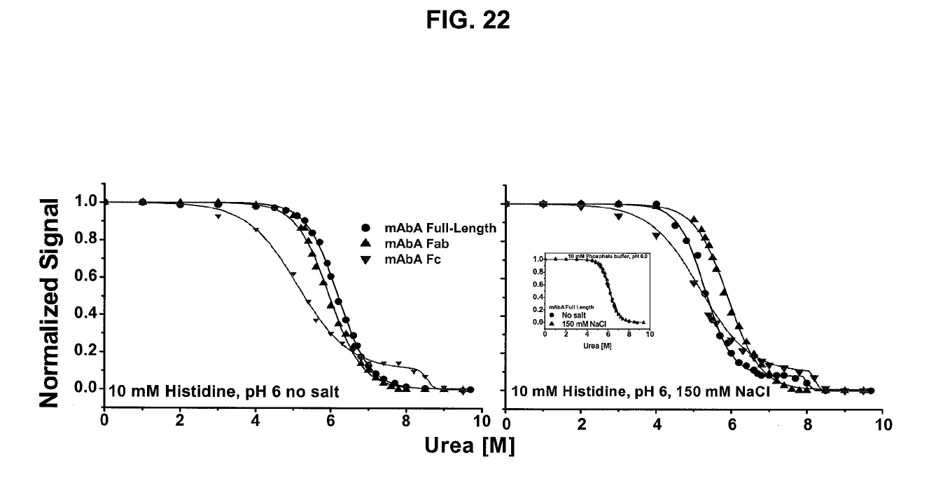

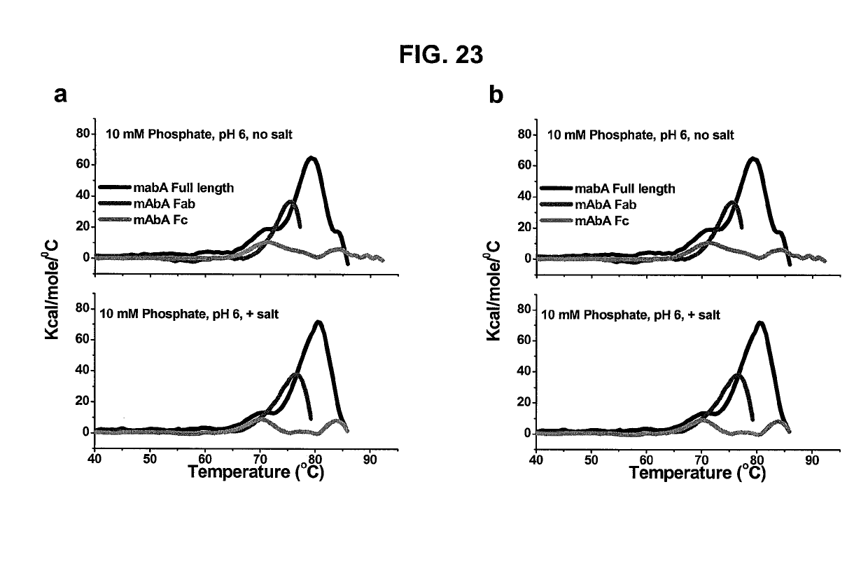

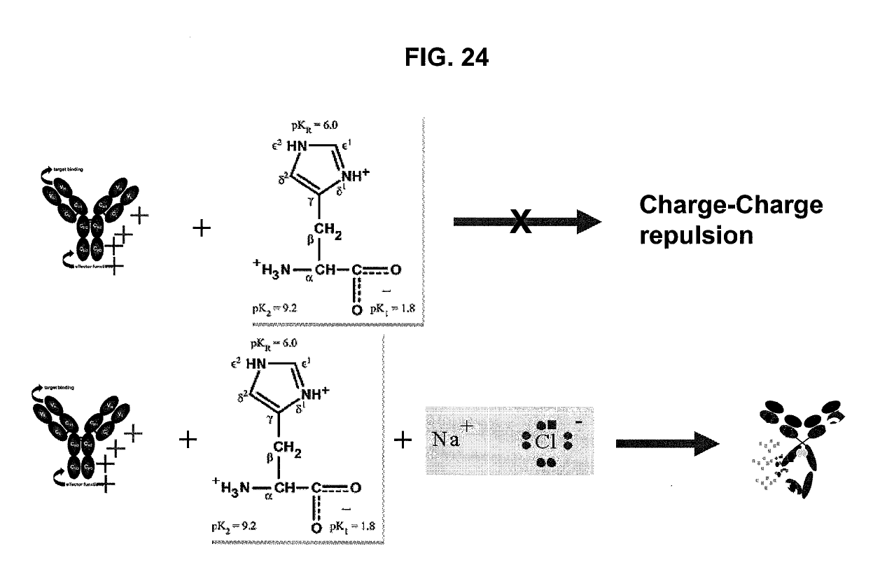

The results from the full length 7Fcom3-2H2 Ab and isolated F_{ab} and F_{c} fragments of the Ab indicated that at pH 6, the interaction of histidine with mAb appeared to require a charge shielding effect by sodium chloride. Urea unfolding studies indicated that interaction resulted in destabilization of some of the domains or the domain-domain interactions in the full-length molecule. See FIG. 22. Further, DSC showed that the CH₂ domain of the F_{c} fragments was the least stable domain and that upon interaction with histidine the Tₘ of the CH₂ domain was decreased by 5° C. from a control value of 70° C. See FIG. 23.

Overall, the fluorescence (FIG. 22) and DSC (FIG. 23) data indicated: strong domain-domain interactions in the full-length 7Fcom3-2H2 at pH 6; NaCl modulated histidine interaction with F_{ab} domains; the unfolding behavior of 7Fcom3-2H2 in presence of histidine and salt was indicative of reduced domain-domain interaction and preferential affect on the stability of some of the domains; the CH₂ domain of the F_{c} fragments was the least stable domain; the interaction between CH₂ and CH₃ domain in F_{c} fragment was weak; there were strong sub-domain interactions in the F_{ab}; and overall, histidine in the presence of salt appreared to effect both stability and interactions of the CH₂ domain.

6.5 Protein-Protein Interactions, Viscosity and Opalescence at a High AB Concentration

This example describes the use of viscometry, membrane osmometry, and light scattering to measure colloidal properties of 7Fcom3-2H2 in the 1-100 g/L concentration range.

6.5.1 Materials and Methods

### Antibody Preparation

The 7Fcom3-2H2 antibody was in a liquid formulation. All buffer conditions were at pH 6 and were achieved by exhaustive dialysis and the protein concentration was determined by UV absorption using an extinction coefficient of 1.61 L/g-cm at 280 nm.

### Viscometry

A Brookfield model LVDV-II+Pro cone/plate viscometer (Middleboro, MA) with external pc control was used with spindle model CPE 40 to measure the viscosity of the mAb solutions. A constant shear rate was applied and the reading was taken after the system stabilized (˜30 seconds). An external water bath was employed to maintain a constant temperature of 23.0±0.1° C.

### Turbidity

Turbidity measurements were carried out on a spectrofluorometer (SLM AMINCO, Urbana, IL, US) equipped with a temperature controlled cell holder at 23.0±0.1° C. The incident beam was set at 510 nm while the scattered light intensity at 90° was measured at the same wavelength. A set of AMCO Clear turbidity standards in 0-100 Nephelometric Turbidity Unit (NTU) range from GFS Chemicals, Inc. (Columbus, Ohio catalog #8255 and 8256) were used to generate a calibration curve that allowed conversion of intensity to NTU for the protein solutions.

### Osmotic Pressure

Osmotic pressure measurements were carried out on a Wescor model 4420

Colloid Osmometer (Logan, Utah) with a 30,000 molecular weight cut-off PM series membrane (part# SS-030) to determine the second virial coefficients and number average molecular weights of the mAb solutions. The corresponding final dialysate was used as the reference solution. BSA standard solutions also form Wescor (part# SS-025) of known osmotic pressure were used to calibrate the osmometer/membrane system.

6.5.2 Results

The results indicated that the solution viscosity and opalescent properties of the high concentration solutions were inversely affected by ionic strength. The combined results suggested that the charge-charge repulsion at low ionic strength was responsible for the high viscosity. The negative second virial coefficients observed at higher ionic strength were associated with large apparent molecular weights at high concentrations (>20 g/L) and an increase in light scattering. This increased light scattering resulted in an opalescent appearance. In addition, the second virial coefficients also suggested that the opalescent appearance may be related to a phase transition.

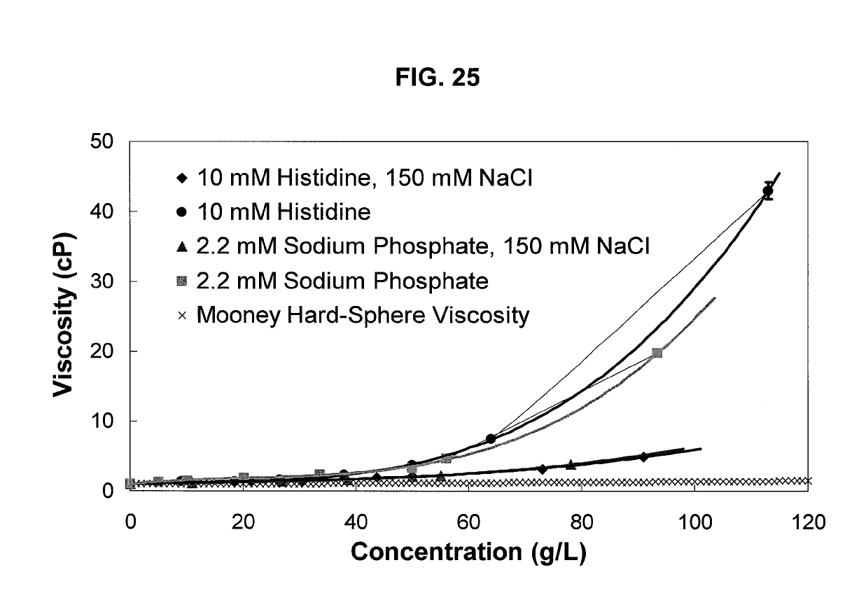

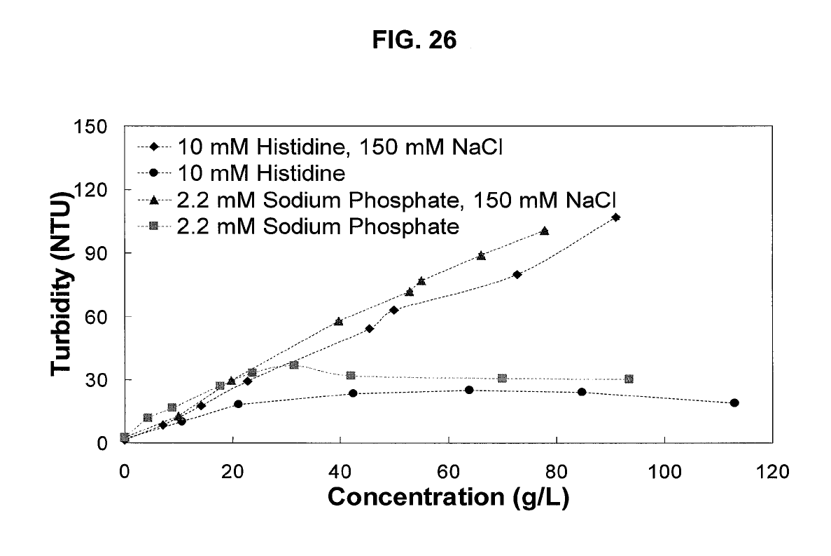

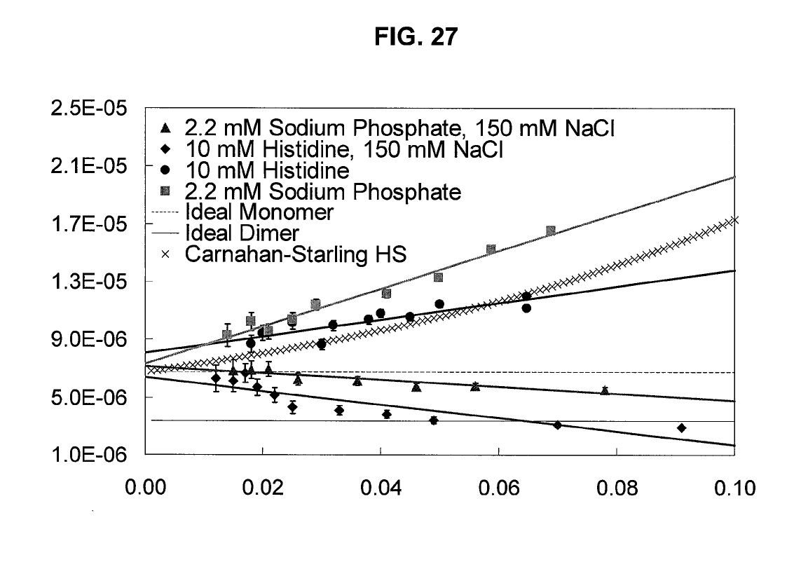

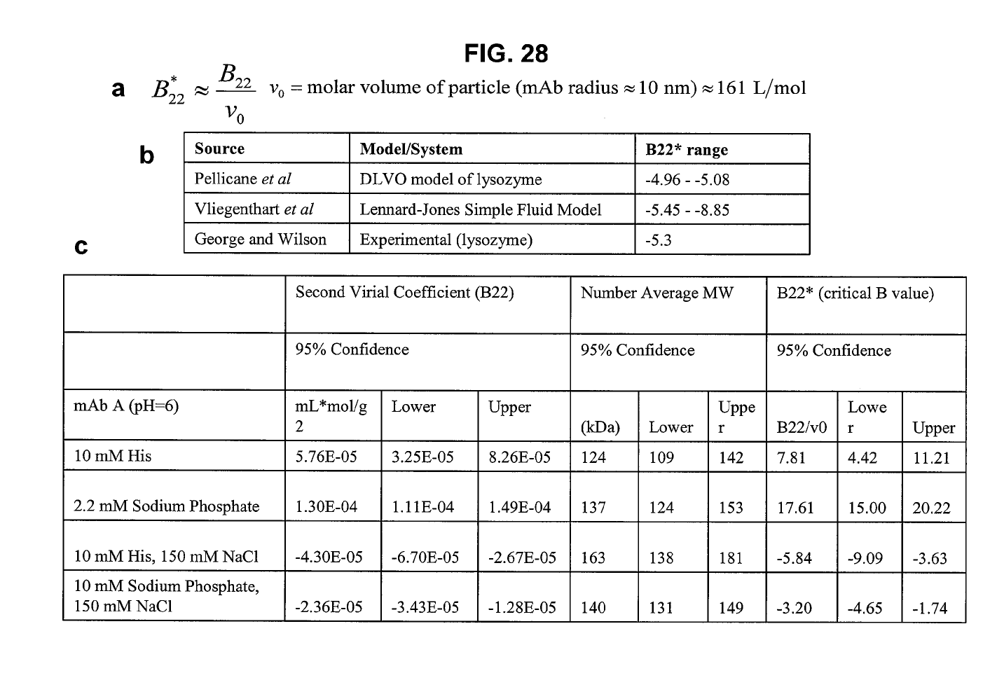

Overall, the viscosity (FIG. 25), turbidity (FIG. 26), and osmotic pressure (FIGS. 27 and 28) showed that charge-charge repulsion was the source of the high solution viscosities at low ionic strength; Large apparent molecular weights, which in this case were associated with negative second virial coefficients, resulted in increased opalescence; Positive second virial coefficients resulted in smaller apparent molecular weights and thus had a less opalescent appearance; Opalescence and viscosity were inversely modified by ionic strength; The observations suggested that balancing viscosity and opalescence could be achieved via intermediate salt concentrations; The solutions with high opalescence had second virial coefficients near the critical values for phase separation determined by Pellicane et al. suggesting that increased opalescence may be associated with phase transitions

6.6 Formulation Development

Formulation studies will be performed to evaluate the chemical and physical stability and solubility of the protein. The goal is to determine the most suitable conditions for long-term storage of the product. Studies will be divided into three main areas: preformulation, concentration, and stability. Preformulation screening studies will be used to evaluate the effects of pH, buffer concentrations, and other excipients on the chemical and physical stability of the protein. These studies will place the protein in a range of buffer, other excipient, and pH conditions at a range of storage temperatures (usually 2-8° C., 23-27° C., and 38-42° C.) and assess chemical and physical stability using assays such as High Pressure Size Exclusion Chromatography (HPSEC) and High-Performance Ion Exchange Chromatography (HPIEC). Concentration studies will provide information on native protein-protein interactions during a concentration step, and information on the impact of high concentration on long-term storage. Further solution pH studies will be performed to examine buffers over a physiologically useful pH range in order to achieve optimal solubility of the protein at high concentration.

Stability of four antibody formulations comprising 5g/L of the 7F3com-2H2 anti-IL-9 antibody was tested under conditions that accelerate antibody degradation. The four formulations enumerated below were prepared using standard laboratory protocols:
1) 10 mM histidine, 150 mM NaCl, pH6.0
2) 2.2 mM Sodium Phosphate, 150 mM NaCl, pH6.0
3) 10 mM histidine, pH 6.0
4) 2.2 mM Sodium Phosphate, pH 6.0

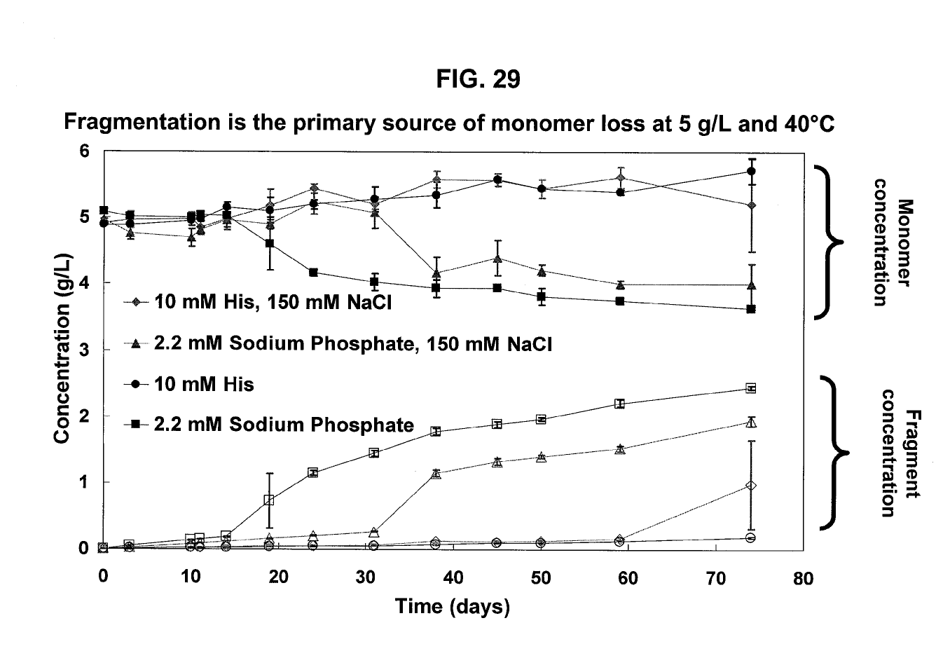

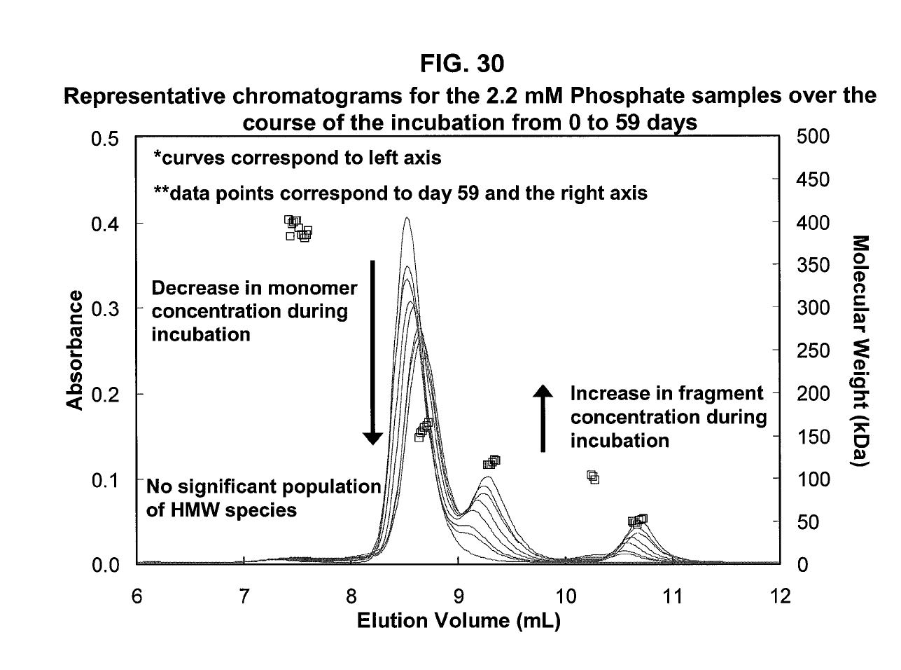

Small aliquots of each formulation were hermetically sealed in glass vials. Vials were stored at 40° C. in an upright position. Individual vials were analyzed by HPSEC at various time points. FIG. 30 shows the superimposed elution profiles of the 2.2 mM Sodium Phosphate, pH 6.0 formulation recorded at different time points. The last data point was obtained after 75 days of incubation. FIG. 29 provides an analysis of total monomer concentration and antibody fragment concentration over time for each of the four formulations shown above.

### 8. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference. The disclosure of U.S. Provisional Application Nos. 60/847,239 filed Sep. 25, 2006, and 60/949,999, filed Jul. 16, 2007 are incorporated by reference herein in their entirety for all purposes.

Citation or discussion of a reference herein shall not be construed as an admission that such is prior art to the present invention.

1. (canceled)

2. An antibody formulation formulated for administration to a human subject, said formulation comprising an aqueous carrier, phosphate, and 10 mg/ml or higher of an antibody or antibody fragment, wherein said antibody or antibody fragment displays a reduction in one or more of the following phase behaviors when formulated in a phosphate buffer at a pH below the pI of said antibody in the presence of salt, as compared to said antibody when formulated in a histidine buffer at said pH in the presence of salt at the same concentration:
(a) formation of unfolded intermediates;
(b) colloidal instability;
(c) soluble association of the antibody molecules; or
(d) precipitation of the antibody molecules;
wherein said at least one or more phase behaviors are measured by techniques selected from the group consisting of high performance size exclusion chromatography (HPSEC), tangential flow filtration (TFF), static light scattering (SLS), Fourier Transform Infrared Spectroscopy (FTIR), circular dichroism (CD), urea-induced protein unfolding techniques, intrinsic tryptophan fluorescence, differential scanning calorimetry (DSC), and 1-anilino-8-naphthalenesulfonic acid (ANS) protein binding techniques.

3. The formulation of claim 2, wherein said antibody or antibody fragment immunospecifically binds to IL-9 polypeptide.

4. The formulation of claim 2, wherein the aqueous carrier is distilled water.

5. The formulation of claim 2, wherein the formulation has a pH in the range of between 4.0 and 8.0.

6. The formulation of claim 5, wherein the pH is in the range of about 6.0 and 6.5.

7. The formulation of claim 2, further comprising salt at a concentration of no more than about 200 mM.

8-9. (canceled)

10. The formulation of claim 2, further comprising a sugar.

11. (canceled)

12. The formulation of claim 10, wherein the sugar is sucrose or trehalose.

13-19. (canceled)

20. The formulation of claim 2, further comprising a surfactant.

21-22. (canceled)

23. The formulation of claim 20, wherein the surfactant is Tween-20 or Tween-80.

24. (canceled)

25. The formulation of claim 23, wherein the surfactant is at a concentration of up to 0.1%.

26. (canceled)

27. The formulation of claim 2, wherein the antibody or antibody fragment is at a concentration of at least 100 mg/ml.

28. (canceled)

29. The formulation of claim 2, wherein phosphate is at a concentration in the range from about 10 mM to about 100 mM.

30-33. (canceled)

34. The formulation of claim 2, wherein less than 5% of the antibody or antibody fragment forms an aggregate during the storage as measured by HPSEC.

35-45. (canceled)

46. The formulation of claim 2, wherein the antibody or the fragment thereof retains at least 85% of binding ability compared to the reference antibody.

47-54. (canceled)

55. The formulation of claim 2, wherein the antibody or antibody fragment is 4D4, 4D4H2-1 D11, 4D4com-XF-9, 4D4com-2F9, 7F3, 71A10, 7F3 22D3, 7F3com-2H2, 7F3com-3H5, or 7F3com-3D4 or an antigen binding fragment thereof.

56-57. (canceled)

58. The formulation of claim 55, wherein the antibody or antibody fragment is 7F3com-2H2.

59. A pharmaceutical unit dosage form suitable for parenteral administration to a human which comprises an antibody formulation of claim 2 in a suitable container.

60. The pharmaceutical unit dosage form of claim 59, wherein the antibody formulation is for intravenous, subcutaneous, or intramuscular injection.

61. A pharmaceutical unit dosage form suitable for aerosol administration to a human which comprises an antibody formulation of claim 2 in a suitable container.

62. The pharmaceutical unit dosage of claim 61, wherein the antibody formulation is administered intranasally.

63-75. (canceled)

76. A method of preventing, managing, treating or ameliorating an inflammatory disease, an autoimmune disease, a disorder associated with aberrant expression and/or activity of an IL-9 polypeptide, a disease or disorder associated with or characterized by aberrant expression and/or activity of an IL-9 polypeptide, a disease or disorder associated with or characterized by aberrant expression and/or activity of the IL-9R or one or more subunits thereof, an autoimmune disease, an inflammatory disease, a proliferative disease, or an infection (preferably, a respiratory infection), or one or more symptoms thereof, said method comprising administering to a subject in need thereof a prophylactically or therapeutically effective amount of an antibody formulation of claim 58.

77-78. (canceled)

79. The method of claim 76, wherein the antibody or antibody fragment thereof polypeptide is stable during storage at 40° C. for at least 15 days as determined by HPSEC.

80-90. (canceled)

91. The method of claim 76, wherein the formulation is administered subcutaneously, orally or intranasally.

92-96. (canceled)

97. An antibody formulation for administration to a subject, said formulation comprising an aqueous carrier, phosphate, and 50 mg/ml or higher of 7F3com-2H2 or an antigen-binding fragment thereof.

98. The formulation of claim 97, wherein the formulation is sterile.

99-133. (canceled)
